(19) United States
(12) Patent Application Publication
(10) Pub. No.: US 2022/0372040 A1
(43) Pub. Date: Nov. 24, 2022

(54) METTL3 MODULATORS

(71) Applicant: Accent Therapeutics, Inc., Lexington, MA (US)

(72) Inventors: Thomas Andrew Wynn, Lexington, MA (US); Brian Lewis Hodous, Cambridge, MA (US); Paula Ann Boriack-Sjodin, Lexington, MA (US); Ernest Allen Sickmier, Needham, MA (US); James Edward John Mills, Sheffield (GB); Andrew Stewart Tasker, Simi Valley, CA (US); Robert A. Copeland, Lexington, MA (US)

(73) Assignees: Accent Therapeutics, Inc., Lexington, MA (US); Accent Therapeutics, Inc., Lexington, MA (US)

(21) Appl. No.: 17/770,714

(22) PCT Filed: Oct. 22, 2020
(86) PCT No.: PCT/US2020/056873

§ 371 Date: Apr. 21, 2022

(51) Int. Cl. C07D 487/04; C07D 519/00; A61P 35/00; A61P 35/02

(52) U.S. Cl. CPC C07D 487/04; C07D 519/00; A61P 35/00; A61P 35/02

(57) ABSTRACT

Provided are compounds of Formula (I′) or (I) below, or pharmaceutically acceptable salts thereof, and methods for their use and production.

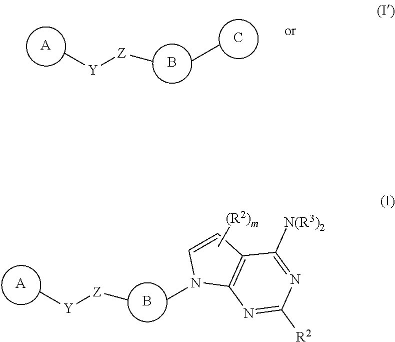

### RELATED APPLICATION

This application claims the benefit of the filing date, under 35 U.S.C. § 119(e), of U.S. Provisional Application No. 62/926,072, filed on Oct. 25, 2019, the entire contents of which are incorporated herein by reference.

### TECHNICAL FIELD

This invention relates to compounds that are METTL3 modulating agents, and methods of making and using such compounds.

### BACKGROUND

Among all RNA modifications, N⁶-methyladenosine (m⁶ A) is the most abundant mRNA internal modification. It plays important roles in the biogenesis and functions of RNA. m⁶ A deposition on mRNA is regulated by the dynamic interplay between RNA specific methylase (“writers”), binding proteins (“readers”), and demethylases (“erasers”) (Ying Yang, Cell Research volume 28, pages 616-624, 2018). m⁶ A methylation is controlled by a large RNA methyltransferase complex (MTase), composed of the methyltransferase-like 3 and 14 (METTL3 and METTL14) proteins and their cofactor, Wilms' tumor 1-associated protein (WTAP). METTL3 is the catalytic component that forms a heterodimer with METTL14, which facilitates the interactions with its target mRNA.

METTL3 has been demonstrated to modulate embryonic development, cell reprogramming, spermatogenesis, regulation of T cell homeostasis and endothelial-to-hematopoietic transition via methylation of specific target transcripts. Aberrant METTL3 expression has been associated with various pathophysiology, such as cancer, obesity, infection, inflammation and immune response (Sibbritt et al., 2013). AML is one of the cancers with the highest expression of both METTL3 and METTL14. Both genes were found upregulated in all subtypes of AML compared to normal hematopoietic cells.

Despite recent advances in METTL3 research, there is still a great need for small molecule METTL3 inhibitors as potential therapeutic agent for treating diseases that are responsive to modulation of METTL3 activities.

### SUMMARY

In accordance with the purpose(s) of the invention, as embodied and broadly described herein, the invention, in an aspect, relates to compounds useful as METTL3 modulators, pharmaceutical compositions, methods of making and methods of treating disorders using the same. In some embodiments, the compounds of the invention are METTL3 inhibitors.

In one aspect, the present invention provides a compound of formula (I′):

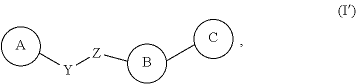

or a pharmaceutically acceptable salt thereof, wherein:
Y is selected from O, S, C(R^{1a})₂, NR^{1b} S(═O), S(═O)₂, NR^{1b}C(═O)NR^{1b}, NR^{1b}C(═O)O with O linked to Z, and C(R^{1b})₂C(═O)NR^{1b} with NR^{1b} linked to Z;
R^{1a}, for each occurrence, is independently selected from H, C₁₋₆alkyl and halo;
Z is selected from O, S, NR^{1b}, C₁₋₆alkyl, C₂₋₆alkenyl and C₂₋₆ alkynyl, each of which is optionally substituted with 1 to 3 halo, provided when Z is O, S or NR^{1b}, then Y is C(R^{1a})₂;
R^{1b}, for each occurrence, is independently H or C₁₋₆alkyl;
Ring A is selected from benzene, naphthalene, benzene fused with 5 to 6-membered heterocycloalkyl, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
Ring C is selected from

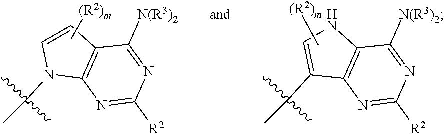

R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —S(═O)R^{2a}, —S(═O)₂R^{2a}, —C(═O)R^{2a}1, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl optionally substituted with —OR^{2a} or —N(R^{2a})₂, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, —N(R^{2a})₂;
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b}, —N(R^{2b})₂, and —S(═O)₂R^{2b};
R^{2b}, for each occurrence, is independently selected from H and C₁₋₆alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, oxo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₁₋₆hydroxyalkyl, C₃₋₈cycloalkyl, phenyl, 5- to 6-membered heteroaryl, oxo, halo, —CN, —OR^{5a}, —N(R^{5a})₂, —C(O)N(R^{5a})₂, —N(R^{5a})C(═O)R^{5a}, —C(O)R^{5a}, and —C(O)OR^{5a};
R^{5a}, for each occurrence, is independently selected from H, C₁₋₆alkyl, C₃₋₈cycloalkyl, 4 to 7-membered heterocycloalkyl, phenyl and 5 to 6-membered heteroaryl, wherein the C₁₋₆alkyl, C₃₋₈cycloalkyl, 4 to 7-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, —S(═O)₂R^{5b}, C₁₋₆haloalkyl, C₃₋₆cycloalkyl, phenyl, and 4 to 7-membered heterocycloalkyl;
or two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₄hydroxyalkyl, —OR^{5b}, —N(R^{5b})₂, and —NR^{5b}C(═O)R^{5b};
R^{5b}, for each occurrence, is independently H or C₁₋₆alkyl optionally substituted with phenyl; and
m is 1 or 2.

The present invention also provides a pharmaceutical composition comprising at least one compound described herein, or a pharmaceutically acceptable salt thereof, and at least one pharmaceutically acceptable excipient.

In one embodiment, the invention is a method of treating a disease or disorder responsive to inhibition of METTL3 activity in a subject comprising administering to said subject an effective amount of at least one compound described herein, or a pharmaceutically acceptable salt thereof.

The present invention also includes the use of at least one compound described herein, or a pharmaceutically acceptable salt thereof, for the manufacture of a medicament for the treatment of a disease or disorder responsive to inhibition of METTL3 activity. Also provided is a compound described herein, or a pharmaceutically acceptable salt thereof for use in treating a disease or disorder responsive to inhibition of METTL3 activity.

Other features or advantages will be apparent from the following detailed description of several embodiments, and also from the appended claims.

### DETAILED DESCRIPTION

It has been found that the compounds of the present invention are useful as METTL3 inhibitors. The compounds according to the invention and compositions thereof, may be useful for the treatment of autoimmune diseases, cancer, inflammatory diseases, and infectious diseases, such as viral infections.

In a first embodiment of the present invention, the compound is represented by formula (I′), or a pharmaceutically acceptable salt thereof, wherein the definitions for the variables are as defined above.

In a second embodiment of the present invention, the compound is represented by formula (I′), or a pharmaceutically acceptable salt thereof, wherein:
Y is selected from O, S, C(R^{1a})₂; and NR^{1b};
R^{1a}, for each occurrence, is independently selected from H, C₁₋₆alkyl and halo;
Z is selected from O, S, NR^{1b}, C₁₋₆alkyl, C₂₋₆alkenyl and C₂₋₆ alkynyl, each of which is optionally substituted with 1 to 3 halo, provided when Z is O, S or NR^{1b}, then Y is C(R^{1a})₂;
R^{1b} is H or C₁₋₆alkyl;
Ring A is selected from benzene, naphthalene, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
Ring C is selected from

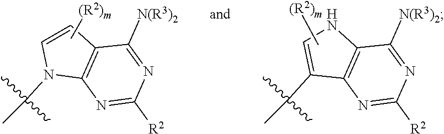

R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂,
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b} and —N(R^{2b})₂;
R^{2b}, for each occurrence, is independently selected from H and C₁₋₃alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{5a}, —N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, C₁₋₆haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl;
R^{5b}, for each occurrence, is H or C₁₋₆alkyl; and
m is 1 or 2.

In a third embodiment of the present invention, the compound is represented by formula (I):

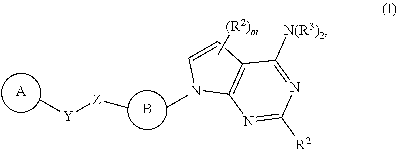

or a pharmaceutically acceptable salt thereof, wherein:
Y is selected from O, S, C(R^{1a})₂; and NR^{b};
R^{1a}, for each occurrence, is independently selected from H, C₁₋₆alkyl and halo;
R^{1b} is H or C₁₋₆alkyl;
Z is selected from C₁₋₆alkyl, C₂₋₆alkenyl and C₂₋₆ alkynyl, each of which is optionally substituted with 1 to 3 halo;
Ring A is selected from benzene, naphthalene, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂,
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b} and —N(R^{2b})₂;
R^{2b}, for each occurrence, is independently selected from H and C₁₋₃alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{5a}, —N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, C₁₋₆haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl;
R^{5b}, for each occurrence, is H or C₁₋₆alkyl; and
m is 1 or 2; and the definitions for the other variables are as defined in the first or second embodiment.

In a fourth embodiment of the present invention, the compound is represented by formula (I′) or (I), or a pharmaceutically acceptable salt thereof, wherein Y is S, S(═O), S(═O)₂, NHC(═O)NH, NHC(═O)O with O linked to Z, or CH₂C(═O)NH with NH linked to Z; and the definitions for the other variables are as defined in the first, second, or third embodiment.

In a fifth embodiment of the present invention, the compound is represented by formula (I′) or (I), or a pharmaceutically acceptable salt thereof, wherein Y is O or C(R^{1a})₂; and R^{1a}, for each occurrence, is independently H or halo; and the definitions for the other variables are as defined in the first, second, or third embodiment.

In a sixth embodiment of the present invention, the compound is represented by formula (I′) or (I), or a pharmaceutically acceptable salt thereof, wherein Y is O or CH₂; and the definitions for the other variables are as defined in the first, second, or third embodiment.

In a seventh embodiment of the present invention, the compound is represented by formula (I′) or (I), or a pharmaceutically acceptable salt thereof, wherein Z is selected from C₁₋₄alkyl and C₂₋₄ alkenyl, each of which is optionally substituted with 1 to 3 halo; and the definitions for the other variables are as defined in the first, second, third, fourth, fifth, or sixth embodiment.

In an eighth embodiment of the present invention, the compound is represented by formula (I′) or (I), or a pharmaceutically acceptable salt thereof, wherein Z is selected from O and NR^{1b}; and Y is C(R^{1a})₂, wherein R^{1a}, for each occurrence, is independently H or halo and R^{1b} is H or C₁₋₆alkyl; and the definitions for the other variables are as defined in the first, second, third, or fifth embodiment.

In a ninth embodiment of the present invention, the compound is represented by formula (I′) or (I), or a pharmaceutically acceptable salt thereof, wherein Z is CH₂; and Y is O or CH₂; and the definitions for the other variables are as defined in the first, second, third, fifth, sixth, or seventh embodiment.

In a tenth embodiment of the present invention, the compound is represented by formula (I′) or (I), or a pharmaceutically acceptable salt thereof, wherein ring C is

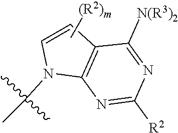

and the definitions for the other variables are as defined in the first, second, third, fourth, fifth, sixth, seventh, eighth, or ninth embodiment.

In an eleventh embodiment of the present invention, the compound is represented by formula (I′) or (I), or a pharmaceutically acceptable salt thereof, wherein ring C is

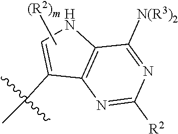

and the definitions for the other variables are as defined in the first, second, third, fourth, fifth, sixth, seventh, eighth, or ninth embodiment.

In a twelfth embodiment of the present invention, the compound is represented by the following formula:

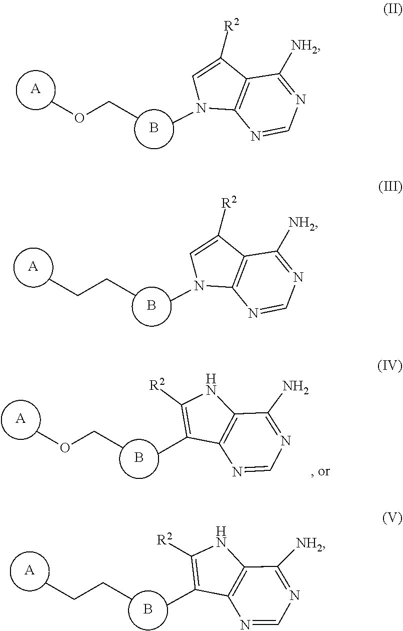

or a pharmaceutically acceptable salt thereof; and the definitions for the variables are as defined in the first, second, or third embodiment.

In a thirteenth embodiment of the present invention, the compound is represented by the following formula:

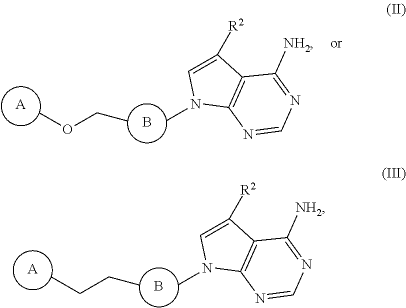

or a pharmaceutically acceptable salt thereof; and the definitions for the variables are as defined in the first, second, or third embodiment.

In a fourteenth embodiment of the present invention, the compound is represented by formula (I′), (I), (II), (III), (IV) or (V), or a pharmaceutically acceptable salt thereof, wherein ring B is selected from benzene, pyridine, pyridazine, pyrimidine, pyrazine, triazine, pyrazole, imidazole, thiazole, oxazole, isoxazole, triazole and tetrazole, each of which is optionally substituted with 1 to 2 independently selected R⁴; and the definitions for the other variables are as defined in the first, second, third, fourth, fifth, sixth, seventh, eighth, ninth, tenth, eleventh, twelfth, or thirteenth embodiment. In certain embodiments, ring C and group Z in formula (I′) or corresponding groups in formula (I), (II), (III), (IV) or (V) are attached to ring B at meta positions.

In a fifteenth embodiment of the present invention, the compound is represented by formula (I′), (I), (II), (III), (IV) or (V), or a pharmaceutically acceptable salt thereof, wherein ring B is selected from benzene, pyridine, pyrazine, pyrimidine, pyrazole, thiazole and thiadiazole, each of which is optionally substituted with 1 to 2 independently selected R⁴; and the definitions for the other variables are as defined in the fourteenth embodiment. In certain embodiments, ring C and group Z in formula (I′) or corresponding groups in formula (I), (II), (III), (IV) or (V) are attached to ring B at meta positions.

In a sixteenth embodiment of the present invention, the compound is represented by formula (I′), (I), (II), (III), (IV) or (V), or a pharmaceutically acceptable salt thereof, wherein ring B is represented by the following formula:

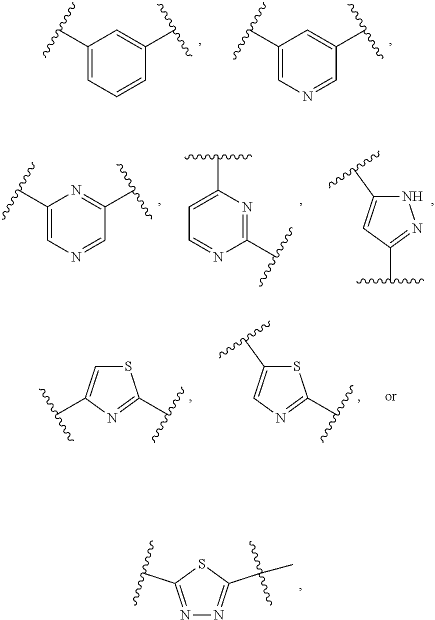

each of which is optionally substituted with 1 to 2 independently selected R⁴, wherein [symbol] indicates the point of connection in which ring B is connected to ring C and the Z group; and the definitions for the other variables are as defined in the fourteenth embodiment.

In a seventeenth embodiment of the present invention, the compound is as defined in the sixteenth embodiment, or a pharmaceutically acceptable salt thereof, wherein ring B is

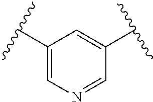 [SMILES: *c1cncc(*)c1]

optionally substituted with 1 to 2 independently selected R⁴.

In an eighteenth embodiment of the present invention, the compound is represented by formula (I′), (I), (II), (III), (IV) or (V), or a pharmaceutically acceptable salt thereof, wherein R⁴ is H, C₁₋₆alkyl, halo, —N(R^{4a})₂; or —C(═O)N(R^{4a})₂, wherein the C₁₋₆alkyl is optionally substituted with 1 to 3 substituents independently selected from halo and —R^{4a}; and R^{4a}, for each occurrence, is independently selected from H and C₁₋₃alkyl; and the definitions for the other variables are as defined in the first, second, third, fourth, fifth, sixth, seventh, eighth, ninth, tenth, eleventh, twelfth, thirteenth, fourteenth, fifteenth, sixteenth, or seventeenth embodiment.

In a nineteenth embodiment of the present invention, the compound is as defined in the eighteenth embodiment, or a pharmaceutically acceptable salt thereof, wherein R⁴ is H.

In a twentieth embodiment of the present invention, the compound is represented by formula (I′), (I), (II), (III), (IV) or (V), or a pharmaceutically acceptable salt thereof, wherein R⁴ is —CH₂OH, —NHCH₃, —C(═O)NH₂, —NH₂, —Br, —F, —OH, and —OCH₃; and the definitions for the other variables are as defined in the first, second, third, fourth, fifth, sixth, seventh, eighth, ninth, tenth, eleventh, twelfth, thirteenth, fourteenth, fifteenth, sixteenth, or seventeenth embodiment.

In a twenty-first embodiment of the present invention, the compound is represented by formula (I′), (I), (II), (III), (IV) or (V), or a pharmaceutically acceptable salt thereof, wherein ring A is a 9- to 10-membered bicyclic heteroaromatic ring optionally substituted with 1 to 4 independently selected R⁵ groups; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-second embodiment of the present invention, the compound is as defined in the twenty-first embodiment, or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵.

In a twenty-third embodiment of the present invention, the compound is represented by formula (I′), (I), (II), (III), (IV) or (V), or a pharmaceutically acceptable salt thereof, wherein ring A is selected from benzene, naphthalene, pyridine, 3,4-dihydro-2H-benzo[b][1,4]oxazine, quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, or twentieth embodiment.

In a twenty-fourth embodiment of the present invention, the compound is represented by formula (I′), (I), (II), (III), (IV) or (V)), or a pharmaceutically acceptable salt thereof, wherein ring A is selected from quinoline, quinozaline, quinoxaline, benzoimidiazole, benzothiazole, napththyridine, indole, pyrrolopyrimidine and indazole, each of which is optionally substituted with 1 to 3 independently selected R⁵; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, or twenty-third embodiment.

In a twenty-fifth embodiment of the present invention, the compound is represented by formula (I′), (I), (II), (III), (IV) or (V), or a pharmaceutically acceptable salt thereof, wherein ring A is quinoline optionally substituted with 1 to 3 independently selected R⁵; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, or twenty-fourth embodiment.

In a twenty-sixth embodiment of the present invention, the compound is represented by formula (I′), (I), (II), (III), (IV) or (V), or a pharmaceutically acceptable salt thereof, wherein ring A is represented by the following formula:

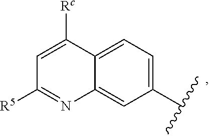

wherein R^{c} is selected from H, halo, C₁₋₆alkyl, —OR^{c1}, —N(R^{c1})₂; —NR^{c1}C(═O)R^{c1}, and 4 to 7-membered heterocycloalkyl, and R^{c1}, for each occurrence, is independently H, C₁₋₆alkyl, or C₃₋₆cycloalkyl, wherein the C₁₋₆alkyl, C₃₋₆cycloalkyl, and 4 to 7-membered heterocycloalkyl are each optionally substituted with 1 to 3 substituents independently selected from halo, C₁₋₄alkyl, C₃₋₆cycloalkyl, 4 to 7-membered heterocycloalkyl, —OR^{c2} and —N(R^{c2})₂; and R^{c2}, for each occurrence, is independently H or C₁₋₆alkyl optionally substituted with phenyl; and the definitions for the other variables are as defined in the first, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, or twenty-fifth embodiment.

In a twenty-seventh embodiment of the present invention, the compound is represented by formula (I′), (I), (II), (III), (IV) or (V), or a pharmaceutically acceptable salt thereof, wherein ring A is represented by the following formula:

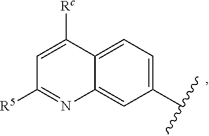

wherein R^{c} is selected from H, halo, C₁₋₄alkyl, —OR^{c1} and —N(R^{c1})₂, and R^{c1}, for each occurrence, is independently H or C₁₋₄alkyl optionally substituted with halo, —OR^{c2} and —N(R^{c2})₂; and R^{c2}, for each occurrence, is independently H or C₁₋₆alkyl; and the definitions for the other variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, or twenty-sixth embodiment.

In a twenty-eighth embodiment of the present invention, the compound is as defined in the twenty-seventh embodiment, or a pharmaceutically acceptable salt thereof, wherein R^{c} is selected from H, —NHCH₂CH₂N(CH₃)₂, and —N(CH₃)CH₂CH₂OCH₃.

In a twenty-ninth embodiment of the present invention, the compound is represented by formula (I′), (I), (II), (III), (IV) or (V), or a pharmaceutically acceptable salt thereof, wherein:

R⁵, for each occurrence, is independently selected from H, C₁₋₆alkyl, C₃₋₆cycloalkyl, 4 to 7-membered heterocycloalkyl, 5 to 6-membered heteroaryl, halo, oxo, —OR^{5a}, —N(R^{5a})₂; —NR^{5a}C(═O)R^{5a}, and —NR^{5a}C(═O)N(R^{5a})₂, wherein the C₁₋₆alkyl, C₃₋₆cycloalkyl, 4 to 7-membered heterocycloalkyl, and 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl, —S(═O)₂R^{5b}, and C₃₋₈cycloalkyl, or
two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₃hydroxyalkyl, —OH, —NH₂, and —NHC(═O)CH₃; and
R^{5b} is H or C₁₋₆alkyl optionally substituted with phenyl; and the definitions for the other variables are as defined in the first, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, or twenty-eighth embodiment.

In a thirtieth embodiment of the present invention, the compound is represented by formula (I′), (I), (II), (III), (IV) or (V), or a pharmaceutically acceptable salt thereof, wherein: R⁵, for each occurrence, is independently selected from H, C₁₋₆alkyl, 4 to 6-membered heterocycloalkyl, 5 to 6-membered heteroaryl, halo, —OR^{5a}, —N(R^{5a})₂; —NR^{5a}C(═O)R^{5a}, and —NR^{5a}C(═O)N(R^{5a})₂, wherein the C₁₋₆alkyl, 4 to 6-membered heterocycloalkyl, 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl and C₃₋₈cycloalkyl, or two R^{5a} together with the N atom from which they are attached form a 4 to 6-membered heterocycloalkyl optionally containing an additional heteroatom selected from O, N and S; and
R^{5b} is H or C₁₋₃alkyl; and the definitions for the other variables are as defined in the first, second, third, fourth, fifth, sixth, seventh, eighth, ninth, tenth, eleventh, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, or twenty-ninth embodiment.

In a thirty-first embodiment of the present invention, the compound is as defined in the thirtieth embodiment, or a pharmaceutically acceptable salt thereof, wherein:
R⁵, for each occurrence, is independently selected from H, C₁₋₆alkyl, —N(R^{5a})₂, and 4 to 6-membered heterocycloalkyl containing 1 or 2 heteroatoms independently selected from O, N and S;
R^{5a}, for each occurrence, is independently H, C₁₋₆alkyl, and C₃₋₆cycloalkyl, wherein the C₁₋₆alkyl and C₃₋₆cycloalkyl are each optionally substituted with one to three substituents independently selected from halo, C₁₋₆alkyl, C₃₋₆cycloalkyl, —OR^{5b} and —N(R^{5b})₂, or two R^{5a} together with the N atom from which they are attached form a 4 to 6-membered heterocycloalkyl optionally containing an additional heteroatom selected from O, N and S; and
R^{5b} is each independently H or C₁₋₃alkyl.

In a thirty-second embodiment of the present invention, the compound is represented by formula (I′), (I), (II), (III), (IV) or (V), or a pharmaceutically acceptable salt thereof, wherein R⁵, for each occurrence, is independently selected from H, —F, —Br, NH₂, —NHCH₃, —N(CH₃)₂, —NHCH₂CH₃, —NHCH(CH₃)₂, —NHCH₂CH₂N(CH₃)₂, —NH(CH₂)₃N(CH₃)₂, —NHCH₂-cyclopropyl, —N(CH₃)CH₂cyclopropyl, —N(CH₃)cyclopropyl, —NHC(═O)CH₃, —N(CH₃)CH₂CH₂SO₂CH₃, —NHCH₂CH₂OCH₃, —N(CH₃)CH₂CH₂OCH₃, —N(CH₃)CH₂CH₂OH, —N(CH₃)CH₂CH₂N(CH₃)₂, —NHCH₂CH₂N(CH₃)(CH₂)₃phenyl, —NHCH₂CH₂N(CH₃)(CH₂)₅phenyl, —CH₃, —CH₂CH₃, —CH₂CH₂CH₃, —CH(CH₃)₂, —C(CH₃)₃, —OCH₃, cyclobutyl, —(CH₂)₃morpholine, azetidine, pyrrolidine, piperazine, piperidine, morpholine, tetrahydro-2H-pyran, 1,3-oxazinane, 5-azaspiro[2.3]hexane, 2-azaspiro[3.3]heptane, and 2-oxa-6-azaspiro[3.3]heptane, wherein the azetidine is optionally substituted with 1 to 3 substituents selected from oxo, —OH, NH₂, —F, —NHC(═O)CH₃, —CH₃, and —CH₂OH; the 1,3-oxazinane and pyrrolidine are each optionally substituted with oxo; the piperazine is optionally substituted with methyl; and the piperidine is optionally substituted with 1 to 3 —F; and the definitions for the other variables are as defined in the first, second, third, fourth, fifth, sixth, seventh, eighth, ninth, tenth, eleventh, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, or twenty-ninth embodiment.

In a thirty-third embodiment of the present invention, the compound is represented by formula (I′), (I), (II), (III), (IV) or (V), or a pharmaceutically acceptable salt thereof, wherein R⁵, for each occurrence, is independently selected from H, —NH₂, —NHCH₃, —NHCH₂CH₃, —NHCH(CH₃)₂, —NHCH₂CH₂N(CH₃)₂, —NHCH₂CH₂OCH₃, —NHCH₂-cyclopropyl, —CH₃, —CH₂CH₃, —CH(CH₃)₂, azetidine, and pyrrolidinyl; and the definitions for the other variables are as defined in the first, second, third, fourth, fifth, sixth, seventh, eighth, ninth, tenth, eleventh, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, or thirty-second embodiment.

In a thirty-fourth embodiment of the present invention, the compound is as defined in the thirty-third embodiment, or a pharmaceutically acceptable salt thereof, wherein R⁵, for each occurrence, is independently selected from —NHCH₃, —NHCH₂CH₃, cyclobutyl, —CH₂CH₃, and azetidine.

In a thirty-fifth embodiment of the present invention, the compound is represented by formula (I′), (I), (II), (III), (IV) or (V), or a pharmaceutically acceptable salt thereof, wherein:
R² is H, halo, —OR^{2a}, —N(R^{2a})₂, —N(R^{2a})—C(═O)—(R^{2a}), C₁₋₆alkyl, C₃₋₈cycloalkyl, C₅₋₈cycloalkenyl, 4 to 6-membered heterocycloalkyl, 4 to 6-membered heterocycloalkenyl, phenyl, 5 to 6-membered heteroaryl, wherein the C₁₋₆alkyl, C₃₋₈cycloalkyl, C₅₋₈cycloalkenyl, 4 to 6-membered heterocycloalkyl, 4 to 6-membered heterocycloalkenyl and 5 to 6-membered heteroaryl are each optionally substituted with 1 to 3 substituents independently selected from halo, N(R^{2a})₂, C₁₋₄alkyl, C₁₋₄haloalkyl and C₃₋₆cycloalkyl;
R^{2a}, for each occurrence, is independently selected from H, C₁₋₆alkyl and C₂₋₆alkenyl, wherein the C₁₋₆alkyl and C₂₋₆alkenyl are each optionally substituted with one to three substituents independently selected from halo, —C(═O)OR^{2b}, —OR^{2b} and —N(R^{2b})₂; and
R^{2b} is H or C₁₋₃alkyl; and the definitions for the other variables are as defined in the first, second, third, fourth, fifth, sixth, seventh, eighth, ninth, tenth, eleventh, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, or thirty-fourth embodiment.

In a thirty-sixth embodiment of the present invention, the compound is as defined in the thirty-fifth embodiment, or a pharmaceutically acceptable salt thereof, wherein R² is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

In a thirty-seventh embodiment of the present invention, the compound is as defined in the thirty-fifth embodiment, or a pharmaceutically acceptable salt thereof, wherein:
R² is H, halo, —N(R^{2a})₂, —N(R^{2a})—C(═O)—(R^{2a}), C₁₋₆alkyl, C₃₋₆cycloalkyl, 4 to 6-membered heterocycloalkyl, and 5 to 6-membered heteroaryl, wherein the C₁₋₆alkyl, C₃₋₆cycloalkyl, 4 to 6-membered heterocycloalkyl, and 5 to 6-membered heteroaryl are each optionally substituted with 1 to 3 substituents independently selected from halo, C₁₋₃alkyl, C₃₋₆cycloalkyl and —N(R^{2a})₂;
R^{2a}, for each occurrence, is independently selected from H, C₁₋₆alkyl and C₂₋₆alkenyl, wherein the C₁₋₆alkyl and C₂₋₆alkenyl are each optionally substituted with one to three substituents independently selected from halo and —C(═O)OR^{2b}; and
R^{2b} is H or C₁₋₃alkyl.

In a thirty-eighth embodiment of the present invention, the compound is represented by formula (I′), (I), (II), (III), (IV) or (V), or a pharmaceutically acceptable salt thereof, wherein R² is selected from H, —F, —Cl, —Br, —CN, —CH₃, —CH₂CH₃, —CH₂OH, —CH₂OCH₃, C(═O)CH₃, SO₂CH₃, —S(═O)CH₃, —N(CH₃)₂, —NHCH₃, —CH₂CH₂N(CH₃)₂, —CH₂-cyclopropyl, cyclopropyl, cyclopentyl, N-difluorocyclohexyl, N-methylpiperidine, —N(C(═O)CH═CH₂)(CH₂CH₂CO₂H), 1-methylpyrazole, 1-(2-methoxyethyl)-1H-pyrazole, tetrahydrofuran, o-methoxyphenyl, 1-(2-methoxyethyl)-1H-pyrazole, 2-methylfuran, furan substituted with —CH₂NHCH₂CH₂SO₂CH₃, oxazole, —CH(CH₃)-cyclopropyl, —NHC(═O)CH₂Cl, and C(═O)CH₂CH₂CH₃; and the definitions for the other variables are as defined in the first, fourth, fifth, sixth, seventh, eighth, ninth, tenth, eleventh, twelfth, thirteenth, fourteen, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, thirty-third, or thirty-fourth embodiment.

In a thirty-ninth embodiment of the present invention, the compound is as defined in the thirty-eighth embodiment, or a pharmaceutically acceptable salt thereof, wherein R² is selected from H, 1-methylpyrazole, and tetrahydrofuran.

In a fortieth embodiment of the present invention, the compound is as defined in the thirty-eighth embodiment, or a pharmaceutically acceptable salt thereof, wherein R² is selected from H, Cl, —Br, —N(CH₃)₂, —NHCH₃, —CH₃, —CH₂CH₃, —CH₂CH₂N(CH₃)₂, —CH₂-cyclopropyl, cyclopropyl, cyclopentyl, N-difluorocyclohexyl, N-methylpiperidine, 1-methylpyrazole and —N(C(═O)CH═CH₂)(CH₂CH₂CO2H).

In a forty-first embodiment of the present invention, the compound is represented by the following formula:

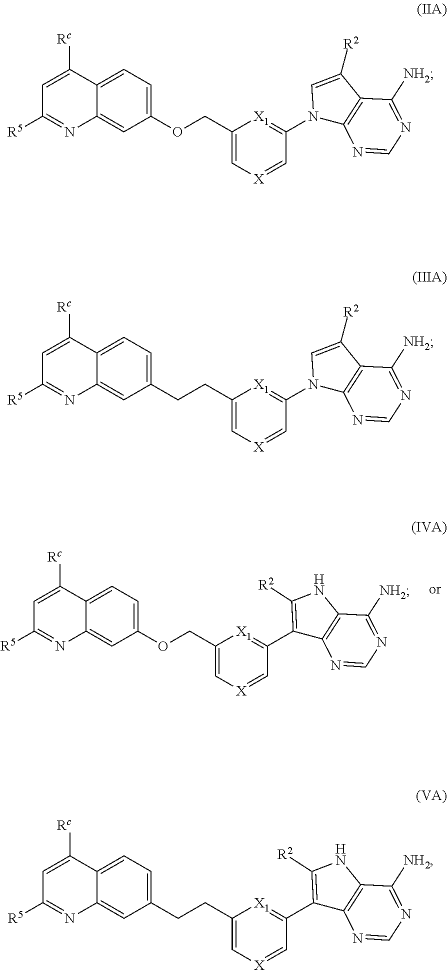

or a pharmaceutically acceptable salt thereof, wherein.
X is N or CH;
X₁ is N or CH;
R⁵, for each occurrence, is independently selected from H, C₁₋₆alkyl, C₃₋₆cycloalkyl, 4 to 7-membered heterocycloalkyl, 5 to 6-membered heteroaryl, halo, —OR^{5a}, —N(R^{5a})₂, —NR^{5a}C(═O)R^{5a}, and —NR^{5a}C(═O)N(R^{5a})₂, wherein the C₁₋₆alkyl, C₃₋₆cycloalkyl, 4 to 7-membered heterocycloalkyl, 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl, —SO₂C₁₋₃alkyl, and C₃₋₈cycloalkyl, or two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₃hydroxyalkyl, —OH, —NH₂, and —NHC(═O)CH₃; and
R^{5b} is H or C₁₋₆alkyl, wherein the C₁₋₆alkyl is optionally substituted with phenyl.
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂; —NR^{c1}C(═O)R^{c1}, —NR^{c1}C(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1};
R^{c1}, for each occurrence, is independently selected from H, C₁₋₆alkyl, C₃₋₈cycloalkyl, and 4 to 7-membered heterocycloalkyl, phenyl and 5 to 6-membered heteroaryl, wherein the C₁₋₆alkyl, C₃₋₈cycloalkyl, 4 to 7-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₃₋₆cycloalkyl, phenyl, 4 to 7-membered heterocycloalkyl, —OR^{c2}, and —N(R^{c2})₂, or two R^{c1} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, C₁₋₄alkyl and C₁₋₄haloalkyl; and
R^{c2}, for each occurrence, is independently H or C₁₋₆alkyl optionally substituted with phenyl; and the definitions for the other variables are as defined in the first embodiment.

In a forty-second embodiment of the present invention, the compound is represented by the following formula:

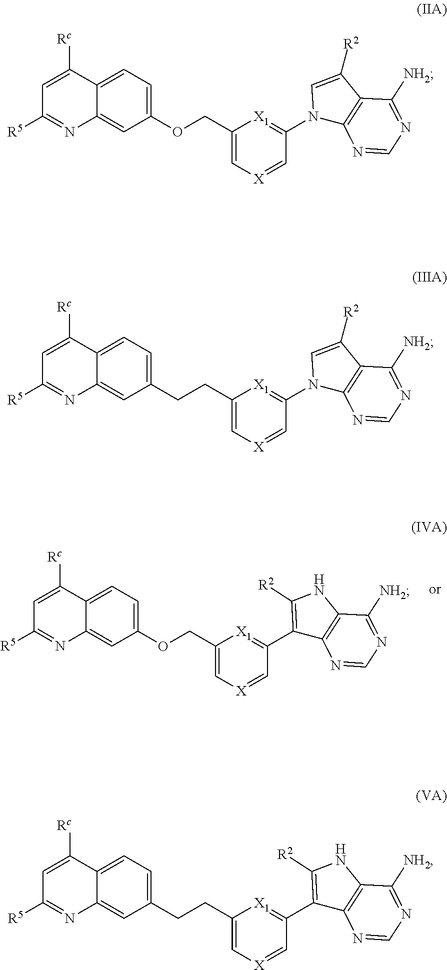

or a pharmaceutically acceptable salt thereof, wherein:
X is N or CH;
X₁ is N or CH;
R² is selected from H, halo, C₁₋₄alkyl, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl; and
R⁵ is —N(R^{5a})₂;
R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂; —NR^{c1}(═O)R^{c1}, —NR^{c1}C(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and
R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl; and the definitions for the other variables are as defined in the first or second embodiment.

In a forty-third embodiment of the present invention, the compound is as defined in the forty-first or forty-second embodiment, or a pharmaceutically acceptable salt thereof, wherein X is CH and X₁ is CH.

In a forty-fourth embodiment of the present invention, the compound is as defined in the forty-first or forty-second embodiment, or a pharmaceutically acceptable salt thereof, wherein X is N and X₁ is CH.

In a forty-fifth embodiment of the present invention, the compound is as defined in the forty-first or forty-second embodiment, or a pharmaceutically acceptable salt thereof, wherein X is N and X₁ is N.

In a forty-sixth embodiment of the present invention, the compound is represented by formula (IIA), (IIIA), (IVA) or (VA), or a pharmaceutically acceptable salt thereof, wherein R^{c} is selected from H, halo, C₁₋₄alkyl, —OR^{c1} and —N(R^{c1})₂, and R^{c1}, for each occurrence, is independently H or C₁₋₄alkyl optionally substituted with halo, —OR^{c2} or —N(R^{c2}); and R^{c2}, for each occurrence, is independently H or C₁₋₄alkyl; and the definitions for the other variables are as defined in the forty-first, forty-second, forty-third, forty-fourth, or forty-fifth embodiment.

In a forty-seventh embodiment of the present invention, the compound is represented by formula (IIA), (IIIA), (IVA) or (VA), or a pharmaceutically acceptable salt thereof, wherein R^{c} is H; and the definitions for the other variables are as defined in the forty-first, forty-second, forty-third, forty-fourth, forty-fifth, or forty-sixth embodiment.

In a forty-eighth embodiment of the present invention, the compound is represented by the following formula:

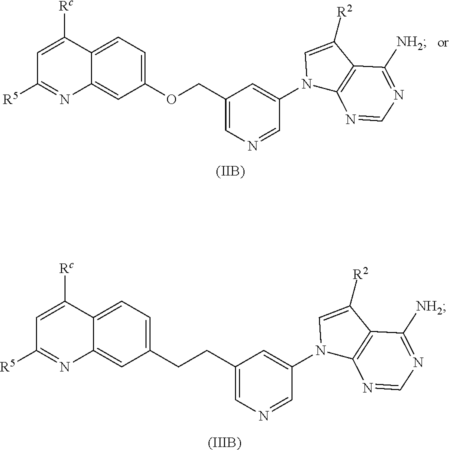

or a pharmaceutically acceptable salt thereof, wherein:
R² is H, 5-membered heteroaryl or 5-membered heterocycloalkyl;
R^{c} is H or —N(R^{c1})₂;
R^{c1} is C₁₋₃alkyl optionally substituted with —OR^{c2} or —N(R^{c2}
R^{c2} is C₁₋₃alkyl;
R⁵ is C₁₋₃alkyl, C₃₋₆cycloalkyl or —N(R^{5a})₂; and
R^{5a}, for each occurrence, is independently H or C₁₋₃alkyl; or two R^{5a} together with the N atom from which they are attached form a 4 to 6-membered heterocycloalkyl; and the definitions for the other variables are as defined in the first or second embodiment.

In a forty-ninth embodiment of the present invention, the compound is represented by formula (IIB) or (IIIB), or a pharmaceutically acceptable salt thereof, wherein R² is H, 1-methylpyrazole, or tetrahydrofuran; and the definitions for the other variables are as defined in the forty-eighth embodiment.

In a fiftieth embodiment of the present invention, the compound is represented by formula (IIB) or (IIIB), or a pharmaceutically acceptable salt thereof, wherein R⁵ is —NHCH₃, —NHCH₂CH₃, cyclobutyl, —CH₂CH₃, or azetidine; and the definitions for the other variables are as defined in the forty-eighth or forty-ninth embodiment.

In a fifty-first embodiment of the present invention, the compound is represented by formula (IIB) or (IIIB), or a pharmaceutically acceptable salt thereof, wherein R^{c} is H, —NHCH₂CH₂N(CH₃)₂, or —N(CH₃)CH₂CH₂OCH₃; and the definitions for the other variables are as defined in the forty-eighth, forty-ninth, fiftieth, or fifty-first embodiment.

In a fifty-second embodiment of the present invention, the compound of the present invention is selected from the compounds of Table 1 or a pharmaceutically acceptable salt thereof.

### Definitions

As used herein, the term “alkyl” refers to a fully saturated branched or unbranched hydrocarbon moiety. Preferably the alkyl comprises 1 to 20 carbon atoms, more preferably 1 to 16 carbon atoms, 1 to 10 carbon atoms, 1 to 6 carbon atoms, or 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

The number of carbon atoms in a group is specified herein by the prefix “Cₓ₋ₓₓ”, wherein x and xx are integers. For example, “C₁₋₄alkyl” is an alkyl group which has from 1 to 4 carbon atoms; and C₁₋₄haloalkyl is a haloalkyl group which has from 1 to 4 carbon atoms.

As used herein, the term “alkenyl” refers to an olefinically unsaturated branched or linear group having at least one double bond. Preferably the alkenyl comprises 2 to 20 carbon atoms, more preferably 2 to 16 carbon atoms, 2 to 10 carbon atoms, 2 to 6 carbon atoms, or 2 to 4 carbon atoms. Alkenyl groups include, but are not limited to, propenyl, 1,3-butadienyl, 1-butenyl, hexenyl, pentenyl, heptenyl, octenyl and the like.

As used herein, the term “alkynyl” refers to an unsaturated branched or linear group having at least one triple bond. Preferably the alkynyl comprises 2 to 20 carbon atoms, more preferably 2 to 16 carbon atoms, 2 to 10 carbon atoms, 2 to 6 carbon atoms, or 2 to 4 carbon atoms. Alkynyl groups include, but are not limited to, propynyl, 1-butynyl, hexynyl, pentynyl, hexynyl, heptynyl, octynyl and the like.

As used herein, the term “carbocyclyl” refers to saturated or partially unsaturated (but not aromatic) monocyclic, bicyclic or tricyclic hydrocarbon groups of 3-14 carbon atoms, preferably 3-9, or more preferably 3-8 carbon atoms. Carbocyclyls include fused, bridged, or spiro ring systems. The term “carbocyclyl” encompasses cycloalkyl groups. The term “cycloalkyl” refers to completely saturated monocyclic, bicyclic or tricyclic hydrocarbon groups of 3-12 carbon atoms, preferably 3-9, or more preferably 3-8 carbon atoms.

Exemplary monocyclic carbocyclyl groups include, but are not limited to, cyclopropyl, cyclobutyl, cyclopentyl, cyclopentenyl, cyclohexyl or cyclohexenyl. Exemplary bicyclic carbocyclyl groups include bornyl, decahydronaphthyl, bicyclo[2.1.1]hexyl, bicycle[1.1.1]pentane, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, or bicyclo[2.2.2]octyl. Exemplary tricyclic carbocyclyl groups include adamantyl.

As used herein, the term “halocycloalkyl” refers to a cycloalkyl, as defined herein, that is substituted by one or more halo groups as defined herein. Preferably the halocycloalkyl can be monohalocycloalkyl, dihalocycloalkyl or polyhalocycloalkyl including perhalocycloalkyl. A monohalocycloalkyl can have one iodo, bromo, chloro or fluoro substituent. Dihalocycloalkyl and polyhalocycloalkyl groups can be substituted with two or more of the same halo groups or a combination of different halo groups.

As used herein, the term “cycloalkenyl” refers to a partially unsaturated monocyclic, bicyclic or tricyclic hydrocarbon groups having 3-12 ring carbon atoms, preferably 3-9, or more preferably 3-8 carbon atoms, and having one or more double bonds. Exemplary monocyclic cycloalkenyl groups include, but are not limited to, cyclopentenyl, cyclopentadienyl, cyclohexenyl, and the like. Exemplary bicyclic cycloalkenyl groups include, but are not limited to, bicyclo[2.2.1]hept-5-enyl and bicycle[2.2.2]oct-2-enyl.

As used herein, the term “haloalkyl” refers to an alkyl, as defined herein, that is substituted by one or more halo groups as defined herein. Preferably, the haloalkyl can be monohaloalkyl, dihaloalkyl or polyhaloalkyl including perhaloalkyl. A monohaloalkyl can have one iodo, bromo, chloro or fluoro substituent. Dihaloalkyl and polyhaloalkyl groups can be substituted with two or more of the same halo groups or a combination of different halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms. Preferred haloalkyl groups are trifluoromethyl and difluoromethyl.

“Halogen” or “halo” may be fluoro, chloro, bromo or iodo.

The term “aryl” refers to monocyclic, bicyclic or tricyclic aromatic hydrocarbon groups having from 6 to 14 ring carbon atoms. In one embodiment, the term aryl refers to monocyclic or bicyclic aromatic hydrocarbon groups having from 6 to 10 carbon atoms. Representative examples of aryl groups include phenyl (Ph), naphthyl, fluorenyl, and anthracenyl.

The term “aryl” also refers to a bicyclic or tricyclic group in which at least one ring is aromatic and is fused to one or two non-aromatic hydrocarbon ring(s). Nonlimiting examples include tetrahydronaphthalene, dihydronaphthalenyl and indanyl.

As used herein, the term “heterocyclyl” refers to a saturated or unsaturated, non-aromatic monocyclic, bicyclic or tricyclic ring system which has from 3- to 15-ring members at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 3-8-membered monocyclic. In another embodiment, a heterocyclyl is a 6-12-membered bicyclic. In yet another embodiment, a heterocyclyl is a 10-15-membered tricyclic ring system. The heterocyclyl group can be attached at a heteroatom or a carbon atom. Heterocyclyls include fused or bridged ring systems. The term “heterocyclyl” encompasses heterocycloalkyl and heterocycloalkenyl groups. The term “heterocycloalkyl” refers to completely saturated monocyclic, bicyclic or tricyclic heterocyclyl comprising 3-15 ring members, at least one of which is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 7-membered heterocycloalkyl. Examples of heterocyclyls include dihydrofuranyl, [1,3]dioxolane, 1,4-dioxane, 1,4-dithiane, piperazinyl, 1,3-dioxolane, imidazolidinyl, imidazolinyl, pyrrolidine, dihydropyran, oxathiolane, dithiolane, 1,3-dioxane, 1,3-dithianyl, oxathianyl, thiomorpholinyl, oxiranyl, aziridinyl, oxetanyl, azetidinyl, tetrahydrofuranyl, pyrrolidinyl, tetrahydropyranyl, piperidinyl, morpholinyl, piperazinyl, azepinyl, oxapinyl, oxazepinyl and diazepinyl. The term “heterocycloalkenyl” refers to partially unsaturated monocyclic, bicyclic or tricyclic heterocyclyl comprising 3-15 ring members, with at least one double bond and at least one of the ring members is a heteroatom, and up to 10 of which may be heteroatoms, wherein the heteroatoms are independently selected from O, S and N, and wherein N and S can be optionally oxidized to various oxidation states. In one embodiment, a heterocyclyl is a 4 to 7-membered heterocycloalkenyl. Examples of heterocycloalkenyl include 1,2,3,4-tetrahydropyridinyl, 1,2-dihydropyridinyl, 1,4-dihydropyridinyl, 1,2, 3, 6-tetrahydro-pyridinyl, 1,4,5,6-tetrahydro-pyrimidinyl, 2-pyrrolinyl, 3-pyrrolinyl, 2-imidazolinyl, 2-pyrazolinyl, 3,4-dihydro-2H-pyran, dihydrofuranyl, fluoro-dihydro-furyl group, dihydro-thienyl and dihydro-thiopyran-yl.

As used herein, the term “heteroaryl” refers to a 5-14 membered monocyclic-, bicyclic-, or tricyclic-ring system, having 1 to 10 heteroatoms independently selected from N, O or S, wherein N and S can be optionally oxidized to various oxidation states, and wherein at least one ring in the ring system is aromatic. In one embodiment, the heteroaryl is a 5 to 6-membered monocyclic heteroaromatic ring. Examples of monocyclic heteroaryl groups include pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is an 8- to 10-membered bicyclic heteroaromatic ring. Examples of bicyclic heteroaryl groups include quinolinyl, quinozalinyl, phthalazinyl, quinoxalinyl, cinnolinyl, naphthyridinyl, pyridoprimidinyl, pyridopyrazinyl, pteridinyl, indolyl, isoindolyl, indolizinyl, indazolyl, benzoimidazolyl, benzotriazolyl, benzooxazolyl, benzoisoxazolyl, benzothiazolyl, benzofuranyl, isobenzofuranyl, benzothiophenyl, benzothiadiazolyl, azaindolyl, purine, imidazopyridinyl, pyrrolopyrimidinyl, imidazopyridazinyl, imidazopyrazinyl, pyrazolopyrimidinyl, pyrazolopyridinyl, pyrazolotriazinyl, oxazolopyridinyl, isoxazolopyridinyl, thiazolopyridinyl, isothiazolopyridinyl, indolyl, benzofuranyl, quinolyl, isoquinolyl indazolyl, indolinyl, isoindolyl, indolizinyl, benzamidazolyl and quinolinyl.

As used herein, the term “alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy, cyclohexyloxy and the like. Preferably, alkoxy groups have about 1-6 carbon atoms, more preferably about 1-4 carbon atoms.

The term “bicyclic” or “bicyclic ring system,” as used herein, can include a fused ring system, a bridged ring system, or a spiro ring system.

The term “fused ring system,” as used herein, is a ring system that has two or three rings (preferably two rings) independently selected from carbocyclyl, heterocyclyl, aryl or heteroaryl rings that share one side A fused ring system may have from 4-15 ring members, preferably form 5-10 ring members. Examples of fused ring systems include octahydroisoquinolin-2(1H)-yl, 2,3-dihydro-1H-indenyl, octahydro-1H-pyrido[1,2-a]pyrazinyl, and decahydroisoquinolinyl).

The term “bridged ring system,” as used herein, is a ring system that has a carbocyclyl or heterocyclyl ring wherein two non-adjacent atoms of the ring are connected (bridged) by one or more (preferably from one to three) atoms. A bridged ring system can have more than one bridge within the ring system (e.g., adamantyl). A bridged ring system may have from 6-10 ring members, preferably from 7-10 ring members. Examples of bridged ring systems include adamantly, 9-azabicyclo[3.3.1]nonan-9-yl, 8-azabicyclo[3.2.1]octanyl, bicyclo[2.2.2]octanyl, 3-azabicyclo[3.1.1]heptanyl, bicyclo[2.2.1]heptanyl, bicycle[1.1.1]pentane, (1R,5S)-bicyclo[3.2.1]octanyl, 3-azabicyclo[3.3.1]nonanyl, and bicyclo[2.2.1]heptanyl. More preferably, the bridged ring system is selected from the group consisting of 9-azabicyclo[3.3.1]nonan-9-yl, 8-azabicyclo[3.2.1]octanyl, and bicyclo[2.2.2]octanyl.

The term “spiro ring system,” as used herein, is a ring system that has two rings each of which are independently selected from a carbocyclyl or a heterocyclyl, wherein the two ring structures having one atom in common. Spiro ring systems have from 5 to 14 ring members. Example of spiro ring systems include 2-azaspiro[3.3]heptanyl, spiropentanyl, 2-oxa-6-azaspiro[3.3]heptanyl, 2,7-diazaspiro[3.5]nonanyl, 2-oxa-7-azaspiro[3.5]nonanyl, 6-oxa-9-azaspiro[4.5]decanyl, 6-oxa-2-azaspiro[3.4]octanyl, 5-azaspiro[2.3]hexanyl and 2,8-diazaspiro[4.5]decanyl.

The term “spiroheterocycloalkyl” as used herein, is a heterocycloalkyl that has one ring atom in common with the group to which it is attached. Spiroheterocycloalkyl groups may have from 3 to 15 ring members. In a preferred embodiment, the spiroheterocycloalkyl has from 3 to 8 ring atoms selected from carbon, nitrogen, sulfur and oxygen and is monocyclic.

In cases where a compound provided herein is sufficiently basic or acidic to form stable nontoxic acid or base salts, preparation and administration of the compounds as pharmaceutically acceptable salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, or α-glycerophosphate. Inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Pharmaceutically-acceptable base addition salts can be prepared from inorganic and organic bases. Salts from inorganic bases, can include but are not limited to, sodium, potassium, lithium, ammonium, calcium or magnesium salts. Salts derived from organic bases can include, but are not limited to, salts of primary, secondary or tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocycloalkyl amines, diheterocycloalkyl amines, triheterocycloalkyl amines, or mixed di- and tri-amines where at least two of the substituents on the amine can be different and can be alkyl, substituted alkyl, alkenyl, substituted alkenyl, cycloalkyl, substituted cycloalkyl, cycloalkenyl, substituted cycloalkenyl, aryl, heteroaryl, or heterocycloalkyl and the like. Also included are amines where the two or three substituents, together with the amino nitrogen, form a heterocycloalkyl or heteroaryl group. Non-limiting examples of amines can include, isopropylamine, trimethyl amine, diethyl amine, tri(iso-propyl) amine, tri(n-propyl) amine, ethanolamine, 2-dimethylaminoethanol, trimethamine, lysine, arginine, histidine, caffeine, procaine, hydrabamine, choline, betaine, ethylenediamine, glucosamine, N-alkylglucamines, theobromine, purines, piperazine, piperidine, morpholine, or N-ethylpiperidine, and the like. Other carboxylic acid derivatives can be useful, for example, carboxylic acid amides, including carboxamides, lower alkyl carboxamides, or dialkyl carboxamides, and the like.

The compounds or pharmaceutically acceptable salts thereof as described herein, can contain one or more asymmetric centers in the molecule. In accordance with the present disclosure any structure that does not designate the stereochemistry is to be understood as embracing all the various stereoisomers (e.g., diastereomers and enantiomers) in pure or substantially pure form, as well as mixtures thereof (such as a racemic mixture, or an enantiomerically enriched mixture). It is well known in the art how to prepare such optically active forms (for example, resolution of the racemic form by recrystallization techniques, synthesis from optically-active starting materials, by chiral synthesis, or chromatographic separation using a chiral stationary phase).

When a particular stereoisomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired stereoisomer relative to the combined weight of all stereoisomers.

When a particular enantiomer of a compound is depicted by name or structure, the stereochemical purity of the compounds is at least 20%, 30%, 40%, 50%, 60%, 70%, 80%, 90%, 95%, 97%, 99%, 99.5% or 99.9%. “Stereochemical purity” means the weight percent of the desired enantiomer relative to the combined weight of all stereoisomers.

When the stereochemistry of a disclosed compound is named or depicted by structure, and the named or depicted structure encompasses more than one stereoisomer (e.g., as in a diastereomeric pair), it is to be understood that one of the encompassed stereoisomers or any mixture of the encompassed stereoisomers are included. It is to be further understood that the stereoisomeric purity of the named or depicted stereoisomers at least 20%, 30%, 40%, 50%, 60%, 70%, 80%, 90%, 95%, 97%, 99%, 99.5% or 99.9%. The stereoisomeric purity the weight percent of the desired stereoisomers encompassed by the name or structure relative to the combined weight of all of the stereoisomers.

When a disclosed compound is named or depicted by structure without indicating the stereochemistry, and the compound has one chiral center, it is to be understood that the name or structure encompasses one enantiomer of compound in pure or substantially pure form, as well as mixtures thereof (such as a racemic mixture of the compound and mixtures enriched in one enantiomer relative to its corresponding optical isomer).

When a disclosed compound is named or depicted by structure without indicating the stereochemistry and, e.g., the compound has at least two chiral centers, it is to be understood that the name or structure encompasses one stereoisomer in pure or substantially pure form, as well as mixtures thereof (such as mixtures of stereoisomers, and mixtures of stereoisomers in which one or more stereoisomers is enriched relative to the other stereoisomer(s)).

The disclosed compounds may exist in tautomeric forms and mixtures and separate individual tautomers are contemplated. In addition, some compounds may exhibit polymorphism.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

In one embodiment, the present invention provides deuterated compounds described herein or a pharmaceutically acceptable salt thereof.

Another embodiment is a pharmaceutical composition comprising at least one compound described herein, or a pharmaceutically acceptable salt thereof, and at least one pharmaceutically acceptable carrier.

The compounds described herein have METTL3 modulating activity. In one embodiment, the compounds described herein have METTL3 inhibitory activity. In one embodiment, the compounds described herein are selective METTL3 inhibitors. In one embodiment, the compounds described herein have inhibitory activities against METTL3 that are higher than inhibitory activities against other protein targets, such as protein arginine N-methyltransferase 5 (PRMT5). In one embodiment, the compounds described herein have METTL3 inhibitory activities that are at least 2, 3, 5, 10, 15, 20, 30, 40, 50, 75, 100, 200, 400 or 1000 times greater than their inhibitory activities towards PRMT5.

In some embodiments, the METTL3 inhibitors described herein have an IC₅₀ value of less than 1 μM, less than 750 nM, less than 500 nM, less than 250 nM or less than 100 nM.

As used herein, “METTL3 modulating activity” refers to the ability of a compound or composition to induce a detectable change in METTL3 activity in vivo or in vitro (e.g., at least 10% increase or decrease in METTL3 activity as measured by a given assay such as the bioassay described in the examples and known in the art). A decrease in METLL3 activity is METTL3 inhibitory activity.

### Methods of Use

In one aspect, the present invention discloses a method of treating a disease or disorder responsive to inhibition of METTL3 activity in a subject comprising administering to the subject an effective amount of the compound described herein, or a pharmaceutically acceptable salt thereof.

In one embodiment, the disease or disorder is an infection, such as, a viral infection.

In a specific embodiment, the viral infection is caused by RNA virus or retrovirus. Examples of viral infections include, but are not limited to, Dengue, Yellow Fever, Japanese encephalitis, Zika virus, Ebola virus, severe acute respiratory syndrome (SARS), rabies, HIV, influenza, hepatitis C, hepatitis E, West Nile fever, polio, measles, COVID-19, and Middle East respiratory syndrome (MERS-CoV).

In one embodiment, the disease or disorder is a cancer.

The term “cancer” includes diseases or disorders involving abnormal cell growth and/or proliferation.

In some embodiments, the cancer is selected from glioblastoma, leukemia, stomach cancer, prostate cancer, colorectal cancer, endometrial cancer, breast cancer, pancreatic cancer, kidney cancer, lung cancer, bladder cancer, ovarian cancer, liver cancer, bone cancer, acute lymphocytic leukemia, esophageal/upper aerodigestive cancer, non-Hodgkin's lymphoma (NHL), multiple myeloma, mesothelioma and sarcoma.

In a specific embodiment, the cancer is acute myeloid leukemia.

As used herein, the term “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

As used herein, the term “treating” or “treatment” refers to obtaining desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

The effective dose of a compound provided herein, or a pharmaceutically acceptable salt thereof, administered to a subject can be 10 μg-500 mg.

Administering a compound described herein, or a pharmaceutically acceptable salt thereof, to a mammal comprises any suitable delivery method. Administering a compound described herein, or a pharmaceutically acceptable salt thereof, to a mammal includes administering a compound described herein, or a pharmaceutically acceptable salt thereof, topically, enterally, parenterally, transdermally, transmucosally, via inhalation, intracisternally, epidurally, intravaginally, intravenously, intramuscularly, subcutaneously, intradermally or intravitreally to the mammal. Administering a compound described herein, or a pharmaceutically acceptable salt thereof, to a mammal also includes administering topically, enterally, parenterally, transdermally, transmucosally, via inhalation, intracisternally, epidurally, intravaginally, intravenously, intramuscularly, subcutaneously, intradermally or intravitreally to a mammal a compound that metabolizes within or on a surface of the body of the mammal to a compound described herein, or a pharmaceutically acceptable salt thereof.

Thus, a compound or pharmaceutically acceptable salt thereof as described herein, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the compound or pharmaceutically acceptable salt thereof as described herein may be combined with one or more excipients and used in the form of ingestible tablets, buccal tablets, troches, capsules, elixirs, suspensions, syrups, or wafers, and the like. Such compositions and preparations should contain at least about 0.1% of active compound. The percentage of the compositions and preparations may, of course, be varied and may conveniently be between about 2 to about 60% of the weight of a given unit dosage form. The amount of active compound in such therapeutically useful compositions can be such that an effective dosage level will be obtained.

The tablets, troches, pills, capsules, and the like can include the following: binders such as gum tragacanth, acacia, corn starch or gelatin; excipients such as dicalcium phosphate; a disintegrating agent such as corn starch, potato starch, alginic acid and the like; a lubricant such as magnesium stearate; or a sweetening agent such as sucrose, fructose, lactose or aspartame or a flavoring agent.

The compounds of the invention may also be administered intravenously or intraperitoneally by infusion or injection. Solutions of the active compound or its salts can be prepared in water, optionally mixed with a nontoxic surfactant.

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation can be vacuum drying and the freeze drying techniques, which can yield a powder of the active ingredient plus any additional desired ingredient present in the previously sterile-filtered solutions.

Exemplary solid carriers can include finely divided solids such as talc, clay, microcrystalline cellulose, silica, alumina and the like. Useful liquid carriers include water, alcohols or glycols or water-alcohol/glycol blends, in which the compounds or pharmaceutically acceptable salts thereof as described herein can be dissolved or dispersed at effective levels, optionally with the aid of non-toxic surfactants.

Useful dosages of a compound or pharmaceutically acceptable salt thereof as described herein can be determined by comparing their in vitro activity, and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound or pharmaceutically acceptable salt thereof as described herein, required for use in treatment can vary not only with the particular salt selected but also with the route of administration, the nature of the condition being treated and the age and condition of the patient and can be ultimately at the discretion of the attendant physician or clinician. In general, however, a dose can be in the range of from about 0.1 to about 10 mg/kg of body weight per day.

Compounds or pharmaceutically acceptable salt thereof as described herein can be conveniently administered in unit dosage form; for example, containing 0.01 to 10 mg, or 0.05 to 1 mg, of active ingredient per unit dosage form. In some embodiments, a dose of 5 mg/kg or less can be suitable.

The desired dose may conveniently be presented in a single dose or as divided doses administered at appropriate intervals.

The disclosed method can include a kit comprising a compound or pharmaceutically acceptable salt thereof as described herein and instructional material which can describe administering a compound or pharmaceutically acceptable salt thereof as described herein or a composition comprising a compound or pharmaceutically acceptable salt thereof as described herein to a cell or a subject. This should be construed to include other embodiments of kits that are known to those skilled in the art, such as a kit comprising a (such as sterile) solvent for dissolving or suspending a compound or pharmaceutically acceptable salt thereof as described herein or composition prior to administering a compound or pharmaceutically acceptable salt thereof as described herein or composition to a cell or a subject. In some embodiments, the subject can be a human.

### Exemplifications

### Instrument Details (and Conditions)

1. ¹H NMR or ¹⁹F NMR, NOESY spectra were recorded on Bruker AVβ 400.
2. LCMS measurement was run on Agilent 1200 HPLC/6100 SQ System using the follow conditions:
Method A: Mobile Phase: A: Water (0.01% TFA) B: Acetonitrile (0.01% TFA); Gradient Phase: 5% B to 95% B within 1.4 min, 95% B with 1.6 min (total runtime: 3 min); Flow Rate: 2.0 mL/min; Column: SunFire C18, 4.6*50 mm, 3.5 μm; Column Temperature: 40° C. Detectors: ADC ELSD, DAD (214 nm and 254 nm), ES-API.
Method B: Mobile Phase: A: Water (10 mM NH₄HCO₃) B: Acetonitrile; Gradient Phase: 5% to 95% B within 1.4 min, 95% B with 1.6 min (total runtime:3 min); Flow Rate: 2.0 mL/min; Column: XBridge C18, 4.6*50 mm, 3.5 um; Column Temperature: 40° C. Detectors: ADC ELSD, DAD (214 nm and 254 nm), MSD (ES-API).
3. HPLC was taken on Agilent LC 1200 series.
Method A: Mobile Phase: A: Water (0.01% TFA) B: Acetonitrile (0.01% TFA); Gradient Phase: 5% B to 95% B within 9.5 min, 95% B with 5 min (total runtime: 14.5 min); Flow Rate: 1.0 mL/min; Column: SunFire C18, 4.6*100 mm, 3.5 μm; Column Temperature: 40° C. Detectors: ADC ELSD, DAD (214 nm and 254 nm), ES-API.
4. Prep-HPLC:
Instrument: Gilson 281 (PHG-009)
Column: Xtimate Prep C18 10 μm 21.2×250 mm
Method A: Mobile Phase: A: water (0.01% FA) B: acetonitrile
Method B: Mobile Phase: A: Water (10 mmol NH₄HCO₃); B: acetonitrile
Flow Rate (ml/min): 30.00
Detective Wavelength (nm): 214/254

### Compound Synthesis

### General Method A

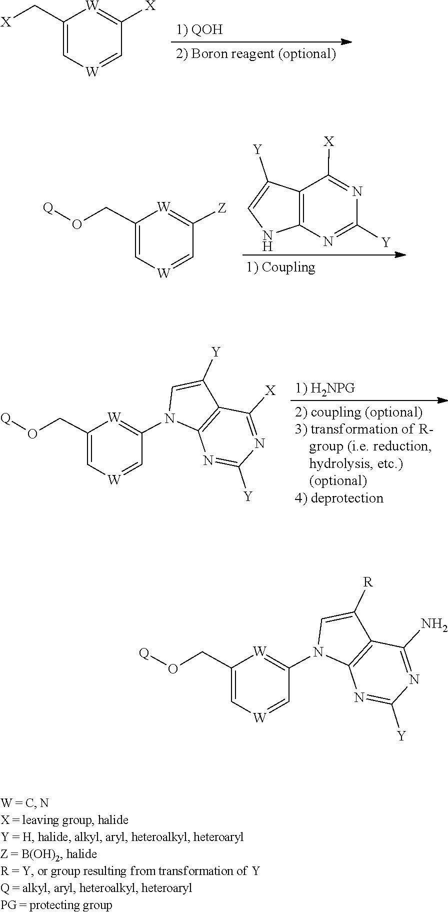

### Method A Example Synthesis

### Example 1: Synthesis of Compound I-9

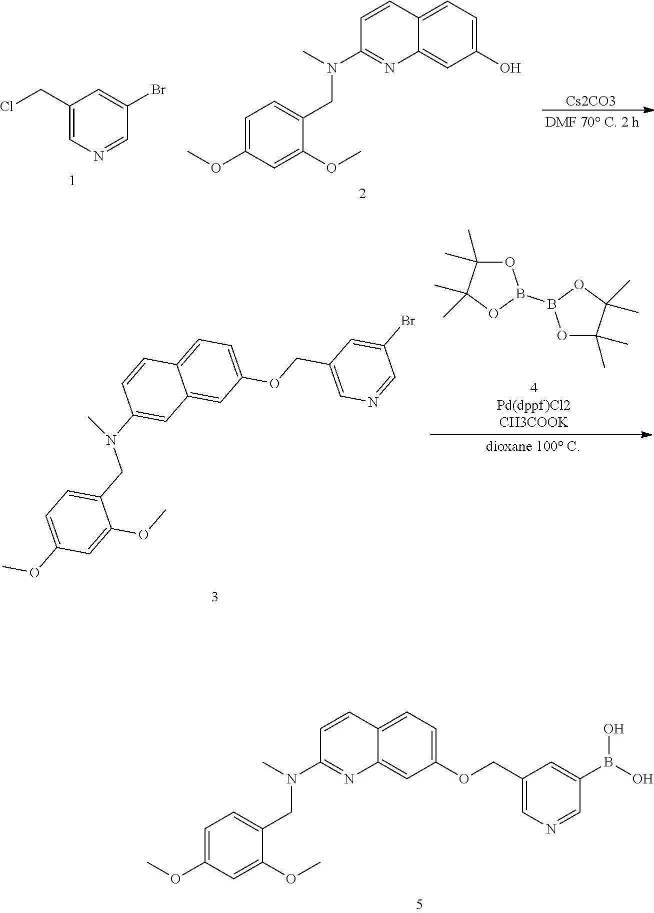

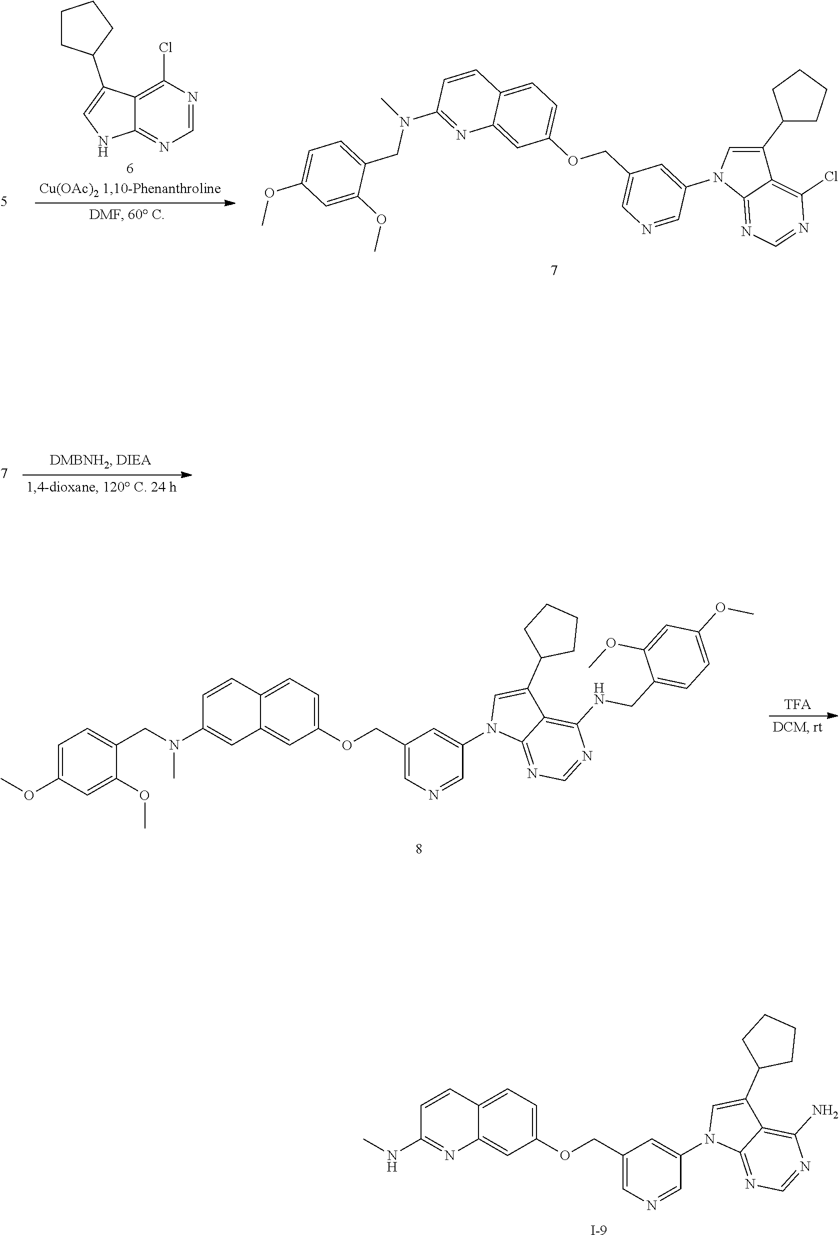

### Synthesis of 7-[(5-bromopyridin-3-yl)methoxy]-N-[(2,4-dimethoxyphenyl)methyl]-N-methylquinolin-2-amine (3)

To a solution of 3-bromo-5-(chloromethyl)pyridine hydrochloride (12.0 g, 49.3 mmol) and 2-{[(2,4-dimethoxyphenyl)methyl](methyl)amino}quinolin-7-ol (13.0 g, 40.0 mmol) in DMF (20 mL) was added Cs2CO3 (80.1 g, 246 mmol) at room temperature. The mixture was stirred at 70° C. for 2 h. Water (400 mL) was added to the cool mixture, oily-solid was formed. Water phase was removed and the oily-solid was washed with water (100 mL×3). The oily-solid was dried to yield 7-[(5-bromopyridin-3-yl)methoxy]-N-[(2,4-dimethoxyphenyl)methyl]-N-methylquinolin-2-amine (15.0 g, 30.3 mmol) as a yellow oily-solid. The crude product was directly used to next step without further purification. ESI LC-MS m/z=494.0 [M+H]+.

### Synthesis of (5-{[(2-{[(2,4-dimethoxyphenyl)methyl](methyl)amino}quinolin-7-yl)oxy]methyl}pyridin-3-yl)boronic acid (5)

7-[(5-bromopyridin-3-yl)methoxy]-N-[(2,4-dimethoxyphenyl)methyl]-N-methylquinolin-2-amine (15 g, 30.3 mmol), 4,4,5,5-tetramethyl-2-(4,4,5,5-tetramethyl-1,3,2-dioxaborolan-2-yl)-1,3,2-dioxaborolane (9.21 g, 36.3 mmol), potassium acetate (8.92 g, 90.9 mmol), Pd(dppf)Cl2 (220 mg, 302 μmol) were mixed in 1,4-dioxane (200 mL). The mixture was stirred at 90° C. for 16 h under N2. The reaction mixture was concentrated and then diluted with EtOAc (200 mL). The EtOAc phase was collected after filtration. The filtrate was concentrated to yield (5-{[(2-{[(2,4-dimethoxyphenyl)methyl](methyl)amino}quinolin-7-yl)oxy]methyl}pyridin-3-yl)boronic acid (13.0 g, 28.3 mmol) as a brown oil. The crude product was directly used to next step without further purification. ESI LC-MS m/z=460.1 [M+H]+.

### Synthesis of 7-[(5-{4-chloro-5-cyclopentyl-7H-pyrrolo[2,3-d]pyrimidin-7-yl}pyridin-3-yl)methoxy]-N-[(2,4-dimethoxyphenyl)methyl]-N-methylquinolin-2-amine (7)

(5-{[(2-{[(2,4-dimethoxyphenyl)methyl](methyl)amino}quinolin-7-yl)oxy]methyl}pyridin-3-yl)boronic acid (1 g, 2.17 mmol), 4-chloro-5-cyclopentyl-7H-pyrrolo[2,3-d]pyrimidine (576 mg, 2.60 mmol), Cu(OAc)2; (781 mg, 4.34 mmol), 1,10-Phenanthroline (391 mg, 2.17 mmol) were mixed in DMF (15 mL). The mixture was stirred at room temperature for 12 h under air. The mixture was diluted with water (120 ml) and extracted with Ethyl Acetate (120 ml×2). Combined organic layers were washed with brine (60 ml), and dried with anhydrous Na2SO4. The solvent was removed under reduced pressure to yield crude product. The residue was purified by silica gel column chromatography (silica, 20 g, EtOAc/PE: 0˜50%)) to yield the target compound 7-[(5-{4-chloro-5-cyclopentyl-7H-pyrrolo[2,3-d]pyrimidin-7-yl}pyridin-3-yl)methoxy]-N-[(2,4-dimethoxyphenyl)methyl]-N-methylquinolin-2-amine (700 mg, 1.1 mol) as a light yellow solid. ESI LC-MS m/z=635.0 [M+H]+.

### Synthesis of 7-{[5-(5-cyclopentyl-4-{[(2,4-dimethoxyphenyl)methyl]amino}-7H-pyrrolo[2,3-d]pyrimidin-7-yl)pyridin-3-yl]methoxy}-N-[(2,4-dimethoxyphenyl)methyl]-N-methylquinolin-2-amine (8)

1-(2,4-dimethoxyphenyl)methanamine (551 mg, 3.30 mmol), 7-[(5-{4-chloro-5-cyclopentyl-7H-pyrrolo[2,3-d]pyrimidin-7-yl}pyridin-3-yl)methoxy]-N-[(2,4-dimethoxyphenyl)methyl]-N-methylquinolin-2-amine (700 mg, 1.10 mmol), DIEA (425 mg, 3.30 mmol) were mixed in 1,4-dioxane (5 mL). The mixture was stirred at 120° C. for 12 h. The mixture was diluted with water (80 ml) and extracted with Ethyl Acetate (EA) (80 ml×2). Combined organic layers were washed with H2O (100 ml×2) and brine (60 ml), and dried with anhydrous Na2SO4. The solvent was removed under reduced pressure to yield the crude product. The residue was purified by silica gel column chromatography (silica, 20 g, EtOAc/PE: 0˜40%)) to yield 7-{[5-(5-cyclopentyl-4-{[(2,4-dimethoxyphenyl)methyl]amino}-7H-pyrrolo[2,3-d]pyrimidin-7-yl)pyridin-3-yl]methoxy}-N-[(2,4-dimethoxyphenyl)methyl]-N-methylquinolin-2-amine (800 mg, 1.0 mmol) as a white solid. ESI LC-MS m/z=766.2[M+H]+.

### Synthesis of 7-((5-(4-amino-5-cyclopentyl-7H-pyrrolo[2,3-d]pyrimidin-7-yl)pyridin-3-yl)methoxy)-N-methylquinolin-2-amine (9, compound I-9)

A solution of 7-{[5-(5-cyclopentyl-4-{[(2,4-dimethoxyphenyl)methyl]amino}-7H-pyrrolo[2,3-d]pyrimidin-7-yl)pyridin-3-yl]methoxy}-N-[(2,4-dimethoxyphenyl)methyl]-N-methylquinolin-2-amine (900 mg, 1.17 mmol) in TFA (10 mL) and DCM (4 mL) was stirred at room temperature for 12 h. Then the mixture was concentrated under reduced pressure, the residue was neutralized by 3 ml 7M NH₃ in MeOH to pH=8, and then filtered. The filtrate was purified by Prep-HPLC to yield 7-((5-(4-amino-5-cyclopentyl-7H-pyrrolo[2,3-d]pyrimidin-7-yl)pyridin-3-yl)methoxy)-N-methylquinolin-2-amine (150 mg, 0.32 mmol) as a white solid. ESI LC-MS m/z=466.0 [M+H]+. 1H NMR (400 MHz, DMSO-d6) δ ppm 9.10 (d, J=2.5 Hz, 1H), 8.63 (d, J=1.7 Hz, 1H), 8.43 (t, J=2.1 Hz, 1H), 8.13 (s, 1H), 7.74 (d, J=8.8 Hz, 1H), 7.53 (d, J=8.8 Hz, 1H), 7.50 (s, 1H), 7.10 (d, J=2.4 Hz, 1H), 6.96-6.91 (m, 1H), 6.88 (dd, J=8.7, 2.5 Hz, 1H), 6.74 (s, 2H), 6.58 (d, J=8.8 Hz, 1H), 5.35 (s, 2H), 3.53-3.40 (m, 1H), 2.88 (d, J=4.7 Hz, 3H), 2.13-2.07 (m, 2H), 1.81-1.49 (m, 6H).

### Example 2: Synthesis of Compound I-23

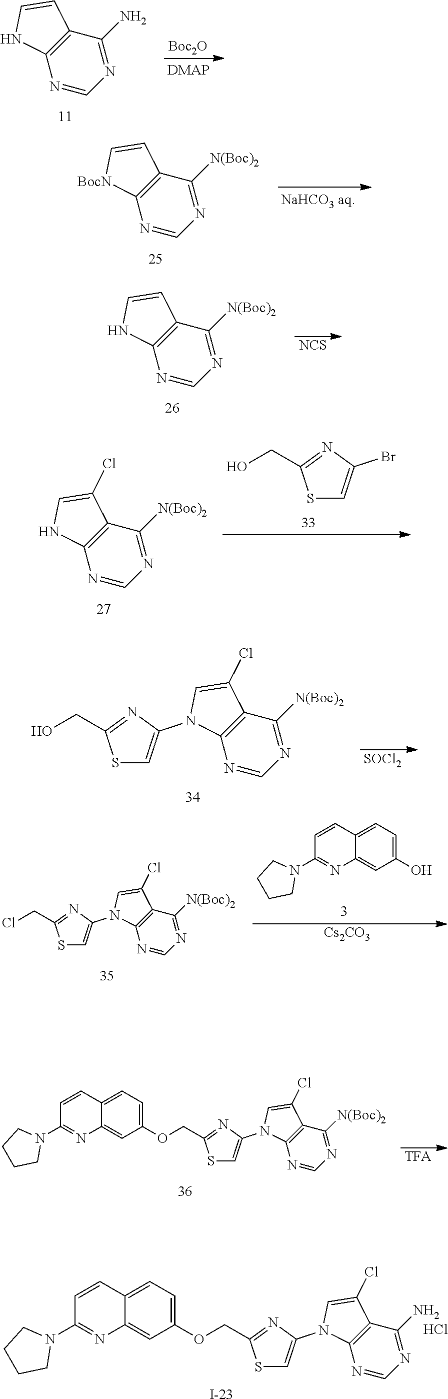

### Synthesis of tert-butyl 4-{bis[(tert-butoxy)carbonyl]amino}-7H-pyrrolo[2,3-d]pyrimidine-7-carboxylate (25)

To a mixture of 7H-pyrrolo[2,3-d]pyrimidin-4-amine (5 g, 37.2 mmol) and DMAP (18.0 g, 148 mmol) in dry THE (100 mL) was added (Boc)₂O (28.3 g, 130 mmol) at 0° C. The reaction was stirred at 25° C. for 16 h. The reaction mixture was concentrated under reduced pressure. The residue was purified by column (petroleum ether/EtOAc=3:1, v/v) to afford tert-butyl 4-{bis[(tert-butoxy)carbonyl]amino}-7H-pyrrolo[2,3-d]pyrimidine-7-carboxylate (8.5 g, 52.7% yield) as a white solid.

### Synthesis of tert-butyl N-[(tert-butoxy)carbonyl]-N-{7H-pyrrolo[2,3-d]pyrimidin-4-yl}carbamate (26)

To a mixture of tert-butyl 4-{bis[(tert-butoxy)carbonyl]amino}-7H-pyrrolo[2,3-d]pyrimidine-7-carboxylate (8.4 g, 19.3 mmol) in MeOH (50 mL) was added saturated aqueous sodium bicarbonate solution (20 mL). The reaction mixture was stirred at 25° C. for 16 h. The reaction mixture was concentrated under reduced pressure. The residue was purified by column (petroleum ether/EtOAc=1:1, v/v) to afford tert-butyl N-[(tert-butoxy)carbonyl]-N-{7H-pyrrolo[2,3-d]pyrimidin-4-yl}carbamate (5 g, 77.5% yield) as a white solid.

### Synthesis of tert-butyl N-[(tert-butoxy)carbonyl]-N-{5-chloro-7H-pyrrolo[2,3-d]pyrimidin-4-yl}carbamate (27)

A solution of tert-butyl N-[(tert-butoxy)carbonyl]-N-{7H-pyrrolo[2,3-d]pyrimidin-4-yl}carbamate (1 g, 2.99 mmol) and NCS (518 mg, 3.88 mmol) in CHCl₃ (10 mL) was stirred at 60° C. for 1 h under a nitrogen atmosphere. After cooling to room temperature, the reaction mixture was concentrated under reduced pressure and the residue was purified by column (petroleum ether/EtOAc=3:1, v/v) to afford tert-butyl N-[(tert-butoxy)carbonyl]-N-{5-chloro-7H-pyrrolo[2,3-d]pyrimidin-4-yl}carbamate (1 g, 90.9% yield) as a white solid.

### Synthesis of tert-butyl N-[(tert-butoxy) carbonyl]-N-{5-chloro-7-[2-(hydroxymethyl)-1,3-thiazol-4-yl]-7H-pyrrolo[2,3-d]pyrimidin-4-yl}carbamate (34)

A mixture of (4-bromo-1,3-thiazol-2-yl)methanol (300 mg, 1.54 mmol), tert-butyl N-[(tert-butoxy)carbonyl]-N-{5-chloro-7H-pyrrolo[2,3-d]pyrimidin-4-yl}carbamate (472 mg, 1.28 mmol), trans-N,N′-dimethylcyclohexane-1,2-diamine (9.11 mg, 64.1 μmol), K₃PO₄ (379 mg, 1.79 mmol) and CuI (9.77 mg, 51.3 μmol) in dioxane (4 mL) was stirred at 110° C. for 16 h under a N₂ atmosphere. After cooling to room temperature, the reaction mixture was concentrated under reduced pressure. The residue was purified by column (Petroleum ether/EtOAc=3:1, v/v) to afford tert-butyl N-[(tert-butoxy) carbonyl]-N-{5-chloro-7-[2-(hydroxymethyl)-1,3-thiazol-4-yl]-7H-pyrrolo[2,3-d]pyrimidin-4-yl}carbamate (150 mg, 24.3% yield) as a yellow solid.

### Synthesis of tert-butyl N-[(tert-butoxy)carbonyl]-N-{5-chloro-7-[2-(chloromethyl)-1,3-thiazol-4-yl]-7H-pyrrolo[2,3-d]pyrimidin-4-yl}carbamate (35)

To a solution of tert-butyl N-[(tert-butoxy) carbonyl]-N-{5-chloro-7-[2-(hydroxymethyl)-1,3-thiazol-4-yl]-7H-pyrrolo[2,3-d]pyrimidin-4-yl}carbamate (120 mg, 248 μmol) and DMAP (15.1 mg, 124 μmol) in DCM (20 mL) was added SOCl₂ (0.054 mL) and the reaction mixture was stirred at 25° C. for 2 h under a N₂ atmosphere. The reaction mixture was concentrated under reduced pressure. The residue was purified by prep-TLC (petroleum ether/EtOAc=2:1, v/v) to afford tert-butyl N-[(tert-butoxy)carbonyl]-N-{5-chloro-7-[2-(chloromethyl)-1,3-thiazol-4-yl]-7H-pyrrolo[2,3-d]pyrimidin-4-yl}carbamate (60 mg, 48.3% yield) as a light yellow solid.

### Synthesis of tert-butyl N-[(tert-butoxy)carbonyl]-(5-chloro-7-(2-(((2-(pyrrolidin-1-yl)quinolin-7-yl)oxy)methyl)thiazol-4-yl)-7H-pyrrolo[2,3-d]pyrimidin-4-yl)carbamate (36)

A mixture of tert-butyl N-[(tert-butoxy)carbonyl]-N-{5-chloro-7-[2-(chloromethyl)-1,3-thiazol-4-yl]-7H-pyrrolo[2,3-d]pyrimidin-4-yl}carbamate (100 mg, 0.200 mmol), 2-(pyrrolidin-1-yl)quinolin-7-ol (51.4 mg, 0.240 mmol), and Cs₂CO₃ (195.5 mg, 0.600 mmol) in DMF (4 mL) was stirred at 25° C. for 3 h. The mixture was concentrated, and the residue was purified by prep-TLC (petroleum ether/EtOAc=1:2, v/v) to afford the tert-butyl N-[(tert-butoxy)carbonyl]-(5-chloro-7-(2-(((2-(pyrrolidin-1-yl)quinolin-7-yl)oxy)methyl)thiazol-4-yl)-7H-pyrrolo[2,3-d]pyrimidin-4-yl)carbamate (40 mg, 29% yield) as a yellow solid.

### Synthesis of 5-chloro-7-(2-(((2-(pyrrolidin-1-yl)quinolin-7-yl)oxy)methyl)thiazol-4-yl)-7H-pyrrolo[2,3-d]pyrimidin-4-amine hydrochloride (37, compound I-23)

To a solution of tert-butyl N-[(tert-butoxy)carbonyl]-(5-chloro-7-(2-(((2-(pyrrolidin-1-yl)quinolin-7-yl)oxy)methyl)thiazol-4-yl)-7H-pyrrolo[2,3-d]pyrimidin-4-yl)carbamate (40 mg, 0.059 mmol) in DCM (2 mL) was added 2 M HCl/EtOAc (3 mL), then the mixture was stirred at 25° C. for 5 h. The mixture was concentrated under reduced pressure to afford the crude solid, then the solid was triturated with Et₂O (10 mL×3) and n-hexane (10 mL×3) to give 5-chloro-7-(2-(((2-(pyrrolidin-1-yl)quinolin-7-yl)oxy)methyl)thiazol-4-yl)-7H-pyrrolo[2,3-d]pyrimidin-4-amine hydrochloride (28 mg, 76% yield) as a yellow solid. ESI LC-MS m/z=478.2 [M+H]+. ¹H NMR (400 MHz, DMSO-d6) δ ppm 13.06 (s, 1H), 8.43 (s, 1H), 8.34 (d, J=9.6 Hz, 1H), 8.23 (s, 1H), 8.15-8.09 (m, 2H), 7.91 (d, J=8.8 Hz, 1H), 7.27 (dd, J₁=8.8 Hz, J₂=2.0 Hz, 1H), 7.11 (d, J=9.6 Hz, 1H), 5.63 (s, 2H), 3.90-3.77 (m, 4H), 2.14-1.99 (m, 4H).

### General Method B

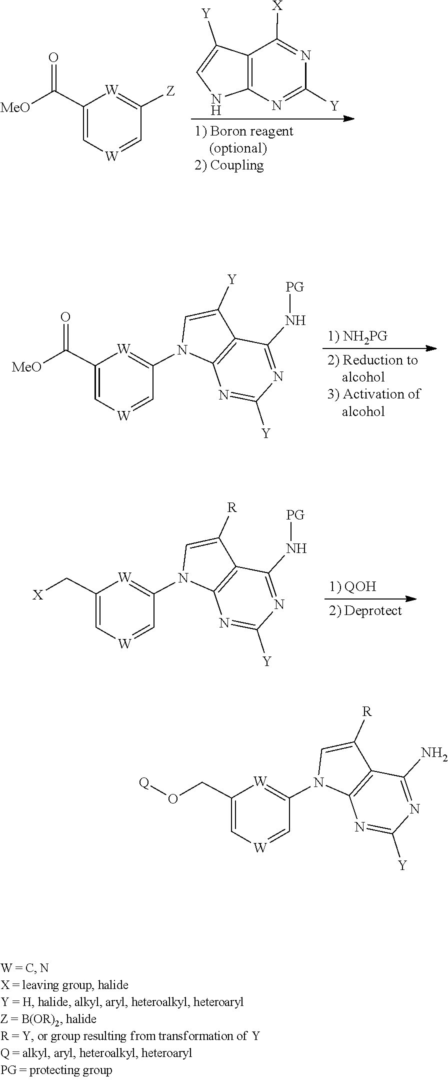

### Method B Example Synthesis

### Example 3: Synthesis of Compound I-74

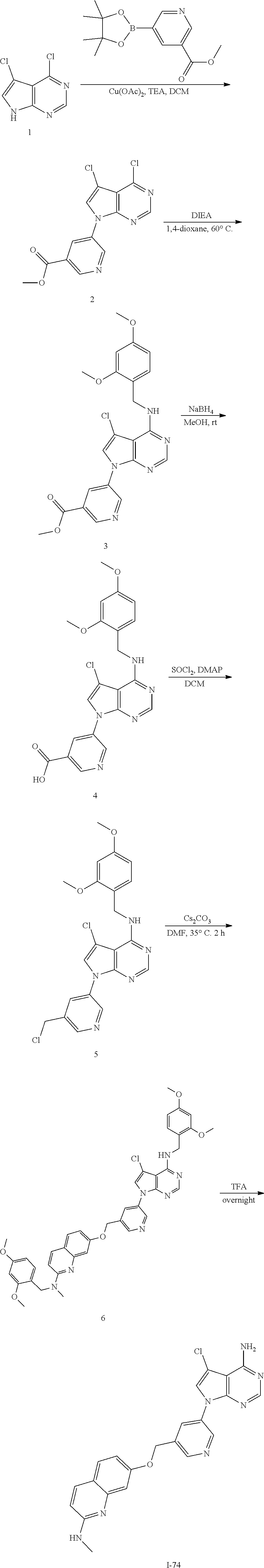

### Synthesis of methyl 5-(4,5-dichloro-7H-pyrrolo[2,3-d]pyrimidin-7-yl)nicotinate (2)

A solution of 4,5-dichloro-7H-pyrrolo[2,3-d]pyrimidine (500 mg, 2.65 mmol) in DCM (15 mL) was added methyl 5-(4,4,5,5-tetramethyl-1,3,2-dioxaborolan-2-yl)pyridine-3-carboxylate (697 mg, 2.65 mmol), Cu(OAc)2; (577 mg, 3.18 mmol) and triethylamine (321 mg, 3.18 mmol). The mixture was stirred at room temperature overnight. Solvent was removed under reduced pressure and the residue was purified by silica gel column chromatography (silica, 10 g, MeOH/DCM: 0˜5%) to yield methyl 5-(4,5-dichloro-7H-pyrrolo[2,3-d]pyrimidin-7-yl)nicotinate (480 mg, 1.48 mmol) as a colorless oil, ESI LC-MS m/z=323.1 [M+H]+.

### Synthesis of methyl 5-(5-chloro-4-(2,4-dimethoxybenzylamino)-7H-pyrrolo[2,3-d]pyrimidin-7-yl)nicotinate (3)

To a solution of methyl 5-{4,5-dichloro-7H-pyrrolo[2,3-d]pyrimidin-7-yl}pyridine-3-carboxylate (480 mg, 1.48 mmol) in dioxane (3 mL) was added 1-(2,4-dimethoxyphenyl)methanamine (369 mg, 2.21 mmol) and ethyl bis(propan-2-yl)amine (590 mg, 4.5 mmol). The mixture was stirred at 60° C. in a sealed tube overnight. Solvent was removed under reduced pressure and the residue was purified by silica gel column chromatography (silica, 10 g, MeOH/DCM: 0˜5%) to afford methyl 5-(5-chloro-4-(2,4-dimethoxybenzylamino)-7H-pyrrolo[2,3-d]pyrimidin-7-yl)nicotinate (250 mg, 550 μmol, yield: 37.2%) as a white solid, ESI LC-MS m/z=454.1 [M+H]+.

### Synthesis of (5-(5-chloro-4-(2,4-dimethoxybenzylamino)-7H-pyrrolo[2,3-d]pyrimidin-7-yl)pyridin-3-yl)methanol (4)

To a solution of methyl 5-(5-chloro-4-{[(2,4-dimethoxyphenyl)methyl]amino}-7H-pyrrolo[2,3-d]pyrimidin-7-yl)pyridine-3-carboxylate (200 mg, 440 μmol) in methanol (10 mL) was added NaBH4 (166 mg, 4.39 mmol) at room temperature. The mixture was stirred at room temperature for 3 h. Solvent was removed under reduced pressure and the residue was purified by prep-TLC (silica, EtOAc/Pet Ether=40%) to yield (5-(5-chloro-4-(2,4-dimethoxybenzylamino)-7H-pyrrolo[2,3-d]pyrimidin-7-yl)pyridin-3-yl)methanol (150 mg, 352 μmol) as a white solid. ESI LC-MS m/z=426.2 [M+H]+.

### Synthesis of 5-chloro-7-(5-(chloromethyl)pyridin-3-yl)-N-(2,4-dimethoxybenzyl)-7H-pyrrolo[2,3-d]pyrimidin-4-amine (5)

To a solution of [5-(5-chloro-4-{[(2,4-dimethoxyphenyl)methyl]amino}-7H-pyrrolo[2,3-d]pyrimidin-7-yl)pyridin-3-yl]methanol (130 mg, 305 μmol) in DCM (8 mL) was added SOCl₂ (362 mg, 3.05 mmol) and N,N-dimethylpyridin-4-amine (37.2 mg, 305 μmol). The mixture was stirred at room temperature for 2 h. Solvent was removed under reduced pressure to yield 5-chloro-7-(5-(chloromethyl)pyridin-3-yl)-N-(2,4-dimethoxybenzyl)-7H-pyrrolo[2,3-d]pyrimidin-4-amine (100 mg, 225 μmol) as a yellow oil. The crude product was directly used to next step without further purification. ESI LC-MS m/z=444.1 [M+H]+.

### Synthesis of 7-((5-(5-chloro-4-(2,4-dimethoxybenzylamino)-7H-pyrrolo[2,3-d]pyrimidin-7-yl)pyridin-3-yl)methoxy)-N-(2,4-dimethoxybenzyl)-N-methylquinolin-2-amine (6)

To a solution of 5-chloro-7-[5-(chloromethyl)pyridin-3-yl]-N-[(2,4-dimethoxyphenyl)methyl]-7H-pyrrolo[2,3-d]pyrimidin-4-amine (100 mg, 225 μmol) in DMF (3 mL) was added 2-{[(2,4-dimethoxyphenyl)methyl](methyl)amino}quinolin-7-ol (58.3 mg, 180 μmol) and Cs2CO3 (219 mg, 675 μmol). The mixture was stirred at 50° C. for 3 h. The mixture was purified by prep-HPLC directly to yield 7-((5-(5-chloro-4-(2,4-dimethoxybenzylamino)-7H-pyrrolo[2,3-d]pyrimidin-7-yl)pyridin-3-yl)methoxy)-N-(2,4-dimethoxybenzyl)-N-methylquinolin-2-amine as a white solid (40 mg, 27.3 μmol), ESI LC-MS m/z=732.6 [M+H]+.

### Synthesis of 7-((5-(4-amino-5-chloro-7H-pyrrolo[2,3-d]pyrimidin-7-yl)pyridin-3-yl)methoxy)-N-methylquinolin-2-amine (7, compound I-74)

A solution of 7-{[5-(5-chloro-4-{[(2,4-dimethoxyphenyl)methyl]amino}-7H-pyrrolo[2,3-d]pyrimidin-7-yl)pyridin-3-yl]methoxy}-N-[(2,4-dimethoxyphenyl)methyl]-N-methylquinolin-2-amine (20 mg, 54.6 μmol) in TFA (5 mL) was stirred at 60° C. for 3 h. Then the mixture was concentrated under reduced pressure, the residue was neutralized by 3 ml 7M NH3 in MeOH to pH=8, and then filtered. The filtrate was purified by Prep-HPLC to yield 7-((5-(4-amino-5-chloro-7H-pyrrolo[2,3-d]pyrimidin-7-yl)pyridin-3-yl)methoxy)-N-methylquinolin-2-amine (14.2 mg, 31.6 μmol) as a white solid, ESI LC-MS m/z=432.1 [M+H]+. 1H NMR (400 MHz, DMSO-d6) δ 9.02 (s, 1H), 8.70 (s, 1H), 8.42 (s, 1H), 8.19 (s, 1H), 7.97 (s, 1H), 7.74 (d, J=8.9 Hz, 1H), 7.53 (d, J=8.7 Hz, 1H), 7.10 (d, J=2.4 Hz, 1H), 6.95 (d, J=4.7 Hz, 1H), 6.88 (dd, J=8.7, 2.5 Hz, 1H), 6.58 (d, J=8.8 Hz, 1H), 5.36 (s, 2H), 2.88 (d, J=4.7 Hz, 3H).

### Example 4: Synthesis of Compound I-38

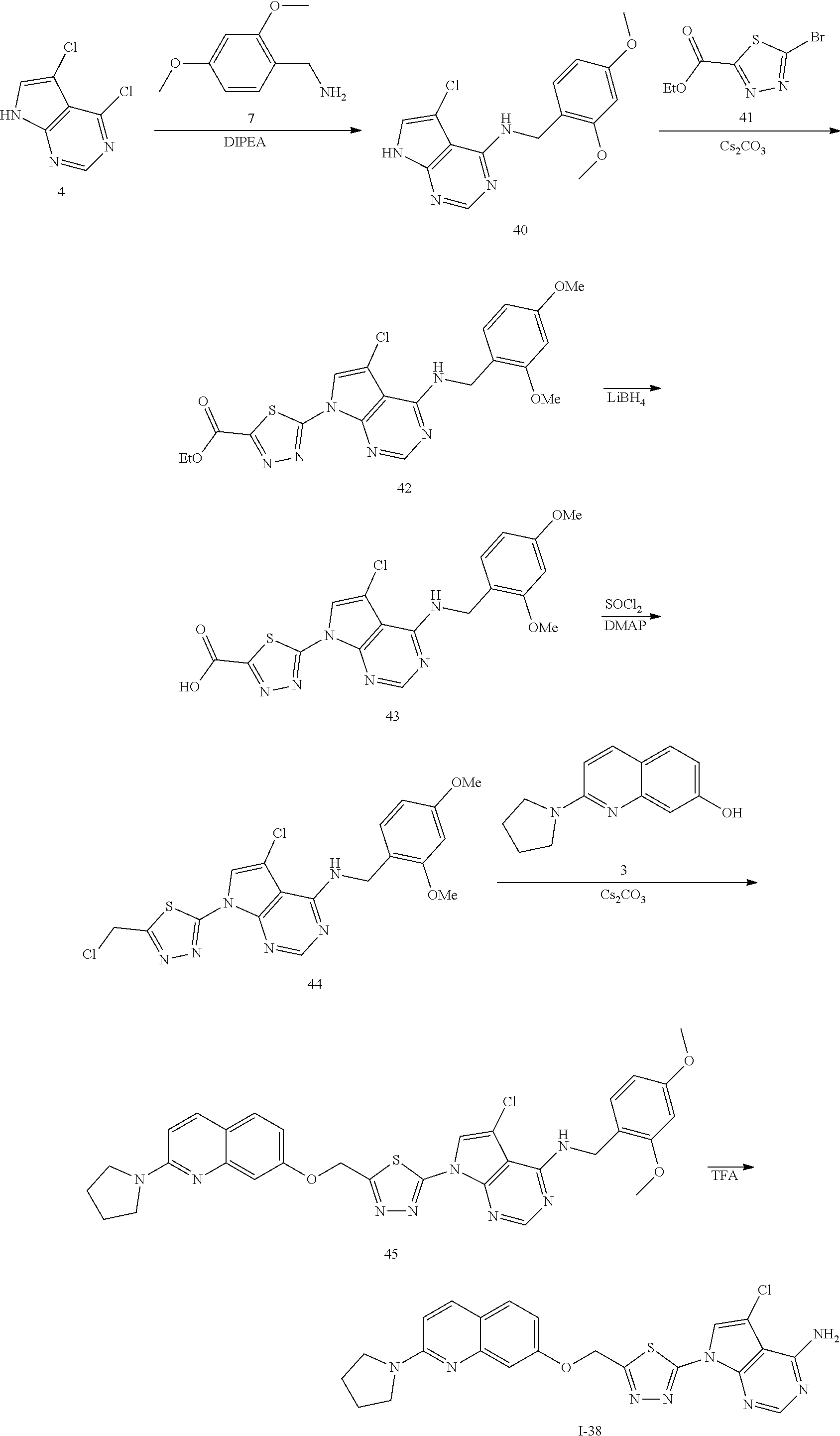

### Synthesis of 5-chloro-N-(2,4-dimethoxybenzyl)-7H-pyrrolo[2,3-d]pyrimidin-4-amine (40)

To a solution of 4,5-dichloro-7H-pyrrolo[2,3-d]pyrimidine (1 g, 5.32 mmol) and 2,4-dimethoxybenzylamine (1.6 mL, 10.64 mmol) in dioxane (20 mL) was added N,N-diisopropylethylamine (8.8 mL, 53.2 mmol). The mixture was stirred at 120° C. in a sealed tube for 16 h. The mixture was cooled to room temperature, concentrated under reduced pressure, and the residue was purified by Reverse Phase-column (H₂O/ACN, 0.1% TFA) to afford 5-chloro-N-(2,4-dimethoxybenzyl)-7H-pyrrolo[2,3-d]pyrimidin-4-amine (1 g, 59% yield) as a yellow solid.

### Synthesis of ethyl 5-(5-chloro-4-((2,4-dimethoxybenzyl)amino)-7H-pyrrolo[2,3-d]pyrimidin-7-yl)-1,3,4-thiadiazole-2-carboxylate (42)

A mixture of 5-chloro-N-(2,4-dimethoxybenzyl)-7H-pyrrolo[2,3-d]pyrimidin-4-amine (500 mg, 1.57 mmol), ethyl 5-bromo-1,3,4-thiadiazole-2-carboxylate (427.6 mg, 1.80 mmol), and Cs₂CO₃ (332.2 mg, 1.02 mmol) in DMSO (20 mL) was stirred at 80° C. for 3.5 h. The mixture was concentrated, and the residue was purified by Reverse Phase-column (H₂O/ACN, 0.1% TFA) to afford the ethyl 5-(5-chloro-4-((2,4-dimethoxybenzyl)amino)-7H-pyrrolo[2,3-d]pyrimidin-7-yl)-1,3,4-thiadiazole-2-carboxylate (360 mg, 48% yield) as a yellow solid.

### Synthesis of (5-(5-chloro-4-((2,4-dimethoxybenzyl)amino)-7H-pyrrolo[2,3-d]pyrimidin-7-yl)-1,3,4-thiadiazol-2-yl)methanol (43)

To a solution of ethyl 5-(5-chloro-4-((2,4-dimethoxybenzyl)amino)-7H-pyrrolo[2,3-d]pyrimidin-7-yl)-1,3,4-thiadiazole-2-carboxylate (350 mg, 0.737 mmol) in dry THE (30 mL) was added 2 M LiBH₄/THF (0.368 mL, 0.737 mmol) at −30° C. under a N₂ atmosphere. The mixture was then stirred controlled at −30° C.˜−20° C. for 40 min. The mixture was quenched by saturated NH₄Cl aqueous (1 mL), then concentrated. The residue was purified by prep-TLC (petroleum ether/EtOAc=1:2, v/v) to afford (5-(5-chloro-4-((2,4-dimethoxybenzyl)amino)-7H-pyrrolo[2,3-d]pyrimidin-7-yl)-1,3,4-thiadiazol-2-yl)methanol (150 mg, 47% yield) as a yellow solid.

### Synthesis of 5-chloro-7-(5-(chloromethyl)-1,3,4-thiadiazol-2-yl)-N-(2,4-dimethoxybenzyl)-7H-pyrrolo[2,3-d]pyrimidin-4-amine (44)

To a solution of (5-(5-chloro-4-((2,4-dimethoxybenzyl)amino)-7H-pyrrolo[2,3-d]pyrimidin-7-yl)-1,3,4-thiadiazol-2-yl)methanol (150 mg, 0.346 mmol) and DMAP (21.2 mg, 0.173 mmol) in DCM (25 mL) was added thionyl chloride (0.076 mL, 1.04 mmol). The mixture was stirred at 25° C. for 3 h. The mixture was concentrated, and the residue was purified by reverse-phase column (H₂O/ACN, 0.1% TFA) to afford the 5-chloro-7-(5-(chloromethyl)-1,3,4-thiadiazol-2-yl)-N-(2,4-dimethoxybenzyl)-7H-pyrrolo[2,3-d]pyrimidin-4-amine (98 mg, 63% yield) as a yellow solid.

### Synthesis of 5-chloro-N-(3,4-dimethylbenzyl)-7-(5-(((2-(pyrrolidin-1-yl)quinolin-7-yl)oxy)methyl)-1,3,4-thiadiazol-2-yl)-7H-pyrrolo[2,3-d]pyrimidin-4-amine (45)

A mixture of 5-chloro-7-(5-(chloromethyl)-1,3,4-thiadiazol-2-yl)-N-(2,4-dimethoxybenzyl)-7H-pyrrolo[2,3-d]pyrimidin-4-amine (80 mg, 0.177 mmol), 2-(pyrrolidin-1-yl)quinolin-7-ol (41.8 mg, 0.195 mmol), and Cs₂CO₃ (173.3 mg, 0.532 mmol) in DMF (2 mL) was stirred at 25° C. for 3 h. LCMS showed target molecule was formed. The mixture was concentrated, and the residue was purified RP-column (H₂O/ACN, 0.1% TFA) to afford the 5-chloro-N-(3,4-dimethylbenzyl)-7-(5-(((2-(pyrrolidin-1-yl)quinolin-7-yl)oxy)methyl)-1,3,4-thiadiazol-2-yl)-7H-pyrrolo[2,3-d]pyrimidin-4-amine (150 mg, crude) as a yellow solid.

### Synthesis of 5-chloro-7-(5-(((2-(pyrrolidin-1-yl)quinolin-7-yl)oxy)methyl)-1,3,4-thiadiazol-2-yl)-7H-pyrrolo[2,3-d]pyrimidin-4-amine trifluoroacetate (46, compound I-38)

To a solution of 5-chloro-N-(3,4-dimethylbenzyl)-7-(5-(((2-(pyrrolidin-1-yl)quinolin-7-yl)oxy)methyl)-1,3,4-thiadiazol-2-yl)-7H-pyrrolo[2,3-d]pyrimidin-4-amine (120 mg, crude) in DCM (4 mL) was added CF₃COOH (2 mL), then the mixture was stirred at 25° C. for 3 h. The mixture was concentrated under reduced pressure, the residue was purified by prep-HPLC (H₂O/CH₃CN, 0.1% TFA) to give 5-chloro-7-(5-(((2-(pyrrolidin-1-yl)quinolin-7-yl)oxy)methyl)-1,3,4-thiadiazol-2-yl)-7H-pyrrolo[2,3-d]pyrimidin-4-amine trifluoroacetate (18 mg, 21.8% yield, 2 steps) as a light yellow solid. ESI LC-MS m/z=479.2 [M+H]+. ¹H NMR (400 MHz, DMSO-d6) δ ppm 12.46 (s, 1H), 8.36-8.23 (m, 2H), 8.17 (s, 1H), 7.88 (d, J=8.4 Hz, 1H), 7.61 (s, 1H), 7.28-7.21 (m, 1H), 7.10-7.02 (m, 1H), 5.76 (s, 2H), 3.75-3.66 (m, 4H), 2.16-1.99 (m, 4H).

### General Method C

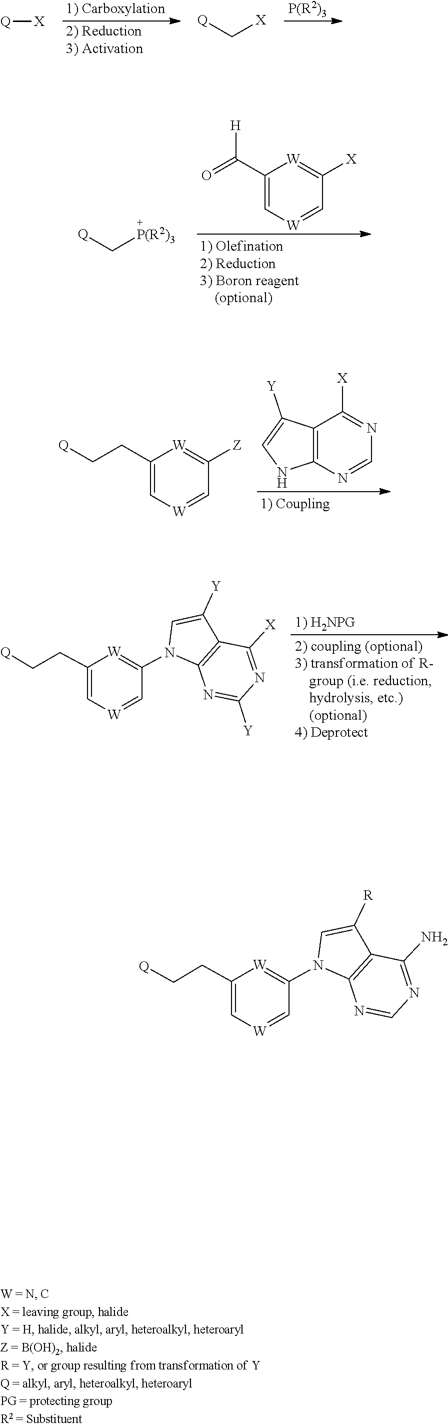

### Method C Example Synthesis

### Example 5: Synthesis of Compound I-1

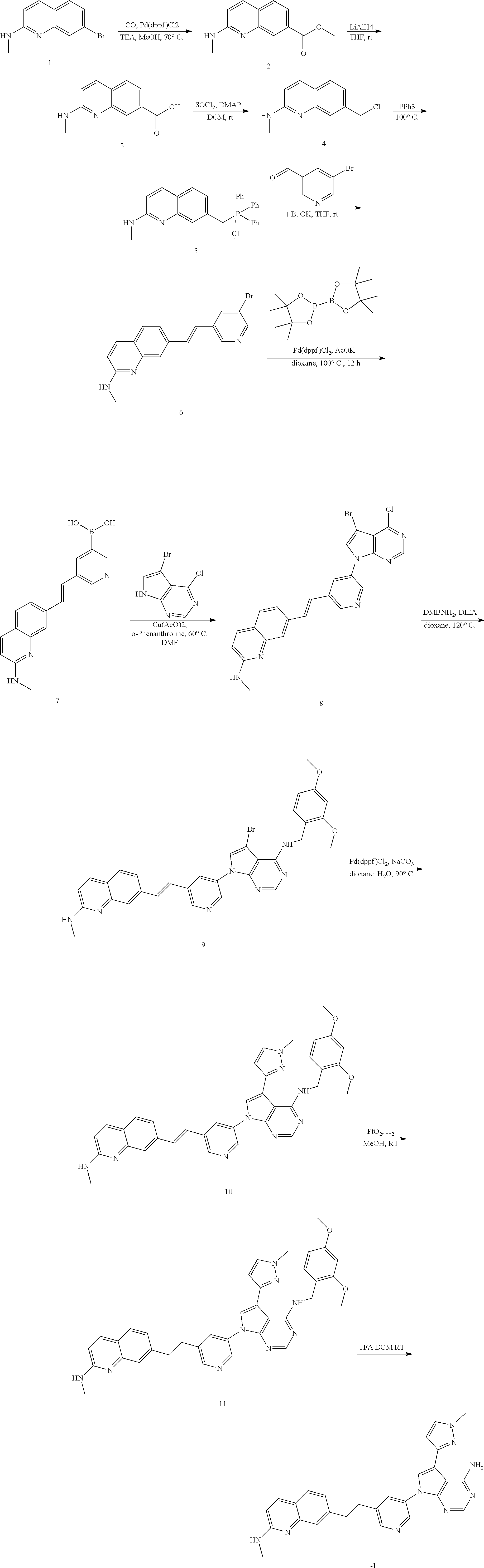

### Synthesis of methyl 2-(methylamino)quinoline-7-carboxylate (2)

7-bromo-N-methylquinolin-2-amine (1 g, 4.21 mmol), Pd(dppf)Cl₂ (343 mg, 421 μmol), TEA (1.27 g, 12.6 mmol) were mixed in MeOH (15 mL). The mixture was stirred at 70° C. for 12 h under CO atmosphere. The mixture was diluted with water (80 ml) and extracted with EtOAc (80 ml×2). Combined organic layers were washed with H2O (100 ml×2) and brine (60 ml), and dried (Na2SO4). The solvent was removed under reduced pressure to yield the crude product. The crude product was purified by flash chromatography (silica, 20 g, EtOAc/PE: 0˜20%) to yield the target compound (800 mg, 87.6%) as a white solid. ESI LC-MS m/z=217.0 [M+H]+.

### Synthesis of [2-(methylamino)quinolin-7-yl]methanol (3)

To a solution of methyl 2-(methylamino)quinoline-7-carboxylate (800 mg, 3.69 mmol) in THE (5 mL) was added LiAlH4 (698 mg, 18.4 mmol) at room temperature. The mixture was stirred at room temperature for 1 h. Na2SO4.10H2O was added to reaction solution and stirred for 30 mins, The LiAlH4 was then quenched, filtered, and the filtrate was concentrated to yield the target compound (600 mg, yield, 86.4%) ESI LC-MS m/z=189.2 [M+H]+.

### Synthesis of 7-(chloromethyl)-N-methylquinolin-2-amine (4)

[2-(methylamino)quinolin-7-yl]methanol (600 mg, 3.18 mmol), thionyl chloride (1.89 g, 15.9 mmol) were mixed in DCM (5 mL). Few DMAP was added and the resulting mixture was stirred at room temperature for 1 h. The mixture was concentrated under reduced pressure to yield the crude product as a white solid. ESI LC-MS m/z=207.0 [M+H]+.

### Synthesis of {[2-(methylamino)quinolin-7-yl]methyl}triphenylphosphanium (5)

7-(chloromethyl)-N-methylquinolin-2-amine (600 mg, 2.90 mmol), triphenylphosphane (7.58 g, 28.9 mmol) was added and heated to 100° C. for 48 h. The mixture was cooled to room temperature and EtOAc (150 ml) was added. The mixture was stirred for 15 min and filtrated to yield the crude product. The crude product was washed with EtOAc (10 ml×3) to yield the target compound as a white solid (860 mg). ESI LC-MS m/z=433.0 [M]+.

### Synthesis of 7-[(1E)-2-(5-bromopyridin-3-yl)ethenyl]-N-methylquinolin-2-amine (6)

To a solution of {[2-(methylamino)quinolin-7-yl]methyl}triphenylphosphanium (1.20 g, 2.76 mmol) in THE (20 mL) was added (tert-butoxy)potassium (1.85 g, 16.5 mmol) at 0° C. The suspension was turned to bright yellow and stirred at 0° C. for 0.5 h. Then 5-bromopyridine-3-carbaldehyde (615 mg, 3.31 mmol) was added and stirred at room temperature for 1 h. The mixture was diluted with water (80 ml) and extracted with EtOAc (80 ml×2). The combined organic layers were washed with H2O (100 ml×2) and brine (60 ml), and dried (Na2SO4). The solvent was removed under reduced pressure to yield the crude product. The crude product was purified by flash chromatography (silica, EtOAc/PE: 0˜35%) to yield the target compound (600 mg, yield 63.8%) as a white solid. ESI LC-MS m/z=340.0 [M+H]+.

### Synthesis of {5-[(1E)-2-[2-(methylamino)quinolin-7-yl]ethenyl]pyridin-3-yl}boronic acid (7)

7-[(1E)-2-(5-bromopyridin-3-yl)ethenyl]-N-methylquinolin-2-amine (600 mg, 1.76 mmol), 4,4,5,5-tetramethyl-2-(4,4,5,5-tetramethyl-1,3,2-dioxaborolan-2-yl)-1,3,2-dioxaborolane (670 mg, 2.64 mmol), AcOK (518 mg, 5.28 mmol), Pd(dppf)Cl2 (142 mg, 176 μmol) were mixed in 1,4-dioxane (15 mL). The mixture was heated to 100° C. and stirred for 12 h under N2 atmosphere. The mixture was concentrated to dryness then ethyl acetate (100 mL) was added. After stirring for 30 min, ethyl acetate solution was collected by filtration and concentrated to yield crude product. The crude product was directly used to next step without further purification. ESI LC-MS m/z=306.1[M+H]+.

### Synthesis of 7-[(1E)-2-(5-{5-bromo-4-chloro-7H-pyrrolo[2,3-d]pyrimidin-7-yl}pyridin-3-yl)ethenyl]-N-methylquinolin-2-amine (8)

{5-[(1E)-2-[2-(methylamino)quinolin-7-yl]ethenyl]pyridin-3-yl}boronic acid (450 mg, 1.47 mmol), 5-bromo-4-chloro-7H-pyrrolo[2,3-d]pyrimidine (341 mg, 1.47 mmol), Cu(AcO)₂; (529 mg, 2.94 mmol), o-Phenanthroline (529 mg, 2.94 mmol) were mixed in DMF (10 mL). The mixture was heated to 60° C. and stirred for 4 h under air atmosphere. The mixture was diluted with water (100 ml) and extracted with EtOAc (80 ml×2). The combined organic layers were washed with H2O (100 ml×2) and brine (60 ml), and dried (Na2SO4). The solvent was removed under reduced pressure to yield the crude product. The crude product was purified by flash chromatography (silica, 10 g, EtOAc/Pet. Ether: 0˜80%) to yield the target compound (250 mg) as a white solid. ESI LC-MS m/z=491.0[M+H]+.

### Synthesis of 7-[(1E)-2-[5-(5-bromo-4-{[(2,4-dimethoxyphenyl)methyl]amino}-7H-pyrrolo[2,3-d]pyrimidin-7-yl)pyridin-3-yl]ethenyl]-N-methylquinolin-2-amine (9)

7-[(1E)-2-(5-{5-bromo-4-chloro-7H-pyrrolo[2,3-d]pyrimidin-7-yl}pyridin-3-yl)ethenyl]-N-methylquinolin-2-amine (250 mg, 508 μmol), 1-(2,4-dimethoxyphenyl)methanamine (168 mg, 1.01 mmol), DIEA (326 mg, 2.53 mmol) were mixed in 1,4-dioxane (3 mL). The mixture was heated to 120° C. and stirred for 24 h. The mixture was diluted with water (80 ml) and extracted with EtOAc (80 ml×2). Combined organic layers were washed with H2O (100 ml×2) and brine (60 ml), and dried (Na2SO4). The solvent was removed under reduced pressure to yield the crude product. The crude product was directly used to next step without further purification. ESI LC-MS m/z=622.2 [M+H]+.

### Synthesis of 7-[(1E)-2-[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]ethenyl]-N-methylquinolin-2-amine (10)

7-[(1E)-2-[5-(5-bromo-4-{[(2,4-dimethoxyphenyl)methyl]amino}-7H-pyrrolo[2,3-d]pyrimidin-7-yl)pyridin-3-yl]ethenyl]-N-methylquinolin-2-amine (200 mg, 321 μmol), 1-methyl-3-(4,4,5,5-tetramethyl-1,3,2-dioxaborolan-2-yl)-1H-pyrazole (66.7 mg, 321 μmol), Pd(dppf)Cl2 (26.0 mg, 32.1 μmol), Na2CO3 (102 mg, 963 μmol) were mixed in THF (15 mL) and H2O (5 mL). The mixture was stirred at 75° C. for 12 h. The mixture was diluted with water (80 ml) and extracted with EtOAc (80 ml×2). Combined organic layers were washed with H2O (100 ml×2) and brine (60 ml), and dried (Na2SO4). The solvent was removed under reduced pressure to yield the crude product. The crude product was purified by flash chromatography (silica, EtOAc/PE: 0˜60%) to yield the target compound (80 mg, yield 30%) as a yellow solid. ESI LC-MS m/z=624.3 [M+H]+.

### Synthesis of 7-{2-[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]ethyl}-N-methylquinolin-2-amine (11)

7-[(1E)-2-[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]ethenyl]-N-methylquinolin-2-amine (80.0 mg, 128 μmol), PtO2 (2.90 mg, 12.8 μmol) were mixed in MeOH (20 mL). The mixture was stirred at room temperature under an H2 atmosphere for 2 h. The reaction mixture was filtered and washed with MeOH. The filtrate was concentrated under reduced pressure to yield the target compound (60 mg yield, 95%) as a yellow solid. ESI LC-MS m/z=626.4 [M+H]+.

### Synthesis of compound 7-(2-(5-(4-amino-5-(1-methyl-1H-pyrazol-3-yl)-7H-pyrrolo[2,3-d]pyrimidin-7-yl)pyridin-3-yl)ethyl)-N-methylquinolin-2-amine (12, compound I-1)

7-{2-[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]ethyl}-N-methylquinolin-2-amine (60.0 mg, 95.8 μmol) was added to DCM (3 mL) and TFA (3 mL). The mixture was stirred at room temperature for 4 h. Then the mixture was concentrated, the residue was neutralized with 7M NH3 in methanol solution to pH=8. The mixture was concentrated, the residue was purified by pre-HPLC to yield the target compound (5.8 mg yield 12.7%) as a white solid. ESI LC-MS m/z=476. [M+H]+. 1H NMR (400 MHz, CD3OD) δ 8.85 (s, 1H), 8.33 (s, 1H), 8.09 (s, 1H), 8.01 (s, 1H), 7.80 (d, J=9.0 Hz, 1H), 7.76-7.72 (m, 1H), 7.61 (s, 1H), 7.55 (d, J=8.0 Hz, 1H), 7.45 (s, 1H), 7.12 (d, J=8.1 Hz, 1H), 6.69 (d, J=9.0 Hz, 1H), 6.64-6.59 (m, 1H), 4.07-3.92 (m, 3H), 3.25-3.13 (m, 4H), 3.00 (s, 3H).

### General Method D

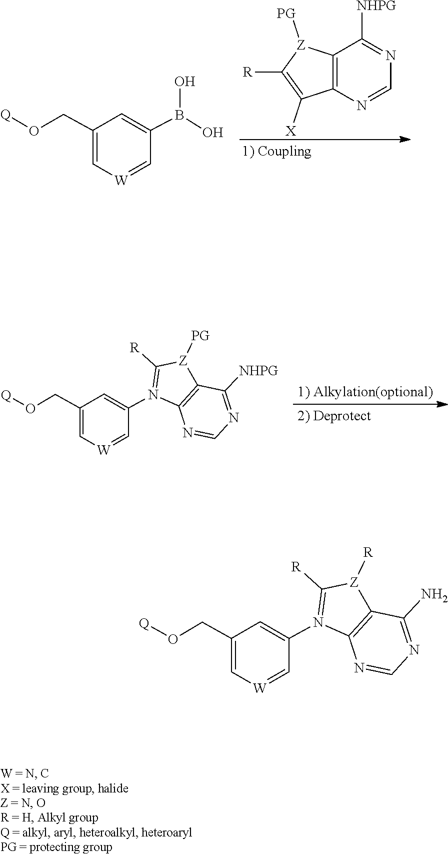

### Method D Example Synthesis

### Example 6: Synthesis of Compound I-47

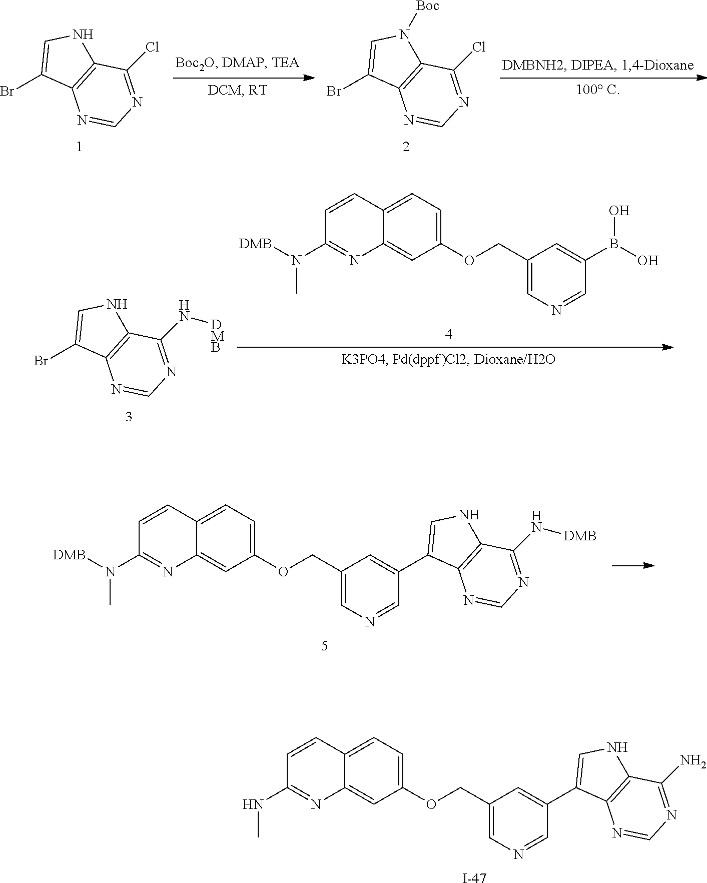

### Synthesis of tert-butyl 7-bromo-4-chloro-5H-pyrrolo[3,2-d]pyrimidine-5-carboxylate (2)

To a solution of 7-bromo-4-chloro-5H-pyrrolo[3,2-d]pyrimidine (500 mg, 2.15 mmol) in DCM (5 mL) was added di-tert-butyl dicarbonate (702 mg, 3.22 mmol), N,N-dimethylpyridin-4-amine (26.2 mg, 0.21 mmol) and triethylamine (895 μL, 6.45 mmol). The reaction mixture was stirred at room temperature overnight. The solvent was removed under reduced pressure. The crude product was purified by flash chromatography (EtOAc/PE: 0-30%) to yield tert-butyl 7-bromo-4-chloro-5H-pyrrolo[3,2-d]pyrimidine-5-carboxylate (510 mg, 1.53 mmol) as a white solid. ESI LCMS m/z=332.0[M+1]+.

### Synthesis of 7-bromo-N-[(2,4-dimethoxyphenyl)methyl]-5H-pyrrolo[3,2-d]pyrimidin-4-amine (3)

To a solution of tert-butyl 7-bromo-4-chloro-5H-pyrrolo[3,2-d]pyrimidine-5-carboxylate (500 mg, 1.50 mmol) in 1,4-dioxane (2 mL) was added 1-(2,4-dimethoxyphenyl)methanamine (627 mg, 3.75 mmol) and ethylbis(propan-2-yl)amine (581 mg, 4.50 mmol). The reaction mixture was heated at 100° C. overnight. The crude product was washed with citric acid aqueous solution, extracted with EtOAc (20 mL×3), dried over Na2SO4, and concentrated under reduced pressure. The crude product was purified by flash chromatography (silica gel, 12 g, EtOAc/PE: 0˜60%) to yield 7-bromo-N-[(2,4-dimethoxyphenyl)methyl]-5H-pyrrolo[3,2-d]pyrimidin-4-amine (348 mg, 0.96 mmol) as a white solid. ESI LCMS m/z=363.1[M+1]+.

### Synthesis of N-[(2,4-dimethoxyphenyl)methyl]-7-{[5-(4-{[(2,4-dimethoxyphenyl)methyl]amino}-5H-pyrrolo[3,2-d]pyrimidin-7-yl)pyridin-3-yl]methoxy}-N-methylquinolin-2-amine (5)

To a solution of 7-bromo-N-[(2,4-dimethoxyphenyl)methyl]-5H-pyrrolo[3,2-d]pyrimidin-4-amine (200 mg, 0.55 mmol) in 1,4-dioxane (4 mL) was added {5-[({2-[methyl(²H)amino]quinolin-7-yl}oxy)methyl]pyridin-3-yl}boronic acid (185 mg, 0.55 mmol), potassium phosphate (350 mg, 1.65 mmol) in water (1 mL) and Pd(dppf)Cl₂ (40.2 mg, 0.05 mmol). The reaction mixture was flushed with nitrogen for 15 min and the heated at 90° C. for 3 h. The resulting reaction mixture was then diluted with water (40 mL), extracted with ethyl acetate (30 mL×3), dried over Na2SO4, filtered, and concentrated under reduced pressure. The crude product was purified by flash chromatography (EtOAc/PE: 0˜60%) to yield N-[(2,4-dimethoxyphenyl)methyl]-7-{[5-(4-{[(2,4-dimethoxyphenyl)methyl]amino}-5H-pyrrolo[3,2-d]pyrimidin-7-yl)pyridin-3-yl]methoxy}-N-methylquinolin-2-amine (78.0 mg, 0.11 mmol) as a yellow solid. ESI LCMS m/z=698.1[M+1]+.

### Synthesis of 7-[(5-{4-amino-5H-pyrrolo[3,2-d]pyrimidin-7-yl}pyridin-3-yl)methoxy]-N-methylquinolin-2-amine (6, compound I-47)

The N-[(2,4-dimethoxyphenyl)methyl]-7-{[5-(4-{[(2,4-dimethoxyphenyl)methyl]amino}-5H-pyrrolo[3,2-d]pyrimidin-7-yl)pyridin-3-yl]methoxy}-N-methylquinolin-2-amine (78 mg, 0.11 mmol) was dissolved in TFA (4 mL). The reaction mixture was stirred at 40° C. for 1 h. The solvent was removed under reduced pressure and neutralized with NH3 in methanol (7 mol/L) to pH>7. The crude product was purified by Prep-HPLC to yield 7-[(5-{4-amino-5H-pyrrolo[3,2-d]pyrimidin-7-yl}pyridin-3-yl)methoxy]-N-methylquinolin-2-amine (15.0 mg, 0.04 mmol) as a white solid. LC-MS m/z=398.0[M+H]+. 1H NMR (400 MHz, CD3OD) δ ppm 9.10 (d, J=1.9 Hz, 1H), 8.65-8.60 (m, 1H), 8.55 (d, J=1.8 Hz, 1H), 8.42 (s, 1H), 8.27 (s, 1H), 8.06 (d, J=9.2 Hz, 1H), 8.03 (s, 1H), 7.74 (d, J=8.8 Hz, 1H), 7.41 (d, J=2.2 Hz, 1H), 7.20 (dd, J=8.8, 2.4 Hz, 1H), 6.80 (d, J=9.2 Hz, 1H), 5.40 (s, 2H), 3.13 (s, 3H).

### General Method E

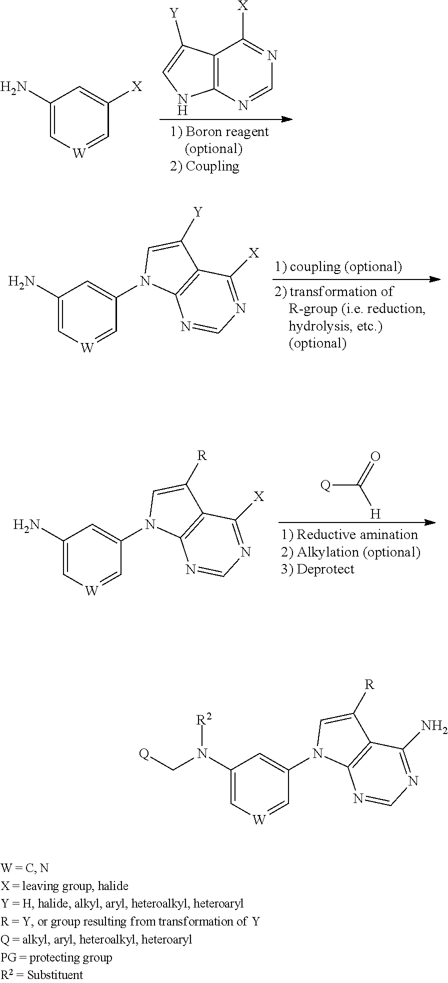

### Method E Example Synthesis

### Example 7: Synthesis of Compound I-72

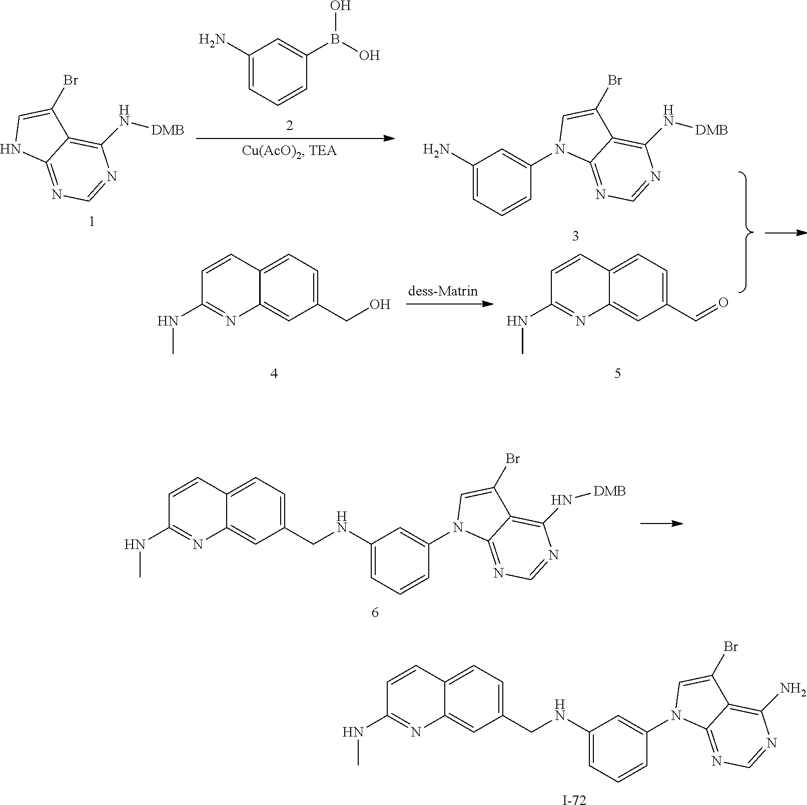

### Synthesis of 7-(3-aminophenyl)-5-bromo-N-[(2,4-dimethoxyphenyl)methyl]-7H-pyrrolo[2,3-d]pyrimidin-4-amine (3)

To a solution of 5-bromo-N-[(2,4-dimethoxyphenyl)methyl]-7H-pyrrolo[2,3-d]pyrimidin-4-amine (500 mg, 1.37 mmol) in DCM (5 mL) was added (3-aminophenyl)boronic acid (187 mg, 1.37 mmol), Cu(AcO)₂; (248 mg, 1.37 mmol) and triethylamine (415 mg, 4.11 mmol). The reaction mixture was stirred at room temperature overnight. The solvent was removed under reduced pressure. The mixture was diluted with water (30 mL), extracted with EtOAc (30 mL×3). The organic layer was washed with brine (50 mL), dried with anhydrous Na2SO4, and concentrated under reduced pressure. The residue was purified by flash chromatography (silica, 20 g, MeOH/DCM: 0˜ 15%) to yield 7-(3-aminophenyl)-5-bromo-N-[(2,4-dimethoxyphenyl)methyl]-7H-pyrrolo[2,3-d]pyrimidin-4-amine (310 mg, 0.6823 mmol) as a light yellow oil. ESI LC-MS m/z=544.1 [M+H]+.

### Synthesis of 2-(methylamino)quinoline-7-carbaldehyde (5)

To a solution of [2-(methylamino)quinolin-7-yl]methanol (690 mg, 3.66 mmol) in DCM (5 mL) Dess-Martin reagent (2.32 g, 5.49 mmol) was added. The reaction mixture was stirred at room temperature for 3 h. The reaction was quenched with saturated aqueous Na2S2O3, and extracted with DCM (20 mL×3). The organic layer was washed with water and brine (50 mL), dried with anhydrous Na2SO4, and concentrated under reduced pressure. The residue was purified by flash chromatography (silica, 12 g, EtOAc/PE: 0˜ 60%) to yield 2-(methylamino)quinoline-7-carbaldehyde (610 mg, 3.27 mmol) as a colorless oil. ESI LC-MS m/z=187.1 [M+H]+.

### Synthesis of 7-({[3-(5-bromo-4-{[(2,4-dimethoxyphenyl)methyl]amino}-7H-pyrrolo[2,3-d]pyrimidin-7-yl)phenyl]amino}methyl)-N-methylquinolin-2-amine (6)

To a solution of 2-(methylamino)quinoline-7-carbaldehyde (100 mg, 0.53 mmol) in methanol (3 mL) was added 7-(3-aminophenyl)-5-bromo-N-[(2,4-dimethoxyphenyl)methyl]-7H-pyrrolo[2,3-d]pyrimidin-4-amine (243 mg, 0.53 mmol) and sodium cyanoborohydride (84.2 mg, 1.34 mmol). The reaction mixture was stirred at room temperature for 4 h. The reaction was quenched with water (1 mL). The mixture was dried with anhydrous Na2SO4, and the filtrate was concentrated under reduced pressure. The residue was purified by Prep-TLC (EtOAc/PE: 20%) to yield 7-({[3-(5-bromo-4-{[(2,4-dimethoxyphenyl)methyl]amino}-7H-pyrrolo[2,3-d]pyrimidin-7-yl)phenyl]amino}methyl)-N-methylquinolin-2-amine (100 mg, 0.16 mmol) as a yellow solid. ESI LC-MS m/z=624.1 [M+H]+.

### Synthesis of 7-{[(3-{4-amino-5-bromo-7H-pyrrolo[2,3-d]pyrimidin-7-yl}phenyl)amino]methyl}-N-methylquinolin-2-amine (7, compound I-72)

The 7-({[3-(5-bromo-4-{[(2,4-dimethoxyphenyl)methyl]amino}-7H-pyrrolo[2,3-d]pyrimidin-7-yl)phenyl]amino}methyl)-N-methylquinolin-2-amine (100 mg, 0.16 mmol) was dissolved in TFA (3 mL). The reaction mixture was stirred at 40° C. for 4 h. Then the mixture was concentrated under reduced pressure, and the residue was neutralized by 3 ml 7M NH3 in MeOH. The crude product was purified by prep-HPLC to yield 7-{[(3-{4-amino-5-bromo-7H-pyrrolo[2,3-d]pyrimidin-7-yl}phenyl)amino]methyl}-N-methylquinolin-2-amine (16.4 mg, 0.03457 mmol) as white solid. ESI LC-MS m/z=476.0 [M+H]⁺. 1H NMR (400 MHz, CD3OD) 8.05 (s, 1H), 7.81 (d, J=8.8 Hz, 1H), 7.68 (s, 1H), 7.57 (d, J=8.1 Hz, 1H), 7.41 (s, 1H), 7.26-7.19 (m, 2H), 6.91-6.89 (m, 1H), 6.80-6.77 (d, 1H), 6.71-6.88 (m, 2H), 4.51 (s, 2H), 3.00 (s, 3H).

### Example 8: Synthesis of Compound I-22

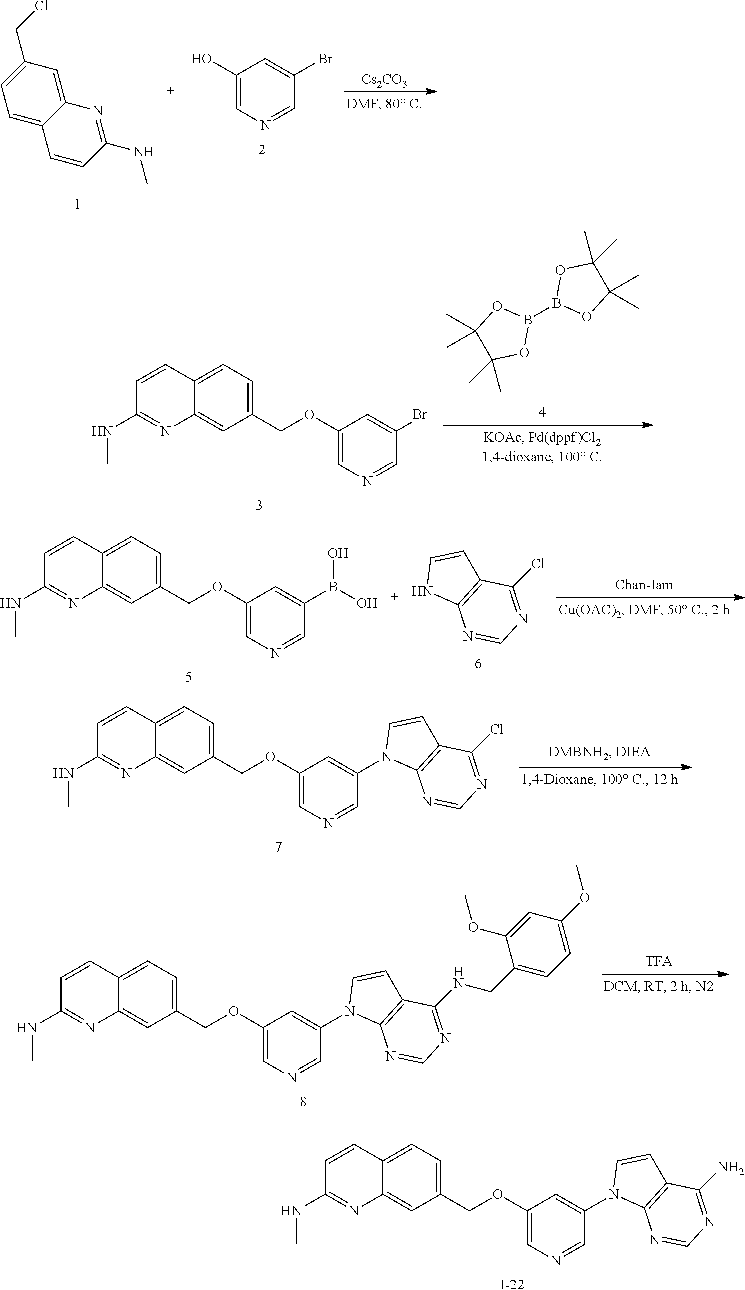

### Synthesis of 7-{[(5-bromopyridin-3-yl)oxy]methyl}-N-methylquinolin-2-amine (3)

7-(chloromethyl)-N-methylquinolin-2-amine (1.0 g, 4.83 mmol), 5-bromopyridin-3-ol (840 mg, 4.83 mmol) and Cs2CO3 (7.85 g, 24.1 mmol) were mixed in DMF (10 mL) and stirred at 80° C. for 3 h. The mixture was diluted with water (50 mL), extracted with EtOAc (30 mL×3). The organic layer was washed with brine (50 mL), and dried with anhydrous Na2SO4. The solution was concentrated under reduced pressure to yield 7-{[(5-bromopyridin-3-yl)oxy]methyl}-N-methylquinolin-2-amine (1.30 g, 3.77 mmol) as a light yellow solid. The crude product was directly used to next step without further purification. ESI LC-MS m/z=345.0 [M+H]+.

### Synthesis of 5-((2-(methylamino)quinolin-7-yl)methoxy)pyridin-3-ylboronic acid (5)

7-((5-bromopyridin-3-yloxy)methyl)-N-methylquinolin-2-amine (1.3 g, 3.77 mmol), 4,4,4′,4′,5,5,5′,5′-octamethyl-2,2′-bi(1,3,2-dioxaborolane) (1.14 g, 4.52 mmol), KOAc (739 mg, 7.54 mmol) and Pd(dppf)Cl2 (27.5 mg, 37.7 μmol) were mixed in 1,4-dioxane (20 mL) and stirred at 100° C. for 12 h under N2. The mixture was concentrated and diluted with EtOAc (100 mL). The EtOAc phase was collected after filter. The filtrate was concentrated under reduced pressure to yield 5-((2-(methylamino)quinolin-7-yl)methoxy)pyridin-3-ylboronic acid (890 mg, 2.87 mmol) as a brown solid. The crude product was directly used to next step without further purification. ESI LC-MS m/z=310.0 [M+H]+.

### Synthesis of 7-{[(5-{4-chloro-7H-pyrrolo[2,3-d]pyrimidin-7-yl}pyridin-3-yl)oxy]methyl}-N-methylquinolin-2-amine (7)

(5-{[2-(methylamino)quinolin-7-yl]methoxy}pyridin-3-yl)boronic acid (890 mg, 2.87 mmol), 4-chloro-7H-pyrrolo[2,3-d]pyrimidine (440 mg, 2.87 mmol), Cu(OAc)2; (677 mg, 3.73 mmol) and 1,10-Phenanthroline (680 mg, 3.73 mmol) were mixed in DMF (10 mL) and stirred at 50° C. for 2 h under air. The mixture was diluted with water (50 ml) and extracted with EtOAc (30 ml×2). Combined organic layers were washed brine (30 ml), and dried with anhydrous Na2SO4. The solvent was removed under reduced pressure to yield crude product. The residue was purified by silica gel column chromatography (silica gel, 20 g, EtOAc/PE: 0˜50%) to yield 7-{[(5-{4-chloro-7H-pyrrolo[2,3-d]pyrimidin-7-yl}pyridin-3-yl)oxy]methyl}-N-methylquinolin-2-amine (200 mg, 2.87 mmol) as a yellow solid. ESI LC-MS m/z=417.1 [M+H]+.

### Synthesis of 7-({[5-(4-{[(2,4-dimethoxyphenyl)methyl]amino}-7H-pyrrolo[2,3-d]pyrimidin-7-yl)pyridin-3-yl]oxy}methyl)-N-methylquinolin-2-amine (8)

7-{[(5-{4-chloro-7H-pyrrolo[2,3-d]pyrimidin-7-yl}pyridin-3-yl)oxy]methyl}-N-methylquinolin-2-amine (200 mg, 479 μmol) and DIEA (30.8 mg, 239 μmol) and 1-(2,4-dimethoxyphenyl)methanamine (120 mg, 718 μmol) were mixed in 1,4-Dioxane (5 mL) and stirred at 100° C. for 12 h. The mixture was concentrated and diluted with water (20 mL), then extracted with EtOAc (20 ml×3). Combined organic layers were washed with brine and dried over anhydrous Na2SO4 and concentrated under reduced pressure. The crude product was directly used to next step without further purification. 7-({[5-(4-{[(2,4-dimethoxyphenyl)methyl]amino}-7H-pyrrolo[2,3-d]pyrimidin-7-yl)pyridin-3-yl]oxy}methyl)-N-methylquinolin-2-amine (30.0 mg, 54.7 μmol) as a brown solid. ESI LC-MS m/z=548.3 [M+H]+.

### Synthesis of 7-{[(5-{4-amino-7H-pyrrolo[2,3-d]pyrimidin-7-yl}pyridin-3-yl)oxy]methyl}-N-methylquinolin-2-amine (9, compound-22)

To a solution of 7-({[5-(4-{[(2,4-dimethoxyphenyl)methyl]amino}-7H-pyrrolo[2,3-d]pyrimidin-7-yl)pyridin-3-yl]oxy}methyl)-N-methylquinolin-2-amine (30 mg, 54.7 μmol) in DCM (5 mL) was added TFA (5 mL). The mixture was stirred at room temperature for 5 h. Then the mixture was concentrated, neutralized by 4 ml 7M NH3 in MeOH, and then concentrated to yield crude product. The residue was purified by Prep-HPLC to yield 7-{[(5-{4-amino-7H-pyrrolo[2,3-d]pyrimidin-7-yl}pyridin-3-yl)oxy]methyl}-N-methylquinolin-2-amine (10.0 mg, 25.1 μmol) as a white solid. ESI LC-MS m/z=398.2 [M+H]+,

1H NMR (400 MHz, DMSO-d6) δ ppm 8.75 (d, J=2.0 Hz, 1H), 8.36 (d, J=2.4 Hz, 1H), 8.16 (s, 1H), 8.12 (t, J=2.2 Hz, 1H), 7.88 (d, J=2.4 Hz, 1H), 7.69-7.63 (m, 3H), 7.26 (t, J=12.0 Hz, 3H), 6.82-6.81 (m, 1H), 6.75 (d, J=7.2 Hz, 1H), 5.39 (s, 2H), 2.90-2.89 (m, 3H).

### General Method F

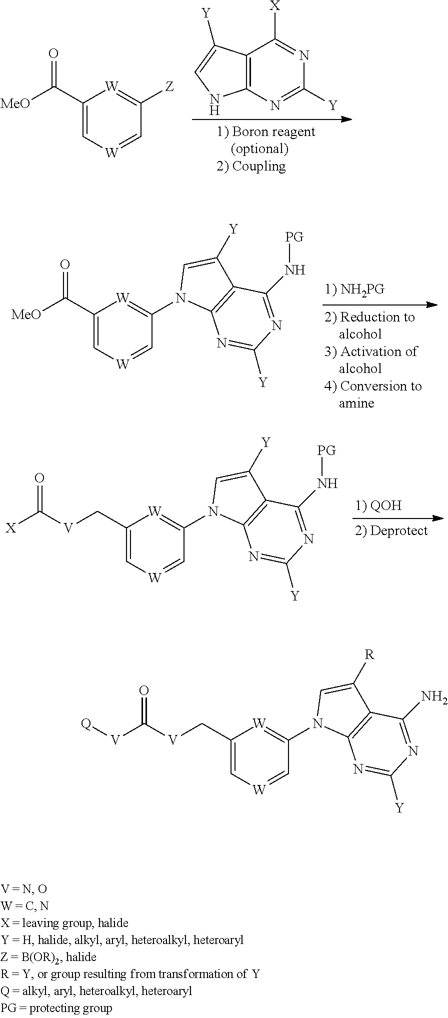

### Method F Example Synthesis

### Example 9: Synthesis of Compound I-224

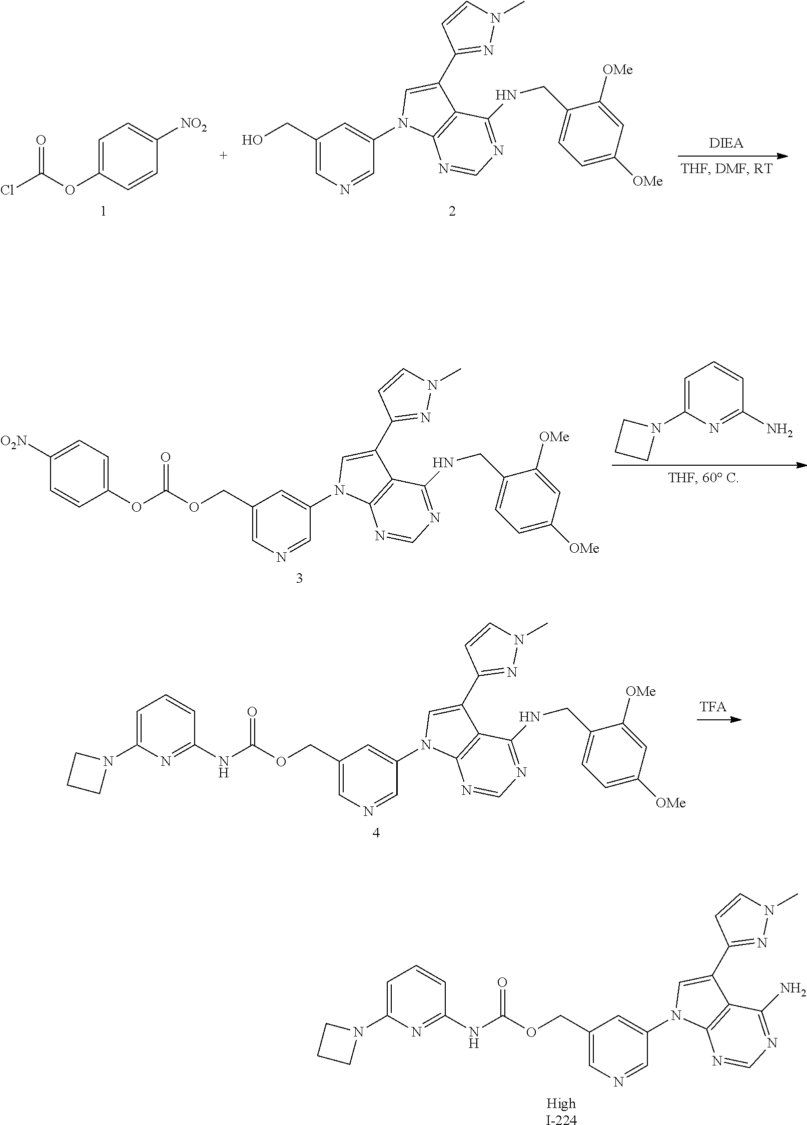

### Synthesis of Compound 3

The solution of 4-nitrophenyl carbonochloridate (170 mg, 848 μmol) in tetrahydrofuran (3 mL) was added to [5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methanol (200 mg, 424 μmol) and N,N-diisopropylethylamine (164 mg, 1.27 mmol) in dimethylformamide (1 mL). The mixture was stirred at rt for 4 h. LC-MS analysis indicated that reaction was well. The mixture was diluted with water (40 mL) and extracted with EA (40 mL×3). The organic layer was separated, washed with H2O (40 mL) and brine (30 mL). The combined organics were dried (Na2SO4) and concentrated to provide the crude product as a mixture of isomers. The cured was purified by silica gel column chromatography to give the target [5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl 4-nitrophenyl carbonate (200 mg, 314 μmol). ESI LCMS m/z=637[M+1]+.

### Synthesis of Compound 4

To a solution of [5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl 4-nitrophenyl carbonate (100 mg, 157 μmol) in tetrahydrofuran (6 mL) was added 6-(azetidin-1-yl)pyridin-2-amine (19.3 mg, 130 μmol) at rt and stirred at 60° C. for 3 days. LC-MS analysis indicated that a small amount of product was got. The mixture was diluted with water (40 mL) and extracted with EA (40 mL×3). The organic layer was separated, washed with H2O (40 mL) and brine (30 mL). Then the mixture was purified by PREP-HPLC to get the target [5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl N-[6-(azetidin-1-yl)pyridin-2-yl]carbamate (8.00 mg, 12.3 μmol), yield: 95%, ESI LCMS m/z=647[M+1]+.

### Synthesis of Compound I-224

[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl N-[6-(azetidin-1-yl)pyridin-2-yl]carbamate (8.00 mg, 12.3 μmol) and trifluoroacetic acid (5 ml) were mixed and stirred at 40° C. for 2 h. The mixture was concentrated under reduced pressure and adjust the pH to 9 with NH3.MeOH. Then the mixture was purified by PREP-HPLC to get the target{5-[4-amino-5-(1-methyl-1H-pyrazol-3-yl)-7H-pyrrolo[2,3-d]pyrimidin-7-yl]pyridin-3-yl}methyl N-[6-(azetidin-1-yl)pyridin-2-yl]carbamate (2.00 mg, 4.02 μmol), yield: 100%, ESI LCMS m/z=497[M+1]+. ¹H NMR (400 MHz, DMSO-d6) δ ppm 9.86 (s, 1H), 9.17 (s, 1H), 9.09 (s, 1H), 8.64 (s, 1H), 8.42 (s, 1H), 8.21 (s, 1H), 8.14 (s, 1H), 7.79 (s, 1H), 7.49-7.39 (m, 2H), 7.08 (d, J=7.6 Hz, 1H), 6.77 (s, 1H), 6.01 (d, J=8.0 Hz, 1H), 5.30 (s, 2H), 3.91 (s, 3H) 3.89-3.87 (m, 4H) 2.34-2.24 (m, 2H).

### Example 10: Synthesis of Compound I-225

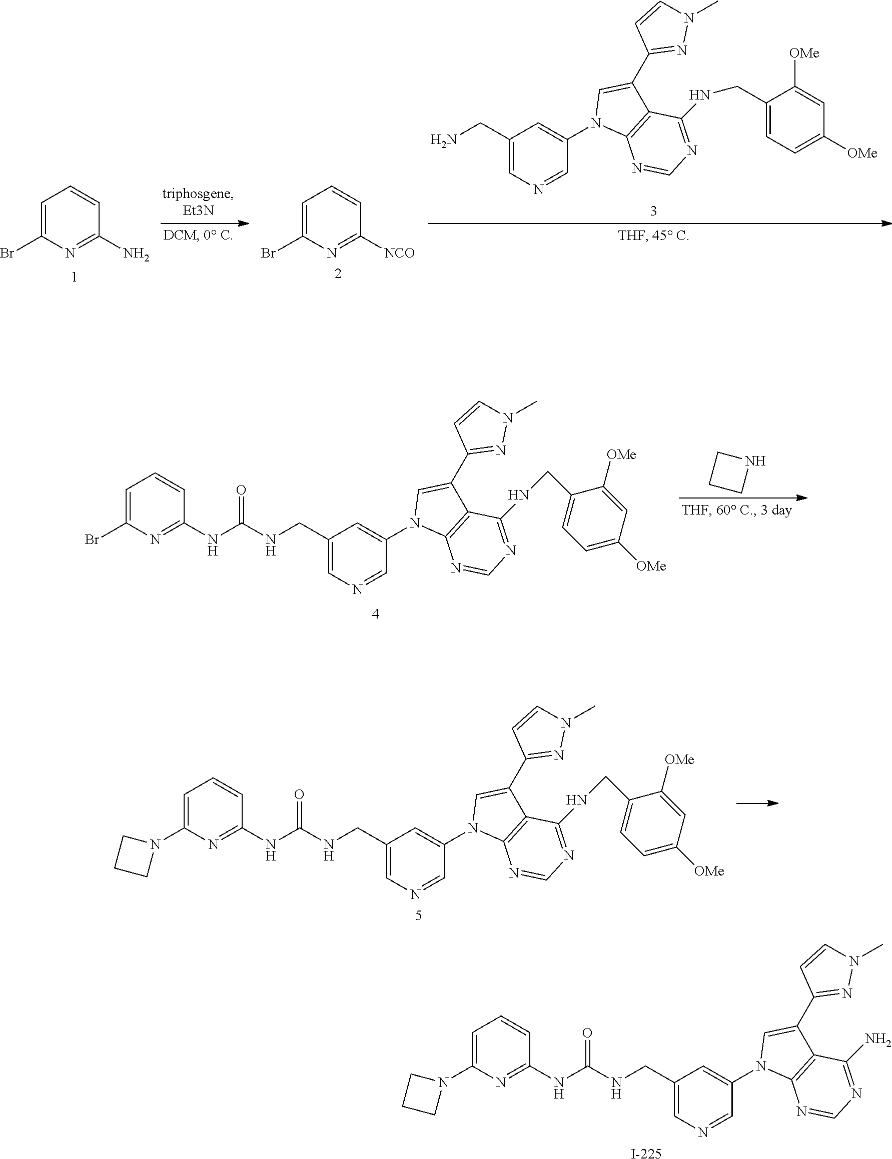

### Synthesis of Compound 2

6-bromopyridin-2-amine (250 mg, 1.44 mmol) was mixed with triethylamine (436 mg, 4.31 mmol) and methylene chloride (1 mL). A solution of triphosgene (510 mg, 1.72 mmol) in methylene chloride (15 mL) was added dropwise to the mixture at 0° C. and stirred at 0° C. for 3 h. LC-MS analysis indicated that reaction was well. Then the mixture was concentrated to get crude2-bromo-6-isocyanatopyridine (250 mg, 1.25 mmol). ESI LCMS m/z=199[M+1]⁺.

### Synthesis of Compound 4

The solution of 2-bromo-6-isocyanatopyridine (200 mg, 1.00 mmol) in trifluoroaceticacid (7 ml) was added to 7-[5-(aminomethyl)pyridin-3-yl]-N-[(2,4-dimethoxyphenyl)methyl]-5-(1-methyl-1Hpyrazol-3-yl)-7H-pyrrolo[2,3-d]pyrimidin-4-amine (564 mg, 1.20 mmol) in dimethylformamide (3 mL) at rt and stirred at 45° C. for 12 h. LC-MS analysis indicated that reaction was well. The mixture was diluted with water (40 mL) and extracted with EA (40 mL×3). The organic layer was separated, washed with H2O (40 mL) and brine (30 mL). The combined organics were dried (Na2SO4) and concentrated to provide the crude product as a mixture of isomers. The cured was purified by silica gel column chromatography to give the target 3-(6-bromopyridin-2-yl)-1-{[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl}urea (100 mg, 149 μmol), yield: 70%, ESI LCMS m/z=671.1[M+1]+.

### Synthesis of Compound 5

To a solution of 3-(6-bromopyridin-2-yl)-1-{[5-(4-{[(2,4 dimethoxyphenyl)methyl]amino}-5-(1-methyl-1H-pyrazol-3-yl)-7H-pyrrolo[2,3-d]pyrimidin-7-yl)pyridin-3-yl]methyl}urea (200 mg, 298 μmol) in tetrahydrofuran (5 ml), azetidine (85.0 mg, 1.49 mmol) was added at rt and stirred at 60° C. for 3 days. LC-MS analysis indicated that reaction was well. The mixture was diluted with water (40 mL) and extracted with EA (40 mL×3). The organic layer was separated, washed with H2O (40 mL) and brine (30 mL). The combined organics were dried (Na2SO4) and concentrated to provide the crude product as a mixture of isomers. The cured was purified by silica gel column chromatography (MeOH:DCM=1:10) to give the target3-[6-(azetidin-1-yl)pyridin-2-yl]-1-{[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl}urea (80.0 mg, 123 μmol), yield: 60%, ESI LCMS m/z=647.3[M+1]+.

### Synthesis of Compound I-225

3-[6-(azetidin-1-yl)pyridin-2-yl]-1-{[5-(4-{[(2,4-dimethoxyphenyl)methyl]amino}-5-(1-methyl-1H-pyrazol-3-yl)-7H-pyrrolo[2,3-d]pyrimidin-7-yl)pyridin-3-yl]methyl}urea (80.0 mg, 123 μmol) and trifluoroacetic acid (5 mL) were mixed and stirred at 40° C. for 3 h. The mixture was concentrated under reduced pressure and adjust the pH to 9 with NH3.MeOH.

Then the mixture was purified by PREP-HPLC to get the target 1-({5-[4-amino-5-(1-methyl-1H-pyrazol-3-yl)-7H-pyrrolo[2,3-d]pyrimidin-7-yl]pyridin-3-yl}methyl)-3-[6-(azetidin-1-yl)pyridin-2-yl]urea (27.6 mg, 55.6 μmol), ESI LCMS m/z=497.1[M+1]+. ¹H NMR (400 MHz, DMSO-d6) δ ppm 9.19 (s, 3H), 9.01 (s, 1H), 8.54 (s, 1H), 8.34 (s, 1H), 8.12 (s, 1H), 8.10 (s, 1H), 7.78 (s, 1H), 7.39-7.34 (m, 2H), 6.76 (s, 1H), 6.39 (d, J=8 Hz, 1H), 5.83 (d, J=8 Hz, 1H), 4.53 (s, 2H), 3.91 (s, 3H), 3.78-3.74 (m, 4H), 2.17-2.11 (m, 2H).

### General Method G

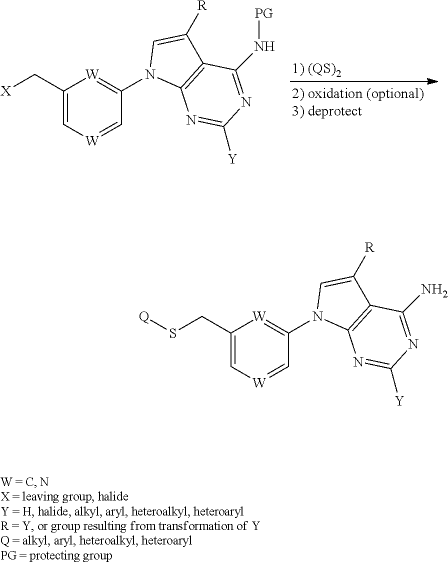

### Method G Example Synthesis

### Example 11: Synthesis of Compound I-219

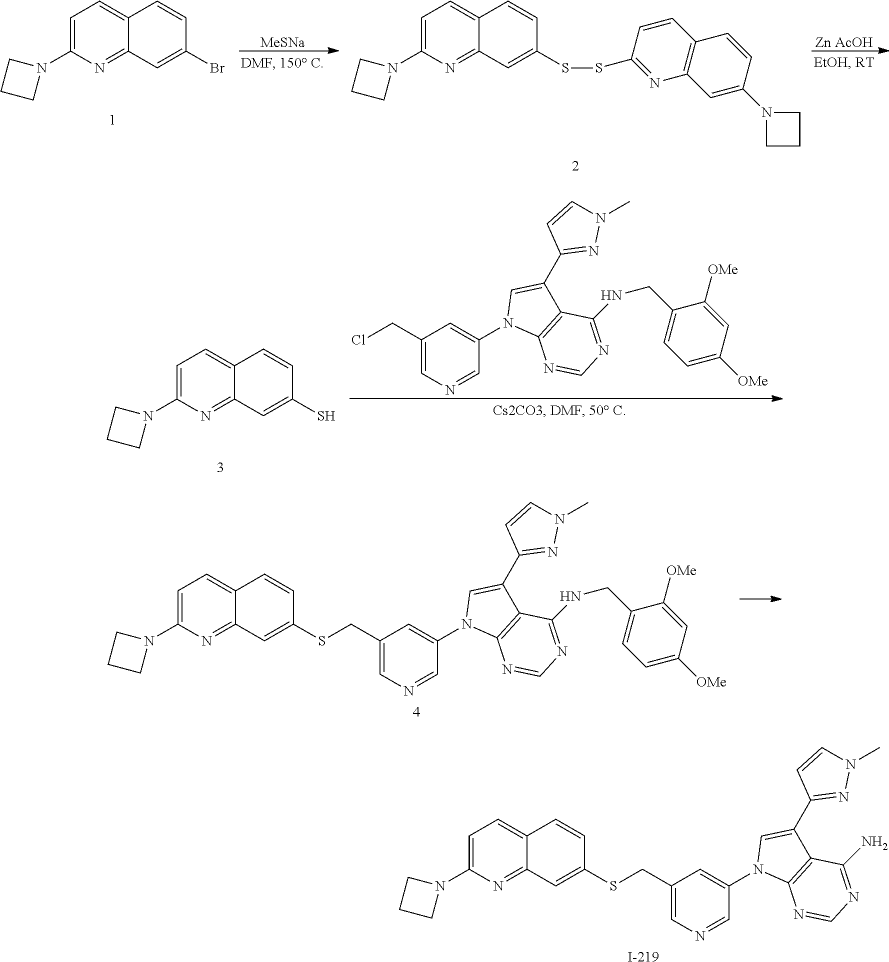

### Synthesis of Compound 2

A mixture of 2-(azetidin-1-yl)-7-bromoquinoline (1 g, 3.80 mmol) in dimethylformamide (6 mL) was added sodium methylsulfanide (1.33 g, 19.0 mmol) at RT. The mixture was heated to 150° C., under N2, for about 12 h. Then cooled to RT, washed with water (10 mL), extracted with EA (20 mL), the organic layer was dried with Na2SO4, filtered and concentrated. The residue was purified by pre-TLC, afford 2-(azetidin-1-yl)-7-{[2-(azetidin-1-yl)quinolin-7-yl]disulfanyl}quinoline (190 mg, 441 μmol) as a yellow solid, LC-MS m/z=431[M+H]⁺.

### Synthesis of Compound 3

A mixture of 2-(azetidin-1-yl)-7-{[7-(azetidin-1-yl)quinolin-2-yl]disulfanyl}quinoline (1 g, 2.32 mmol) and zinc (455 mg, 6.96 mmol) and acetic acid (278 mg, 4.64 mmol) in ethyl alcohol (30 mL) and tetrahydrofuran (30 mL) was stirred at rt for 12 h. then filtered and concentrated to afford 2-(azetidin-1-yl)quinoline-7-thiol (250 mg, 1.15 mmol) as a yellow solid. LC-MS m/z=217.2 [M+H]⁺.

### Synthesis of Compound 4

A mixture of 2-(azetidin-1-yl)quinoline-7-thiol (40 mg, 184 μmol) and 7-[5-(chloromethyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (90.1 mg, 184 μmol) and caesium carbonate (59.9 mg, 184 μmol) in dimethylformamide (3 mL) was stirred at 80° C., under N2, for about 2 h. Then cooled to RT, washed with water (20 mL), extracted with EA (60 mL), the organic layer was dried with Na2SO4, filtered and concentrated, the residue was purified by flash chromatography, eluent:(DCM\MeOH: 10\1), afford 7-[5-({[2-(azetidin-1-yl)quinolin-7-yl]sulfanyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (24.0 mg, 35.8 μmol) as a white solid. LC-MS m/z=670.3 [M+H]⁺.

### Synthesis of Compound I-219

A mixture of 7-[5-({[2-(azetidin-1-yl)quinolin-7-yl]sulfanyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (30 mg, 44.7 μmol) in trifluoroacetic acid (3 mL) was stirred at RT, for about 3 h. Then the mixture was concentrated under reduced pressure, the residue was neutralized with 7M NH3 in MeOH to PH=8, filtered and the residue was concentrated, the residue was purified by pre-HPLC, base condition to afford 7-[5-({[2-(azetidin-1-yl)quinolin-7-yl]sulfanyl}methyl)pyridin-3-yl]-5-(1-methyl-1H-pyrazol-3-yl)-7H-pyrrolo[2,3-d]pyrimidin-4-amine (5.5 mg, 10 umol). LC-MS m/z=520.6 [M+H]⁺. ¹H NMR (400 MHz, DMSO-d) δ ppm 9.24 (br, 1H), 8.98 (d, J=2.4 Hz, 1H), 8.56 (s, 1H), 8.39 (s, 1H), 8.16 (s, 1H), 8.10 (s, 1H), 8.04-8.01 (m, 1H), 7.79 (d, J=2 Hz, 1H), 7.69-7.68 (m, 1H), 7.55 (s, 1H), 7.46 (br, 1H), 7.32-7.28 (m, 1H), 6.74 (d, J=2 Hz, 1H), 6.69-6.68 (m, 1H), 4.51 (s, 2H), 4.15 (br, 4H), 3.91 (s, 3H), 2.39-2.33 (m, 2H).

### Example 12: Synthesis of Compounds I-220 and I-221

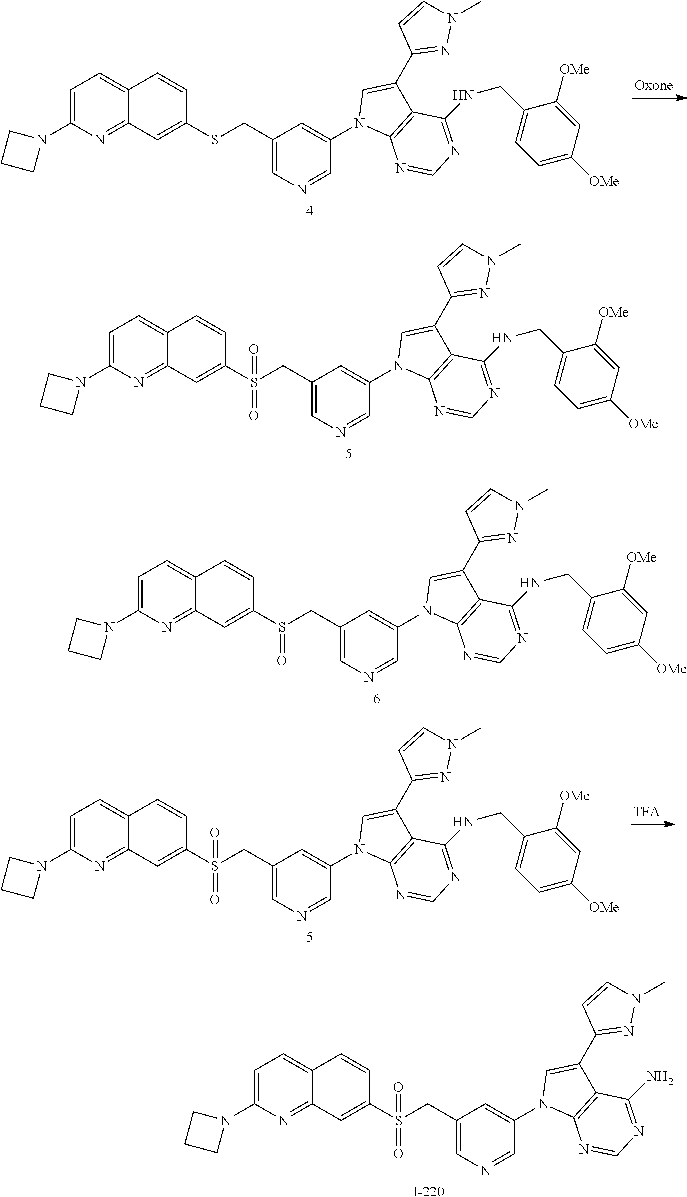

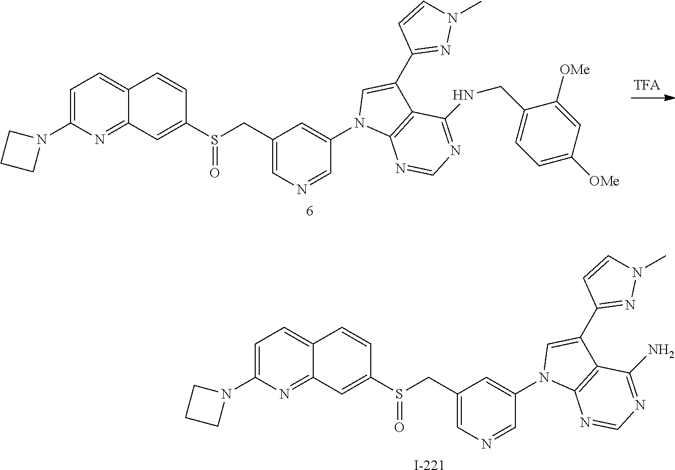

### Synthesis of Compound 5 and 6

A mixture of 7-[5-({[2-(azetidin-1-yl)quinolin-7-yl]sulfanyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (300 mg, 447 μmol) and pentapotassium sulfuric acid diperoxymonosulfate hydrogen sulfate (411 mg, 670 μmol) in tetrahydrofuran (3 mL) and water (3 mL) was stirred at RT, for 4 h. Then the mixture was washed with water (20 mL), extracted with EA (100 mL), the organic layer was dried with Na2SO4, filtered and concentrated. The residue was purified by pre-HPLC, base condition, afford 7-[5-({[2-(azetidin-1-yl)quinolin-7-yl]sulfonyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (72.0 mg, 102 μmol) as a yellow solid. LC-MS m/z=702.2[M+H]⁺. and 7-[5-({[2-(azetidin-1-yl)quinolin-7-yl]sulfinyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (9.50 mg, 13.8 μmol), as a yellow solid. LC-MS m/z=686.2[M+H]⁺.

### Synthesis of Compound I-220

A mixture of 7-[5-({[2-(azetidin-1-yl)quinolin-7-yl]sulfonyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (80 mg, 113 μmol) in trifluoroacetic acid (3 mL) was stirred at RT for about 4 h. Then concentrated under reduced pressure, the residue was purified by pre-HPLC, acid condition, afford 7-[5-({[2-(azetidin-1-yl)quinolin-7-yl]sulfonyl}methyl)pyridin-3-yl]-5-(1-methyl-1H-pyrazol-3-yl)-7H-pyrrolo[2,3-d]pyrimidin-4-amine (10.3 mg, 18.6 μmol), LC-MS m/z=551.4[M+H]⁺. ¹H NMR (400 MHz, DMSO-d) δ ppm 9.14 (br, 1H), 9.05 (d, J=2.4 Hz, 1H), 8.33 (d, J=2 Hz, 1H), 8.12-8.11 (m, 1H), 8.06 (d, J=8 Hz, 1H), 8.00 (s, 1H), 7.97 (s, 1H), 7.91 (d, J=8.4 Hz, 1H), 7.85 (s, 1H), 7.79 (d, J=2.4 Hz, 1H), 7.46 (dd, J=8.0, 1.6 hz, 1H), 7.38 (br, 1H), 6.81 (d, J=9.2 Hz, 1H), 6.69 (d, J=2.4 Hz, 1H), 4.94 (s, 2H), 4.07 (t, J=7.6 Hz, 4H), 3.92 (s, 3H), 2.34-2.30 (m, 2H).

### Synthesis of Compound I-221

A mixture of 7-[5-({[2-(azetidin-1-yl)quinolin-7-yl]sulfinyl}methyl)pyridin-3-yl]-N-[(2,4-dimethoxyphenyl)methyl]-5-(1-methyl-1H-pyrazol-3-yl)-7H-pyrrolo[2,3-d]pyrimidin-4-amine (10 mg, 14.5 μmol) in trifluoroacetic acid (4 mL) was stirred at RT for 4 h. Then the mixture was concentrated under reduced pressure, the residue was neutralized with 7M NH3 in MeOH solution to PH=8, and concentrated, the residue was purified by pre-HPLC, base condition, afford 7-[5-({[2-(azetidin-1-yl)quinolin-7-yl]sulfinyl}methyl)pyridin-3-yl]-5-(1-methyl-1H-pyrazol-3-yl)-7H-pyrrolo[2,3-d]pyrimidin-4-amine (750 μg, 1.40 μmol) as a yellow solid. LC-MS m/z=535.8[M+H]⁺. ¹H NMR (400 MHz, DMSO-d) δ ppm 9.05-8.99 (m, 1H), 8.19-8.16 (m, 4H), 8.20-8.02 (m, 2H), 7.88-7.85 (m, 3H), 7.68-7.67 (m, 1H), 7.56-7.52 (m, 1H), 6.81-6.80 (m, 1H), 6.75-6.74 (m, 1H), 4.93 (s, 2H), 4.25-4.15 (m, 2H), 3.96 (s, 2H), 3.95 (s, 3H), 2.34-2.32 (m, 2H)

### Building Block Synthesis

### Example 13: Synthesis of Building Block 1

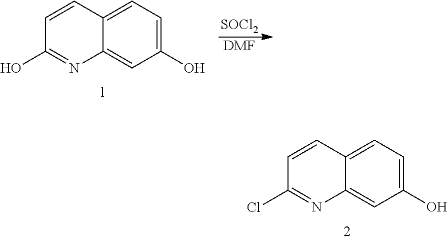

To a solution of compound 1 (120 g, 745 mmol, 1.00 eq) in DMF (1.20 L) was added SOCl₂ (354 g, 2.98 mol, 216 mL, 4.00 eq) at 0° C. The mixture was stirred at 25° C. for 0.5 hr and then stirred at 70° C. for 2 hrs. TLC (Petroleum ether: Ethyl acetate=2:1) showed compound 1 (R_{f}=0.3) was consumed and new spot (Rf=0.5) was detected. The mixture was poured into the water (5.00 L), adjusted to pH=8 with Na2CO3(sat), filtered and the yellow solid was collected. Compound 2 (120 g, 664.8 mmol, 89.2% yield, 99.5% purity) was obtained as yellow solid. LCMS: RT=0.604 min, MS+1=179.8

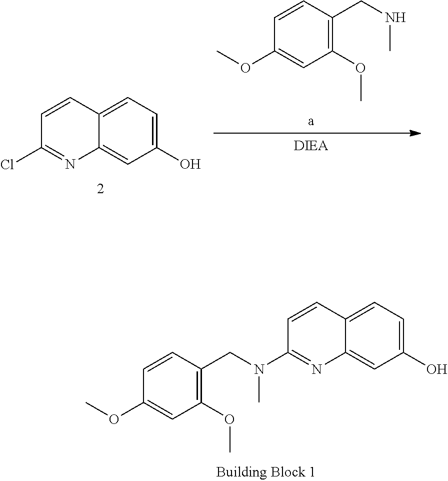

A mixture of compound 2 (150 g, 835 mmol, 1.00 eq), compound a (151 g, 835 mmol, 1.00 eq) and DIEA (324 g, 2.51 mol, 436 mL, 3.00 eq) was stirred at 120° C. for 12 hrs. LCMS showed compound 2 was consumed and desired mass was detected. The mixture was dissolved with MeOH (200 mL), then water (100 mL) and EtOAc (100 mL) were added to the mixture and stirred at 25° C. for 0.5 hrs, then filtered and off-white solid was collected. The crude product was triturated with EtOAc (100 mL) at 25° C. for 1 hr. Building Block 1 (105 g, 311 mmol, 37.2% yield, and 96.0% purity) was obtained as white solid. LCMS: RT=0.785, MS+1=325.0

### Example 14: Synthesis of 2-(pyrrolidin-1-yl)quinolin-7-ol (3)

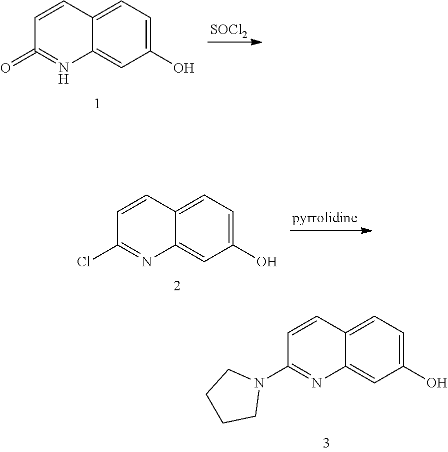

### Synthesis of 2-chloroquinolin-7-ol (2)

To a solution of 7-hydroxy-1,2-dihydroquinolin-2-one (5 g, 31.0 mmol) in DMF (50 mL) was added SOCl₂ (8.96 mL, 124 mmol) at 0° C. The reaction mixture was stirred at 70° C. for 1 h. After cooling to room temperature, the mixture was taken into ice water (300 mL), extracted with EtOAc (200 mL×3) and the combined organic layers were washed with water (100 mL), dried over Na2SO4 and concentrated under reduced pressure to afford 2-chloroquinolin-7-ol (4.5 g, 80.9% yield) as a yellow solid.

### Synthesis of 2-(pyrrolidin-1-yl)quinolin-7-ol (3)

A mixture of 2-chloroquinolin-7-ol (3.5 g, 19.4 mmol) and pyrrolidine (1.51 g, 21.3 mmol) in EtOH (20 mL) was stirred at 80° C. for 48 h. After cooling to room temperature, the reaction mixture was concentrated under reduced pressure. The residue was washed with EtOAc (50 mL) and filtered to afford 2-(pyrrolidin-1-yl)quinolin-7-ol (2.4 g, 57.8% yield) as a yellow solid.

### Example 15: Synthesis of Building Block 2

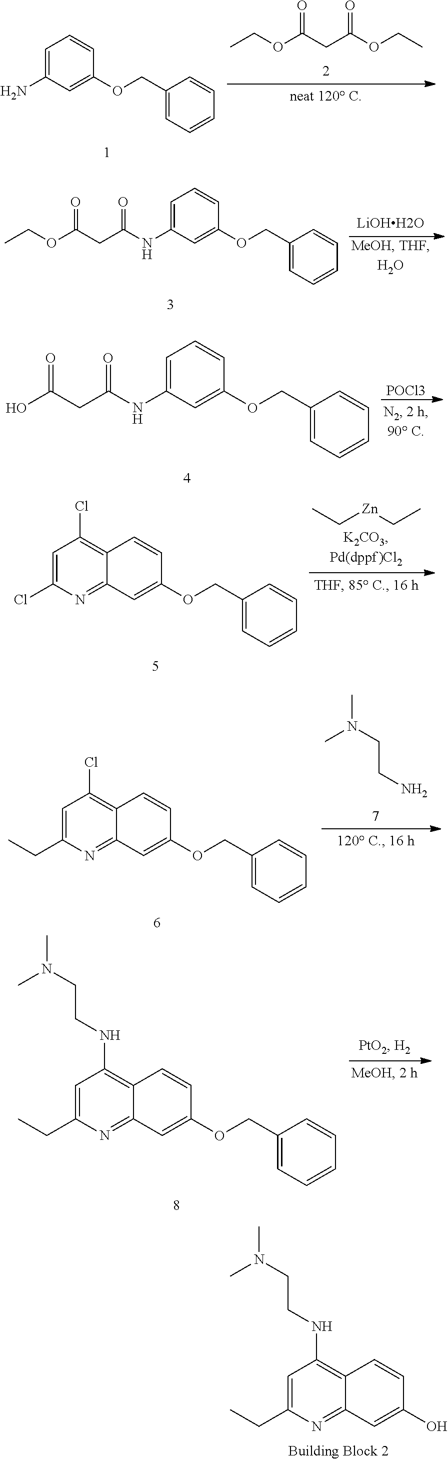

### Synthesis of 2-{[3-(benzyloxy)phenyl]carbamoyl}acetate (3)

3-(benzyloxy)aniline (5.0 g, 25.0 mmol) and 1,3-diethyl propanedioate (16.0 g, 100 mmol) were mixed and stirred at 120° C. for 13 h. The mixture was concentrated under reduced pressure and purified by silica gel column chromatography (silica, 120 g, EtOAc/PE: 0˜30%) to yield ethyl 2-{[3-(benzyloxy)phenyl]carbamoyl}acetate (5.00 g, 15.9 mmol) as a colorless oil. ESI LC-MS m/z=314.1 [M+H]+

### Synthesis of 2-{[3-(benzyloxy)phenyl]carbamoyl}acetic acid (4)

Methyl 2-{[3-(benzyloxy)phenyl]carbamoyl}acetate (5.0 g, 15.9 mmol), LiOH.H2O (839 mg, 20.0 mmol) were mixed in MeOH (30 mL), THE (30 mL) and H2O (30 mL). The mixture was stirred at 50° C. for 12 h. The solvent was removed under reduced pressure. The water solution was adjust to pH=6 by HCl aq. White solid was precipitated, filter to yield the solid and washed with water. The solid was collected and dried to yield 2-{[3-(benzyloxy)phenyl]carbamoyl}acetic acid (3.00 g, 10.5 mmol) as a white solid. ESI LC-MS m/z=286.1 [M+H]+.

### Synthesis of 7-(benzyloxy)-2,4-dichloroquinoline (5)

To a solution of 2-{[3-(benzyloxy)phenyl]carbamoyl}acetic acid (3.0 g, 10.5 mmol) and POCl3 (10 mL) and stirred at 90° C. for 2 h. The solvent was removed under reduced pressure. The mixture was diluted with water (50 mL) and extracted with EtOAc (50 mL×2). The organic layer was separated, washed with brine (20 mL), and dried with anhydrous Na2SO4. The solution was concentrated under reduced pressure to yield crude product. The residue was purified by silica gel column chromatography (silica, 40 g, EtOAc/PE: 0˜30%) to yield 7-(benzyloxy)-2,4-dichloroquinoline (2.5 g, 8.21 mmol) as a light yellow solid. ESI LC-MS m/z=305.0 [M+H]+.

### Synthesis of 7-(benzyloxy)-4-chloro-2-ethylquinoline (6)

A mixture of 7-(benzyloxy)-2,4-dichloroquinoline (2.0 g, 6.57 mmol), diethyl-zinc (6.7 ml, 1M in hexane, 1.02 eq), K2CO3 (2.72 g, 19.7 mmol) and Pd(dppf)Cl2 (50 mg) in THE (50 mL) was purged with N2 for 5 min before heating to reflux. After 16 h, the reaction was quenched with NH₄Cl aq. and extracted with EtOAc (50 ml×3). The organic layer was washed with water and brine (50 mL), and dried with anhydrous Na2SO4 and concentrated under reduced pressure. The residue was purified by silica gel column chromatography (silica, 40 g, EtOAc/PE: 0˜30%) to yield 7-(benzyloxy)-4-chloro-2-ethylquinoline (1.40 g, 4.70 mmol) as a white solid. ESI LC-MS m/z=298.1 [M+H]+.

### Synthesis of 7-(benzyloxy)-N-[2-(dimethylamino)ethyl]-2-ethylquinolin-4-amine (8)

7-(benzyloxy)-4-chloro-2-ethylquinoline (300 mg, 1.00 mmol) and (2-aminoethyl)dimethylamine (1.76 g, 20.0 mmol) were mixed and stirred at 120° C. for 48 h. The mixture was diluted with water (50 mL) and extracted with EtOAc (50 mL×2). The organic layer was separated, washed with H2O (20 mL) and brine (20 mL), and dried with anhydrous Na2SO4. The solution was concentrated under reduced pressure to 7-(benzyloxy)-N-[2-(dimethylamino)ethyl]-2-ethylquinolin-4-amine (200 mg, 572 μmol) as a yellow oil. The crude product was directly used to next step without further purification. ESI LC-MS m/z=350.1 [M+H]+.

### Synthesis of 4-{[2-(dimethylamino)ethyl]amino}-2-ethylquinolin-7-ol (Building Block 2)

A mixture of 7-(benzyloxy)-N-[2-(dimethylamino)ethyl]-2-ethylquinolin-4-amine (300 mg, 858 μmol) and PtO2 (50 mg) was stirred at room temperature for 2 h. The mixture was filtered through celite and the filtrate was concentrated to yield 4-{[2-(dimethylamino)ethyl]amino}-2-ethylquinolin-7-ol (200 mg, 771 μmol) as a yellow oil. ESI LC-MS m/z=260.1 [M+H]+.

### Example 16: Synthesis of N-{7H-pyrrolo[2,3-d]pyrimidin-4-yl}benzamide (13)

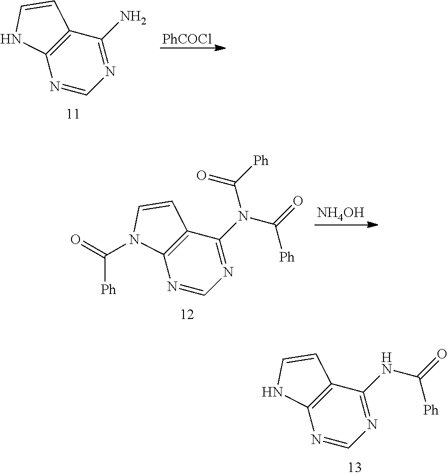

### Synthesis of N-benzoyl-N-{7-benzoyl-7H-pyrrolo[2,3-d]pyrimidin-4-yl}benzamide (12)

A solution of 7H-pyrrolo[2,3-d]pyrimidin-4-amine (10 g, 74.5 mmol) and triethylamine (56.4 g, 558 mmol) in CH₃CN (200 mL) was stirred at 25° C. for 1 h. Benzoyl chloride (52.2 g, 372 mmol) was added and the reaction mixture was stirred at 25° C. for 16 h. The reaction mixture was filtered and the filtrate was concentrated under reduced pressure to afford N-benzoyl-N-{7-benzoyl-7H-pyrrolo[2,3-d]pyrimidin-4-yl}benzamide (13 g, 39.1%) as a brown solid.

### Synthesis of N-{7H-pyrrolo[2,3-d]pyrimidin-4-yl}benzamide (13)

A mixture of N-benzoyl-N-{7-benzoyl-7H-pyrrolo[2,3-d]pyrimidin-4-yl}benzamide (20.5 g, 45.9 mmol) in NH₄OH (80 mL) was stirred at 25° C. for 1 h. The reaction mixture was concentrated under reduced pressure then filtered to afford N-{7H-pyrrolo[2,3-d]pyrimidin-4-yl}benzamide (10.4 g, 95%) as a white solid.

Compounds in Table 1 were prepared according to general methods A-G shown above using above shown building blocks and experimental procedures. MS and ¹H NN/R data are shown below.

TABLE 1

| | | | General |
|---|---|---|---|
| | | | method |
| | MS (ESI) | | of |
| Compound No. | [M + H]⁺ | ¹H NMR | synthesis |
| 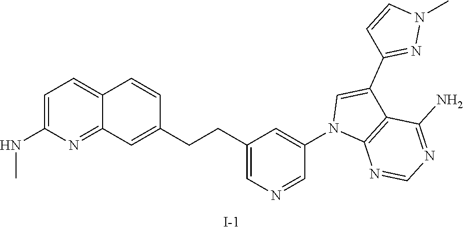 [SMILES: CNc1ccc2ccc(CCc3cncc(-n4cc(-c5ccn(C)n5)c5c(N)ncnc54)c3)cc2n1] | 476.0 | ¹H NMR (400 MHz, CD3OD) δ 8.85 (s, 1H), 8.33 (s, 1H), 8.09 (s, 1H), 8.01 (s, 1H), 7.80 (d, J = 9.0 Hz, 1H), 7.76-7.72 (m, 1H), 7.61 (s, 1H), 7.55 (d, J = 8.0 Hz, 1H), 7.45 (s, 1H), 7.12 (d, J = 8.1 Hz, 1H), 6.69 (d, J = 9.0 Hz, 1H), 6.64-6.59 (m, 1H), 4.07-3.92 (m, 3H), 3.25-3.13 (m, 4H), 3.00 (s, 3H). | C |
| 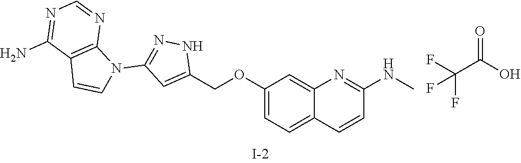 | 387.2 | ¹H NMR (400 MHz, DMSO-d6) δ ppm 13.33 (s, 1H), 9.55 (s, 2H), 8.39-8.15 (m, 3H), 7.92-7.77 (m, 2H), 7.53 (s, 1H), 7.23 (d, J = 9.2 Hz, 1H), 7.08 (s, 1H), 7.01-6.85 (m, 2H), 5.35 (s, 2H), 3.10 (d, J = 4.8 Hz, 3H). | B |
| 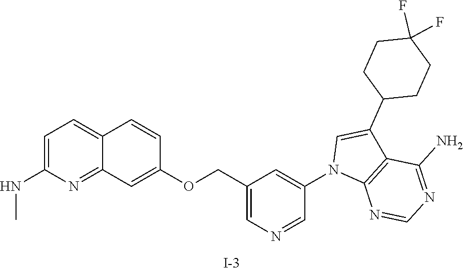 [SMILES: CNc1ccc2ccc(OCc3cncc(-n4cc(C5CCC(F)(F)CC5)c5c(N)ncnc54)c3)cc2n1] | 516.1 | ¹H NMR (400 MHz, DMSO-d6) δ ppm 9.11 (d, J = 2.4 Hz, 1H), 8.63 (d, J = 1.6 Hz, 1H), 8.44 (d, J = 2.1 Hz, 1H), 8.14 (s, 1H), 7.74 (d, J = 8.8 Hz, 1H), 7.57 (s, 1H), 7.53 (d, J = 8.8 Hz, 1H), 7.10 (d, J = 2.4 Hz, 1H), 6.93 (d, J = 4.9 Hz, 1H), 6.90-6.82 (m, 3H), 6.58 (d, J = 8.9 Hz, 1H), 5.34 (s, 2H), 3.30 (s, 1H), 2.88 (d, J = 4.7 Hz, 3H), 2.10-2.06 (m, 6H), 1.69-1.65 (m, 2H). | A |
| 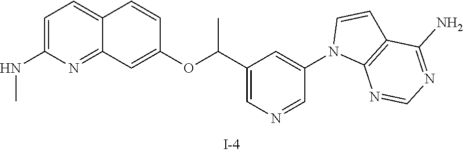 [SMILES: CNc1ccc2ccc(OC(C)c3cncc(-n4ccc5c(N)ncnc54)c3)cc2n1] | 412.1 | ¹H NMR (400 MHz, DMSO-d6) δ ppm 9.04 (d, J = 2.4 Hz, 1H), 8.68 (d, J = 2 Hz, 1H), 8.43 (t, J = 2 Hz, 1H), 8.17 (s, 1H), 7.73-7.71 (m, 2H), 7.52 (d, J = 8.8 Hz, 1H), 7.25 (s, 2H), 6.98 (d, J = 2.4 Hz, 1H), 6.93-6.92 (m, 2H), 6.90 (d, J = 4 Hz, 1H), 6.58 (d, J = 8.8 Hz, 1H), 2.86 (d, J = 4.8 Hz 3H), 1.73 (d, J = 6.8 Hz, 3H). | A |
| 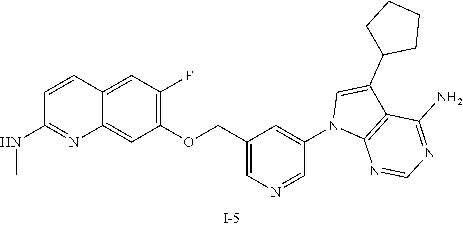 [SMILES: CNc1ccc2cc(F)c(OCc3cncc(-n4cc(C5CCCC5)c5c(N)ncnc54)c3)cc2n1] | 484.1 | ¹H NMR (400 MHz, DMSO-d₆) δ ppm 9.15 (d, J = 2.0 Hz, 1H), 8.67 (d, J = 2.0 Hz, 1H), 8.50 (t, J = 1.2 Hz, 1H), 8.37 (d, J = 5.2 Hz, 1H), 8.13 (s, 1H), 8.04 (d, J = 13.2 Hz, 1H), 7.56 (t, J = 8.8 Hz, 2H), 7.17 (d, J = 4.4 Hz, 1H), 6.76 (s, 1H), 6.36 (d, J = 5.6 Hz, 1H), 5.47 (s, 2H), 2.89 (d, J = 4.8 Hz, 3H), 2.11-2.09 (m, 2H), 1.76-1.75 (m, 6H). | B |
| 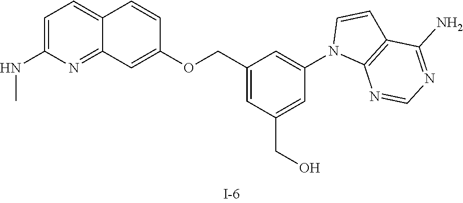 [SMILES: CNc1ccc2ccc(OCc3cc(CO)cc(-n4ccc5c(N)ncnc54)c3)cc2n1] | 427.1 | ¹H NMR (400 MHz, DMSO-d₆) δ ppm 8.13 (s, 1H), 7.80 (s, 1H), 7.74-7.72 (m, 2H), 7.55-7.51 (m, 2H), 7.42 (s, 1H), 7.15 (s, 2H), 7.06 (d, J = 2.4 Hz, 1H), 6.98-6.83 (m, 2H), 6.79 (d, J = 3.7 Hz, 1H), 6.57 (d, J = 8.8 Hz, 1H), 5.37 (t, J = 5.7 Hz, 1H), 5.28 (s, 2H), 4.61 (d, J = 5.5 Hz, 2H), 2.87 (d, J = 4.8 Hz, 3H). | B |
| 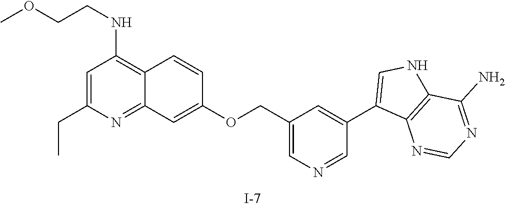 [SMILES: CCc1cc(NCCOC)c2ccc(OCc3cncc(-c4c[nH]c5c(N)ncnc45)c3)cc2n1] | 470.2 | ¹H NMR (400 MHz, DMSO-d6) δ ppm 11.36 (s, 1H), 9.32 (d, J = 1.9 Hz, 1H), 8.68 (s, 1H), 8.52 (s, 1H), 8.24 (s, 1H), 8.20 (s, 1H), 8.08 (d, J = 9.3 Hz, 1H), 7.27 (d, J = 2.6 Hz, 1H), 7.08 (dd, J = 9.1, 2.6 Hz, 1H), 6.96-6.94 (m, 1H), 6.83 (s, 1H), 6.29 (s, 1H), 5.31 (s, 2H), 3.60-3.57 (m, 2H), 3.49-3.39 (m, 2H), 3.30 (s, 3H), 2.75-2.61 (m, 2H), 1.25 (t, J = 7.6 Hz, 3H). | D |
| 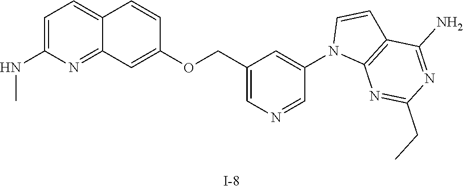 [SMILES: CCc1nc(N)c2ccn(-c3cncc(COc4ccc5ccc(NC)nc5c4)c3)c2n1] | 426.1 | ¹H NMR (400 MHz, DMSO-d₆) δ ppm 9.12 (d, J = 2.0 Hz, 1H), 8.64 (d, J = 1.6 Hz, 1H), 8.53 (t, J = 4.0 Hz, 1H), 7.74 (d, J = 8.8 Hz, 1H), 7.63 (d, J = 3.6 Hz, 1H), 7.53 (d, J = 8.8 Hz, 1H), 7.09-7.10 (m, 3H), 6.93 (d, J = 4.8 Hz, 1H), 6.88 (dd, J = 8.8, 2.4 Hz, 1H), 6.78 (d, J = 3.6 Hz, 1H), 6.58 (d, J = 9.2 Hz, 1H), 5.37 (s, 2H), 2.87 (d, J = 4.4 Hz, 3H), 2.67-2.61 (m, 2H), 1.23 (t, J = 7.6 Hz, 3H). | A |
| 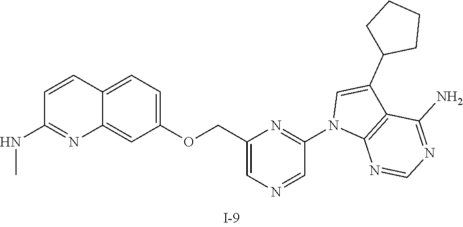 [SMILES: CNc1ccc2ccc(OCc3cncc(-n4cc(C5CCCC5)c5c(N)ncnc54)n3)cc2n1] | 467.2 | ¹H NMR (400 MHz, DMSO-d₆) δ ppm 9.97 (s, 1H), 8.67 (s, 1H), 8.25 (s, 1H), 7.76 (d, J = 8.8 Hz, 1H), 7.65 (s, 1H), 7.55 (d, J = 8.8 Hz, 1H), 7.12 (s, 1H), 6.96-6.92 (m, 2H), 6.86 (br, 1H), 6.59 (d, J = 8.8 Hz, 1H), 5.44 (s, 2H), 2.87 (d, J = 4.8 Hz, 3H), 2.07-2.04 (m, 2H), 1.71-1.69 (m, 4H), 1.53-1.51 (m, 2H). | A |
| 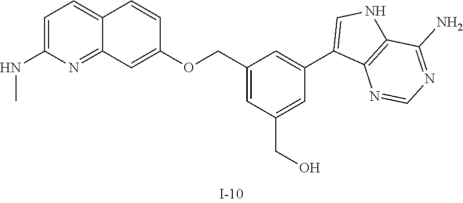 [SMILES: CNc1ccc2ccc(OCc3cc(CO)cc(-c4c[nH]c5c(N)ncnc45)c3)cc2n1] | 427.1 | ¹H NMR (400 MHz, DMSO-d₆) δ ppm 8.22 (s, 1H), 8.16 (s, 1H), 8.09 (s, 1H), 8.05 (s, 1H), 7.74 (d, J = 8.9 Hz, 1H), 7.52 (d, J = 8.8 Hz, 1H), 7.26 (s, 1H), 7.06 (d, J = 2.4 Hz, 1H), 6.96-6.90 (m, 1H), 6.88-6.86 (m, 1H), 6.82 (s, 1H), 6.57 (d, J = 8.8 Hz, 1H), 5.21 (s, 3H), 4.56 (s, 2H), 2.87 (d, J = 4.7 Hz, 3H). | D |
| 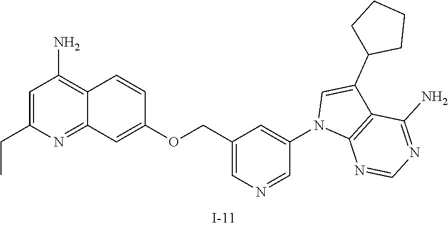 [SMILES: CCc1cc(N)c2ccc(OCc3cncc(-n4cc(C5CCCC5)c5c(N)ncnc54)c3)cc2n1] | 480.3 | ¹H NMR (400 MHz, DMSO) δ ppm 9.11 (d, J = 2.3 Hz, 1H), 8.64 (d, J = 1.5 Hz, 1H), 8.45 (d, J = 2.0 Hz, 1H), 8.13 (s, 1H), 8.01 (d, J = 9.2 Hz, 1H), 7.50 (s, 1H), 7.24 (d, J = 2.6 Hz, 1H), 7.07-7.04 (m, 1H), 6.74 (s, 2H), 6.57 (s, 2H), 6.34 (s, 1H), 5.36 (s, 2H), 2.66-2.61 (m, 2H), 2.10-2.05 (m, 2H), 1.77-1.61 (m, 6H), 1.24-1.21 (m, 4H). | B |
| 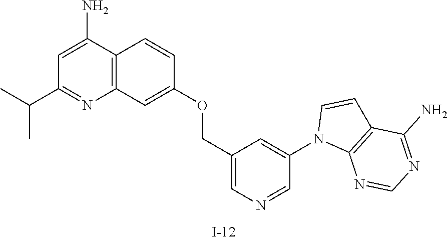 [SMILES: CC(C)c1cc(N)c2ccc(OCc3cncc(-n4ccc5c(N)ncnc54)c3)cc2n1] | 426.0 | ¹H NMR (400 MHz, DMSO-d₆) δ ppm 9.07 (d, J = 2.5 Hz, 1H), 8.68 (d, J = 1.7 Hz, 1H), 8.48-8.44 (m, 1H), 8.15 (s, 1H), 8.01 (d, J = 9.2 Hz, 1H), 7.70 (d, J = 3.7 Hz, 1H), 7.28-7.19 (m, 3H), 7.06 (dd, J = 9.1, 2.6 Hz, 1H), 6.84 (d, J = 3.7 Hz, 1H), 6.55 (s, 2H), 6.35 (s, 1H), 5.39 (s, 2H), 2.91-2.87 (m, 1H), 1.23 (d, J = 6.9 Hz, 6H). | B |
| 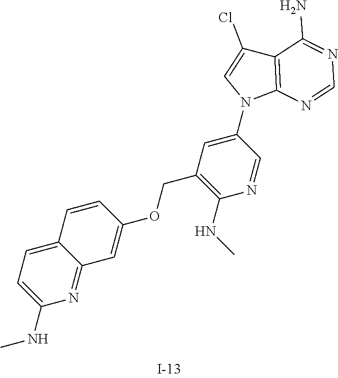 [SMILES: CNc1ccc2ccc(OCc3cc(-n4cc(Cl)c5c(N)ncnc54)cnc3NC)cc2n1] | 461.2 | ¹H NMR (400 MHz, DMSO-d₆) δ ppm 8.26 (d, J = 2.6 Hz, 1H), 8.10 (s, 1H), 7.87 (d, J = 2.6 Hz, 1H), 7.74 (d, J = 8.8 Hz, 1H), 7.67 (s, 1H), 7.52 (d, J = 8.8 Hz, 1H), 7.17 (d, J = 2.4 Hz, 1H), 7.04-6.92 (m, 2H), 6.88 (dd, J = 8.7, 2.5 Hz, 1H), 6.64-6.52 (m, 2H), 5.06 (s, 2H), 2.93 (d, J = 4.5 Hz, 3H), 2.88 (d, J = 4.7 Hz, 3H). | B |
| 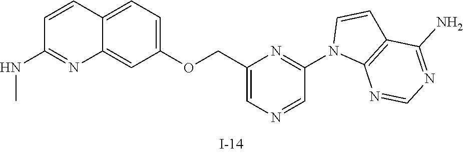 [SMILES: CNc1ccc2ccc(OCc3cncc(-n4ccc5c(N)ncnc54)n3)cc2n1] | 399.1 | ¹H NMR (400 MHz, DMSO-d₆) δ ppm 9.99 (s, 1H), 8.73 (s, 1H), 8.27 (s, 1H), 8.20 (s, 0.3H), 7.97 (d, J = 3.6 Hz, 1H), 7.75 (d, J = 8.8 Hz, 1H), 7.55 (d, J = 8.8 Hz, 1H), 7.36 (br, 2H), 7.14 (s, 1H), 6.96-6.90 (m, 3H), 6.59 (d, J = 8.8 Hz, 1H), 5.41 (s, 2H), 2.87 (d, J = 4.4 Hz, 3H). | A |
| 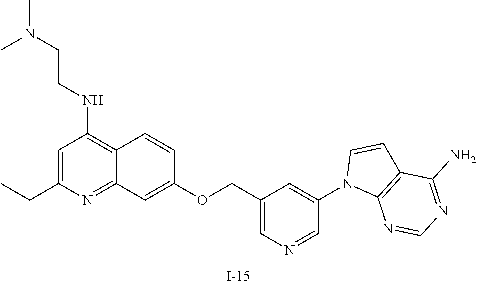 [SMILES: CCc1cc(NCCN(C)C)c2ccc(OCc3cncc(-n4ccc5c(N)ncnc54)c3)cc2n1] | 483.0 | ¹H NMR (400 MHz, DMSO-d₆) δ ppm 9.08 (d, J = 2.5 Hz, 1H), 8.69 (d, J = 1.7 Hz, 1H), 8.47-8.46 (m, 1H), 8.16 (s, 1H), 8.03 (d, J = 9.3 Hz, 1H), 7.70 (d, J = 3.7 Hz, 1H), 7.31-7.19 (m, 3H), 7.12-7.09 (m, 1H), 6.85-6.78 (m, 2H), 6.28 (s, 1H), 5.39 (s, 2H), 2.73-2.67 (m, 2H), 2.22 (d, J = 3.8 Hz, 6H), 1.26 (t, J = 7.6 Hz, 3H). | B |
| 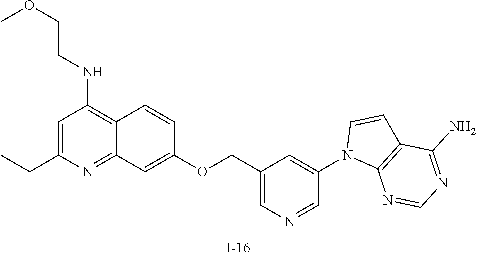 [SMILES: CCc1cc(NCCOC)c2ccc(OCc3cncc(-n4ccc5c(N)ncnc54)c3)cc2n1] | 470.1 | ¹H NMR (400 MHz, CD3OH) δ 8.32 ppm 9.01 (s, 1H), 8.73 (s, 1H), 8.44 (s, 1H), 8.17 (s, 1H), 8.03 (d, J = 9.2 Hz, 1H), 7.57 (d, J = 3.7 Hz, 1H), 7.32 (d, J = 2.5 Hz, 1H), 7.32-7.19 (m, 1H), 6.87 (d, J = 3.7 Hz, 1H), 6.45 (s, 1H), 5.44 (s, 2H), 3.72 (t, J = 5.5 Hz, 2H), 3.59 (t, J = 5.5 Hz, 2H), 3.43 (s, 3H), 2.86-2.78 (m, 2H), 1.36 (t, J = 7.6 Hz, 3H). | B |
| 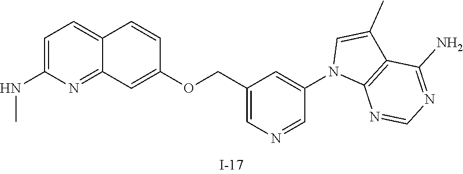 [SMILES: CNc1ccc2ccc(OCc3cncc(-n4cc(C)c5c(N)ncnc54)c3)cc2n1] | 412.1 | ¹H NMR (400 MHz, DMSO) δ ppm 9.05 (d, J = 2.5 Hz, 1H), 8.63 (d, J = 1.7 Hz, 1H), 8.42 (t, J = 2.1 Hz, 1H), 8.12 (s, 1H), 7.74 (d, J = 8.9 Hz, 1H), 7.53 (d, J = 8.7 Hz, 1H), 7.48 (d, J = 1.1 Hz, 1H), 7.10 (d, J = 2.4 Hz, 1H), 6.95 (d, J = 4.6 Hz, 1H), 6.88 (dd, J = 8.7, 2.5 Hz, 1H), 6.80 (s, 2H), 6.58 (d, J = 8.8 Hz, 1H), 5.35 (s, 2H), 2.88 (d, J = 4.7 Hz, 3H), 2.43 (d, J = 0.9 Hz, 3H). | A |
| 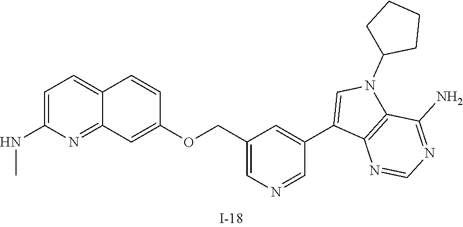 [SMILES: CNc1ccc2ccc(OCc3cncc(-c4cn(C5CCCC5)c5c(N)ncnc45)c3)cc2n1] | 466.0 | ¹H NMR (400 MHz, DMSO-d₆) δ ppm 9.40 (d, J = 2.0 Hz, 1H), 8.73-8.72 (m, 1H), 8.49 (d, J = 1.8 Hz, 1H), 8.31 (s, 1H), 8.22 (s, 1H), 7.75 (d, J = 8.9 Hz, 1H), 7.53 (d, J = 8.7 Hz, 1H), 7.11 (d, J = 2.2 Hz, 1H), 6.96 (s, 1H), 6.88 (dd, J = 8.7, 2.5 Hz, 1H), 6.81 (s, 2H), 6.58 (d, J = 8.8 Hz, 1H), 5.27 (s, 2H), 5.20-5.12 (m, 1H), 2.22-2.10 (m, 2H), 1.96-1.91 (m, 2H), 1.88-1.77 (m, 2H), 1.76-1.66 (m, 2H). | D |
| 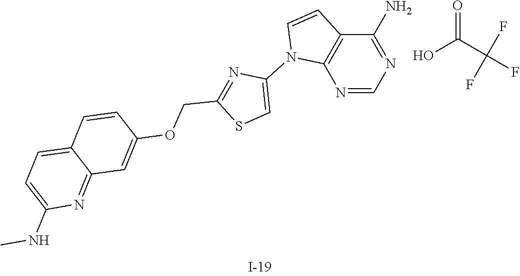 | 404.2 | ¹H NMR (400 MHz, DMSO-d6) δ ppm 9.69 (s, 1H), 8.88 (s, 2H), 8.48 (s, 1H), 8.28-8.14 (m, 2H), 7.99 (d, J = 3.6 Hz, 1H), 7.89 (d, J = 8.8 Hz, 1H), 7.62 (s, 1H), 7.28 (dd, J₁ = 8.8 Hz, J₂ = 2.4 Hz, 1H), 7.07 (d, J = 3.6 Hz, 1H), 6.99-6.88 (m, 1H), 5.67 (s, 2H), 3.10 (d, J = 3.6 Hz, 3H). | A |
| 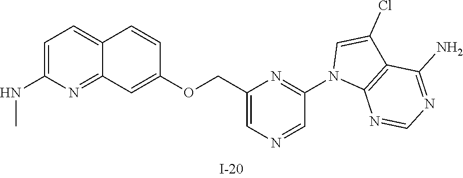 [SMILES: CNc1ccc2ccc(OCc3cncc(-n4cc(Cl)c5c(N)ncnc54)n3)cc2n1] | 434.0 | ¹H NMR (400 MHz, DMSO-d₆) δ ppm 9.89 (s, 1H), 8.76 (s, 1H), 8.32 (s, 1H), 8.30 (s, 0.3H), 8.05 (s, 1H), 7.76 (d, J = 8.8 Hz, 1H), 7.55 (d, J = 8.8 Hz, 1H), 7.13 (s, 1H), 6.95-6.91 (m, 2H), 6.59 (d, J = 8.8 Hz, 1H), 5.41 (s, 2H), 2.88 (d, J = 4.8 Hz, 3H). | A |
| 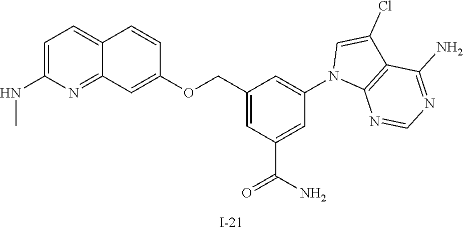 [SMILES: CNc1ccc2ccc(OCc3cc(C(N)=O)cc(-n4cc(Cl)c5c(N)ncnc54)c3)cc2n1] | 474.1 | ¹H NMR (400 MHz, DMSO-d₆) δ ppm 8.18-8.17 (m, 2H), 8.15 (s, 1H), 8.11 (s, 1H), 7.98 (s, 1H), 7.90 (s, 1H), 7.74 (d, J = 8.8 Hz, 1H), 7.52 (d, J = 8.4 Hz, 2H), 7.07 (d, J = 2.0 Hz, 1H), 6.92-6.88 (m, 1H), 6.574 (d, J = 8.8 Hz, 1H), 5.33 (s, 2H), 2.87 (d, J = 4.8 Hz, 3). | A |
| 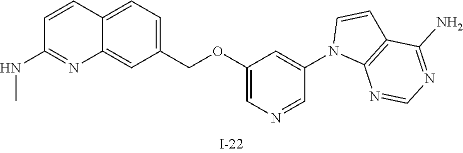 [SMILES: CNc1ccc2ccc(COc3cncc(-n4ccc5c(N)ncnc54)c3)cc2n1] | 398.2 | ¹H NMR (400 MHz, DMSO) δ ppm 8.75 (m, J = 2.0 Hz, 1H), 8.36 (d, J = 2.4 Hz, 1H), 8.16 (s, 1H), 8.12 (t, J = 2.2 Hz, 1H), 7.88 (d, J = 2.4 Hz, 1H), 7.69-7.63 (m, 3H), 7.26 (t, J = 12.0 Hz, 3H), 6.82-6.81 (m, 1H), 6.75 (d, J = 7.2 Hz, 1H), 5.39 (s, 2H), 2.90-2.89 (m, 3H). | |
| 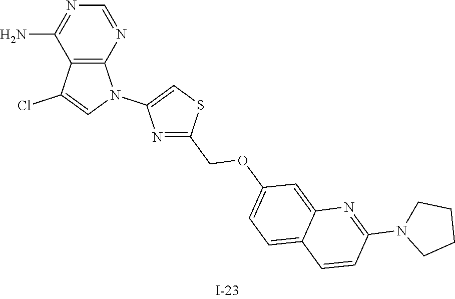 [SMILES: Nc1ncnc2c1c(Cl)cn2-c1csc(COc2ccc3ccc(N4CCCC4)nc3c2)n1] | 438.2 | ¹H NMR (400 MHz, DMSO-d6) δ ppm 12.50 (s, 2H), 9.61 (s, 1H), 8.30 (s, 1H), 8.26-8.17 (m, 2H), 7.97 (s, 1H), 7.89 (d, J = 8.8 Hz, 1H), 7.59 (s, 1H), 7.28 (dd, J₁ = 8.8 Hz, J₂ = 2.4 Hz, 1H), 6.91 (d, J = 8.8 Hz, 1H), 5.67 (s, 2H), 3.10 (d, J = 3.6 Hz, 3H). | A |
| 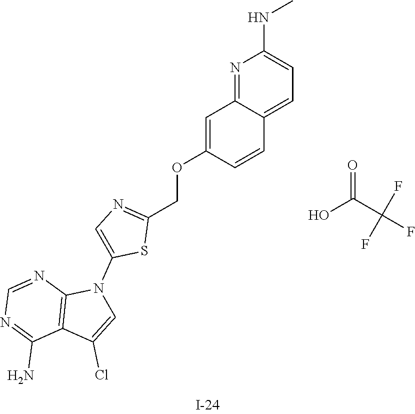 [SMILES: CNc1ccc2ccc(OCc3ncc(-n4cc(Cl)c5c(N)ncnc54)s3)cc2n1 ; O=C(O)C(F)(F)F] | 438.1 | ¹H NMR (400 MHz, DMSO-d6) δ ppm 12.47 (s, 1H), 9.59 (s, 2H), 8.25-8.16 (m, 3H), 8.10 (s, 1H), 7.88 (d, J = 8.8 Hz, 1H), 7.57 (s, 1H), 7.28 (dd, J₁ = 8.8 Hz, J₂ = 2.4 Hz, 1H), 6.90 (d, J = 7.2 Hz, 1H), 5.61 (s, 2H), 3.09 (d, J = 4.8 Hz, 3H). | A |
| 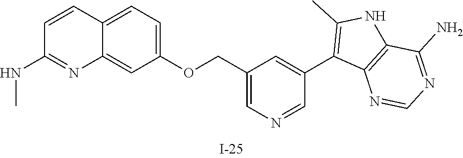 [SMILES: CNc1ccc2ccc(OCc3cncc(-c4c(C)[nH]c5c(N)ncnc45)c3)cc2n1] | 412.1 | ¹H NMR (400 MHz, DMSO-d₆): δ ppm 11.18 (s, 1H), 8.91 (d, J = 2.1 Hz, 1H), 8.56 (d, J = 2.0 Hz, 1H), 8.24-8.23 (m, 1H), 8.14 (s, 1H), 7.75-7.73 (m, 1H), 7.53-7.51 (m, 1H), 7.10 (d, J = 2.4 Hz, 1H), 6.94-6.92 (m, 1H), 6.89-6.86 (m, 1H), 6.63 (s, 2H), 6.59-6.56 (m, 1H), 5.33 (s, 2H), 2.88 (d, J = 4.7 Hz, 3H), 2.58 (s, 3H). | D |
| 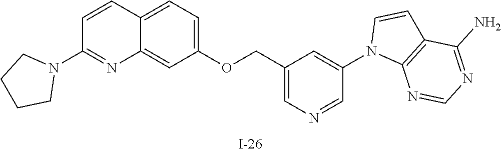 [SMILES: Nc1ncnc2c1ccn2-c1cncc(COc2ccc3ccc(N4CCCC4)nc3c2)c1] | 438.3 | ¹H NMR (400 MHz, DMSO δ 9.07 (d, J = 2.2 Hz, 1H), 8.67 (s, 1H), 8.45 (s, 1H), 8.15 (s, 1H), 7.91 (d, J = 8.8 Hz, 1H), 7.70 (d, J = 3.7 Hz, 1H), 7.59 (d, J = 8.8 Hz, 1H), 7.24 (s, 2H), 7.12 (s, 1H), 6.91 (d, J = 6.7 Hz, 1H), 6.84 (d, J = 3.7 Hz, 1H), 6.71 (d, J = 8.9 Hz, 1H), 5.37 (s, 2H), 3.52 (s, 4H), 1.98-1.95 (m, 4H). | A |
| 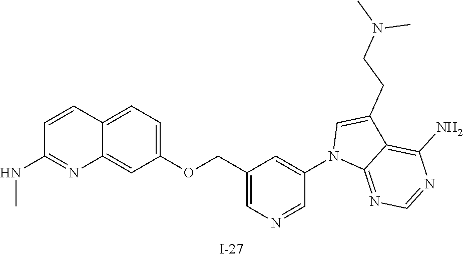 [SMILES: CNc1ccc2ccc(OCc3cncc(-n4cc(CCN(C)C)c5c(N)ncnc54)c3)cc2n1] | 469.2 | ¹H NMR (400 MHz, DMSO-d₆) δ ppm 9.06 (d, J = 2.0 Hz, 1H), 8.63 (d, J = 1.6 Hz, 1H), 8.42-8.41 (m, 1H), 8.12 (s, 1H), 7.74 (d, J = 8.8 Hz, 1H), 7.54-7.51 (m, 2H), 7.10-7.08 (m, 3H), 6.94-6.86 (m, 2H), 6.58 (d, J = 9.2 Hz, 1H), 5.35 (s, 2H), 4.11-4.10 (m, 1H), 2.96-2.92 (m, 2H), 2.87 (d, J = 4.8 Hz, 3H), 2.61-2.57 (m, 2H), 2.24 (s, 6H). | A |
| 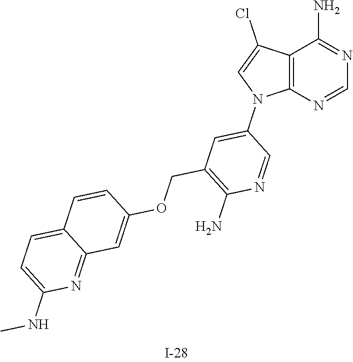 [SMILES: CNc1ccc2ccc(OCc3cc(-n4cc(Cl)c5c(N)ncnc54)cnc3N)cc2n1] | 447.1 | ¹H NMR (400 MHz, DMSO-d₆) δ ppm 8.18 (d, J = 2.6 Hz, 1H), 8.10 (s, 1H), 7.88 (d, J = 2.6 Hz, 1H), 7.74 (d, J = 8.9 Hz, 1H), 7.67 (s, 1H), 7.52 (d, J = 8.7 Hz, 1H), 7.16 (d, J = 2.3 Hz, 1H), 7.00-6.82 (m, 3H), 6.58 (d, J = 8.8 Hz, 1H), 6.27 (s, 2H), 5.07 (s, 2H), 2.88 (d, J = 4.7 Hz, 3H). | A |
| 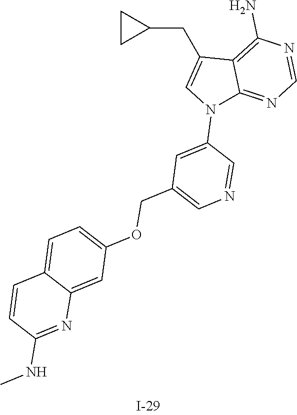 [SMILES: CNc1ccc2ccc(OCc3cncc(-n4cc(CC5CC5)c5c(N)ncnc54)c3)cc2n1] | 452.2 | ¹H NMR (400 MHz, DMSO-d₆) δ ppm 9.08 (d, J = 2.4 Hz, 1H), 8.64 (d, J = 1.7 Hz, 1H), 8.51-8.31 (m, 1H), 8.13 (s, 1H), 7.74 (d, J = 8.8 Hz, 1H), 7.60-7.48 (m, 2H), 7.10 (d, J = 2.4 Hz, 1H), 7.03-6.82 (m, 2H), 6.73 (s, 2H), 6.58 (d, J = 8.9 Hz, 1H), 5.36 (s, 2H), 2.88 (d, J = 4.7 Hz, 3H), 2.79 (d, J = 6.7 Hz, 2H), 1.18-1.00 (m, 1H), 0.63-0.37 (m, 2H), 0.32-0.13 (m, 2H). | A |
| 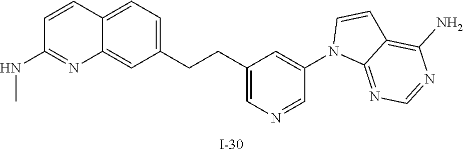 [SMILES: CNc1ccc2ccc(CCc3cncc(-n4ccc5c(N)ncnc54)c3)cc2n1] | 396.3 | ¹H NMR (400 MHz, DMSO-d₆) δ ppm 8.00 (d, J = 1.2 Hz, 1H), 7.55 (s, 1H), 7.32 (s, 1H), 7.21 (s, 1H), 7.00 (d, J = 8.8 Hz, 1H), 6.75 (d, J = 8.0 Hz, 1H), 6.65 (s, 1H), 6.57 (d, J = 4.0 Hz, 1H), 6.32 (d, J = 8.0 Hz, 1H), 6.00 (d, J = 4.0 Hz, 1H), 5.90 (d, J = 8.8 Hz, 1H), 3.16-3.15 (m, 4H), 2.97 (s, 3H). | C |
| 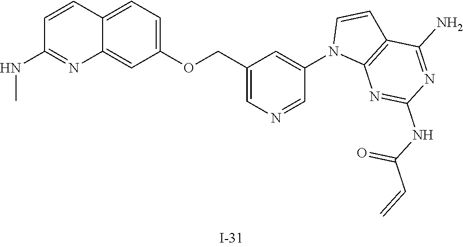 [SMILES: C=CC(=O)Nc1nc(N)c2ccn(-c3cncc(COc4ccc5ccc(NC)nc5c4)c3)c2n1] | 467.2 | ¹H NMR (400 MHz, DMSO-d₆) δ ppm 10.11 (s, 1H), 9.25 (d, J = 2.5 Hz, 1H), 8.64 (d, J = 1.7 Hz, 1H), 8.55-8.51 (m, 1H), 7.74 (d, J = 8.8 Hz, 1H), 7.64 (d, J = 3.7 Hz, 1H), 7.53 (d, J = 8.8 Hz, 1H), 7.21 (s, 2H), 7.11 (d, J = 2.4 Hz, 1H), 6.93-6.90 (m, 2H), 6.82 (d, J = 3.7 Hz, 1H), 6.58 (d, J = 8.9 Hz, 1H), 6.26-6.21 (m, 1H), 5.70-5.67 (m, 1H), 5.34 (s, 2H), 2.88 (d, J = 4.7 Hz, 3H). | A |
| 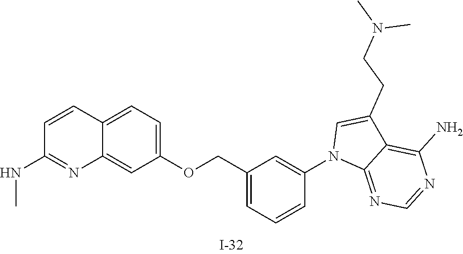 [SMILES: CNc1ccc2ccc(OCc3cccc(-n4cc(CCN(C)C)c5c(N)ncnc54)c3)cc2n1] | 469.2 | ¹H NMR (400 MHz, CD3OD) δ ppm 8.11 (s, 1H), 7.92 (br, 1H), 7.80 (d, J = 8.4 Hz, 1H), 7.73 (d, J = 8.4 Hz, 1H), 7.56-7.51 (m, 2H), 7.44-7.41 (m, 2H), 7.06 (s, 1H), 6.97-6.86 (m, 4H), 6.57 (d, J = 8.8 Hz, 1H), 5.29 (s, 2H), 3.10-2.98 (m, 2H), 2.87 (s, 3H), 2.71-2.68 (m, 2H), 2.28 (2, 6H). | A |
| 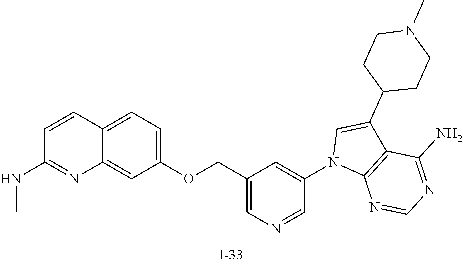 [SMILES: CNc1ccc2ccc(OCc3cncc(-n4cc(C5CCN(C)CC5)c5c(N)ncnc54)c3)cc2n1] | 495.0 | ¹H NMR (400 MHz, CD3OD) δ ppm 8.96 (s, 1H), 8.70 (d, J = 1.6 Hz, 1H), 8.40 (s, 2H), 8.16 (s, 1H), 7.91-7.89 (m, 1H), 7.64-7.62 (m, 1H), 7.45 (s, 1H), 7.31 (d, J = 1.6 Hz, 1H), 7.08-7.05 (m, 1H), 7.71-7.68 (m, 1H), 5.41 (s, 2H), 4.60-4.54 (m, 2H), 3.58-3.55 (m, 2H), 3.27-3.24 (m, 1H), 3.05 (s, 3H), 2.88 (s, 3H), 2.38-2.35 (m, 2H), 2.02-1.95 (m, 2H), | A |
| 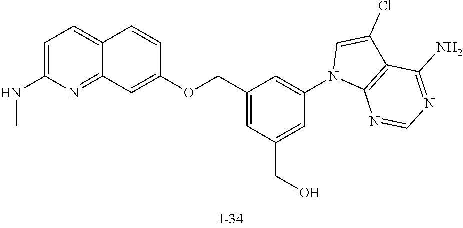 [SMILES: CNc1ccc2ccc(OCc3cc(CO)cc(-n4cc(Cl)c5c(N)ncnc54)c3)cc2n1] | 461.1 | ¹H NMR (400 MHz, DMSO-d₆) δ ppm 8.16 (s, 1H), 7.80 (s, 1H), 7.77-7.69 (m, 3H), 7.52 (d, J = 8.8 Hz, 1H), 7.46 (s, 1H), 7.06 (d, J = 2.5 Hz, 1H), 6.93-6.86 (m, 2H), 6.57 (d, J = 8.8 Hz, 1H), 5.41-5.38 (m, 1H), 5.27 (s, 2H), 4.60 (d, J = 5.5 Hz, 2H), 2.87 (d, J = 4.7 Hz, 3H). | B |
| 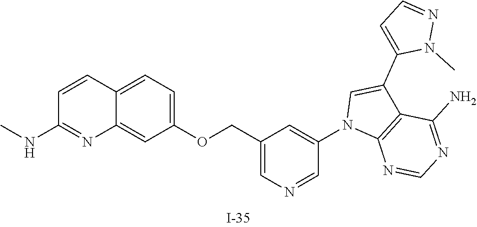 [SMILES: CNc1ccc2ccc(OCc3cncc(-n4cc(-c5ccnn5C)c5c(N)ncnc54)c3)cc2n1] | 478.2 | ¹H NMR (400 MHz, DMSO-d₆): δ ppm 9.10 (d, J = 2.4 Hz, 1H), 8.73 (d, J = 1.6 Hz, 1H), 8.50 (t, J = 2.0 Hz, 1H), 8.26 (s, 1H), 8.04 (s, 1H), 7.75 (d, J = 8.8 Hz, 1H), 7.59 (d, J = 1.8 Hz, 1H), 7.54 (d, J = 8.7 Hz, 1H), 7.12 (d, J = 2.0 Hz, 1H), 6.99-6.85 (m, 2H), 5.38 (s, 2H), 3.87 (s, 3H), 2.88 (d, J = 4.7 Hz, 3H). | A |
| 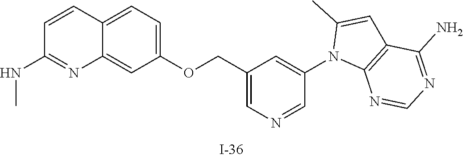 [SMILES: CNc1ccc2ccc(OCc3cncc(-n4c(C)cc5c(N)ncnc54)c3)cc2n1] | 412.1 | ¹H NMR (400 MHz, DMSO-d₆) δ ppm 8.79 (brs, 1H), 8.64 (d, J = 2.0 Hz, 1H), 8.03 (brs, 1H), 7.96-7.97 (m, 1H), 7.54 (d, J = 7.6 Hz, 1H), 7.10-7.12 (m, 1H), 7.02 (s, 1H), 6.94-6.98 (m, 3H), 6.88 (d, J = 4.4 Hz, 1H), 6.61 (d, J = 2.8 Hz, 1H), 6.52 (s, 1H), 5.37 (s, 2H), 2.89 (d, J = 4.4 Hz, 3H). 3.26 (s, 3H). | A |
| 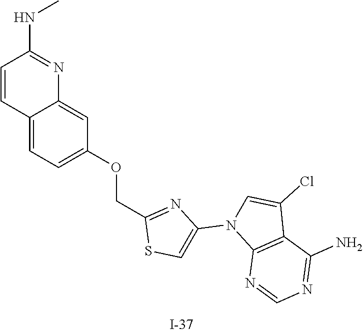 [SMILES: CNc1ccc2ccc(OCc3nc(-n4cc(Cl)c5c(N)ncnc54)cs3)cc2n1] | 438.2 | ¹H NMR (400 MHz, DMSO-d6) δ ppm 12.50 (s, 2H), 9.61 (s, 1H), 8.30 (s, 1H), 8.26-8.17 (m, 2H), 7.97 (s, 1H), 7.89 (d, J = 8.8 Hz, 1H), 7.59 (s, 1H), 7.28 (dd, J₁ = 8.8 Hz, J₂ = 2.4 Hz, 1H), 6.91 (d, J = 8.8 Hz, 1H), 5.67 (s, 2H), 3.10 (d, J = 3.6 Hz, 3H). | B |
| 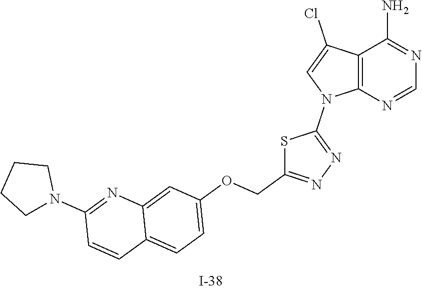 [SMILES: Nc1ncnc2c1c(Cl)cn2-c1nnc(COc2ccc3ccc(N4CCCC4)nc3c2)s1] | 479.2 | ¹H NMR (400 MHz, DMSO-d6) δ ppm 12.46 (s, 1H), 8.36-8.23 (m, 2H), 8.17 (s, 1H), 7.88 (d, J = 8.4 Hz, 1H), 7.61 (s, 1H), 7.28-7.21 (m, 1H), 7.10-7.02 (m, 1H), 5.76 (s, 2H), 3.75-3.66 (m, 4H), 2.16-1.99 (m, 4H). | B |
| 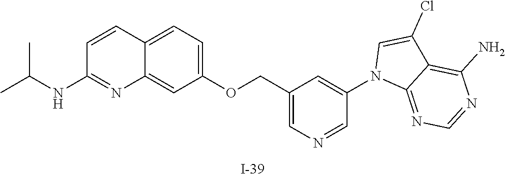 [SMILES: CC(C)Nc1ccc2ccc(OCc3cncc(-n4cc(Cl)c5c(N)ncnc54)c3)cc2n1] | 460.1 | ¹H NMR (400 MHz, DMSO-d₆) δ ppm 9.01 (d, J = 2.0 Hz, 1H), 8.69 (d, J = 2.0 Hz, 1H), 8.40-8.41 (m, 1H), 8.18 (s, 1H), 7.95 (s, 1H), 7.72 (d, J = 8.8 Hz, 1H), 7.51 (d, J = 8.8 Hz, 1H), 7.07 (d, J = 2.4 Hz, 2H), 6.87 (dd, J = 8.8 Hz, 2.8 Hz, 1H), 6.80 (d, J = 7.6 Hz, 1H), 6.56 (d, J = 8.8 Hz, 1H), 5.35 (s, 2H), 4.22 (d, J = 7.6, 1H), 1.18-1.22 (m, 6H). | B |
| 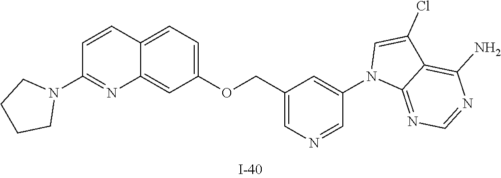 [SMILES: Nc1ncnc2c1c(Cl)cn2-c1cncc(COc2ccc3ccc(N4CCCC4)nc3c2)c1] | 472.1 | ¹H NMR (400 MHz, DMSO-d₆) δ ppm 9.01 (d, J = 2.4 Hz, 1H), 8.70 (d, J = 1.6 Hz, 1H), 8.39-8.41 (m, 1H), 8.18 (s, 1H), 7.95 (s, 1H), 7.90 (d, J = 8.8 Hz, 1H), 7.59 (d, J = 8.8 Hz, 1H), 7.11 (d, J = 2.4 Hz, 1H), 6.90 (dd, J = 8.8 Hz, 2.4 Hz, 1H), 6.70 (d, J = 9.2 Hz, 1H), 5.36 (s, 2H), 4.13 (s, 4H), 1.95-1.98 (m, 4H). | B |
| 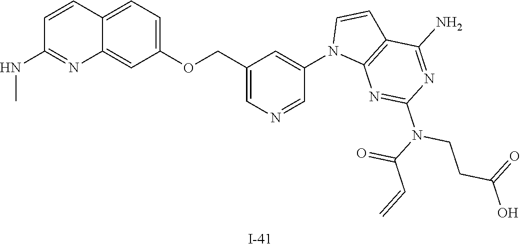 [SMILES: C=CC(=O)N(CCC(=O)O)c1nc(N)c2ccn(-c3cncc(COc4ccc5ccc(NC)nc5c4)c3)c2n1] | 539.1 | ¹H NMR (400 MHz, DMSO-d₆) δ ppm 11.13 (s, 1H), 10.57 (s, 1H), 9.18 (s, 1H), 9.13 (s, 1H), 7.81 (d, J = 3.9 Hz, 1H), 7.77 (d, J = 8.9 Hz, 1H), 7.57 (d, J = 8.8 Hz, 1H), 7.38 (s, 2H), 7.16 (d, J = 2.4 Hz, 1H), 7.03-6.93 (m, 2H), 6.90 (d, J = 3.8 Hz, 1H), 6.81-6.77 (m, 1H), 6.61 (d, J = 8.9 Hz, 1H), 6.30 (dd, J = 17.1, 2.0 Hz, 1H), 5.72 (dd, J = 10.3, 1.9 Hz, 1H), 5.44 (s, 2H), 4.87 (t, J = 5.6 Hz, 2H), 2.89 (d, J = 4.7 Hz, 3H), 2.68-2.65 (m, 1H). | A |
| 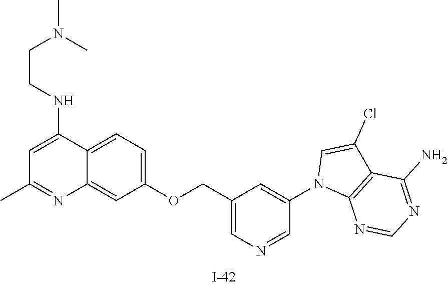 [SMILES: Cc1cc(NCCN(C)C)c2ccc(OCc3cncc(-n4cc(Cl)c5c(N)ncnc54)c3)cc2n1] | 503.2 | ¹H NMR (400 MHz, CD3OD) δ ppm 8.99 (d, J = 2.4 Hz, 1H), 8.72 (d, J = 1.7 Hz, 1H), 8.43 (d, J = 2.1 Hz, 1H), 8.26 (d, J = 9.3 Hz, 1H), 8.15 (s, 1H), 7.69 (s, 1H), 7.40 (dd, J = 9.3, 2.5 Hz, 1H), 7.25 (d, J = 2.4 Hz, 1H), 6.67 (s, 1H), 5.47 (s, 2H), 3.69 (t, J = 6.5 Hz, 2H), 2.88-2.74 (m, 2H), 2.66 (s, 3H), 2.42 (d, J = 3.3 Hz, 6H). | B |
| 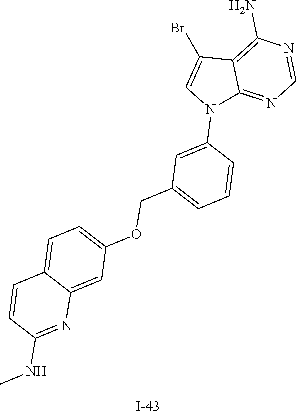 [SMILES: CNc1ccc2ccc(OCc3cccc(-n4cc(Br)c5c(N)ncnc54)c3)cc2n1] | 475.0 | ¹H NMR (400 MHz, DMSO-d₆) δ ppm 8.16 (s, 1H), 7.95-7.90 (m, 1H), 7.88 (s, 1H), 7.80-7.68 (m, 2H), 7.56 (t, J = 7.8 Hz, 1H), 7.53-7.46 (m, 2H), 7.06 (d, J = 2.5 Hz, 1H), 6.96-6.82 (m, 3H), 6.57 (d, J = 8.8 Hz, 1H), 5.29 (s, 2H), 2.87 (d, J = 4.7 Hz, 3H). | B |
| 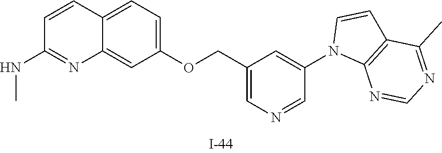 [SMILES: CNc1ccc2ccc(OCc3cncc(-n4ccc5c(C)ncnc54)c3)cc2n1] | 397.1 | ¹H NMR (400 MHz, DMSO-d₆) δ ppm 9.11 (d, J = 1.6 Hz 1H), 8.76 (d, J = 11.6 Hz, 2H), 8.50 (t, J = 2.2 Hz, 1H), 8.11 (m, 1H), 7.76 (d, J = 7.6 Hz, 1H), 7.55 (d, J = 9.2 Hz, 1H), 7.12 (d, J = 2.4 Hz, 1H), 7.02 (d, J = 4.0 Hz, 1H), 6.91-6.88 (m, 1H), 6.60 (d, J = 8.8 Hz, 1H), 5.39 (s, 2H), 2.88-2.87 (m, 3H), 2.73 (s, 3H). | A |
| 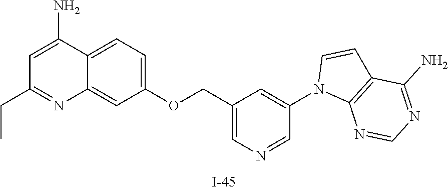 [SMILES: CCc1cc(N)c2ccc(OCc3cncc(-n4ccc5c(N)ncnc54)c3)cc2n1] | 412.0 | ¹H NMR (400 MHz, DMSO) δ ppm 9.08 (d, J = 2.4 Hz, 1H), 8.69 (d, J = 1.6 Hz, 1H), 8.46-8.47 (m, 1H), 8.16 (s, 1H), 8.02 (d, J = 9.2 Hz, 1H), 7.70 (d, J = 3.7 Hz, 1H), 7.24 (d, J = 2.5 Hz, 3H), 7.09-7.06 (m, 1H), 6.85 (d, J = 3.7 Hz, 1H), 6.62 (s, 2H), 6.34 (s, 1H), 5.38 (s, 2H), 2.64 (q, J = 7.6 Hz, 2H), 1.23 (t, J = 7.6 Hz, 3H). | B |
| 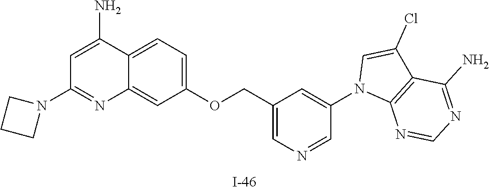 [SMILES: Nc1cc(N2CCC2)nc2cc(OCc3cncc(-n4cc(Cl)c5c(N)ncnc54)c3)ccc12] | 458.2 | ¹H NMR (400 MHz, DMSO) δ ppm 9.02 (d, J = 2.5 Hz, 1H), 8.69 (d, J = 1.7 Hz, 1H), 8.42 (t, J = 2.1 Hz, 1H), 8.18 (s, 1H), 7.97 (s, 1H), 7.92 (d, J = 8.8 Hz, 1H), 7.62 (d, J = 8.8 Hz, 1H), 7.14 (d, J = 2.4 Hz, 1H), 6.95 (dd, J = 8.7, 2.5 Hz, 1H), 6.53 (d, J = 8.8 Hz, 1H), 5.36 (s, 2H), 4.06 (t, J = 7.4 Hz, 4H), 2.38-2.30 (m, 2H). | B |
| 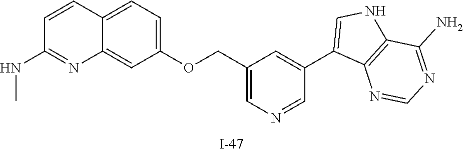 [SMILES: CNc1ccc2ccc(OCc3cncc(-c4c[nH]c5c(N)ncnc45)c3)cc2n1] | 398.0 | ¹H NMR (400 MHz, CD₃OD) δ ppm 9.10 (d, J = 1.9 Hz, 1H), 8.62-8.59 (m, 1H), 8.55 (d, J = 1.8 Hz, 1H), 8.42 (s, 1H), 8.27 (s, 1H), 8.08-8.01 (m, 2H), 7.74 (d, J = 8.8 Hz, 1H), 7.41 (d, J = 2.2 Hz, 1H), 7.20 (dd, J = 8.8, 2.4 Hz, 1H), 6.80 (d, J = 9.2 Hz, 1H), 5.40 (s, 2H), 3.13 (s, 3H). | D |
| 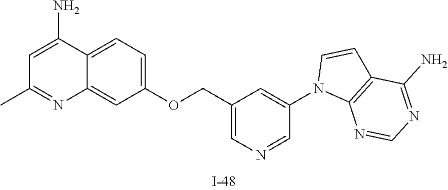 [SMILES: Cc1cc(N)c2ccc(OCc3cncc(-n4ccc5c(N)ncnc54)c3)cc2n1] | 398.2 | ¹H NMR (400 MHz, DMSO-d₆) δ ppm ¹H 9.08 (d, J = 2.5 Hz, 1H), 8.68 (d, J = 1.7 Hz, 1H), 8.47-8.46 (m, 1H), 8.16 (s, 1H), 8.01 (d, J = 9.2 Hz, 1H), 7.70 (d, J = 3.7 Hz, 1H), 7.28-7.19 (m, 3H), 7.07-7.04 (m, 1H), 6.84 (d, J = 3.7 Hz, 1H), 6.57 (s, 2H), 6.31 (s, 1H), 5.37 (s, 2H), 2.36 (s, 3H). | B |
| 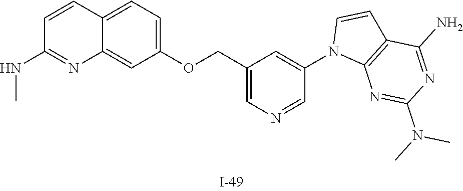 [SMILES: CNc1ccc2ccc(OCc3cncc(-n4ccc5c(N)nc(N(C)C)nc54)c3)cc2n1] | 442.1 | ¹H NMR (400 MHz, DMSO) ppm 9.12 (d, J = 2.5 Hz, 1H), 8.66 (t, J = 2.1 Hz, 1H), 8.57 (d, J = 1.8 Hz, 1H), 7.74 (d, J = 8.9 Hz, 1H), 7.53 (d, J = 8.7 Hz, 1H), 7.36 (d, J = 3.8 Hz, 1H), 7.08 (d, J = 2.4 Hz, 1H), 6.96-6.82 (m, 1H), 6.88-6.77 (m, 3H), 6.65 (d, J = 3.7 Hz, 1H), 6.58 (d, J = 8.8 Hz, 1H), 5.35 (s, 2H), 3.06 (s, 6H), 2.87 (d, J = 4.7 Hz, 3H). | A |
| 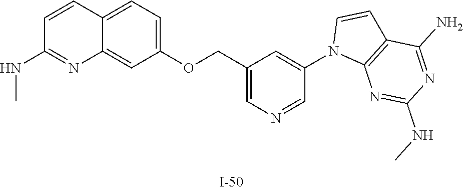 [SMILES: CNc1ccc2ccc(OCc3cncc(-n4ccc5c(N)nc(NC)nc54)c3)cc2n1] | 427.1 | ¹H NMR (400 MHz, DMSO-d₆) δ ppm 9.15 (d, J = 2.4 Hz, 1H), 8.59-8.57 (m, 2H), 7.74 (d, J = 8.8 Hz, 1H), 7.53 (d, J = 8.8 Hz, 1H), 7.08 (d, J = 2.4 Hz, 1H), 6.95-6.93 (m, 1H), 6.87 (dd, J = 8.7, 2.5 Hz, 1H), 6.74 (s, 2H), 6.64 (d, J = 3.7 Hz, 1H), 6.58 (d, J = 8.8 Hz, 1H), 6.16-6.15 (m, 1H), 5.34 (s, 2H), 2.87 (d, J = 4.7 Hz, 3H), 2.74 (d, J = 4.8 Hz, 3H). | A |
| 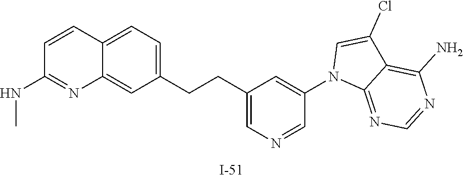 [SMILES: CNc1ccc2ccc(CCc3cncc(-n4cc(Cl)c5c(N)ncnc54)c3)cc2n1] | 430.1 | ¹H NMR (400 MHz, DMSO-d₆) δ ppm 8.80 (s, 1H), 8.34 (s, 1H), 8.13 (s, 1H), 7.91 (d, J = 2.0 Hz, 1H), 7.81 (d, J = 9.2 Hz, 1H), 7.55 (t, J = 4.0 Hz, 1H), 7.42 (d, J = 4.4 Hz, 2H), 7.12-7.10 (m, 1H), 6.71 (t, J = 9.2 Hz, 1H), 3.26-3.20 (m, 4H), 3.18 (s, 3H). | C |
| 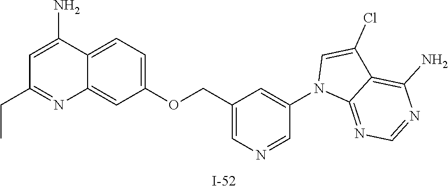 [SMILES: CCc1cc(N)c2ccc(OCc3cncc(-n4cc(Cl)c5c(N)ncnc54)c3)cc2n1] | 446.2 | ¹H NMR (400 MHz, DMSO) δ ppm 9.03 (d, J = 2.5 Hz, 1H), 8.71 (d, J = 1.7 Hz, 1H), 8.44-8.43 (m, 1H), 8.19 (s, 1H), 8.03 (d, J = 9.2 Hz, 1H), 7.98 (s, 1H), 7.24 (d, J = 2.6 Hz, 1H), 7.09-7.06 (m, 1H), 6.65 (s, 1H), 6.34 (s, 1H), 5.38 (s, 2H), 2.65 (q, J = 7.6 Hz, 2H), 1.23 (t, J = 7.6 Hz, 3H). | B |
| 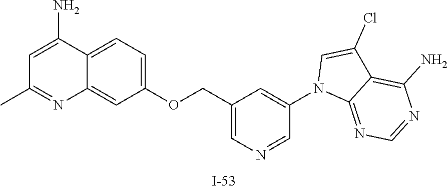 [SMILES: Cc1cc(N)c2ccc(OCc3cncc(-n4cc(Cl)c5c(N)ncnc54)c3)cc2n1] | 432.1 | ¹H NMR (400 MHz, DMSO-d₆) δ ppm 9.02 (d, J = 2.5 Hz, 1H), 8.71 (d, J = 1.7 Hz, 1H), 8.45-8.39 (m, 1H), 8.19 (s, 1H), 8.01 (d, J = 9.1 Hz, 1H), 7.97 (s, 1H), 7.21 (d, J = 2.6 Hz, 1H), 7.06 (dd, J = 9.1, 2.6 Hz, 1H), 6.60 (s, 1H), 6.31 (s, 1H), 5.36 (s, 2H), 3.31 (s, 2H), 2.53-2.51 (m, 1H), 2.36 (s, 3H). | B |
| 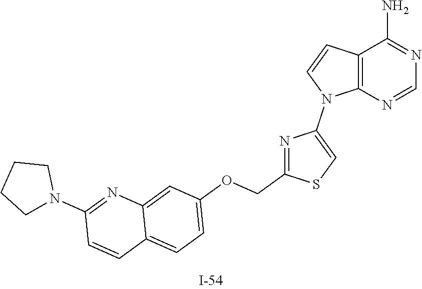 [SMILES: Nc1ncnc2c1ccn2-c1csc(COc2ccc3ccc(N4CCCC4)nc3c2)n1] | 444.2 | ¹H NMR (400 MHz, DMSO-d6) δ ppm 12.39 (s, 2H), 8.41 (s, 1H), 8.35 (d, J = 9.6 Hz, 1H), 8.25 (s, 1H), 7.96-7.90 (m, 2H), 7.62 (s, 1H), 7.28 (dd, J₁ = 9.0 Hz, J₂ = 2.2 Hz, 1H), 7.12 (d, J = 9.0 Hz, 1H), 6.99 (d, J = 3.6 Hz, 1H), 5.68 (s, 2H), 3.73-3.66 (m, 4H), 2.13-2.00 (m, 4H). | A |
| 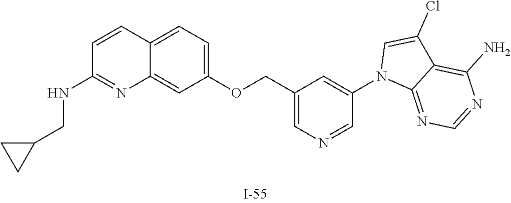 [SMILES: Nc1ncnc2c1c(Cl)cn2-c1cncc(COc2ccc3ccc(NCC4CC4)nc3c2)c1] | 472.1 | ¹H NMR (400 MHz, DMSO-d₆) δ ppm 9.01 (d, J = 2.5 Hz, 1H), 8.69 (d, J = 1.7 Hz, 1H), 8.41-8.40 (m, 1H), 8.18 (s, 1H), 7.96 (s, 1H), 7.74 (d, J = 8.9 Hz, 1H), 7.52 (d, J = 8.8 Hz, 1H), 7.07-7.04 (m, 2H), 6.89-6.86 (m, 1H), 6.63 (d, J = 8.9 Hz, 1H), 5.34 (s, 2H), 3.26-3.23 (m, 2H), 1.11-1.06 (m, 1H), 0.47-0.44 (m, 2H), 0.26-0.22 (m, 2H). | B |
| 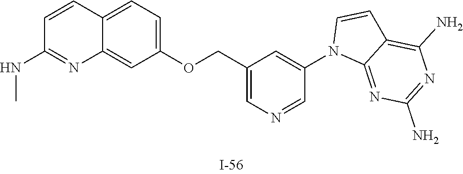 [SMILES: CNc1ccc2ccc(OCc3cncc(-n4ccc5c(N)nc(N)nc54)c3)cc2n1] | 413.0 | ¹H NMR (400 MHz, DMSO-d₆) δ 9.12 (d, J = 2.5 Hz, 1H), 8.59 (d, J = 1.7 Hz, 1H), 7.74 (d, J = 8.9 Hz, 1H), 8.38-8.37 (m, 1H), 7.74 (d, J = 8.8 Hz), 7.53 (d, J = 8.7 Hz, 1H), 7.25 (d, J = 3.7 Hz, 1H), 7.10 (d, J = 2.4 Hz, 1H), 6.95-6.94 (m, 1H), 6.89 (dd, J = 8.7, 2.5 Hz, 1H), 6.73 (s, 2H), 6.63 (d, J = 3.7 Hz, 1H), 6.58 (d, J = 8.8 Hz, 1H), 5.73 (s, 2H), 5.33 (s, 2H), 2.88 (d, J = 4.7 Hz, 3H). | A |
| 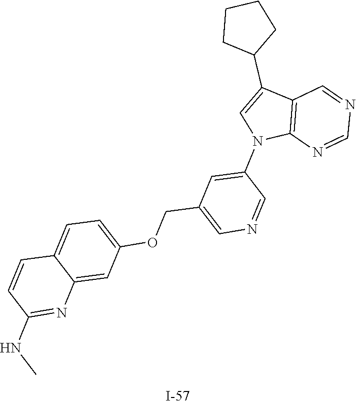 [SMILES: CNc1ccc2ccc(OCc3cncc(-n4cc(C5CCCC5)c5cncnc54)c3)cc2n1] | 451.3 | ¹H NMR (400 MHz, CD3OD) δ ppm 9.26-9.00 (m, 2H), 8.85 (s, 1H), 8.70 (s, 1H), 8.53 (s, 1H), 7.88-7.66 (m, 2H), 7.63-7.46 (m, 1H), 7.25 (s, 1H), 7.03-6.95 (m, 1H), 6.60 (d, J = 8.9 Hz, 1H), 5.40 (s, 2H), 3.44-3.36 (m, 1H), 3.00 (s, 3H), 2.43-2.17 (m, 2H), 2.00-1.70 (m, 6H), 1.42-1.25 (m, 1H). | A |
| 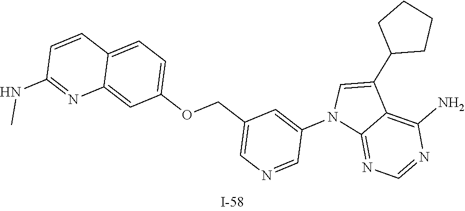 [SMILES: CNc1ccc2ccc(OCc3cncc(-n4cc(C5CCCC5)c5c(N)ncnc54)c3)cc2n1] | 466.2 | ¹H NMR (400 MHz, DMSO-d₆) δ ppm 9.10 (d, J = 2.5 Hz, 1H), 8.63 (d, J = 1.7 Hz, 1H), 8.43 (t, J = 2.1 Hz, 1H), 8.13 (s, 1H), 7.74 (d, J = 8.8 Hz, 1H), 7.53 (d, J = 8.8 Hz, 1H), 7.50 (s, 1H), 7.10 (d, J = 2.4 Hz, 1H), 6.96-6.91 (m, 1H), 6.88 (dd, J = 8.7, 2.5 Hz, 1H), 6.74 (s, 2H), 6.58 (d, J = 8.8 Hz, 1H), 5.35 (s, 2H), 3.53-3.40 (m, 1H), 2.88 (d, J = 4.7 Hz, 3H), 2.13-2.07 (m, 2H), 1.81-1.49 (m, 6H). | B |
| 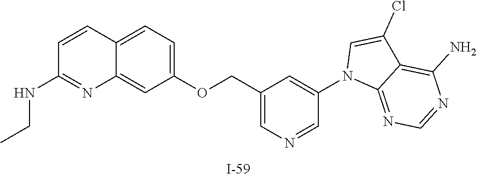 [SMILES: CCNc1ccc2ccc(OCc3cncc(-n4cc(Cl)c5c(N)ncnc54)c3)cc2n1] | 446.1 | ¹H NMR (400 MHz, DMSO-d₆) δ ppm δ 9.02 (d, J = 2.5 Hz, 1H), 8.70 (d, J = 1.7 Hz, 1H), 8.19 (s, 1H), 7.97 (s, 1H), 7.74 (d, J = 8.8 Hz, 1H), 7.52 (d, J = 8.7 Hz, 1H), 7.08 (d, J = 2.4 Hz, 1H), 6.93-6.89 (m, 1H), 6.88 (dd, J = 8.7, 2.5 Hz, 1H), 6.58 (d, J = 8.8 Hz, 1H), 5.35 (s, 2H), 3.44-3.37 (m, 2H), 1.18 (t, J = 7.2 Hz, 3H). | B |
| 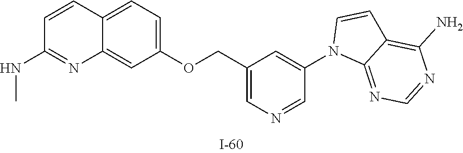 [SMILES: CNc1ccc2ccc(OCc3cncc(-n4ccc5c(N)ncnc54)c3)cc2n1] | 398.0 | ¹H NMR (400 MHz, DMSO-d₆) δ ppm 9.07 (d, J = 2.4 Hz, 1H), 8.67 (d, J = 2.0 Hz, 1H), 8.45-8.44 (m, 1H), 8.15 (s, 1H), 7.74 (d, J = 8.8 Hz, 1H), 7.70 (d, J = 4.0 Hz, 1H), 7.53 (d, J = 8.4 Hz, 1H), 7.24 (br, 2H), 7.10 (s, 1H), 7.95-7.94 (m, 1H), 6.89 (dd, J = 8.4, 2.4 Hz, 1H), 6.83 (d, J = 3.6 Hz, 1H), 6.59 (d, J = 8.8 Hz, 1H), 5.36 (s, 2H), 2.88 (d, J = 4.8 Hz, 3H) | B |
| 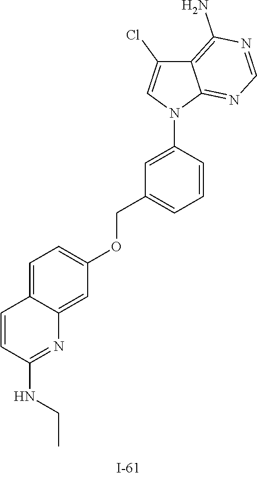 [SMILES: CCNc1ccc2ccc(OCc3cccc(-n4cc(Cl)c5c(N)ncnc54)c3)cc2n1] | 445.1 | ¹H NMR (400 MHz, DMSO-d6) δ 8.16 (s, 1H), 7.93 (s, 1H), 7.85 (s, 1H), 7.75 (t, J = 9.2 Hz, 2H), 7.57 (t, J = 7.8 Hz, 1H), 7.53-7.46 (m, 2H), 7.04 (d, J = 2.3 Hz, 2H), 6.93 (s, 2H), 6.88-6.83 (m, 1H), 6.57 (d, J = 8.8 Hz, 1H), 5.28 (s, 2H), 3.42-3.38 (m, 2H), 1.18 (t, J = 7.2 Hz, 3H). | B |
| 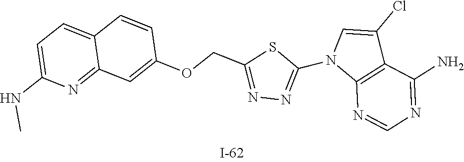 [SMILES: CNc1ccc2ccc(OCc3nnc(-n4cc(Cl)c5c(N)ncnc54)s3)cc2n1] | 439.0 | ¹H NMR (400 MHz, DMSO-d6) δ 8.33 (s, 1H), 8.26 (s, 0.5H), 8.19 (s, 1H), 7.75 (d, J = 8.8 Hz, 1H), 7.55 (d, J = 8.8 Hz, 1H), 7.14 (d, J = 2 Hz, 1H), 6.99 (d, J = 4.8 Hz, 1H), 6.90 (dd, J = 8.4 Hz, 2.4 Hz, 2H), 6.60 (d, J = 8.8 Hz, 1H), 5.71 (s, 2H), 2.87 (d, J = 4.8 Hz, 3H). | B |
| 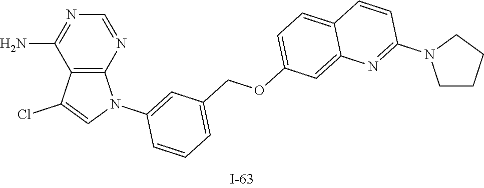 [SMILES: Nc1ncnc2c1c(Cl)cn2-c1cccc(COc2ccc3ccc(N4CCCC4)nc3c2)c1] | 471.15 | ¹H NMR (400 MHz, DMSO-d6) δ ppm 12.45 (s, 1H), 8.37-8.27 (m, 2H), 8.02 (s, 1H), 7.93-7.86 (m, 2H), 7.76 (d, J = 8.0 Hz, 1H), 7.66-7.53 (m, 3H), 7.23 (dd, J₁ = 8.8 Hz, J₂ = 2.4 Hz, 1H), 7.09 (d, J = 9.6 Hz, 1H), 5.34 (s, 2H), 3.75-3.62 (m, 4H), 2.15-1.98 (m, 4H). | B |
| 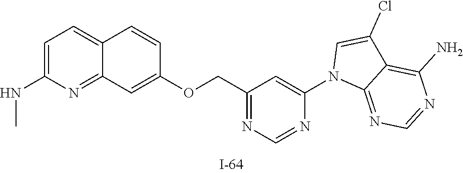 [SMILES: CNc1ccc2ccc(OCc3cc(-n4cc(Cl)c5c(N)ncnc54)ncn3)cc2n1] | 434.0 | ¹H NMR (400 MHz, DMSO-d6) δ 9.10 (s, 1H), 8.30 (s, 1H), 8.19 (s, 1H), 7.76 (d, J = 8.8 Hz, 1H), 7.58 (d, J = 8.8 Hz, 1H), 7.05-7.04 (m, 1H), 6.98-6.95 (m, 4H), 6.59 (d, J = 8.8 Hz, 1H) 5.41 (s, 2H), 2.86 (d, J = 4.8 Hz, 3H). | A |
| 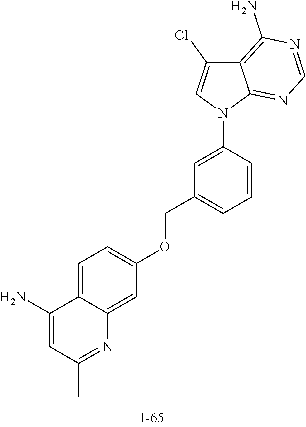 [SMILES: Cc1cc(N)c2ccc(OCc3cccc(-n4cc(Cl)c5c(N)ncnc54)c3)cc2n1] | 431.0 | ¹H NMR (400 MHz, DMSO-d6) δ 8.16 (s, 1H), 8.00 (d, J = 9.2 Hz, 1H), 7.94 (s, 1H), 7.85 (s, 1H), 7.77 (d, J = 8.9 Hz, 1H), 7.56 (t, J = 7.8 Hz, 1H), 7.49 (d, J = 7.7 Hz, 1H), 7.17 (d, J = 2.6 Hz, 1H), 7.05 (dd, J = 9.1, 2.4 Hz, 2H), 6.62 (s, 2H), 6.30 (s, 1H), 5.29 (s, 2H), 2.36 (m, 3H). | B |
| 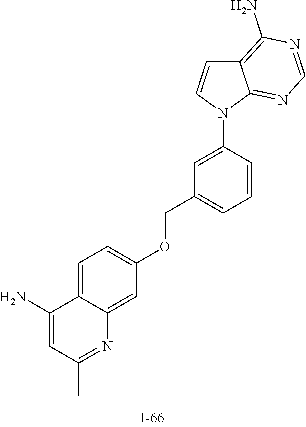 [SMILES: Cc1cc(N)c2ccc(OCc3cccc(-n4ccc5c(N)ncnc54)c3)cc2n1] | 397.3 | ¹H NMR (400 MHz, DMSO-d6) δ 8.13 (s, 1H), 8.07-7.92 (m, 2H), 7.81 (d, J = 8.0 Hz, 1H), 7.63-7.51 (m, 2H), 7.47 (d, J = 7.7 Hz, 1H), 7.17 (d, J = 2.6 Hz, 3H), 7.05 (dd, J = 9.1, 2.6 Hz, 1H), 6.79 (d, J = 3.7 Hz, 1H), 6.58 (s, 2H), 6.30 (s, 1H), 5.30 (s, 2H), 2.35 (s, 3H). | B |
| 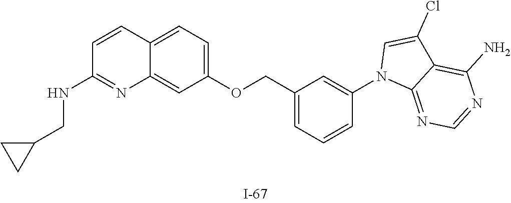 [SMILES: Nc1ncnc2c1c(Cl)cn2-c1cccc(COc2ccc3ccc(NCC4CC4)nc3c2)c1] | 471.1 | ¹H NMR (400 MHz, DMSO) δ 8.15 (s, 1H), 7.92 (s, 1H), 7.84 (s, 1H), 7.76-7.72 (m, 2H), 7.58-7.47 (m, 3H), 7.03-7.02 (m, 2H), 6.87-6.85 (m, 1H), 6.62 (d, J = 8.9 Hz, 1H), 5.27 (s, 2H), 3.25-3.22 (m, 2H), 1.09-1.06 (m, 1H), 0.46-0.44 (m, 2H), 0.24-0.23 (m, 2H). | B |
| 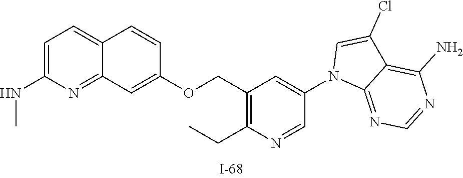 [SMILES: CCc1ncc(-n2cc(Cl)c3c(N)ncnc32)cc1COc1ccc2ccc(NC)nc2c1] | 460.0 | ¹H NMR (400 MHz, DMSO-d6) δ ppm 8.89 (d, J = 2.4 Hz, 1H), 8.32 (d, J = 2.8 Hz, 1H), 8.15 (s, 1H), 7.91 (s, 1H), 7.76 (d, J = 8.8 Hz, 1H), 7.54 (d, J = 8.4 Hz, 1H), 7.16 (d, J = 8.8 Hz, 1H)), 6.98 (brs, 2H), 6.89 (d, J = 8.8 Hz, 1H), 6.59 (d, J = 8.8 Hz, 1H), 5.32 (s, 2H), 2.88-2.94 (m, 5H), 1.30 (t, J = 9.2 Hz, 3H). | B |
| 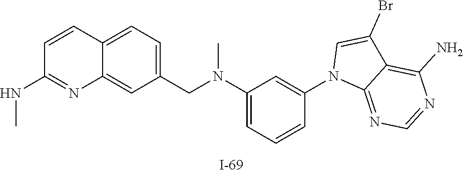 [SMILES: CNc1ccc2ccc(CN(C)c3cccc(-n4cc(Br)c5c(N)ncnc54)c3)cc2n1] | 488.0 | ¹H NMR (400 MHz, DMSO-d6) δ 8.12 (s, 1H), 7.81 (s, 1H), 7.77 (d, J = 8.9 Hz, 1H), 7.56 (d, J = 8.1 Hz, 1H), 7.33 (s, 1H), 7.28-7.23 (m, 1H), 7.12-7.11 (m, 1H), 7.05 (dd, J = 8.0, 1.6 Hz, 1H), 7.00 (d, J = 7.8 Hz, 1H), 6.97-6.92 (m, 1H), 6.73 (dd, J = 8.4, 2.2 Hz, 1H), 6.67 (d, J = 8.9 Hz, 1H), 4.73 (s, 2H), 3.32 (s, 2H), 3.12 (s, 3H), 2.84 (d, J = 4.7 Hz, 3H). | E |
| 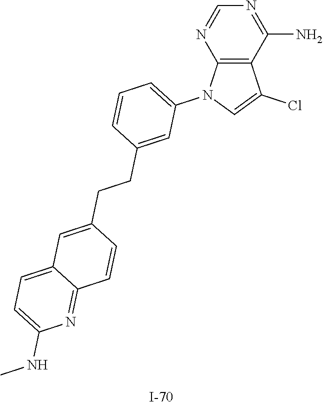 [SMILES: CNc1ccc2cc(CCc3cccc(-n4cc(Cl)c5c(N)ncnc54)c3)ccc2n1] | 429.0 | ¹H NMR (400 MHz, DMSO-d6) δ 8.16 (s, 1H), 7.83-7.72 (m, 2H), 7.70-7.60 (m, 2H), 7.52 (d, J = 8.1 Hz, 1H), 7.48-7.37 (m, 2H), 7.27 (d, J = 7.7 Hz, 1H), 7.08 (dd, J = 8.1, 1.5 Hz, 1H), 7.06-6.80 (m, , 2H), 6.67 (d, J = 8.9 Hz, 1H), 3.03 (s, 4H), 2.88 (d, J = 4.7 Hz, 3H). | C |
| 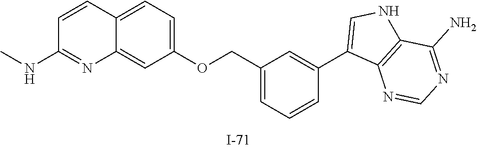 [SMILES: CNc1ccc2ccc(OCc3cccc(-c4c[nH]c5c(N)ncnc45)c3)cc2n1] | 397.0 | ¹H NMR (400 MHz, CD₃OD) δ 8.22 (s, 1H), 8.03 (s, 1H), 7.88 (d, J = 7.6 Hz, 1H), 7.84 (s, 1H), 7.76 (d, J = 8.9 Hz, 1H), 7.51 (d, J = 8.7 Hz, 1H), 7.48-7.44 (m, 1H), 7.39 (d, J = 7.7 Hz, 1H), 7.24 (d, J = 2.3 Hz, 1H), 6.95 (dd, J = 8.7, 2.4 Hz, 1H), 6.58 (d, J = 8.9 Hz, 1H), 5.26 (s, 2H), 2.99 (s, 3H). | D |
| 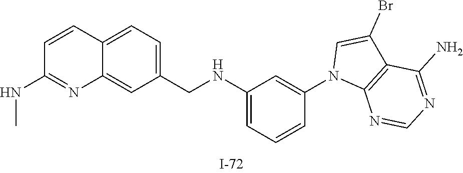 [SMILES: CNc1ccc2ccc(CNc3cccc(-n4cc(Br)c5c(N)ncnc54)c3)cc2n1] | 476.0 | ¹H NMR (400 MHz, CD₃OD) 8.05 (s, 1H), 7.81 (d, J = 8.8 Hz, 1H), 7.68 (s, 1H), 7.57 (d, J = 8.1 Hz, 1H), 7.41 (s, 1H), 7.26-7.19 (m, 2H), 6.91-6.89 (m, 1H), 6.80-6.77 (d, 1H), 6.71-6.88 (m, 2H), 4.51 (s, 2H), 3.00 (s, 3H). | E |
| 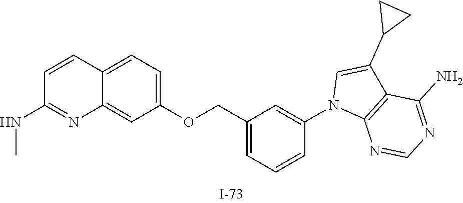 [SMILES: CNc1ccc2ccc(OCc3cccc(-n4cc(C5CC5)c5c(N)ncnc54)c3)cc2n1] | 437.1 | ¹H NMR (400 MHz, CD3OD) δ 8.07 (s, 1H), 7.79-7.72 (m, 2H), 7.62-7.60 (m, 1H), 7.56-7.50 (m, 3H), 7.20 (d, J = 2.1 Hz, 1H), 7.15 (s, 1H), 6.94 (dd, J = 8.7, 2.2 Hz, 1H), 6.58 (d, J = 8.9 Hz, 1H), 5.29 (s, 2H), 2.98 (s, 3H), 1.29 (s, 1H), 1.00-0.88 (m, 2H), 0.74-0.72 (m, 2H). | B |
| 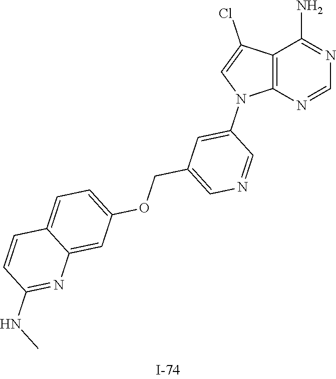 [SMILES: CNc1ccc2ccc(OCc3cncc(-n4cc(Cl)c5c(N)ncnc54)c3)cc2n1] | 432.1 | ¹H NMR (400 MHz, DMSO) δ 9.02 (s, 1H), 8.70 (s, 1H), 8.42 (s, 1H), 8.19 (s, 1H), 7.97 (s, 1H), 7.74 (d, J = 8.9 Hz, 1H), 7.53 (d, J = 8.7 Hz, 1H), 7.10 (d, J = 2.4 Hz, 1H), 6.95 (d, J = 4.7 Hz, 1H), 6.88 (dd, J = 8.7, 2.5 Hz, 1H), 6.58 (d, J = 8.8 Hz, 1H), 5.36 (s, 2H), 2.88 (d, J = 4.7 Hz, 3H). | B |
| 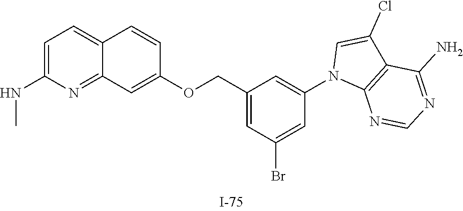 [SMILES: CNc1ccc2ccc(OCc3cc(Br)cc(-n4cc(Cl)c5c(N)ncnc54)c3)cc2n1] | 510 | ¹H NMR (400 MHz, DMSO-d6) δ 8.23 (s, 1H), 8.14 (s, 1H), 8.03 (s, 1H), 7.97 (s, 1H), 7.78 (d, J = 6 Hz, 1H), 7.70 (s, 1H), 7.57 (d, J = 4.2 Hz, 1H), 7.08-7.07 (m, 1H), 6.93 (d, J = 4 Hz, 1H), 6.90-6.88 (m, 1H), 6.62 (d, J = 4.4 Hz, 1H), 5.33 (s, 2H), 2.91-2.90 (m, 3H). | B |
| 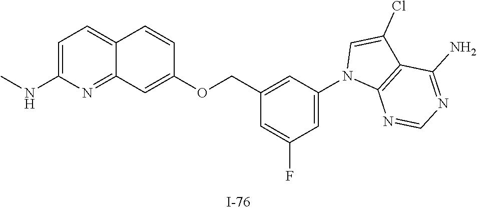 [SMILES: CNc1ccc2ccc(OCc3cc(F)cc(-n4cc(Cl)c5c(N)ncnc54)c3)cc2n1] | 449.0 | ¹H NMR (400 MHz, DMSO-d₆) δ 8.20 (s, 1H), 7.93-7.89 (m, 2H), 7.80-7.73 (m, 2H), 7.53 (d, J = 8.7 Hz, 1H), 7.33 (d, J = 8.9 Hz, 1H), 7.05 (s, 1H), 6.95-6.88 (m, 2H), 6.58 (d, J = 8.8 Hz, 1H), 5.30 (s, 2H), 2.87 (d, J = 4.5 Hz, 3H), 1.23 (s, 1H). | B |
| 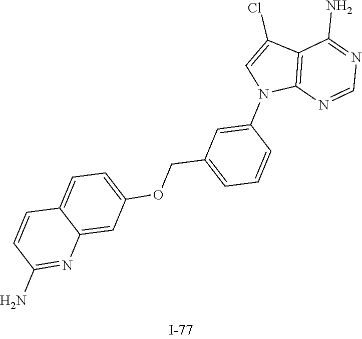 [SMILES: Nc1ccc2ccc(OCc3cccc(-n4cc(Cl)c5c(N)ncnc54)c3)cc2n1] | 417.0 | ¹H NMR (400 MHz, DMSO-d6) δ 8.16 (s, 1H), 7.93 (s, 1H), 7.85 (s, 1H), 7.79-7.75 (m, 2H), 7.58-7.47 (m, 3H), 6.95 (d, J = 2.4 Hz, 1H), 6.88 (dd, J = 8.8 Hz, 2.4 Hz, 1H), 6.57 (d, J = 8.8 Hz, 1H), 6.32 (s, 2H), 5.27 (s, 2H). | B |
| 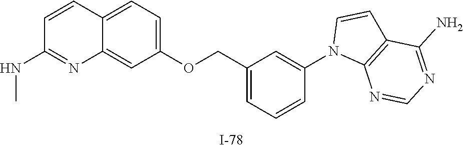 [SMILES: CNc1ccc2ccc(OCc3cccc(-n4ccc5c(N)ncnc54)c3)cc2n1] | 397.7 | ¹H NMR (400 MHz, DMSO-d6) δ 8.13 (s, 1H), 7.96 (s, 1H), 7.80 (d, J = 8.0 Hz, 1H), 7.73 (d, J = 8.4 Hz, 1H), 7.58-7.50 (m, 4H), 7.45 (d, J = 7.6 Hz, 1H), 7.17 (s, 1H), 7.06 (s, 1H), 6.93-6.91 (m, 1H), 6.88-6.86 (m, 1H), 6.78 (d, J = 3.6 Hz, 1H), 6.57 (d, J = 8.8 Hz, 1H), 5.29 (s, 2H), 2.86 (d, J = 4.8 Hz, 3H). | B |
| 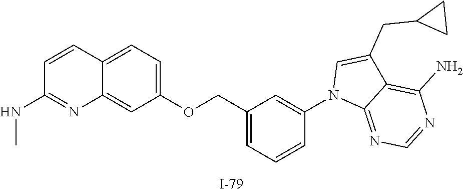 [SMILES: CNc1ccc2ccc(OCc3cccc(-n4cc(CC5CC5)c5c(N)ncnc54)c3)cc2n1] | 451.1 | ¹H NMR (400 MHz, DMSO-d6) δ 8.07 (s, 1H), 7.91 (s, 1H), 7.78 (d, J = 8.3 Hz, 1H), 7.71 (d, J = 8.8 Hz, 1H), 7.54-7.48 (m, 2H), 7.42-7.37 (m, 2H), 7.04 (d, J = 1.9 Hz, 1H), 6.91-6.83 (m, 2H), 6.64 (s, 2H), 6.55 (d, J = 8.9 Hz, 1H), 5.27 (s, 2H), 2.85 (d, J = 4.6 Hz, 3H), 2.76 (d, J = 6.6 Hz, 2H), 1.09-1.06 (m, 1H), 0.51-0.46 (m, 2H), 0.19-0.18 (m, 2H). | B |
| 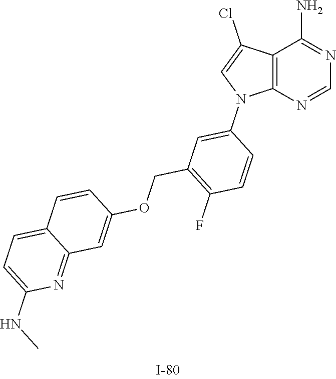 [SMILES: CNc1ccc2ccc(OCc3cc(-n4cc(Cl)c5c(N)ncnc54)ccc3F)cc2n1] | 449.0 | ¹H NMR (400 MHz, DMSO-d6) δ 8.14 (s, 1H), 8.02 (dd, J = 6.4, 2.7 Hz, 1H), 7.89-7.78 (m, 2H), 7.74 (d, J = 8.9 Hz, 1H), 7.52 (d, J = 8.7 Hz, 1H), 7.46 (t, J = 9.2 Hz, 1H), 7.11 (d, J = 2.4 Hz, 1H), 7.08-6.88 (m, 2H), 6.85 (dd, J = 8.7, 2.5 Hz, 1H), 6.58 (d, J = 8.8 Hz, 1H), 5.28 (s, 2H), 2.88 (d, J = 4.7 Hz, 3H). | B |
| 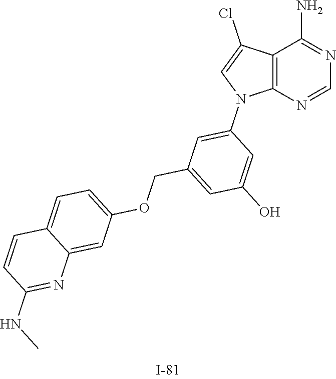 [SMILES: CNc1ccc2ccc(OCc3cc(O)cc(-n4cc(Cl)c5c(N)ncnc54)c3)cc2n1] | 447.1 | ¹H NMR (400 MHz, CD₃OD) δ 9.91 (s, 1H), 8.16 (s, 1H), 7.78 (s, 1H), 7.73 (d, J = 8.9 Hz, 1H), 7.51 (d, J = 8.7 Hz, 1H), 7.29 (s, 1H), 7.22 (d, J = 2.0 Hz, 1H), 7.09-6.82 (m, 5H), 6.57 (d, J = 8.8 Hz, 1H), 5.20 (s, 2H), 2.86 (d, J = 4.7 Hz, 3H). | B |
| 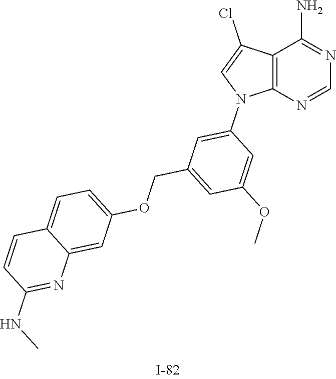 [SMILES: CNc1ccc2ccc(OCc3cc(OC)cc(-n4cc(Cl)c5c(N)ncnc54)c3)cc2n1] | 461.1 | ¹H NMR (400 MHz, DMSO-d6) δ 8.16 (s, 1H), 7.87 (s, 1H), 7.73 (d, J = 8.8 Hz, 1H), 7.59-7.46 (m, 2H), 7.36 (s, 1H), 7.09-7.02 (m, 2H), 6.94-6.91 (m, 1H), 6.89-6.86 (m, 1H), 6.57 (d, J = 8.9 Hz, 1H), 5.25 (s, 2H), 3.84 (s, 3H), 2.87 (d, J = 4.7 Hz, 3H). | B |
| 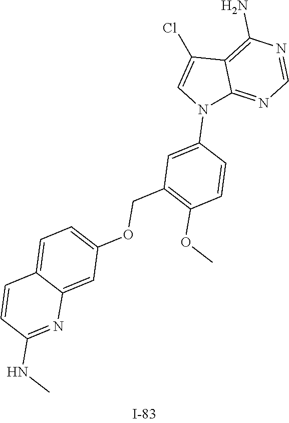 [SMILES: CNc1ccc2ccc(OCc3cc(-n4cc(Cl)c5c(N)ncnc54)ccc3OC)cc2n1] | 461.0 | ¹H NMR (400 MHz, DMSO-d6) δ 8.18 (s, 1H), 8.11 (s, 1H), 7.80 (d, J = 2.6 Hz, 1H), 7.73 (t, J = 4.4 Hz, 2H), 7.68 (dd, J = 8.8, 2.7 Hz, 1H), 7.50 (d, J = 8.7 Hz, 1H), 7.22 (d, J = 8.9 Hz, 1H), 7.02 (d, J = 2.3 Hz, 1H), 7.00-6.88 (m, 2H), 6.84 (dd, J = 8.7, 2.4 Hz, 1H), 6.57 (d, J = 8.8 Hz, 1H), 5.19 (s, 2H), 3.92 (s, 3H), 2.87 (d, J = 4.7 Hz, 3H). | B |
| 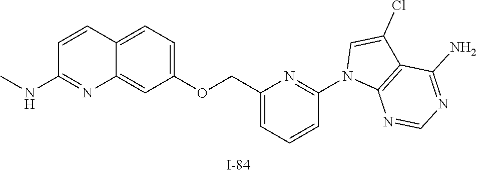 [SMILES: CNc1ccc2ccc(OCc3cccc(-n4cc(Cl)c5c(N)ncnc54)n3)cc2n1] | 432 | ¹H NMR (400 MHz, DMSO-d6 ) δ ppm δ 8.64-8.62 (d, J = 8.2 Hz, 1H), 8.27 (s, 1H), 8.11 (s, 1H), 8.05-8.04 (t, J = 2.2 Hz, 1H), 7.75-7.73 (d, J = 8.1 Hz, 2H), 7.55-7.52 (d, J = 12.0 Hz, 1H), 7.48-7.47 (d, J = 4.2 Hz, 1H), 7.06-7.05 (m, 1H), 6.92-6.89 (m, 2H), 6.58-6.56 (d, J = 8.2 Hz, 1H), 5.33 (s, 2H), 2.86-2.85 (d, J = 4.1 Hz, 3H). | B |
| 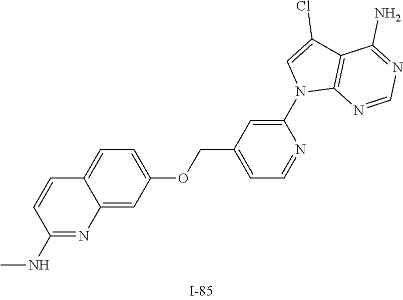 [SMILES: CNc1ccc2ccc(OCc3ccnc(-n4cc(Cl)c5c(N)ncnc54)c3)cc2n1] | 432.1 | ¹H NMR (400 MHz, DMSO-d6) δ ppm 8.83 (s, 1H), 8.52 (d, J = 5.1 Hz, 1H), 8.27 (s, 1H), 8.11 (s, 1H), 7.74 (d, J = 9.0 Hz, 1H), 7.74 (d, J = 9.0 Hz, 1H), 7.55 (d, J = 8.7 Hz, 1H), 7.44 (d, J = 5.2 Hz, 1H), 7.26-7.02 (m, 2H), 6.98-6.87 (m, 2H), 6.58 (d, J = 8.8 Hz, 1H), 5.42 (s, 2H), 2.86 (d, J = 4.7 Hz, 3H). | B |
| 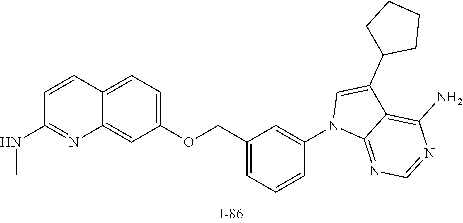 [SMILES: CNc1ccc2ccc(OCc3cccc(-n4cc(C5CCCC5)c5c(N)ncnc54)c3)cc2n1] | 465.7 | ¹H NMR (400 MHz, DMSO-d6) δ ppm 8.09 (s, 1H), 7.91 (s, 1H), 7.84-7.81 (s, 1H), 7.73 (d, J = 8.8 Hz, 1H), 7.55-7.48 (m, 2H), 7.41 (d, J = 7.6 Hz, 1H), 7.34 (s, 1H), 7.06-7.05 (m, 1H), 6.94-6.90 (m, 1H), 6.88-6.85 (m, 1H), 6.66 (s, 2H), 6.57 (d, J = 8.8 Hz, 1H), 5.28 (s, 2H), 3.48-3.41 (m, 1H), 2.86 (d, J = 4.8 Hz, 3H), 2.08-2.05 (m, 2H), 1.76-1.57 (m, 6H). | B |
| 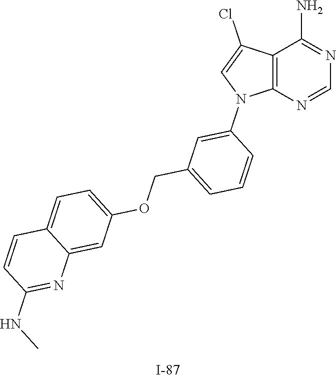 [SMILES: CNc1ccc2ccc(OCc3cccc(-n4cc(Cl)c5c(N)ncnc54)c3)cc2n1] | 431.7 | ¹H NMR (400 MHz, DMSO-d6) δ ppm 8.16 (s, 1H), 7.93 (s, 1H), 7.84 (d, 1H), 7.77-7.72 (m, 2H), 7.58-7.47 (m, 3H), 7.06 (d, J = 2.4 Hz, 1H), 6.94-6.91 (m, 1H), 6.87 (dd, J₁ = 2.4 Hz, J₂ = 8.4 Hz, 1H), 6.57 (d, J = 8.8 Hz, 1H), 5.28 (s, 2H), 2.87 (d, J = 4.4 Hz, 3H). | B |
| 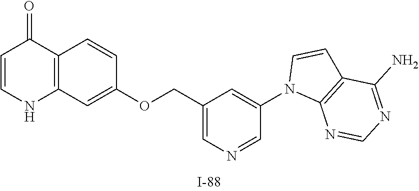 [SMILES: Nc1ncnc2c1ccn2-c1cncc(COc2ccc3c(=O)cc[nH]c3c2)c1] | 385.1 | ¹H NMR (400 MHz, DMSO-d6) δ ppm 9.09 (d, J = 2.4 Hz, 1H), 8.68 (d, J = 1.7 Hz, 1H), 8.48-8.46 (m, 1H), 8.15 (s, 1H), 8.02-7.99 (m, 1H), 7.81 (d, J = 7.5 Hz, 1H), 7.70 (d, J = 3.7 Hz, 1H), 7.24 (s, 3H), 7.05 (s, 2H), 6.84 (d, J = 3.7 Hz, 1H), 5.94 (d, J = 7.4 Hz, 1H), 5.36 (s, 2H). | B |
| 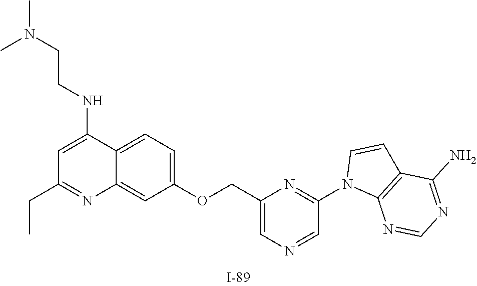 [SMILES: CCc1cc(NCCN(C)C)c2ccc(OCc3cncc(-n4ccc5c(N)ncnc54)n3)cc2n1] | 484.2 | ¹H NMR (400 MHz, DMSO-d6) δ ppm 9.99 (s, 1H), 8.74 (s, 1H), 8.27 (s, 1H), 8.20 (s, 1H), 8.09 (d, J = 9.2 Hz, 1H), 7.97 (d, J = 4 Hz, 1H), 7.35 (br, 1H), 7.31 (d, J = 2.8 Hz, 1H), 7.19 (dd, J = 9.6, 2.8 Hz, 1H), 7.04 (br, 1H), 6.90 (d, J = 3.6 Hz, 1H), 6.33 (s, 1H), 5.44 (s, 2H), 2.74-2.67 (m, 3H), 2.58-2.51 (m, 3H), 2.23 (s, 6H), 1.26 (t, J = 7.6 Hz, 3H) | B |
| 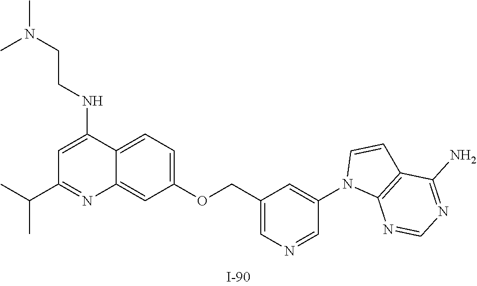 [SMILES: CC(C)c1cc(NCCN(C)C)c2ccc(OCc3cncc(-n4ccc5c(N)ncnc54)c3)cc2n1] | 498.2 | ¹H NMR (400 MHz, DMSO-d6) δ ppm 11.38 (s, 1H), 9.32 (d, J = 2.1 Hz, 1H), 8.69 (t, J = 2.0 Hz, 1H), 8.51 (d, J = 2.0 Hz, 1H), 8.24 (s, 1H), 8.20 (d, J = 2.9 Hz, 1H), 8.02 (d, J = 8.8 Hz, 1H), 7.25 (d, J = 2.5 Hz, 1H), 7.06 (d, J = 8.0 Hz, 1H), 6.85 (s, 2H), 6.58-6.55 (m, 1H), 6.36 (s, 1H), 5.31 (s, 2H), 2.93-2.87 (m, 1H), 1.24 (d, J = 6.9 Hz, 6H). | B |
| 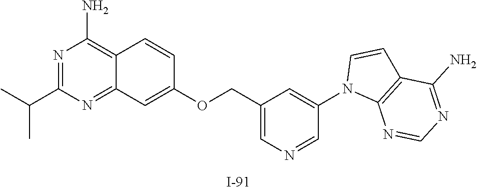 [SMILES: CC(C)c1nc(N)c2ccc(OCc3cncc(-n4ccc5c(N)ncnc54)c3)cc2n1] | 427.0 | ¹H NMR (400 MHz, DMSO-d6) δ ppm 9.08 (d, J = 2.5 Hz, 1H), 8.67 (d, J = 1.8 Hz, 1H), 8.52-8.42 (m, 1H), 8.15 (m, 1H), 8.09 (d, J = 9.0 Hz, 1H), 7.70 (d, J = 3.7 Hz, 1H), 7.48 (s, 2H), 7.24 (s, 2H), 7.18 (d, J = 2.6 Hz, 1H), 7.12 (dd, J = 9.0, 2.6 Hz, 1H), 6.84 (d, J = 3.7 Hz, 1H), 5.41 (s, 2H), 2.95-2.87 (m, , 1H), 1.24 (d, J = 6.9 Hz, 6H). | B |
| 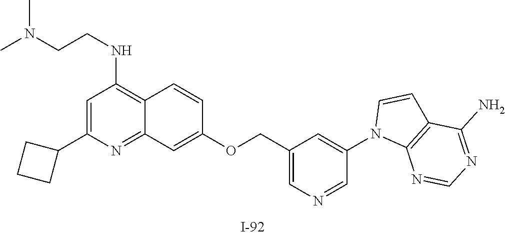 [SMILES: CN(C)CCNc1cc(C2CCC2)nc2cc(OCc3cncc(-n4ccc5c(N)ncnc54)c3)ccc12] | 509.2 | ¹H NMR (400 MHz, DMSO-d6) δ ppm 9.08 (d, J = 2.4 Hz, 1H), 8.70 (d, J = 2.0 Hz, 1H), 8.49-8.50 (m, 1H), 8.30 (d, J = 8.4 Hz, 1H), 8.14 (s, 1H), 7.71 (s, J = 3.6 Hz, 1H), 7.37-7.40 (m, 2H), 7.27 (s, 2H), 6.85 (d, J = 3.6 Hz, 1H), 6.59 (s, 1H), 5.45 (s, 2H), 3.73-3.77 (m, 1H), 3.65 (s, 2H), 2.83 (s, 2H), 2.41-2.43 (m, 6H), 2.32-2.38 (m, 4H), 2.06-2.11 (m, 1H). 1.87-1.90 (m, 1H). | B |
| 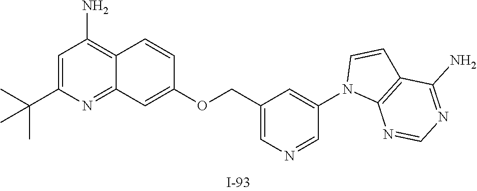 [SMILES: CC(C)(C)c1cc(N)c2ccc(OCc3cncc(-n4ccc5c(N)ncnc54)c3)cc2n1] | 440.0 | ¹H NMR (400 MHz, DMSO-d6) δ ppm 9.09 (d, J = 2.4 Hz, 1H), 8.70 (d, J = 2.0 Hz, 1H), 8.48 (s, 1H), 8.17 (s, 1H), 8.02 (d, J = 1.2 Hz, 1H), 7.72 (d, J = 3.6 Hz, 1H), 7.26 (s, 3H), 7.17 (s,, 1H), 6.86 (d, J = 3.6 Hz, 1H), 6.58 (s, 2H), 5.41 (s, 2H), 1.34 (s, 9H). | B |
| 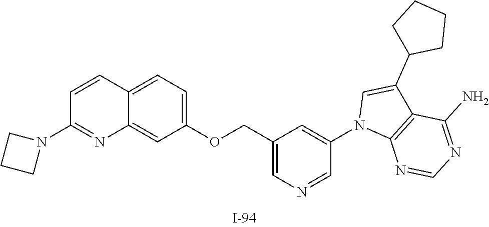 [SMILES: Nc1ncnc2c1c(C1CCCC1)cn2-c1cncc(COc2ccc3ccc(N4CCC4)nc3c2)c1] | 492.1 | ¹H NMR (400 MHz, DMSO-d6) δ ppm 9.10 (d, J = 2.3 Hz, 1H), 8.62 (d, J = 1.4 Hz, 1H), 8.43 (s, 1H), 8.13 (s, 1H), 7.92 (d, J = 8.8 Hz, 1H), 7.61 (d, J = 8.8 Hz, 1H), 7.49 (s, 1H), 7.14 (d, J = 2.3 Hz, 1H), 6.94 (dd, J = 8.8, 2.5 Hz, 1H), 6.74 (s, 2H), 6.52 (d, J = 8.8 Hz, 1H), 5.35 (s, 2H), 4.06 (t, J = 7.4 Hz, 4H), 3.51-3.38 (m, 1H), 2.39-2.28 (m, 2H), 2.08-2.06 (m, 2H), 1.81-1.55 (m, 6H). | B |
| 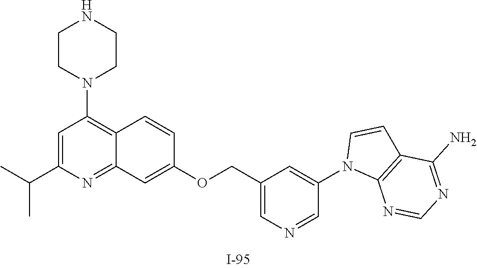 [SMILES: CC(C)c1cc(N2CCNCC2)c2ccc(OCc3cncc(-n4ccc5c(N)ncnc54)c3)cc2n1] | 495.0 | ¹H NMR (400 MHz, DMSO-d6) δ ppm 9.08 (d, J = 2.4 Hz, 1H), 8.69 (d, J = 1.6 Hz, 1H), 8.48 (t, J = 2.0 Hz, 1H), 8.15 (s, 1H), 7.87 (d, J = 9.2 Hz, 1H), 7.71 (d, J = 3.7 Hz, 1H), 7.43 (d, J = 2.5 Hz, 1H), 7.30-7.17 (m, 3H), 6.84 (d, J = 3.7 Hz, 1H), 6.75 (s, 1H), 5.42 (s, 2H), 3.12-3.02 (m, 5H), 2.97-2.93 (m, 4H), 1.29 (d, J = 6.9 Hz, 6H). | B |
| 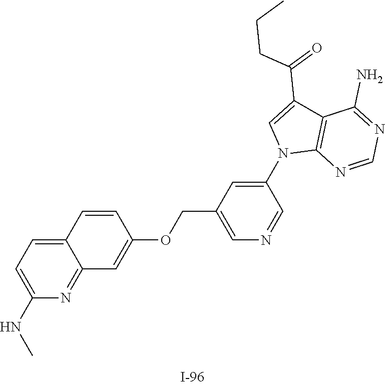 [SMILES: CCCC(=O)c1cn(-c2cncc(COc3ccc4ccc(NC)nc4c3)c2)c2ncnc(N)c12] | 468.1 | ¹H NMR (400 MHz, DMSO-d6) δ ppm 9.07 (d, J = 2.4 Hz, 1H), 8.85 (s, 1H), 8.79 (d, J = 1.7 Hz, 1H), 8.45 (t, J = 2.0 Hz, 1H), 8.42 (s, 1H), 8.20 (s, 1H), 7.75 (d, J = 8.9 Hz, 1H), 7.69-7.57 (m, 1H), 7.54 (d, J = 8.7 Hz, 1H), 7.12 (d, J = 2.4 Hz, 1H), 7.03-6.92 (m, 1H), 6.89 (dd, J = 8.7, 2.5 Hz, 1H), 6.59 (d, J = 8.8 Hz, 1H), 5.39 (s, 2H), 2.99 (t, J = 7.3 Hz, 2H), 2.88 (d, J = 4.7 Hz, 3H), 1.80-1.60 (m, 2H), 0.96 (t, J = 7.4 Hz, 3H). | B |
| 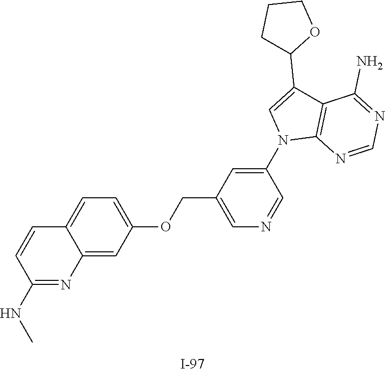 [SMILES: CNc1ccc2ccc(OCc3cncc(-n4cc(C5CCCO5)c5c(N)ncnc54)c3)cc2n1] | 468.1 | ¹H NMR (400 MHz, DMSO-d6) δ ppm 9.07 (d, J = 2.5 Hz, 1H), 8.67 (d, J = 1.8 Hz, 1H), 8.43 (t, J = 2.1 Hz, 1H), 8.17 (s, 1H), 7.74 (d, J = 8.8 Hz, 1H), 7.70 (s, 1H), 7.53 (d, J = 8.8 Hz, 1H), 7.10 (d, J = 2.5 Hz, 1H), 7.04 (s, 2H), 6.98-6.92 (m, 1H), 6.88 (dd, J = 8.7, 2.5 Hz, 1H), 6.58 (d, J = 8.8 Hz, 1H), 5.36 (s, 2H), 5.12-4.98 (m, 1H), 4.03-3.93 (m, 1H), 3.94-3.84 (m, 1H), 2.88 (d, J = 4.8 Hz, 3H), 2.37-2.22 (m, 1H), 2.14-1.96 (m, 3H). | B |
| 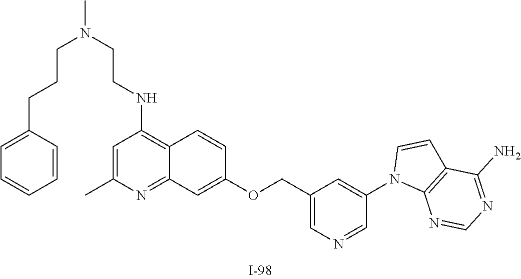 [SMILES: Cc1cc(NCCN(C)CCCc2ccccc2)c2ccc(OCc3cncc(-n4ccc5c(N)ncnc54)c3)cc2n1] | 573.3 | ¹H NMR (400 MHz, DMSO-d6) δ ppm 9.10 (d, J = 2.4 Hz, 1H), 8.70 (d, J = 1.6 Hz, 1H), 8.49 (t, J = 1.6 Hz, 1H), 8.31 (s, 2H), 8.22 (d, J = 8.8 Hz, 1H), 8.16 (s, 1H), 7.71 (d, J = 4 Hz, 1H), 7.43 (s, 1H), 7.27 (s, 1H), 7.20 (t, J = 7.6 Hz, 2H), 7.14-7.08 (m, 2H), 6.86 (d, J = 3.6 Hz, 1H), 6.50 (s, 1H), 5.41 (s, 2H), 3.48 (s, 2H), 2.66 (t, J = 6.4 Hz, 2H), 2.51 (t, J = 1.6 Hz, 3H), 2.41 (t, J = 7.2 Hz, 2H), 2.29 (s, 3H), 1.73-1.65 (m, 2H). | B |
| 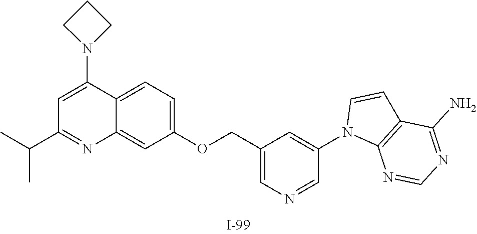 [SMILES: CC(C)c1cc(N2CCC2)c2ccc(OCc3cncc(-n4ccc5c(N)ncnc54)c3)cc2n1] | 466.2 | ¹H NMR (400 MHz, DMSO-d6) δ ppm 9.07 (d, J = 2.5 Hz, 1H), 8.68 (d, J = 1.8 Hz, 1H), 8.48-8.45 (m, 1H), 8.19 (s, 1H), 8.15 (s, 1H), 7.87 (d, J = 9.2 Hz, 1H), 7.70 (d, J = 3.7 Hz, 1H), 7.33 (d, J = 1.9 Hz, 1H), 7.24 (s, 2H), 7.07 (dd, J = 9.2, 2.4 Hz, 1H), 6.84 (d, J = 3.7 Hz, 1H), 6.06 (s, 1H), 5.40 (s, 2H), 4.34 (t, J = 7.2 Hz, 4H), 2.42-2.36 (m, 2H), 1.26 (d, J = 6.9 Hz, 6H). | B |
| 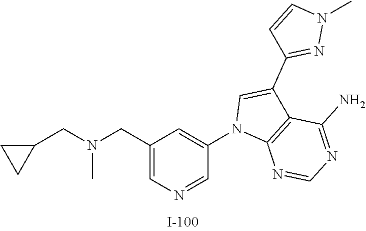 [SMILES: CN(Cc1cncc(-n2cc(-c3ccn(C)n3)c3c(N)ncnc32)c1)CC1CC1] | 389.1 | ¹H NMR (400 MHz, DMSO-d6) δ ppm 9.18 (s, 1H), 9.01 (d, J = 2.0 Hz, 1H), 8.50 (s, 1H), 8.27 (s, 1H), 8.21 (s, 1H), 8.14 (s, 1H), 7.78 (d, J = 2.4 Hz, 1H), 7.39 (s, 1H), 6.79 (d, J = 2.4 Hz, 1H), 3.91 (s, 3H), 3.69-3.74 (m, 2H), 2.28-2.32 (m, 5H), 0.91-0.93 (m, 1H), 0.46-0.50 (m, 2H), 0.11 (d, J = 4.0 Hz, 1H). | B |
| 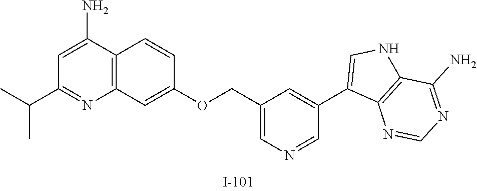 [SMILES: CC(C)c1cc(N)c2ccc(OCc3cncc(-c4c[nH]c5c(N)ncnc45)c3)cc2n1] | 426.0 | ¹H NMR (400 MHz, DMSO-d6) δ ppm 11.37 (s, 1H), 9.32 (d, J = 2.1 Hz, 1H), 8.69 (t, J = 2.0 Hz, 1H), 8.51 (d, J = 2.0 Hz, 1H), 8.24 (s, 1H), 8.20 (d, J = 2.9 Hz, 1H), 8.02 (d, J = 8.9 Hz, 1H), 7.25 (d, J = 2.5 Hz, 1H), 7.07 (d, J = 7.6 Hz, 1H), 6.85 (s, 2H), 6.61-6.53 (m, 1H), 6.36 (s, 1H), 5.31 (s, 2H), 2.93-2.87 (m, 1H), 1.24 (d, J = 6.9 Hz, 6H). | D |
| 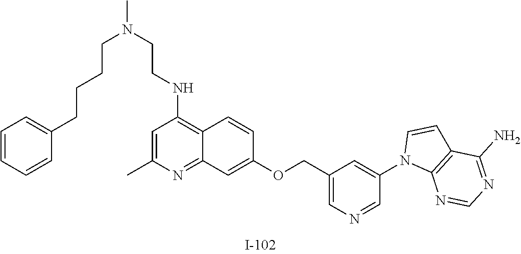 [SMILES: Cc1cc(NCCN(C)CCCCc2ccccc2)c2ccc(OCc3cncc(-n4ccc5c(N)ncnc54)c3)cc2n1] | 294.2 | ¹H NMR (400 MHz, DMSO-d6) δ ppm 9.08 (d, J = 2.0 Hz, 1H), 8.69 (s, 1H), 8.48 (s, 1H), 8.16 (s, 3H), 7.70 (d, J = 3.6 Hz, 1H), 7.29-7.25 (m, 7H), 7.20-7.18 (m, 3H), 6.84 (d, J = 3.6 Hz, 1H), 6.49 (s, 1H), 5.40 (s, 2H), 3.44 (s, 2H), 2.63 (s, 1H), 2.42 (t, J = 3.4 Hz, 4H), 2.39 (s, 5H), 1.51 (t, J = 3.4 Hz, 2H), 1.42 (m, 2H). | B |
| 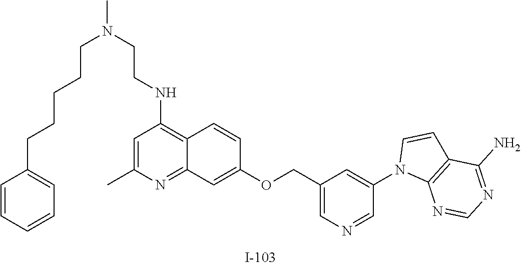 [SMILES: Cc1cc(NCCN(C)CCCCCc2ccccc2)c2ccc(OCc3cncc(-n4ccc5c(N)ncnc54)c3)cc2n1] | 301.3 | ¹H NMR (400 MHz, DMSO-d6) δ ppm 9.08 (d, J = 2.4 Hz, 1H), 8.68 (d, J = 1.6 Hz, 1H), 8.48 (t, J = 1.0 Hz, 1H), 8.20 (s, 1H), 8.17 (s, 5H), 8.15 (t, J = 1.8 Hz, 1H), 7.70 (d, J = 1.8 Hz, 2H), 7.30-7.26 (m, 6H), 7.25-7.24 (m, 3H), 6.84 (d, J = 3.6 Hz, 1H), 6.46 (s, 1H), 5.40 (s, 2H), 3.42 (d, J = 2.4 Hz, 2H), 2.51 (t, J = 0.8 Hz, 1H), 2.50-2.49 (m, 3H), 2.43 (s, 3H), 1.49-1.41 (m, 4H), 1.24-1.23 (m, 2H). | B |
| 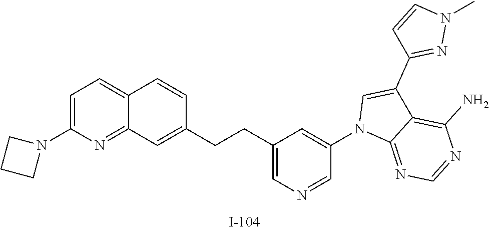 [SMILES: Cn1ccc(-c2cn(-c3cncc(CCc4ccc5ccc(N6CCC6)nc5c4)c3)c3ncnc(N)c23)n1] | 502.0 | ¹H NMR (400 MHz, DMSO-d6) δ ppm 9.16 (s, 1H), 8.97 (d, J = 2.4 Hz, 1H), 8.41 (d, J = 1.7 Hz, 1H), 8.21 (t, J = 2.1 Hz, 1H), 8.13 (d, J = 3.6 Hz, 2H), 7.95 (d, J = 8.9 Hz, 1H), 7.79 (d, J = 2.2 Hz, 1H), 7.61 (d, J = 8.2 Hz, 1H), 7.46 (s, 1H), 7.38 (s, 1H), 7.15 (dd, J = 8.1, 1.6 Hz, 1H), 6.74 (d, J = 2.2 Hz, 1H), 6.62 (d, J = 8.9 Hz, 1H), 4.05 (t, J = 7.4 Hz, 4H), 3.91 (s, 3H), 3.10 (s, 4H), 2.33 (dt, J = 14.8, 7.3 Hz, 2H). | C |
| 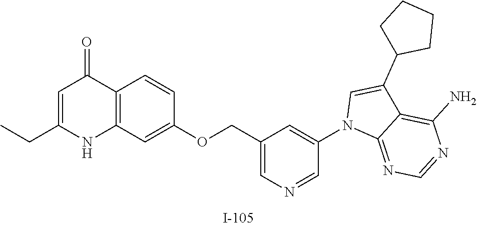 [SMILES: CCc1cc(=O)c2ccc(OCc3cncc(-n4cc(C5CCCC5)c5c(N)ncnc54)c3)cc2[nH]1] | 481.3 | ¹H NMR (400 MHz, DMSO-d6) δ ppm 11.34 (s, 1H), 9.13 (d, J = 2.4 Hz, 1H), 8.63 (d, J = 2.0 Hz, 1H), 8.45 (t, J = 2.0 Hz, 1H), 8.12 (s, 1H), 7.95 (d, J = 8.8 Hz, 1H), 7.51 (s, 1H), 7.03 (d, J = 2.4 Hz, 1H), 7.01-6.98 (m, 1H), 6.76 (s, 1H), 5.83 (s, 1H), 5.34 (s, 1H), 3.50-3.41 (m, 1H), 2.60-2.54 (m, 2H), 2.07-2.04 (m, 2H), 1.74-1.60 (m, 6H), 1.23 (t, J = 7.6 Hz, 3H). | B |
| 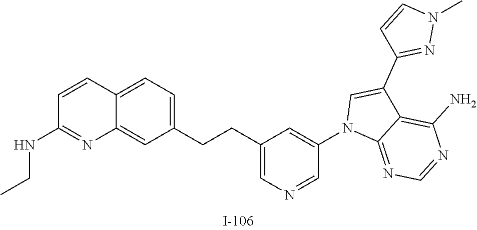 [SMILES: CCNc1ccc2ccc(CCc3cncc(-n4cc(-c5ccn(C)n5)c5c(N)ncnc54)c3)cc2n1] | 490.0 | ¹H NMR (400 MHz, DMSO-d6) δ ppm 9.16 (s, 1H), 8.98 (d, J = 2.4 Hz, 1H), 8.42 (d, J = 1.8 Hz, 1H), 8.23 (t, J = 2.1 Hz, 1H), 8.14 (d, J = 5.0 Hz, 2H), 7.79 (d, J = 2.3 Hz, 1H), 7.77 (d, J = 8.9 Hz, 1H), 7.52 (d, J = 8.1 Hz, 1H), 7.47-7.35 (m, 2H), 7.08 (dd, J = 8.1, 1.6 Hz, 1H), 6.97-6.87 (m, 1H), 6.75 (d, J = 2.3 Hz, 1H), 6.66 (d, J = 8.9 Hz, 1H), 3.92 (s, 3H), 3.44-3.35 (m, 2H), 3.08 (s, 4H), 1.17 (t, J = 7.2 Hz, 3H). | C |
| 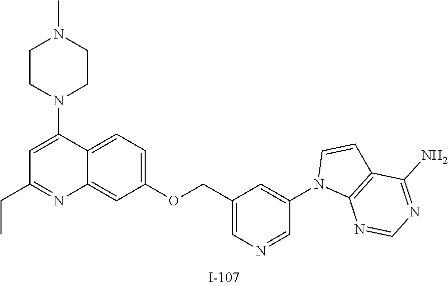 [SMILES: CCc1cc(N2CCN(C)CC2)c2ccc(OCc3cncc(-n4ccc5c(N)ncnc54)c3)cc2n1] | 495.3 | ¹H NMR (400 MHz, DMSO-d6) δ ppm 9.08 (d, J = 2.8 Hz, 1H), 8.70 (d, J = 1.6 Hz, 1H), 8.50 (t, J = 2.4 Hz, 1H), 8.15 (s, 1H), 8.14 (s, 0.55H), 7.93 (d, J = 9.2 Hz, 1H), 7.93 (d, J = 9.2 Hz, 1H), 7.71 (d, J = 3.6 Hz, 1H), 7.46 (d, J = 2.4 Hz, 1H), 7.22-7.28 (m, 3H), 6.89 (s, 1H), 6.85 (d, J = 3.6 Hz, 1H), 5.44 (s, 2H), 2.79-3.08 (m, 10H), 2.67-2.72 (m, 3H), 1.30 (t, J = 7.6 Hz, 3H). | B |
| 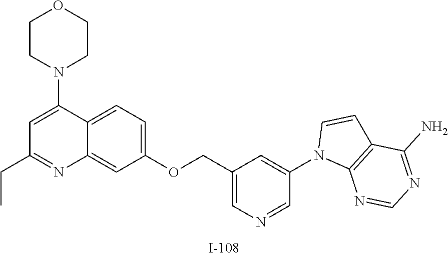 [SMILES: CCc1cc(N2CCOCC2)c2ccc(OCc3cncc(-n4ccc5c(N)ncnc54)c3)cc2n1] | 482.3 | ¹H NMR (400 MHz, DMSO-d6) δ ppm 9.08 (d, J = 2.8 Hz, 1H), 8.70 (d, J = 2.4 Hz, 1H), 8.48-8.49 (m, 1H), 8.14 (d, J = 5.2 Hz, 1H), 7.93 (d, J = 8.8 Hz, 1H), 7.70 (d, J = 4 Hz, 1H),7.44 (d, J = 2.8 Hz, 1H), 7.22-7.25 (m, 2H), 6.84 (d, J = 4 Hz, 1H), 6.81 (s, 1H), 5.42 (s, 2H), 3.84-3.86 (m, 4H), 3.16-3.21 (m, 4H), 2.80-2.86 (m, 2H), 1.29 (t, J = 7.6 Hz, 3H). | B |
| 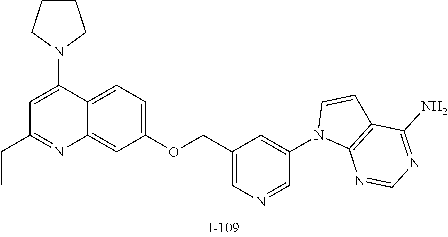 [SMILES: CCc1cc(N2CCCC2)c2ccc(OCc3cncc(-n4ccc5c(N)ncnc54)c3)cc2n1] | 466.3 | ¹H NMR (400 MHz, DMSO-d6) δ ppm 9.09 (d, J = 2.4 Hz, 1H), 8.70 (d, J = 2.4 Hz, 1H), 8.47-8.48 (m, 1H), 8.22 (s, 1H), 8.20 (s, 0.48H), 8.15 (s, 1H), 7.70 (d, J = 3.6 Hz, 1H), 7.34 (d, J = 2.4 Hz, 1H), 7.24 (s, 1H), 7.08 (dd, J = 2.8, 9.6 Hz, 1H), 6.84 (d, J = 3.6 Hz, 1H), 6.39 (s, 1H), 5.40 (s, 2H), 3.64-3.67 (m, 4H), 2.70-2.76 (m, 2H), 1.93-1.98 (m, 4H ), 1.27 (t, J = 7.6 Hz, 3H). | B |
| 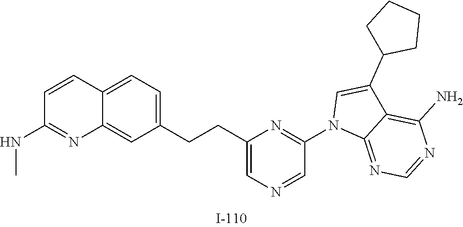 [SMILES: CNc1ccc2ccc(CCc3cncc(-n4cc(C5CCCC5)c5c(N)ncnc54)n3)cc2n1] | 465.1 | ¹H NMR (400 MHz, DMSO-d6) δ ppm 9.77 (s, 1H), 8.41 (s, 1H), 8.22 (s, 1H), 7.75 (d, J = 8.8 Hz, 1H), 7.61 (s, 1H), 7.56 (d, J = 8.0 Hz, 1H), 7.39 (s, 1H), 7.04 (d, J = 8.4 Hz, 1H), 6.92 (d, J = 4.8 Hz,, 1H), 6.82 (s, 1H), 6.66 (d, J = 8.8 Hz, 1H), 3.24-3.18 (dd, J = 17.6, 6.4 Hz, 5H), 2.85 (d, J = 4.8 Hz, 3H), 2.08-2.03 (m, 2H), 1.74-1.68 (m, 4H), 1.53-1.51 (m, 3H). | C |
| 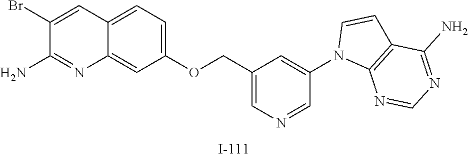 [SMILES: Nc1nc2cc(OCc3cncc(-n4ccc5c(N)ncnc54)c3)ccc2cc1Br] | 462.0 | ¹H NMR (400 MHz, DMSO-d6) δ ppm 9.08 (d, J = 2.4 Hz, 1H), 8.67 (d, J = 1.7 Hz, 1H), 8.46 (m, 1H), 8.29 (s, 1H), 8.15 (s, 1H), 7.70 (d, J = 3.7 Hz, 1H), 7.60 (d, J = 8.9 Hz, 1H), 7.30-7.20 (br, 2H), 7.06-6.95 (m, 2H), 6.84 (d, J = 3.7 Hz, 1H), 6.63-6.53 (br, 2H), 5.36 (s, 2H). | B |
| 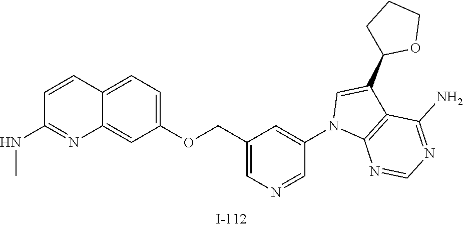 [SMILES: CNc1ccc2ccc(OCc3cncc(-n4cc([C@H]5CCCO5)c5c(N)ncnc54)c3)cc2n1] | 468.1 | ¹H NMR (400 MHz, DMSO-d6) δ ppm 9.07 (d, J = 2.5 Hz, 1H), 8.67 (d, J = 1.8 Hz, 1H), 8.43 (t, J = 2.1 Hz, 1H), 8.17 (s, 1H), 7.74 (d, J = 8.8 Hz, 1H), 7.70 (s, 1H), 7.53 (d, J = 8.8 Hz, 1H), 7.10 (d, J = 2.5 Hz, 1H), 7.04 (s, 2H), 6.98-6.92 (m, 1H), 6.88 (dd, J = 8.7, 2.5 Hz, 1H), 6.58 (d, J = 8.8 Hz, 1H), 5.36 (d, 2H), 5.12-4.98 (m, 1H), 4.03-3.93 (m, 1H), 3.94-3.84 (m, 1H), 2.88 (d, J = 4.8 Hz, 3H), 2.37-2.22 (m, 1H), 2.14-1.96 (m, 3H). | B |
| 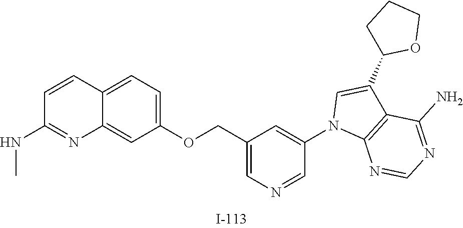 [SMILES: CNc1ccc2ccc(OCc3cncc(-n4cc([C@@H]5CCCO5)c5c(N)ncnc54)c3)cc2n1] | 468.1 | ¹H NMR (400 MHz, DMSO-d6) δ ppm 9.07 (d, J = 2.5 Hz, 1H), 8.67 (d, J = 1.8 Hz, 1H), 8.43 (t, J = 2.1 Hz, 1H), 8.17 (s, 1H), 7.74 (d, J = 8.8 Hz, 1H), 7.70 (s, 1H), 7.53 (d, J = 8.8 Hz, 1H), 7.10 (d, J = 2.5 Hz, 1H), 7.04 (s, 2H), 6.98-6.92 (m, 1H), 6.88 (dd, J = 8.7, 2.5 Hz, 1H), 6.58 (d, J = 8.8 Hz, 1H), 5.36 (s, 2H), 5.12-4.98 (m, 1H), 4.03-3.93 (m, 1H), 3.94-3.84 (m, 1H), 2.88 (d, J = 4.8 Hz, 3H), 2.37-2.22 (m, 1H), 2.14-1.96 (m, 3H). | B |
| 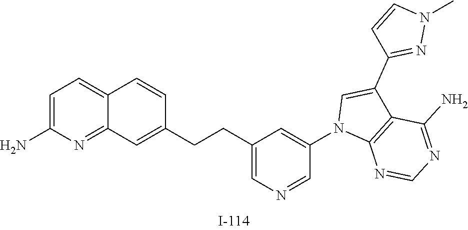 [SMILES: Cn1ccc(-c2cn(-c3cncc(CCc4ccc5ccc(N)nc5c4)c3)c3ncnc(N)c23)n1] | 462.0 | ¹H NMR (400 MHz, DMSO-d6) δ ppm 9.16 (s, 1H), 8.97 (d, J = 2.4 Hz, 1H), 8.40 (d, J = 1.8 Hz, 1H), 8.22 (m, J = 2.2 Hz, 1H), 8.14 (d, J = 5.0 Hz, 2H), 7.81 (dd, J = 12.9, 5.5 Hz, 2H), 7.54 (d, J = 8.1 Hz, 1H), 7.38 (s, 1H), 7.30 (d, 1H), 7.10 (dd, J = 8.1, 1.6 Hz, 1H), 6.75 (d, J = 2.3 Hz, 1H), 6.68 (d, J = 8.8 Hz, 1H), 6.34 (s, 2H), 3.92 (s, 3H), 3.09 (s, 4H). | C |
| 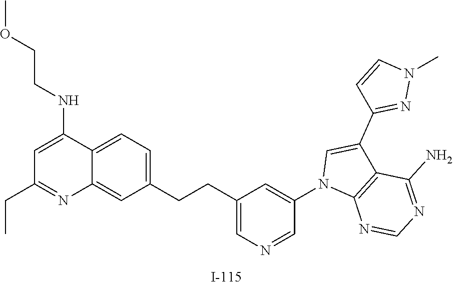 [SMILES: CCc1cc(NCCOC)c2ccc(CCc3cncc(-n4cc(-c5ccn(C)n5)c5c(N)ncnc54)c3)cc2n1] | 548.8 | ¹H NMR (400 MHz, DMSO-d6) δ ppm 9.16 (s, 1H), 8.97 (d, J = 2.4 Hz, 1H), 8.40 (d, J = 1.8 Hz, 1H), 8.21 (t, J = 2.1 Hz, 1H), 8.12 (d, J = 2.8 Hz, 2H), 8.06 (d, J = 8.7 Hz, 1H), 7.79 (d, J = 2.3 Hz, 1H), 7.59 (d, J = 1.6 Hz, 1H), 7.38 (s, 1H), 7.28 (dd, J = 8.5, 1.6 Hz, 1H), 7.01-6.92 (m, 1H), 6.73 (d, J = 2.3 Hz, 1H), 6.34 (s, 1H), 3.92 (s, 3H), 3.59 (t, J = 5.7 Hz, 2H), 3.51-3.40 (m, 2H), 3.29 (s, 3H), 3.12 (s, 4H), 2.70 (q, J = 7.6 Hz, 2H), 1.25 (t, J = 7.6 Hz, 3H). | C |
| 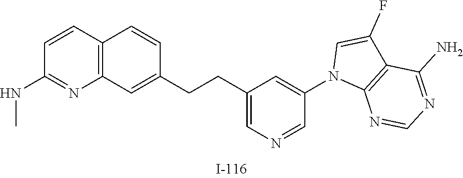 [SMILES: CNc1ccc2ccc(CCc3cncc(-n4cc(F)c5c(N)ncnc54)c3)cc2n1] | 414.0 | ¹H NMR (400 MHz, DMSO-d6) δ ppm 8.90 (d, J = 2.4 Hz, 1H), 8.39 (d, J = 1.8 Hz, 1H), 8.18-8.12 (m, 2H), 7.77 (d, J = 8.0 Hz, 1H), 7.70 (d, J = 1.9 Hz, 1H), 7.52 (d, J = 8.1 Hz, 1H), 7.42 (s, 1H), 7.19 (s, 2H), 7.07 (dd, J = 8.1, 1.6 Hz, 1H), 6.95-6.92 (m, 1H), 6.67 (d, J = 8.8 Hz, 1H), 3.06 (s, 4H), 2.87 (d, J = 4.7 Hz, 3H). | C |
| 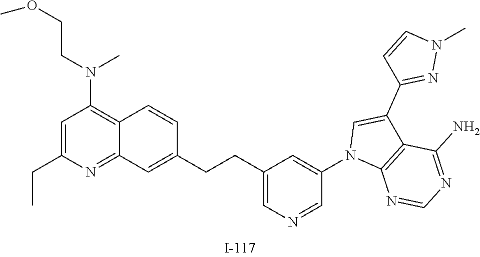 | 562.3 | ¹H NMR (400 MHz, DMSO-d6) δ ppm 9.16 (s, 1H), 8.97 (d, J = 2.4 Hz, 1H), 8.44 (d, J = 1.7 Hz, 1H), 8.21 (t, J = 2.1 Hz, 1H), 8.14 (s, 1H), 8.11 (s, 1H), 8.05 (d, J = 8.6 Hz, 1H), 7.79 (d, J = 2.2 Hz, 1H), 7.72 (d, J = 1.5 Hz, 1H), 7.38 (dd, J = 8.6, 1.7 Hz, 2H), 6.80 (s, 1H), 6.74 (d, J = 2.3 Hz, 1H), 3.92 (s, 3H), 3.65 (t, J = 5.5 Hz, 2H), 3.41 (t, J = 5.5 Hz, 3H), 3.15 (s, 4H), 2.96 (s, 3H), 2.80 (q, J = 7.5 Hz, 2H), 1.33-1.24 (m, 3H). | C |
| 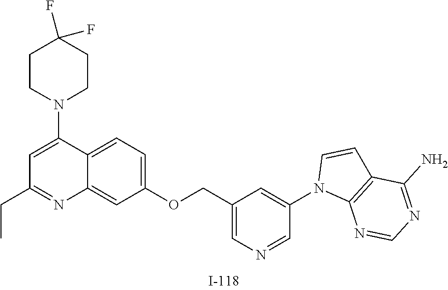 [SMILES: CCc1cc(N2CCC(F)(F)CC2)c2ccc(OCc3cncc(-n4ccc5c(N)ncnc54)c3)cc2n1] | 516.2 | ¹H NMR (400 MHz, DMSO-d6) δ ppm 9.09-9.08 (m, 1H), 8.70 (s, 1H), 8.48 (s, 1H), 8.16 (s, 1H), 7.93 (d, J = 9.2 Hz, 1H), 7.71 (d, J = 3.6 Hz, 1H), 7.46-7.45 (m, 1H), 7.24-7.23 (m, 3H), 6.86-6.84 (m, 2H), 5.43 (s, 2H), 3.33-3.25 (m, 4H), 2.84-2.82 (m, 2H), 2.26 (s, 4H), 1.29 (t, J = 7.6 Hz, 3H). | B |
| 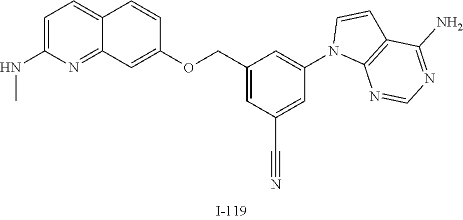 [SMILES: CNc1ccc2ccc(OCc3cc(C#N)cc(-n4ccc5c(N)ncnc54)c3)cc2n1] | 422.0 | ¹H NMR (400 MHz, DMSO-d6) δ ppm 8.42 (d, J = 13.4 Hz, 2H), 8.18 (s, 1H), 7.89 (s, 1H), 7.75-7.72 (m, 2H), 7.54 (d, J = 8.7 Hz, 1H), 7.30-7.20 (br, 2H), 7.07 (d, J = 2.3 Hz, 1H), 6.98-6.88 (m, 2H), 6.84 (d, J = 3.7 Hz, 1H), 6.58 (d, J = 8.8 Hz, 1H), 5.35 (s, 2H), 2.87 (d, J = 4.7 Hz, 3H). | B |
| 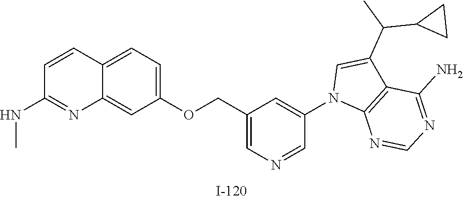 [SMILES: CNc1ccc2ccc(OCc3cncc(-n4cc(C(C)C5CC5)c5c(N)ncnc54)c3)cc2n1] | 466.0 | ¹H NMR (400 MHz, DMSO-d6) δ ppm 9.11 (d, J = 2.5 Hz, 1H), 8.64 (d, J = 1.7 Hz, 1H), 8.43 (t, J = 2.1 Hz, 1H), 8.13 (s, 1H), 7.74 (d, J = 8.9 Hz, 1H), 7.58 (s, 1H), 7.53 (d, J = 8.7 Hz, 1H), 7.10 (d, J = 2.4 Hz, 1H), 6.95-6.92 (m, 1H), 6.88 (dd, J = 8.7, 2.5 Hz, 1H), 6.70 (s, 2H), 6.58 (d, J = 8.8 Hz, 1H), 5.36 (s, 2H), 2.88 (d, J = 4.7 Hz, 3H), 2.79-2.72 (m, 1H), 1.31 (d, J = 6.8 Hz, 3H), 1.12 (dd, J = 12.3, 4.0 Hz, 1H), 0.53-0.41 (m, 2H), 0.22-0.19 (m, 2H). | B |
| 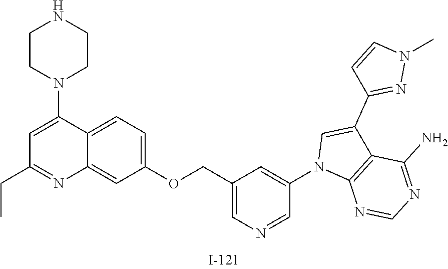 [SMILES: CCc1cc(N2CCNCC2)c2ccc(OCc3cncc(-n4cc(-c5ccn(C)n5)c5c(N)ncnc54)c3)cc2n1] | 561.1 | ¹H NMR (400 MHz, DMSO-d6) δ ppm 9.18 (brs, 1H), 9.13 (d, 1H), 8.53 (t, J = 2.0 Hz, 1H), 8.26 (s, 1H), 8.15 (s, 1H), 7.88 (d, J = 9.2 Hz, 1H), 7.78 (d, J = 2 Hz, 1H), 7.43 (d, J = 2.8 Hz, 1H), 7.41 (brs, 1H) 7.21 (dd, J = 2.8 Hz, 9.2 Hz 1H), 6.77 (d, J = 2.0 Hz 1H), 6.74 (s, 1H), 5.24 (s, 2H), 3.92 (s, 3H), 3.05-2.95 (m, 8H), 2.81 (q, J = 7.2 Hz, 14.8 Hz 2H), 1.28 (t, J = 7.2 Hz 3H). | B |
| 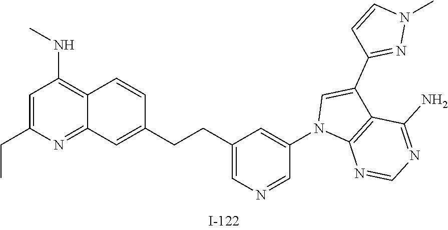 [SMILES: CCc1cc(NC)c2ccc(CCc3cncc(-n4cc(-c5ccn(C)n5)c5c(N)ncnc54)c3)cc2n1] | 504.1 | ¹H NMR (400 MHz, DMSO-d6) δ ppm 9.16 (s, 1H), 8.97 (s, 1H), 8.41 (s, 1H), 8.20 (s, 1H), 8.12 (d, J = 5.5 Hz, 2H), 8.00 (d, J = 8.3 Hz, 1H), 7.79 (s, 1H), 7.59 (s, 1H), 7.39 (s, 1H), 7.29 (d, J = 8.1 Hz, 1H), 7.16 (s, 1H), 6.73 (s, 1H), 6.25 (s, 1H), 3.92 (s, 3H), 3.12 (s, 3H), 2.87 (d, J = 3.9 Hz, 4H), 2.72 (m, 2H), 1.26 (t, J = 7.5 Hz, 3H). | C |
| 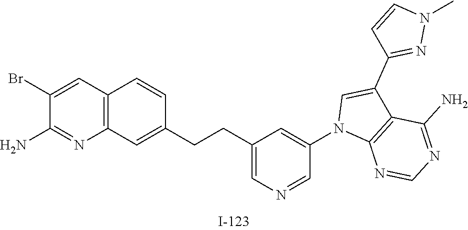 [SMILES: Cn1ccc(-c2cn(-c3cncc(CCc4ccc5cc(Br)c(N)nc5c4)c3)c3ncnc(N)c23)n1] | 540.1 | ¹H NMR (400 MHz, DMSO-d6) δ ppm 9.21-9.12 (br, 1H), 8.97 (d, J = 2.4 Hz, 1H), 8.39 (d, J = 1.7 Hz, 1H), 8.33 (s, 1H), 8.20 (t, J = 2.1 Hz, 1H), 8.13-8.11 (m, 2H), 7.79 (d, J = 2.2 Hz, 1H), 7.60 (d, J = 8.3 Hz, 1H), 7.43-7.37 (br, 1H), 7.35-7.33 (br, 1H), 7.18 (dd, J = 8.2, 1.5 Hz, 1H), 6.73 (d, J = 2.3 Hz, 1H), 6.63-6.57 (s, 2H), 3.92 (s, 3H), 3.09 (s, 4H). | C |
| 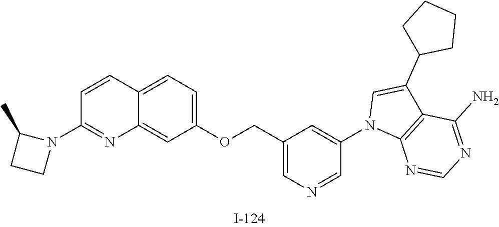 [SMILES: C[C@@H]1CCN1c1ccc2ccc(OCc3cncc(-n4cc(C5CCCC5)c5c(N)ncnc54)c3)cc2n1] | 506.4 | ¹H NMR (400 MHz, DMSO-d6) δ ppm 9.15 (d, J = 2.0 Hz, 1H), 8.63 (d, J = 1.6 Hz, 1H), 8.43-8.44 (m, 1H), 8.13 (s, 1H), 7.91 (d, J = 8.8 Hz, 1H), 7.61 (d, J = 8.8 Hz, 1H), 7.50 (s, 1H), 7.13 (d, J = 2.4 Hz, 1H), 6.94 (dd, J = 8.4, 1.6 Hz, 1H), 6.75 (brs, 2H), 6.53 (d, J = 8.8 Hz, 1H), 5.38 (s, 2H), 4.41-4.46 (m, 1H), 3.98-4.04 (m, 1H), 3.86-3.92 (m, 1H), 3.44-3.51 (m, 1H), 1.95-2.13 (m, 4H), 1.58-1.77 (m, 6H), 1.50 (d, J = 3.2 Hz, 3H) | B |
| 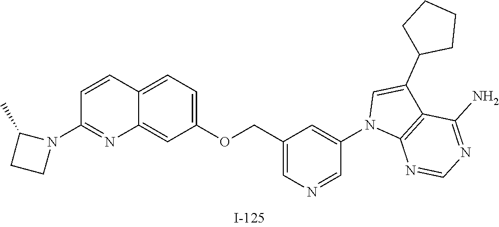 [SMILES: C[C@H]1CCN1c1ccc2ccc(OCc3cncc(-n4cc(C5CCCC5)c5c(N)ncnc54)c3)cc2n1] | 506.4 | ¹H NMR (400 MHz, DMSO-d6) δ ppm 9.15 (d, J = 2.0 Hz, 1H), 8.63 (d, J = 1.6 Hz, 1H), 1H), 7.91 (d, J = 8.8 Hz, 1H), 8.43-8.44 (m, 1H), 8.13 (s, 7.61 (d, J = 8.8 Hz, 1H), 7.50 (s, 1H), 7.13 (d, J = 2.4 Hz, 1H), 6.94 (dd, J = 8.4, 1.6 Hz, 1H), 6.75 (brs, 2H), 6.53 (d, J = 8.8 Hz, 1H), 5.38 (s, 2H), 4.46-4.41 (m, 1H), 4.04-3.98 (m, 1H), 3.92-3.86 (m, 1H), 3.51-3.44 (m, 1H), 2.13-1.95 (m, 4H), 1.77-1.58 (m, 6H), 1.50 (d, J = 3.2 Hz, 3H) | B |
| 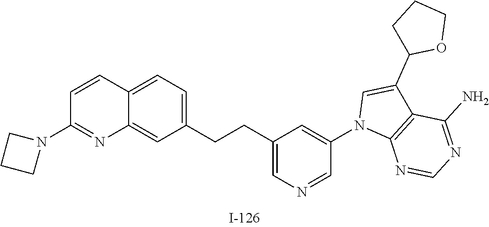 [SMILES: Nc1ncnc2c1c(C1CCCO1)cn2-c1cncc(CCc2ccc3ccc(N4CCC4)nc3c2)c1] | 492.3 | ¹H NMR (400 MHz, DMSO-d6) δ ppm 8.93 (d, J = 2.0 Hz, 1H), 8.39 (d, J = 1.6 Hz, 1H), 8.15 (s, 1H), 8.11 (t, J = 2.0 Hz, 1H), 7.95 (d, J = 8.8 Hz, 1H), 7.61 (d, J = 8.0 Hz, 1H), 7.56 (s, 1H), 7.45 (s, 1H), 7.14 (m, J = 8.4 Hz, 1H), 7.05 (s, 1H), 6.63 (d, J = 8.8 Hz, 1H), 5.04 (t, J = 6.4 Hz, 1H), 4.05 (t, J = 7.2 Hz, 4H), 4.00-3.95 (m, 1H), 3.92-3.86 (m, 1H), 3.08 (s, 4H), 2.43-2.36 (m, 2H), 2.34-1.97 (m, 4H). | C |
| 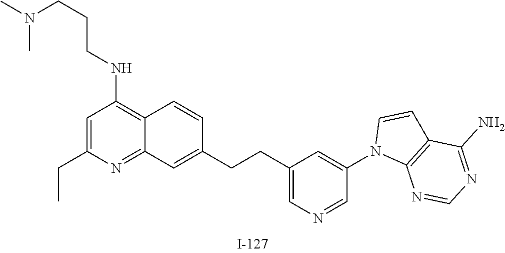 [SMILES: CCc1cc(NCCCN(C)C)c2ccc(CCc3cncc(-n4ccc5c(N)ncnc54)c3)cc2n1] | 495.4 | ¹H NMR (400 MHz, DMSO-d6) δ ppm 8.92 (d, J = 2.3 Hz, 1H), 8.39 (d, J = 1.4 Hz, 1H), 8.25-8.15 (m, 2H), 8.14-8.10 (m, 1H), 7.84 (s, 1H), 7.70-7.52 (m, 2H), 7.41 (d, J = 8.8 Hz, 1H), 7.22 (s, 2H), 6.81 (d, J = 3.6 Hz, 1H), 6.46 (s, 1H), 3.41-3.35 (m, 2H), 3.20-3.04 (m, 4H), 2.76 (q, J = 7.5 Hz, 2H), 2.44 (t, J = 6.8 Hz, 2H), 2.25 (s, 6H), 1.90-1.72 (m, 2H), 1.27 (t, J = 7.6 Hz, 3H). | C |
| 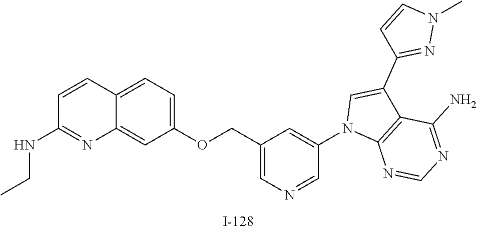 [SMILES: CCNc1ccc2ccc(OCc3cncc(-n4cc(-c5ccn(C)n5)c5c(N)ncnc54)c3)cc2n1] | 492.1 | ¹H NMR (400 MHz, DMSO-d6) δ ppm 9.18 (brs, 1H), 9.12 (d, J = 2.4 Hz 1H), 8.69 (d, J = 2.0 Hz, 1H), 8.49 (t, J = 2.0 Hz, 1H), 8.26 (s, 1H), 8.15 (s, 1H), 7.79 (d, J = 2.4 Hz, 1H), 7.74 (brd, J = 8.8 Hz, 1H), 7.52 (brd, J = 8.8 Hz, 1H), 7.42 (brs, 1H), 7.09 (d, J = 2.4 Hz, 1H), 6.95-6.87 (m, 2H), 6.78 (d, J = 2.0 Hz 1H), 6.57 (t, J = 4.8 Hz 1H), 5.37 (s, 2H), 3.92 (s, 3H), 3.42-3.37 (m, 2H), 3.39 (q, J = 7.2 Hz, 2H), 1.18 (t, J = 7.2 Hz, 3H). | B |
| 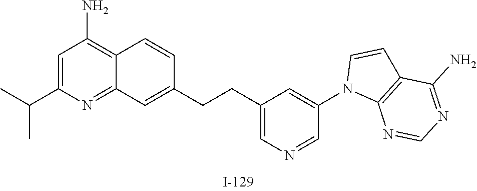 [SMILES: CC(C)c1cc(N)c2ccc(CCc3cncc(-n4ccc5c(N)ncnc54)c3)cc2n1] | 424.1 | ¹H NMR (400 MHz, DMSO-d6) δ ppm 8.92 (d, J = 2.4 Hz, 1H), 8.40 (d, J = 1.8 Hz, 1H), 8.17 (t, J = 2.1 Hz, 1H), 8.13 (s, 1H), 8.00 (d, J = 8.5 Hz, 1H), 7.58 (dd, J = 4.5, 2.6 Hz, 2H), 7.26 (d, J = 8.5 Hz, 1H), 7.21 (s, 2H), 6.81 (d, J = 3.7 Hz, 1H), 6.64 (s, 2H), 6.42 (s, 1H), 3.11 (s, 4H), 2.91 (dt, J = 13.7, 6.9 Hz, 1H), 1.23 (d, J = 6.9 Hz, 6H). | C |
| 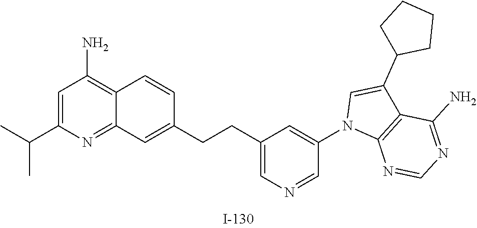 [SMILES: CC(C)c1cc(N)c2ccc(CCc3cncc(-n4cc(C5CCCC5)c5c(N)ncnc54)c3)cc2n1] | 492.1 | ¹H NMR (400 MHz, DMSO-d6) δ ppm 8.96 (d, J = 2.4 Hz, 1H), 8.36 (d, J = 1.6 Hz, 1H), 8.11 (s, 2H), 8.00 (d, J = 8.5 Hz, 1H), 7.58 (s, 1H), 7.35 (s, 1H), 7.25 (d, J = 8.5 Hz, 1H), 6.68 (d, J = 29.1 Hz, 4H), 6.42 (d, 1H), 3.45-3.40 (m, 1H), 3.10 (s, 4H), 2.91 (dt, J = 13.7, 6.9 Hz, 1H), 2.06 (d, J = 5.1 Hz, 2H), 1.84-1.51 (m, 6H), 1.23 (d, J = 6.9 Hz, 6H). | C |
| 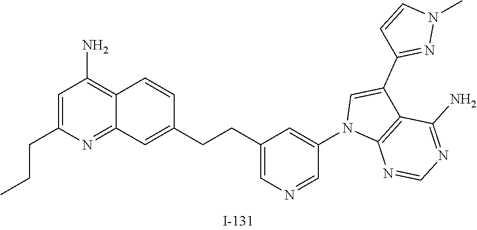 [SMILES: CCCc1cc(N)c2ccc(CCc3cncc(-n4cc(-c5ccn(C)n5)c5c(N)ncnc54)c3)cc2n1] | 252.7 | ¹H NMR (400 MHz, DMSO-d6) δ ppm 9.16 (s, 1H), 8.97 (d, J = 2.4 Hz, 1H), 8.41 (d, J = 1.7 Hz, 1H), 8.23 (s, 1H), 8.13 (d, J = 7.7 Hz, 2H), 8.01 (d, J = 8.7 Hz, 1H), 7.79 (d, J = 2.2 Hz, 1H), 7.56 (s, 1H), 7.38 (s, 1H), 7.27 (d, J = 8.4 Hz, 1H), 6.75 (d, J = 2.3 Hz, 1H), 6.70 (s, 2H), 6.39 (s, 1H), 3.92 (s, 3H), 3.12 (s, 4H), 2.64-2.57 (m, 2H), 1.69 (dd, J = 15.0, 7.4 Hz, 2H), 0.91 (t, J = 7.3 Hz, 3H). | C |
| 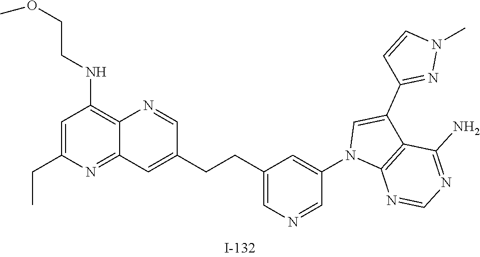 | 549.2 | ¹H NMR (400 MHz, DMSO-d6) δ ppm 9.16 (br, 1H), 8.97 (d, J = 2.4 Hz, 1H), 8.57 (s, 1H), 8.40 (s, 1H), 8.24-8.23 (m, 1H), 8.14 (s, 1H), 8.10 (s, 1H), 7.99 (s, 1H), 7.79 (s, 1H), 7.37 (br, 1H), 7.11-7.08 (m, 1H), 6.74 (s, 1H), 6.56 (s, 1H), 3.91 (s, 3H), 3.61-3.58 (m, 2H), 3.49-3.45 (m, 2H), 3.33 (s, 3H), 3.22-3.15 (m, 4H), 2.77-2.71 (m, 2H), 1.26 (t, J = 7.6 Hz, 3H) | C |
| 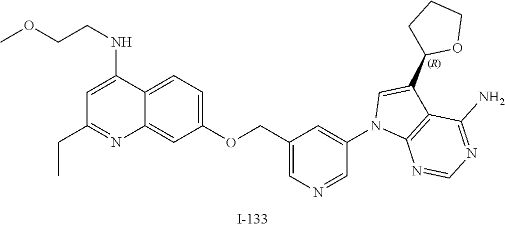 | 270.8 | ¹H NMR (400 MHz, DMSO-d6) δ ppm 9.08 (d, J = 2.5 Hz, 1H), 8.68 (d, J = 1.7 Hz, 1H), 8.45 (t, J = 2.1 Hz, 1H), 8.17 (s, 1H), 8.09 (d, J = 9.3 Hz, 1H), 7.70 (d, J = 0.8 Hz, 1H), 7.27 (d, J = 2.6 Hz, 1H), 7.14-6.93 (m, 4H), 6.30 (s, 1H), 5.38 (s, 2H), 5.06 (t, J = 6.3 Hz, 1H), 3.98 (dd, J = 15.3, 6.8 Hz, 1H), 3.93-3.85 (m, 1H), 3.58 (t, J = 5.7 Hz, 2H), 3.44 (dd, J = 11.1, 5.5 Hz, 2H), 3.29 (s, 3H), 2.69 (q, J = 7.6 Hz, 2H), 2.28 (dd, J = 12.8, 5.8 Hz, 1H), 2.09-1.99 (m, 3H), 1.25 (t, J = 7.6 Hz, 3H). | B |
| 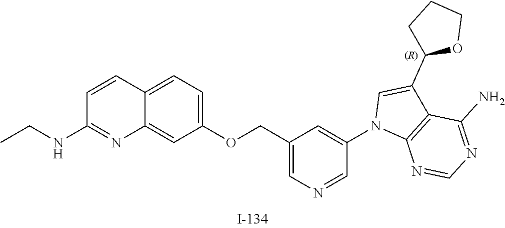 | 482.1 | ¹H NMR (400 MHz, DMSO-d6) δ ppm 9.07 (m, J = 2.5 Hz, 1H), 8.66 (d, J = 1.7 Hz, 1H), 8.43 (t, J = 2.1 Hz, 1H), 8.17 (s, 1H), 7.74 (d, J = 8.9 Hz, 1H), 7.70 (d, J = 0.8 Hz, 1H), 7.52 (d, J = 8.8 Hz, 1H), 7.12-6.91 (m, 3H), 6.88 (dd, J = 8.7, 2.5 Hz, 1H), 6.58 (d, J = 8.9 Hz, 1H), 5.35 (s, 2H), 5.08-5.06 (m, 1H), 4.03-3.84 (m, 2H), 3.41-3.33 (m, 2H), 3.30 (s, 1H), 2.29-2.27 (m, 1H), 2.12-1.96 (m, 3H), 1.18 (t, J = 7.2 Hz, 3H). | B |
| 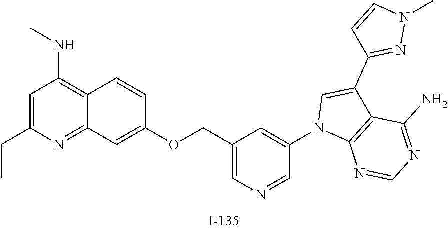 [SMILES: CCc1cc(NC)c2ccc(OCc3cncc(-n4cc(-c5ccn(C)n5)c5c(N)ncnc54)c3)cc2n1] | 506.2 | ¹H NMR (400 MHz, DMSO-d6) δ ppm 9.13 (d, J = 2.4 Hz, 1H), 8.70 (d, J = 1.6 Hz, 1H), 8.51-8.52 (m, 1H), 8.26 (s, 1H), 8.15 (s, 1H), 8.02 (d, J = 5.2 Hz, 1H), 7.79 (d, J = 2 Hz, 1H), 7.38-7.42 (m, 1H), 7.29 (d, J = 4 Hz, 1H), 7.12 (dd, J = 8, 5.6 Hz, 2H), 6.78 (d, J = 2.4 Hz, 1H), 6.22 (s, 1H), 5.41 (s, 2H), 3.92 (s, 3H), 2.86 (d, J = 8.8 Hz, 3H), 2.70 (dd, J = 14.4, 7.6 Hz, 2H), 1.23-1.28 (m, 3H). | B |
| 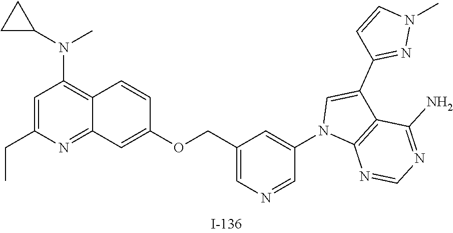 [SMILES: CCc1cc(N(C)C2CC2)c2ccc(OCc3cncc(-n4cc(-c5ccn(C)n5)c5c(N)ncnc54)c3)cc2n1] | 546.3 | ¹H NMR (400 MHz, DMSO-d6) δ ppm 9.21 (br, 1H), 9.17 (s, 1H), 8.75 (s, 1H), 8.56 (s, 1H), 8.31 (s, 1H), 8.18 (s, 1H), 7.96 (d, J = 12.4 Hz, 1H), 7.83 (s, 1H), 7.48-7.42 (m, 2H), 7.19 (d, 1H), 6.90 (s, 1H), 6.81 (s, 1H), 5.46 (s, 2H), 3.96 (s, 3H), 3.15 (s, 3H), 2.86-2.81 (m, 3H), 1.34 (t, J = 7.6 Hz, 3H), 0.84-0.80 (m, 2H), 0.49-0.45 (m, 2H) | B |
| 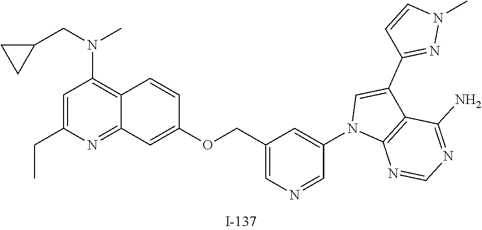 [SMILES: CCc1cc(N(C)CC2CC2)c2ccc(OCc3cncc(-n4cc(-c5ccn(C)n5)c5c(N)ncnc54)c3)cc2n1] | 560.1 | ¹H NMR (400 MHz, DMSO-d6) δ ppm 9.18 (s, 1H), 9.13 (d, J = 1.6 Hz, 1H), 8.72 (s, 1H), 8.53 (s, 1H), 8.26 (s, 1H), 8.04 (d, J = 8.2 Hz 1H), 7.79 (d, J = 1.6 Hz, 1H), 7.42 (s, 1H), 7.39 (d, J = 2 Hz, 1H), 7.26-7.24 (m, 1H), 6.78 (d, J = 1.6 Hz, 2H), 5.44 (s, 2H), 3.91 (s, 3H), 3.33 (s, 2H), 3.16 (s, 3H), 2.84-2.79 (m, 2H), 1.29 (t, J = 7.6 Hz, 3H), 1.20-1.08 (m, 1H), 0.54 (d, J = 8.4 Hz, 2H), 0.21 (d, J = 3.2 Hz, 2H). | B |
| 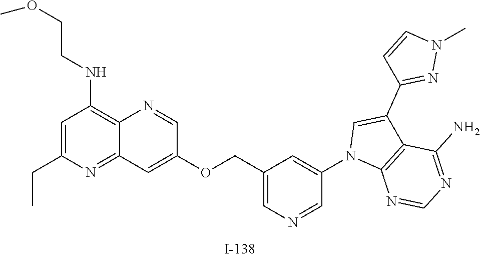 | 551.2 | ¹H NMR (400 MHz, DMSO-d6) δ ppm 9.19 (brs, 1H), 9.15 (d, J = 2.4 Hz, 1H), 8.73 (d, J = 2.0 Hz, 1H), 8.55 (t, J = 1.6 Hz, 1H), 8.53 (d, J = 2.8 Hz, 1H), 8.26 (s, 1H), 8.15 (s, 1H), 7.79 (d, J = 2.4 Hz, 1H), 7.71 (d, J = 2.8 Hz, 1H), 7.42 (brs, 1H), 7.01 (t, J = 5.8 Hz, 1H), 6.78 (d, J = 2.0 Hz, 1H), 6.51 (s, 1H), 5.47 (s, 2H), 3.92 (s, 3H), 3.60 (t, J = 5.4 Hz, 2H), 3.49-3.45 (m, 2H), 3.31 (s, 3H), 2.77-2.71 (m, 2H), 1.27 (t, J = 7.6 Hz, 3H). | B |
| 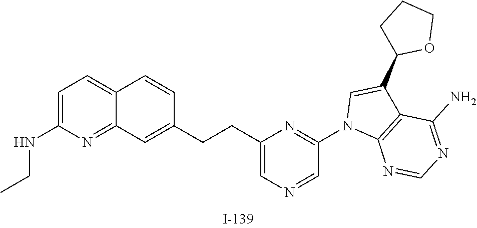 [SMILES: CCNc1ccc2ccc(CCc3cncc(-n4cc([C@H]5CCCO5)c5c(N)ncnc54)n3)cc2n1] | 481.1 | ¹H NMR (400 MHz, DMSO-d6) δ ppm 9.76 (s, 1H), 8.46 (s, 1H), 8.27 (s, 1H), 7.85 (s, 1H), 7.76 (s, 1H), 7.56 (d, J = 7.4 Hz, 1H), 7.44 (s, 1H), 7.11-7.09 (m, 3H), 6.76-6.69 (m, 1H), 5.41 (s, 1H), 5.03-4.96 (m, 1H), 4.02-3.97 (m, 1H), 3.92-3.86 (m, 1H), 3.42-3.38 (m, 2H), 3.26-3.19 (m, 4H), 2.29-2.23 (m, 1H), 2.08-2.00 (m, 2H), 1.94-1.86 (m, 1H), 1.19 (t, J = 7.2 Hz, 3H). | C |
| 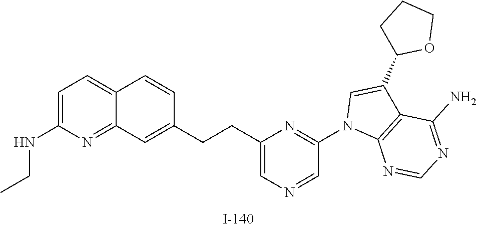 [SMILES: CCNc1ccc2ccc(CCc3cncc(-n4cc([C@@H]5CCCO5)c5c(N)ncnc54)n3)cc2n1] | 481.1 | ¹H NMR (400 MHz, DMSO-d6) δ ppm 9.76 (s, 1H), 8.46 (s, 1H), 8.27 (s, 1H), 7.85 (s, 1H), 7.76 (s, 1H), 7.56 (d, J = 7.4 Hz, 1H), 7.44 (s, 1H), 7.11-7.09 (m, 3H), 6.76-6.69 (m, 1H), 5.41 (s, 1H), 5.03-4.96 (m, 1H), 4.02-3.97 (m, 1H), 3.92-3.86 (m, 1H), 3.42-3.38 (m, 2H), 3.26-3.19 (m, 4H), 2.29-2.23 (m, 1H), 2.08-2.00 (m, 2H), 1.94-1.86 (m, 1H), 1.19 (t, J = 7.2 Hz, 3H). | C |
| 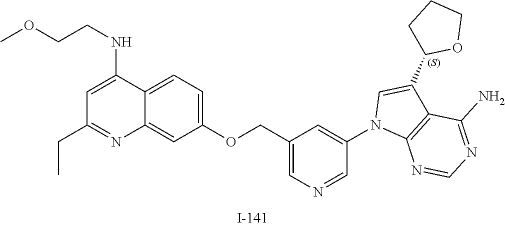 | 540.1 | ¹H NMR (400 MHz DMSO-d6) δ ppm 9.08 (d, J = 2.5 Hz, 1H), 8.68 (d, J = 1.7 Hz, 1H), 8.45 (t, J = 2.1 Hz, 1H), 8.17 (s, 1H), 8.11 (d, J = 9.2 Hz, 1H), 7.71 (s, 1H), 7.28 (d, J = 2.6 Hz, 1H), 7.18-6.95 (m, 4H), 6.33 (s, 1H), 5.38 (s, 2H), 5.06 (t, J = 6.3 Hz, 1H), 3.98 (dd, J = 15.1, 6.6 Hz, 1H), 3.93-3.85 (m, 1H), 3.59 (t, J = 5.7 Hz, 2H), 3.45 (dd, J = 11.0, 5.5 Hz, 2H), 3.29 (s, 3H), 2.70 (q, J = 7.6 Hz, 2H), 2.34-2.24 (m, 1H), 2.11-1.97 (m, 3H), 1.26 (t, J = 7.6 Hz, 3H). | B |
| 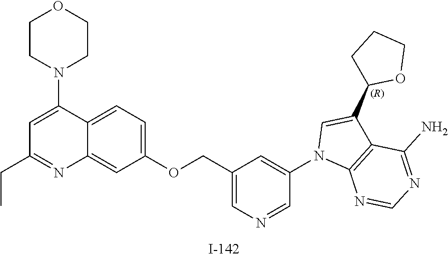 | 552.1 | ¹H NMR (400 MHz, DMSO-d6) δ ppm 9.09 (d, J = 2.8 Hz, 1H), 8.69 (d, J = 1.6 Hz, 1H), 8.46-8.47 (m, 1H), 8.17 (s, 1H), 7.92 (d, J = 9.2 Hz, 1H), 7.71 (s, 1H), 7.45 (d, J = 4.8 Hz, 1H), 7.21 (dd, J = 8.8, 2.4 Hz, 1H), 7.00-7.05 (m, 2H), 6.79 (s, 1H), 5.41 (s, 2H), 5.04-5.08 (s, 1H), 3.92-4.00 (m, 2H), 3.64-3.86 (m, 4H), 3.12-3.13 (m, 4H), 2.83 (dd, J = 15.2,7.6 Hz, 2H), 2.24-2.33 (m, 1H), 1.99-2.11 (m, 3H), 1.27-1.31 (m, 3H) | B |
| 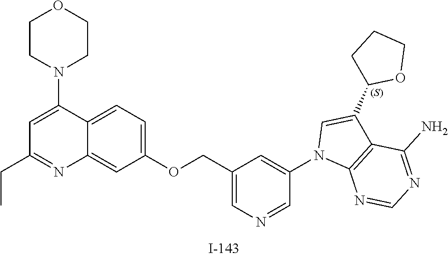 | 552.1 | ¹H NMR (400 MHz, DMSO-d6) δ ppm 9.09 (d, J = 2.8 Hz, 1H), 8.69 (d, J = 1.6 Hz, 1H), 8.47-8.46 (m, 1H), 8.17 (s, 1H), 7.92 (d, J = 9.2 Hz, 1H), 7.71 (s, 1H), 7.45 (d, J = 4.8 Hz, 1H), 7.21 (dd, J = 8.8, 2.4 Hz, 1H), 7.05-7.00 (m, 2H), 6.79 (s, 1H), 5.41 (s, 2H), 5.08-5.04 (m, 1H), 4.00-3.92 (m, 2H), 3.64-3.86 (m, 4H), 3.13-3.12 (m, 4H), 2.83 (dd, J = 15.2,7.6 Hz, 2H), 2.33-2.24 (m, 1H), 2.11-1.99 (m, 3H), 1.31-1.27 (m, 3H) | B |
| 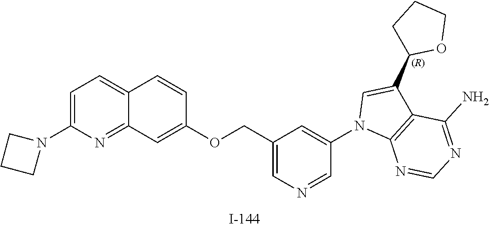 | 494.0 | ¹H NMR (400 MHz, DMSO-d6) δ ppm 9.08 (d, J = 2 Hz, 1H), 8.67 (d, J = 1.6 Hz, 1H), 8.44 (t, J = 2 Hz, 1H), 8.17 (s, 1H), 7.92 (d, J = 8.8 Hz, 1H), 7.70 (s, 1H), 7.62 (d, J = 8.8, 1H), 7.15 (d, J = 5.6, 1H), 7.05 (s, 1H), 6.95 (dd, J = 8 Hz, 2 Hz, 1H), 6.53 (d, J = 8.4 Hz, 1H), 5.37 (s, 2H), 5.06 (t, J = 6.2 Hz, 1H), 4.07 (t, J = 7.4 Hz, 4H), 4.01-3.86 (m, 2H), 2.38-2.32 (m, 2H), 2.31-1.99 (m, 4H). | B |
| 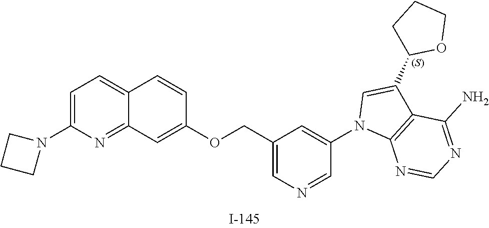 | 494.0 | ¹H NMR (400 MHz, DMSO-d6) δ ppm 9.08 (d, J = 2 Hz, 1H), 8.67 (d, J = 1.6 Hz, 1H), 8.43 (t, J = 2 Hz, 1H), 8.17 (s, 1H), 7.92 (d, J = 8.8 Hz, 1H), 7.70 (s, 1H), 7.62 (d, J = 8.8, 1H), 7.15 (d, J = 5.6, 1H), 7.05 (s, 1H), 6.95 (dd, J = 8 Hz, 2 Hz, 1H), 6.53 (d, J = 8.4 Hz, 1H), 5.37 (s, 2H), 5.06 (t, J = 6.2 Hz, 1H), 4.07 (t, J = 7.4 Hz 4H), 4.01-3.86 (m, 2H), 2.38-2.32 (m, 2H), 2.31-1.99 (m, 4H). | B |
| 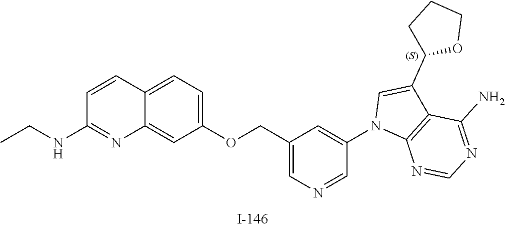 | 482.1 | ¹H NMR (400 MHz, DMSO-d6) δ ppm 9.07 (s, 1H), 8.67 (s, 1H), 8.43 (s, 1H), 8.17 (s, 1H), 7.79-7.66 (m, 2H), 7.52 (d, J = 8.6 Hz, 1H), 7.08-7.05 (m, 3H), 6.95 (s, 1H), 6.88 (d, J = 6.9 Hz, 1H), 6.58 (d, J = 8.7 Hz, 1H), 5.35 (s, 2H), 5.06-5.05 (m, 1H), 4.06-3.78 (m, 2H), 3.48-3.36 (m, 2H), 2.28-2.27 (m, 1H), 2.04 (s, 3H), 1.18 (t, J = 7.1 Hz, 3H). | B |
| 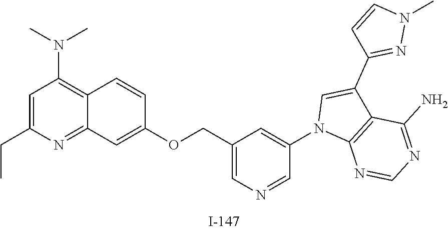 [SMILES: CCc1cc(N(C)C)c2ccc(OCc3cncc(-n4cc(-c5ccn(C)n5)c5c(N)ncnc54)c3)cc2n1] | 520.2 | ¹H NMR (400 MHz, DMSO-d6) δ 9.18 (s, 1H), 9.13 (d, J = 2.3 Hz, 1H), 8.72 (s, 1H), 8.53 (s, 1H), 8.26 (s, 1H), 8.15 (s, 1H), 7.95 (d, J = 9.2 Hz, 1H), 7.79 (d, J = 2.0 Hz, 1H), 7.40 (d, J = 2.4 Hz, 2H), 7.24-7.14 (m, 1H), 6.78 (d, J = 2.2 Hz, 1H), 6.68 (s, 1H), 5.43 (s, 2H), 3.91 (s, 3H), 2.97 (s, 6H), 2.79 (q, J = 7.5 Hz, 2H), 1.28 (t, J = 7.6 Hz, 3H). | B |
| 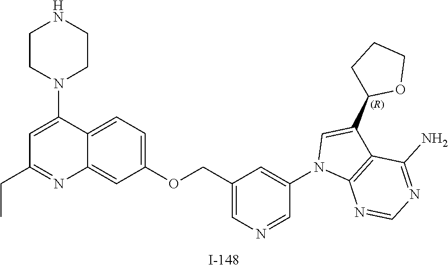 | 551.0 | ¹H NMR (400 MHz, DMSO-d6) δ ppm 9.08 (d, J = 2.4 Hz, 1H), 8.69 (d, J = 1.6 Hz, 1H), 8.46 (t, J = 2.0 Hz, 1H), 8.17 (s, 1H), 7.88 (d, J = 9.2 Hz, 1H), 7.71 (s, 1H), 7.42 (d, J = 2.8 Hz, 1H), 7.20 (dd, J = 2.8 Hz, 9.2 Hz, 1H), 7.05 (brs, 2H), 6.75 (s, 1H), 5.41 (s, 2H), 5.06 (t, J = 6.4 Hz, 1H), 4.00-3.86 (m, 2H), 3.07-2.98 (m, 8H), 2.81 (q, J = 7.2 Hz, 14.8 Hz 2H), 2.30-2.00 (m, 4H), 1.28 (t, J = 7.2 Hz, 3H). | B |
| 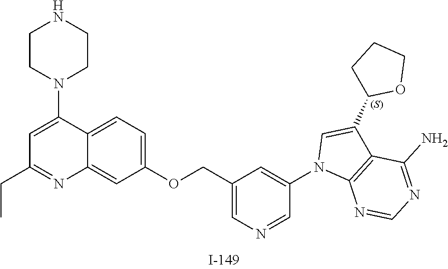 | 551.3 | ¹H NMR (400 MHz, DMSO-d6) δ ppm 9.08 (d, J = 2.4 Hz, 1H), 8.69 (d, J = 2.0 Hz, 1H), 8.47 (t, J = 2 Hz, 1H), 8.20 (s, 1H), 8.16 (s, 1H), 7.92 (d, J = 9.6 Hz, 1H), 7.71 (s, 1H), 7.45 (d, J = 2.8 Hz, 1H), 7.23 (dd, J = 9.2, 2.4 Hz, 1H), 7.04 (s, 2H), 6.81 (s, 1H), 5.41 (s, 2H), 5.07-5.04 (m, 1H), 4.00-3.96 (m, 1H), 3.91-3.86 (m, 1H), 3.19 (s, 8H), 2.85-2.80 (m, 2H), 2.32-2.30 (m, 1H), 2.08-2.05 (m, 3H), 1.30 (t, J = 3.8 Hz, 3H). | B |
| 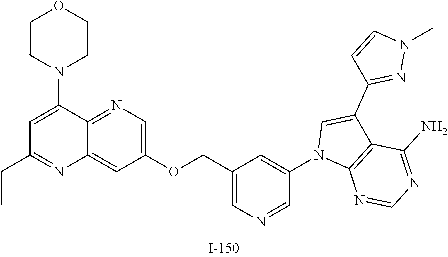 [SMILES: CCc1cc(N2CCOCC2)c2ncc(OCc3cncc(-n4cc(-c5ccn(C)n5)c5c(N)ncnc54)c3)cc2n1] | 563.2 | ¹H NMR (400 MHz, DMSO-d6) δ ppm 9.18 (br, 1H), 9.14 (d, J = 2.8 Hz, 1H), 8.73 (s, 1H), 8.62 (d, J = 2.8 Hz, 1H), 8.55-8.54 (m, 1H), 8.26 (s, 1H), 8.14 (s, 1H), 7.79-7.78 (m, 2H), 7.42 (br, 1H), 6.79-6.77 (m, 2H), 5.48 (s, 2H), 3.91 (s, 3H), 3.83-3.81 (m, 4H), 3.64-3.62 (m, 4H), 2.85-2.78 (m, 2H), 1.29 (t, J = 7.2 Hz, 3H). | B |
| 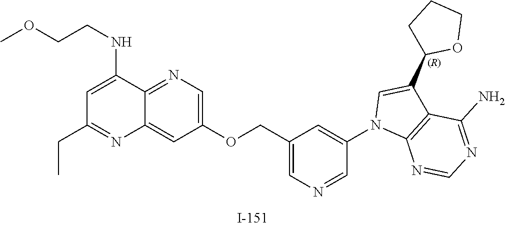 | 541.3 | ¹H NMR (400 MHz, DMSO-d6) δ ppm 9.09 (d, J = 2.4 Hz, 1H), 8.70 (d, J = 1.6 Hz, 1H), 8.52 (d, J = 2.8 Hz, 1H), 8.48 (t, J = 2.0 Hz, 1H), 8.16 (s, 1H), 7.71 (s, 1H), 7.69 (d, J = 2.8 Hz, 1H), 7.04 (brs, 2H), 6.53 (s, 1H), 6.45 (s, 2H), 5.06 (t, J = 6.4 Hz, 1H), 4.00-3.86 (m, 2H), 3.59 (t, J = 5.6 Hz, 2H), 3.48 (t, J = 5.6 Hz, 2H), 3.30 (s, 1H), 2.74 (q, J = 7.6 Hz, 13.6 Hz, 2H), 2.30-2.00 (m, 4H), 1.27 (t, J = 7.6 Hz, 3H). | B |
| 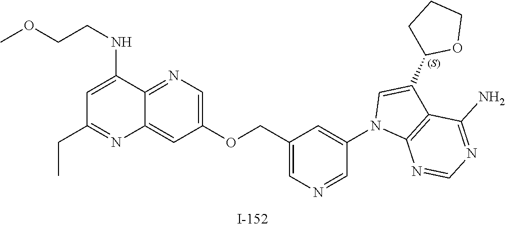 | 541.2 | ¹H NMR (400 MHz, DMSO-d6) δ 9.09 (d, J = 2.4 Hz, 1H), 8.70 (d, J = 2 Hz, 1H), 8.53 (d, J = 2.8 Hz, 1H), 8.48 (t, J = 4.4 Hz, 2H), 8.15 (s, 1H), 7.70 (s, 1H), 7.68 (d, J = 2.8 Hz, 1H), 7.04 (s, 2H), 6.54 (s, 1H), 5.45 (s, 2H), 5.05 (t, J = 6.4 Hz, 1H), 3.98-3.96 (m 1H), 3.89-3.87 (m, 1H), 3.60 (t, J = 5.6 Hz, 2H), 3.50 (m, 2H), 3.33 (d, J = 9.6 Hz, 3H), 2.76 (dd, J = 15.2, 7.6 Hz, 2H), 2.32-2.29 (m, 1H), 2.07-2.05 (m, 3H), 1.28 (t, J = 7.6 Hz, 3H). | B |
| 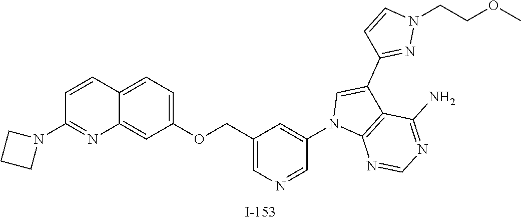 [SMILES: COCCn1ccc(-c2cn(-c3cncc(COc4ccc5ccc(N6CCC6)nc5c4)c3)c3ncnc(N)c23)n1] | 587.2 | ¹H NMR (400 MHz, DMSO-d6) δ ppm 8.18 (s, 1H), 9.13 (d, J = 2 Hz, 1H), 8.69 (s, 1H), 8.50 (s, 1H), 8.26 (s, 1H), 8.15 (s, 1H), 7.93 (d, J = 9.2 Hz, 1H), 7.81 (s, J = 2 Hz, 1H), 7.63 (d, J = 8.8, 1H), 7.41 (s, 1H), 7.16 (d, J = 2 Hz, 1H), 6.96 (dd, J = 8.8 Hz, 2.4 Hz, 1H), 6.76 (d, J = 8.8 Hz, 1H), 5.38 (s, 2H), 4.33 (t, J = 5.2 Hz, 2H), 4.07 (t, J = 7.2 Hz, 4H), 3.72 (t, J = 5 Hz, 2H), 3.24 (s, 3H), 2.43-2.31 (m, 2H). | B |
| 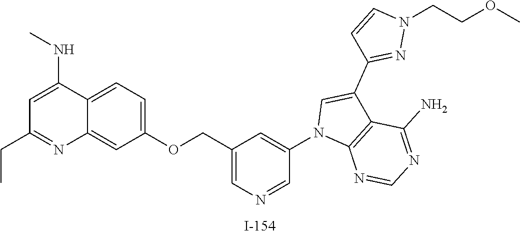 [SMILES: CCc1cc(NC)c2ccc(OCc3cncc(-n4cc(-c5ccn(CCOC)n5)c5c(N)ncnc54)c3)cc2n1] | 550.3 | ¹H NMR (400 MHz, DMSO-d6) δ 9.25-9.14 (m, 1H), 9.13 (d, J = 2.4 Hz, 1H), 8.71 (d, J = 1.7 Hz, 1H), 8.55-8.45 (m, 1H), 8.25 (s, 1H), 8.21-8.17 (m, 1H), 8.15 (s, 1H), 8.06 (d, J = 9.1 Hz, 1H), 7.81 (d, J = 2.3 Hz, 1H), 7.45-7.40 (m, 1H), 7.35-7.30 (m, 1H), 7.25-7.10 (m, 1H), 6.76 (d, J = 2.3 Hz, 1H), 6.31-6.25 (m, 1H), 5.41 (s, 2H), 4.33 (t, J = 5.1 Hz, 2H), 3.73 (t, J = 5.2 Hz, 2H), 3.27 (s, 3H), 2.90 (d, J = 4.6 Hz, 3H), 2.73 (q, J = 7.5 Hz, 2H), 1.27 (t, J = 7.6 Hz, 3H). | B |
| 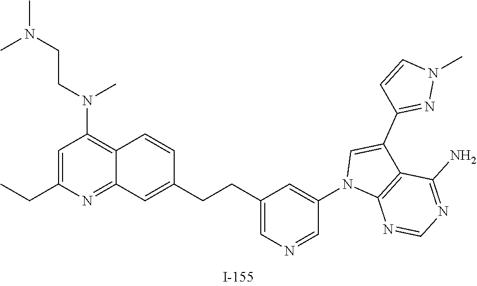 [SMILES: CCc1cc(N(C)CCN(C)C)c2ccc(CCc3cncc(-n4cc(-c5ccn(C)n5)c5c(N)ncnc54)c3)cc2n1] | 575.3 | ¹H NMR (400 MHz, DMSO-d6) δ ppm 9.16 (br, 1H), 8.97 (s, 1H), 8.43 (s, 1H), 8.22 (s, 1H), 8.14 (s, 1H), 8.11 (s, 1H), 7.97 (d, J = 8.4 Hz, 1H), 7.8 (s, 1H), 7.73 (s, 1H), 7.40-7.37 (m, 2H), 6.80 (s, 1H), 6.72 (s, 1H), 3.91 (s, 3H), 3.15 (s, 4H), 2.99 (s, 3H), 2.80 (q, 2H), 2.67 (s, 2H), 2.33 (s, 2H), 2.15-2.24 (m, 6H), 1.28 (t, J = 7.6 Hz, 3H). | C |
| 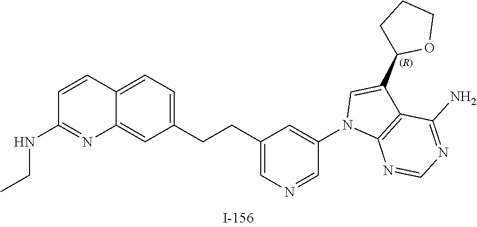 | 480.2 | ¹H NMR (400 MHz, DMSO-d6) δ ppm 8.94 (d, J = 2.4 Hz, 1H), 8.40 (d, J = 1.6 Hz, 1H), 8.16 (s, 1H), 8.11 (t, J = 2.2 Hz, 1H), 7.77 (d, J = 9.2 Hz, 1H), 7.56 (s, 1H), 7.52 (d, J = 8 Hz, 1H), 7.39 (s, 1H), 7.07 (dd, J = 8 Hz, 1.6 Hz, 1H), 7.07 (s, 2H), 6.95 (t, J = 5.2 Hz, 1H), 6.67 (d, J = 8.8 Hz, 1H), 5.04 (t, J = 6 Hz, 1H), 3.99-3.88 (m, 2H), 3.06 (s, 4H), 2.27-2.01 (m, 3H), 1.17 (t, J = 7.2 Hz, 3H). | C |
| 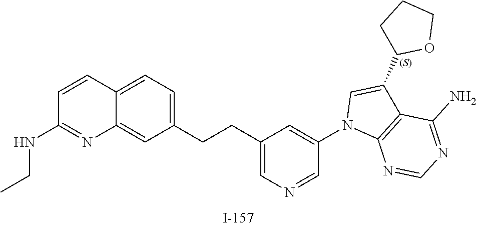 | 480.2 | ¹H NMR (400 MHz, DMSO-d6) δ ppm 8.94 (d, J = 2.4 Hz, 1H), 8.40 (d, J = 1.6 Hz, 1H), 8.16 (s, 1H), 8.11 (t, J = 2.2 Hz, 1H), 7.77 (d, J = 9.2 Hz, 1H), 7.56 (s, 1H), 7.52 (d, J = 8 Hz, 1H), 7.39 (s, 1H), 7.07 (dd, J = 8 Hz, 1.6 Hz, 1H), 7.07 (s, 2H), 6.95 (t, J = 5.2 Hz, 1H), 6.67 (d, J = 8.8 Hz, 1H), 5.03 (t, J = 6 Hz, 1H), 3.99-3.88 (m, 2H), 3.06 (s, 4H), 2.27-2.01 (m, 4H), 1.17 (t, J = 7.2 Hz, 3H). | C |
| 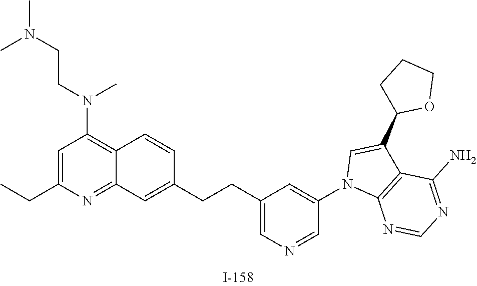 [SMILES: CCc1cc(N(C)CCN(C)C)c2ccc(CCc3cncc(-n4cc([C@H]5CCCO5)c5c(N)ncnc54)c3)cc2n1] | 551.3 | ¹H NMR (400 MHz, DMSO-d6) δ 8.93 (d, J = 2.4 Hz, 1H), 8.38 (d, J = 1.7 Hz, 1H), 8.17-8.09 (m, 4H), 7.58 (d, J = 4.6 Hz, 2H), 7.55-7.35 (m, 1H), 7.02 (s, 2H), 6.66-6.46 (m, 1H), 5.04 (t, J = 6.4 Hz, 1H), 4.05-3.95 (m, 1H), 3.92-3.85 (m, 1H), 3.55-3.45 (m, 4H), 3.20-3.06 (m, 3H), 2.85-2.70 (m, 2H), 2.73-2.58 (m, 2H), 2.28 (s, 6H), 2.10-1.95 (m, 3H), 1.35-1.23 (m, 3H). | C |
| 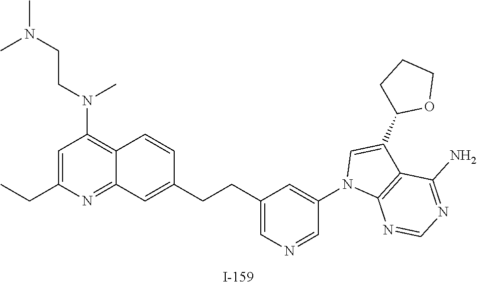 [SMILES: CCc1cc(N(C)CCN(C)C)c2ccc(CCc3cncc(-n4cc([C@@H]5CCCO5)c5c(N)ncnc54)c3)cc2n1] | 551.3 | ¹H NMR (400 MHz, DMSO-d6) δ 8.93 (d, J = 2.4 Hz, 1H), 8.38 (d, J = 1.7 Hz, 1H), 8.17-8.09 (m, 4H), 7.58 (d, J = 4.6 Hz, 2H), 7.55-7.35 (m, 1H), 7.02 (s, 2H), 6.66-6.46 (m, 1H), 5.04 (t, J = 6.4 Hz, 1H), 4.05-3.95 (m, 1H), 3.92-3.85 (m, 1H), 3.55-3.45 (m, 4H), 3.20-3.06 (m, 3H), 2.85-2.70 (m, 2H), 2.73-2.58 (m, 2H), 2.28 (s, 6H), 2.10-1.95 (m, 3H), 1.35-1.23 (m, 3H). | C |
| 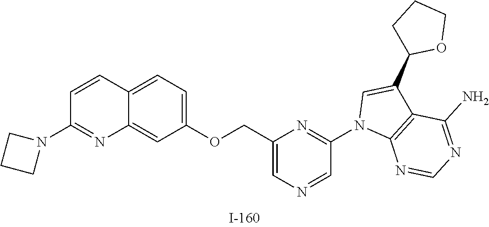 [SMILES: Nc1ncnc2c1c([C@H]1CCCO1)cn2-c1cncc(COc2ccc3ccc(N4CCC4)nc3c2)n1] | 495.2 | ¹H NMR (400 MHz, DMSO-d6) δ ppm 9.96 (s, 1H), 8.72 (s, 1H), 8.29 (s, 1H), 7.94 (s, 1H), 7.81 (s, 1H), 7.66 (s, 1H), 7.15 (s, 3H), 7.00 (s, 1H), 6.55 (s, 1H), 5.46 (s, 2H), 5.04 (t, J = 6.8 Hz, 1H), 4.07 (s, 4H), 4.00-3.95 (d, J = 5.7 Hz, 1H), 3.87 (s, 1H), 2.34-2.31 (m, 2H), 2.26 (s, 1H), 2.50-1.98 (m, 2H), 1.93-1.86 (m, 1H). | B |
| 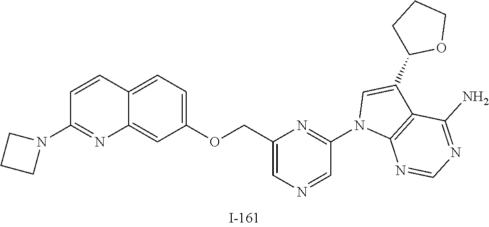 [SMILES: Nc1ncnc2c1c([C@@H]1CCCO1)cn2-c1cncc(COc2ccc3ccc(N4CCC4)nc3c2)n1] | 495.2 | ¹H NMR (400 MHz, DMSO-d6) δ ppm 9.96 (s, 1H), 8.72 (s, 1H), 8.29 (s, 1H), 7.94 (s, 1H), 7.81 (s, 1H), 7.66 (s, 1H), 7.15 (s, 3H), 7.00 (s, 1H), 6.55 (s, 1H), 5.46 (s, 2H), 5.04 (t, J = 6.8 Hz, 1H), 4.07 (s, 4H), 4.00-3.95 (d, J = 5.7 Hz, 1H), 3.87 (s, 1H), 2.34-2.31 (m, 2H), 2.26 (s, 1H), 2. 5-1.980 (m, 2H), 1.93-1.86 (m, 1H). | B |
| 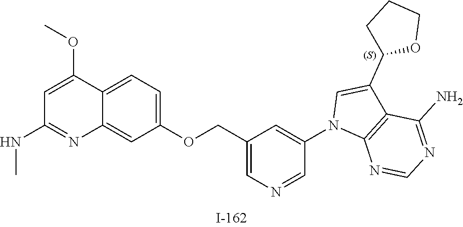 | 497.0 | ¹H NMR (400 MHz, DMSO-d6) δ ppm 9.08 (d, J = 2.4 Hz, 1H), 8.68 (d, J = 2 Hz 1H), 8.46 (t, J = 2.2 Hz, 1H), 8.16 (s, 1H), 7.98 (d, J = 8.8 Hz, 1H), 7.70 (s, 1H), 7.42 (d, J = 2.4 Hz, 1H), 7.21 (dd, J = 9.2 Hz, 2.4 Hz, 1H), 7.04 (s, 2H), 6.82 (s, 1H), 5.4 (s, 2H), 5.06 (t, J = 6.4 Hz, 1H), 4.00 (s, 3H), 3.98-3.87 (m, 2H), 2.87-2.81 (q, J = 7.6 Hz, 2H), 2.27-2.01 (m, 4H), 1.30 (t, J = 7.6 Hz, 3H). | B |
| 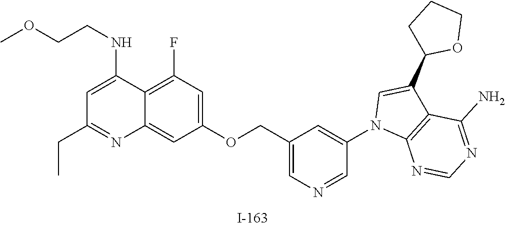 [SMILES: CCc1cc(NCCOC)c2c(F)cc(OCc3cncc(-n4cc([C@H]5CCCO5)c5c(N)ncnc54)c3)cc2n1] | 558.2 | ¹H NMR (400 MHz, DMSO-d6): δ ppm 9.09 (d, J = 2.5 Hz, 1H), 8.67 (d, J = 1.7 Hz, 1H), 8.44 (t, J = 2.1 Hz, 1H), 8.17 (s, 1H), 7.70 (s, 1H), 7.16 (d, J = 2.4 Hz, 1H), 7.05 (s, 1H), 6.96 (dd, J = 15.4, 2.4 Hz, 1H), 6.55 (s, 1H), 6.36 (s, 1H), 5.39 (s, 2H), 5.06 (t, J = 6.4 Hz, 1H), 3.99-3.95 (m, 1H), 3.92-3.88 (m, 1H), 3.60 (t, J = 5.5 Hz, 2H), 3.46-3.42 (m, 2H), 3.32 (s, 3H), 2.34-2.30 (m, 2H), 2.07-2.02 (m, 3H), 1.25 (t, J = 7.6 Hz, 3H). | B |
| 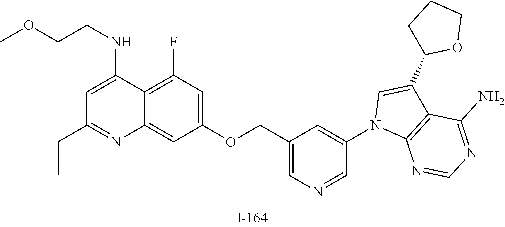 [SMILES: CCc1cc(NCCOC)c2c(F)cc(OCc3cncc(-n4cc([C@@H]5CCCO5)c5c(N)ncnc54)c3)cc2n1] | 558.2 | ¹H NMR (400 MHz, DMSO-d6): δ ppm 9.09 (d, J = 2.5 Hz, 1H), 8.67 (d, J = 1.7 Hz, 1H), 8.44 (t, J = 2.1 Hz, 1H), 8.17 (s, 1H), 7.70 (s, 1H), 7.16 (d, J = 2.4 Hz, 1H), 7.05 (s, 1H), 6.96 (dd, J = 15.4, 2.4 Hz, 1H), 6.55 (s, 1H), 6.36 (s, 1H), 5.39 (s, 2H), 5.06 (t, J = 6.4 Hz, 1H), 3.99-3.95 (m, 1H), 3.92-3.88 (m, 1H), 3.60 (t, J = 5.5 Hz, 2H), 3.46-3.42 (m, 2H), 3.32 (s, 3H), 2.34-2.30 (m, 2H), 2.07-2.02 (m, 3H), 1.25 (t, J = 7.6 Hz, 3H). | B |
| 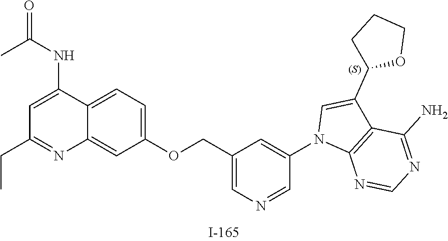 | 524.2 | ¹H NMR (400 MHz, DMSO-d6) δ ppm 10.10 (s, 1H), 9.09 (d, J = 2.4 Hz, 1H), 8.70 (d, J = 2.0 Hz, 1H), 8.47 (t, J = 2.0 Hz, 1H), 8.25 (d, J = 9.2 Hz, 1H), 8.17 (s, 1H), 7.90 (s, 1H), 7.71 (s, 1H), 7.48 (d, J = 2.4 Hz, 1H), 7.31 (dd, J = 2.8 Hz, 9.2 Hz, 1H), 7.05 (brs, 2H), 5.43 (s, 2H), 5.06 (t, J = 6.0 Hz, 1H), 3.98-3.88 (m, 2H), 2.83 (q, J = 7.6 Hz, 15.2 Hz 2H), 2.33-2.32 (m, 1H), 2.23 (s, 3H), 2.03-2.01 (m, 3H), 1.28 (t, J = 7.2 Hz, 3H). | B |
| 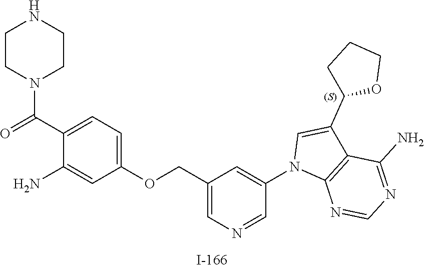 | 514.2 | ¹H NMR (400 MHz, DMSO-d6) δ ppm 9.06 (d, J = 2.0 Hz, 1H), 8.61 (d, J = 1.6 Hz, 1H), 8.39 (t, J = 2.4 Hz, 1H), 8.16 (s, 1H), 7.70 (s, 1H), 7.05 (brs, 2H), 6.93 (d, J = 8.4 Hz, 1H), 6.37 (d, J = 2.0 Hz, 1H), 6.27 (dd, J = 2.4 Hz, 8.4 Hz 1H), 5.31 (s, 2H), 5.17 (s, 2H), 5.06 (t, J = 6.8 Hz, 1H), 4.00-3.86 (m, 2H), 3.3 (t, J = 4.4 Hz, 4H), 2.65 (t, J = 4.4 Hz, 4H), 2.33-1.99 (m, 4H). | B |
| 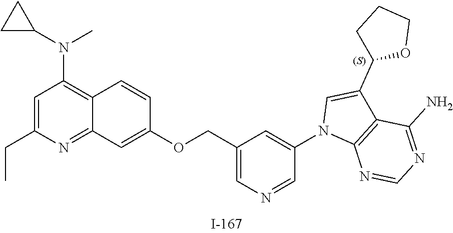 | 536.0 | ¹H NMR (400 MHz, DMSO-d6) δ ppm 9.08 (d, J = 2.8 Hz, 1H), 8.69 (d, J = 2.0 Hz, 1H), 8.46 (t, J = 2.0 Hz, 1H), 8.17 (s, 1H), 7.92 (d, J = 9.2 Hz, 1H), 7.71 (s, 1H), 7.37 (d, J = 2.4 Hz, 1H), 7.14 (dd, J = 2.4, 9.2 Hz, 1H), 7.05 (brs, 2H), 6.87 (s, 1H), 5.40 (s, 2H), 5.06 (t, J = 6.8 Hz, 1H), 4.01-3.86 (m, 2H), 3.08 (s, 3H), 2.80 (q, J = 7.6, 15.2 Hz, 3H), 2.34-2.02 (m, 4H), 1.29 (t, J = 7.6 Hz, 3H), 0.791 (dt, J = 7.2, 12.0 Hz, 2H), 0.44 (dq, J = 4.4, 6.8 Hz, 2H). | B |
| 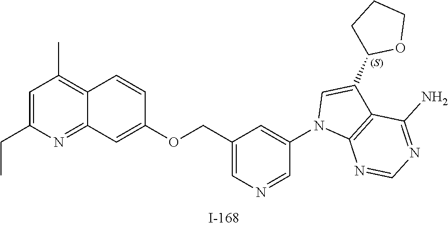 | 581.2 | ¹H NMR (400 MHz, DMSO-d6) δ ppm 9.08 (d, J = 2.8 Hz, 1H), 8.70 (d, J = 2 Hz, 1H), 8.47 (t, J = 2.2 Hz, 1H), 8.17 (s, 1H), 7.97 (d, J = 9.2 Hz, 1H), 7.71 (d, J = 0.8 Hz, 1H), 7.49 (d, J = 5.2 Hz, 1H), 7.31 (dd, J = 9.2, 2.4 Hz, 1H), 7.16 (s, 1H), 7.05 (s, 1H), 5.44 (s, 2H), 5.06 (t, J = 2.4 Hz, 1H), 3.99-3.88 (m, 2H), 2.84 (q, J = 7.6 Hz, 2H), 2.61 (s, 3H), 2.28-2.01 (m, 4H), 1.29 (t, J = 7.4 Hz, 3H). | B |
| 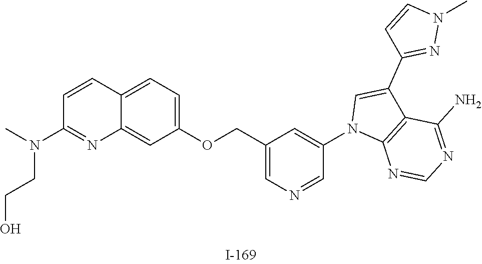 [SMILES: CN(CCO)c1ccc2ccc(OCc3cncc(-n4cc(-c5ccn(C)n5)c5c(N)ncnc54)c3)cc2n1] | 523.0 | ¹H NMR (400 MHz, DMSO-d6) δ ppm 7.18 (s, 1H), 9.12 (d, J = 2.4 Hz, 1H), 8.70 (d, J = 1.6 Hz, 1H), 8.50 (t, J = 4.4 Hz, 1H), 8.25 (s, 1H), 8.15 (s, 1H), 7.74 (d, J = 8.8 Hz, 1H), 7.78 (d, J = 2.4 Hz, 1H), 7.60 (d, J = 8.8 Hz, 1H), 7.37 (s, 1H), 7.12 (s, 1H), 6.94-6.90 (m, 2H), 6.78 (d, J = 2.4 Hz, 1H), 5.36 (s, 2H), 3.90 (s, 3H), 3.70-3.67 (m, 2H), 3.63-3.61 (m, 2H), 3.17 (s, 3H). | B |
| 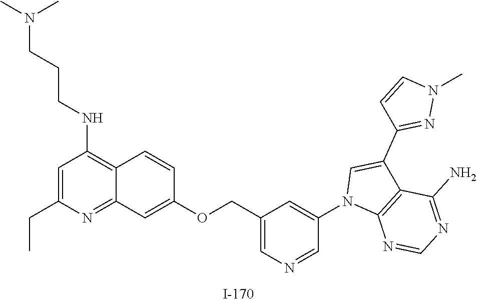 | 289.3 | ¹H NMR (400 MHz, DMSO-d6) δ ppm 9.15 (t, J = 10.2 Hz, 2H), 8.71 (s, 1H), 8.52 (s, 1H), 8.25 (s, 1H), 8.15 (s, 1H), 8.08 (d, J = 9.3 Hz, 1H), 7.79 (d, J = 2.0 Hz, 1H), 7.40 (s, 2H), 7.29 (d, J = 2.1 Hz, 1H), 7.16 (d, J = 8.6 Hz, 1H), 6.77 (d, J = 2.1 Hz, 1H), 6.32 (s, 1H), 5.41 (s, 2H), 3.92 (s, 3H), 2.72 (q, J = 7.5 Hz, 2H), 2.38 (t, J = 6.7 Hz, 2H), 2.20 (s, 6H), 1.86-1.74 (m, 2H), 1.26 (t, J = 7.6 Hz, 3H). | B |
| 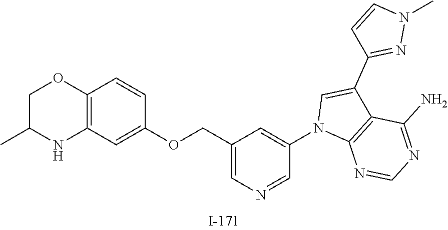 | 469.0 | ¹H NMR (400 MHz, DMSO-d6) δ 9.18 (s, 1H), 9.09 (d, J = 2.4 Hz, 1H), 8.60 (d, J = 1.6 Hz, 1H), 8.41 (s, 1H), 8.25 (s, 1H), 8.15 (s, 1H), 7.79 (d, J = 2.2 Hz, 1H), 7.42 (s, 1H), 6.78 (d, J = 2.2 Hz, 1H), 6.56 (d, J = 8.6 Hz, 1H), 6.25 (d, J = 2.8 Hz, 1H), 6.19-6.16 (m, 1H), 5.85 (s, 1H), 5.11 (s, 2H), 4.06 (d, J = 10.1 Hz, 1H), 3.91 (s, 3H), 3.57-3.52 (m, 7.8 Hz, 1H), 3.31 (s, 1H), 1.05 (d, J = 6.3 Hz, 3H). | B |
| 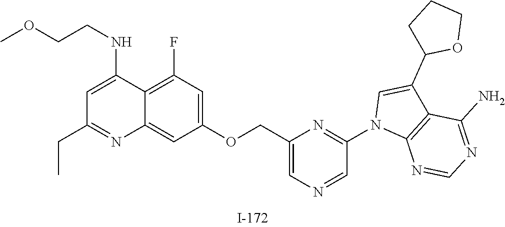 [SMILES: CCc1cc(NCCOC)c2c(F)cc(OCc3cncc(-n4cc(C5CCCO5)c5c(N)ncnc54)n3)cc2n1] | 559.2 | ¹H NMR (400 MHz, DMSO-d6) δ ppm 8.36 (s, 1H), 8.09 (s, 1H), 7.50 (s, 1H), 7.29 (d, J = 1.6 Hz, 1H), 7.10 (dd, J = 14.5, 2.2 Hz, 1H), 6.98 (s, 2H), 6.69-6.65 (m, 1H), 6.48 (s, 1H), 5.04 (t, J = 6.6 Hz, 1H), 3.99-3.93 (m, 1H), 3.90-3.84 (m, 1H), 3.61 (t, J = 5.5 Hz, 2H), 3.50-3.44 (m, 2H), 3.35 (s, 3H), 2.74-2.68 (m, 2H), 2.44 (s, 3H), 2.30-2.22 (m, 1H), 2.04-1.92 (m, 3H), 1.25 (t, J = 7.6 Hz, 3H). | B |
| 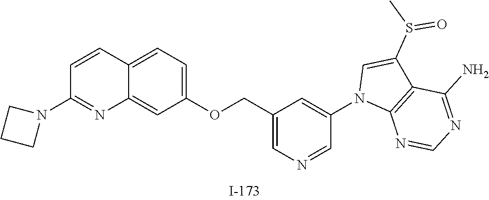 [SMILES: CS(=O)c1cn(-c2cncc(COc3ccc4ccc(N5CCC5)nc4c3)c2)c2ncnc(N)c12] | 486.1 | ¹H NMR (400 MHz, DMSO-d6) δ ppm 9.02 (d, J = 2.4 Hz, 1H), 8.75 (d, J = 1.6 Hz, 1H), 8.41 (t, J = 2.0 Hz, 1H), 8.29 (d, J = 3.2 Hz, 2H), 7.93 (d, J = 8.8 Hz, 1H), 7.72 (s, 2H), 7.62 (d, J = 8.8 Hz, 1H), 7.15 (s, J = 2.4 Hz, 1H), 6.95 (dd, J = 8.8, 2.4 Hz, 1H), 6.53 (d, J = 8.8 Hz, 1H), 5.38 (s, 2H), 4.07 (t, J = 7.2 Hz, 4H), 2.92 (s, 3H), 2.39-2.31 (m, 2H). | B |
| 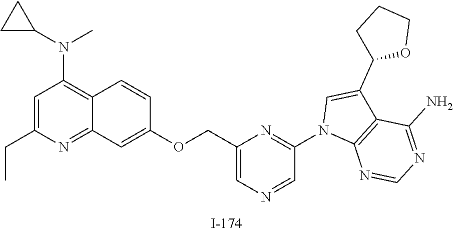 [SMILES: CCc1cc(N(C)C2CC2)c2ccc(OCc3cncc(-n4cc([C@@H]5CCCO5)c5c(N)ncnc54)n3)cc2n1] | 537.3 | ¹H NMR (400 MHz, DMSO-d6) δ 9.96 (s, 1H), 8.74 (s, 1H), 8.29 (s, 1H), 7.94 (d, J = 9.2 Hz, 1H), 7.83 (s, 1H), 7.40 (s, 1H), 7.20-7.17 (m, 3H), 6.88 (s, 1H), 5.45 (s, 2H), 5.04 (t, J = 6.8 Hz, 1H), 3.98-3.87 (m, 2H), 3.09 (s, 3H), 2.82-2.76 (m, 3H), 2.51-2.24 (m, 1H), 2.03-1.99 (m, 2H), 1.87 (s, 1H), 1.30-1.23 (m, 3H), 0.80-0.79 (m, 2H), 0.44 (s, 2H). | B |
| 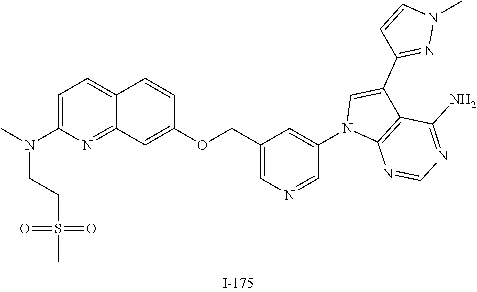 | 585.1 | ¹H NMR (400 MHz, DMSO-d6) δ ppm 9.20 (s, 1H), 9.13 (d, J = 2.8 Hz, 1H), 8.71 (d, J = 1.6 Hz, 1H), 8.51 (s, 1H), 8.27 (s, 1H), 8.15 (s, 1H), 7.99 (s, J = 2.4 Hz, 1H), 7.79 (d, J = 2.4 Hz, 1H), 7.65 (d, J = 8.8 Hz, 1H), 7.44 (s, 1H), 7.16 (d, J = 2 Hz, 1H), 7.49 (dd, J = 8.8 Hz, 2.4 Hz, 1H), 6.93 (d, J = 8.8 Hz, 1H), 6.78 (d, J = 2.4 Hz, 1H), 5.39 (s, 2H), 4.07 (t, J = 7 Hz, 2H), 3.92 (s, 3H), 3.49 (t, J = 7 Hz, 2H), 3.15 (s, 3H), 3.07 (s, 3H). | B |
| 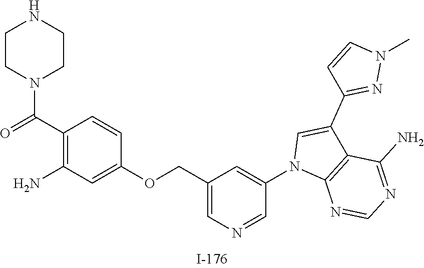 [SMILES: Cn1ccc(-c2cn(-c3cncc(COc4ccc(C(=O)N5CCNCC5)c(N)c4)c3)c3ncnc(N)c23)n1] | 525.0 | ¹H NMR (400 MHz, DMSO-d6) δ 9.18 (s, 1H), 9.12 (d, J = 2.5 Hz, 1H), 8.64 (d, J = 1.7 Hz, 1H), 8.46 (t, J = 2.1 Hz, 1H), 8.26 (s, 1H), 8.15 (s, 1H), 7.78 (d, J = 5.0 Hz, 1H), 7.43 (s, 1H), 6.95 (d, J = 8.5 Hz, 1H), 6.78 (d, J = 2.3 Hz, 1H), 6.39 (d, J = 2.4 Hz, 1H), 6.29 (dd, J = 8.5, 2.4 Hz, 1H), 5.33-5.31 (m 2H), 5.19 (s, 2H), 3.92-3.86 (m, J = 7.8 Hz, 4H), 3.38 (s, 5H), 2.71-2.67 (m, 4H). | B |
| 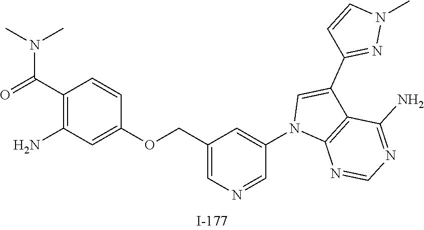 [SMILES: CN(C)C(=O)c1ccc(OCc2cncc(-n3cc(-c4ccn(C)n4)c4c(N)ncnc43)c2)cc1N] | 484.0 | ¹H NMR (400 MHz, DMSO-d6) δ 9.18-9.17 (br, 1H), 9.12 (d, J = 2.5 Hz, 1H), 8.64 (d, J = 1.7 Hz, 1H), 8.46 (t, J = 2.1 Hz, 1H), 8.25 (s, 1H), 8.16 (s, 1H), 7.79 (d, J = 2.3 Hz, 1H), 7.45-7.39 (br, 1H), 6.98 (d, J = 8.5 Hz, 1H), 6.78 (d, J = 2.3 Hz, 1H), 6.39 (d, J = 2.4 Hz, 1H), 6.28 (dd, J = 8.5, 2.4 Hz, 1H), 5.35 (s, 2H), 5.19 (s, 2H), 3.92 (s, 3H), 2.92 (s, 6H). | B |
| 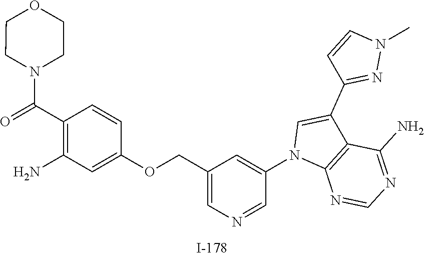 [SMILES: Cn1ccc(-c2cn(-c3cncc(COc4ccc(C(=O)N5CCOCC5)c(N)c4)c3)c3ncnc(N)c23)n1] | 526.0 | ¹H NMR (400 MHz, DMSO-d6) δ 9.19-9.18 (br, 1H), 9.12 (d, J = 2.5 Hz, 1H), 8.64 (d, J = 1.7 Hz, 1H), 8.46 (t, J = 2.1 Hz, 1H), 8.25 (s, 1H), 8.15 (s, 1H), 7.79 (d, J = 2.3 Hz, 1H), 7.44-7.39 (br, 1H), 6.98 (d, J = 8.5 Hz, 1H), 6.78 (d, J = 2.3 Hz, 1H), 6.40 (d, J = 2.4 Hz, 1H), 6.29 (dd, J = 8.5, 2.4 Hz, 1H), 5.38 (s, 2H), 5.20 (s, 2H), 3.92 (s, 3H), 3.62-3.55 (m, 4H), 3.45-3.44 (m, 4H). | B |
| 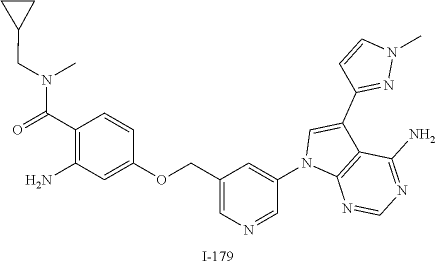 [SMILES: CN(CC1CC1)C(=O)c1ccc(OCc2cncc(-n3cc(-c4ccn(C)n4)c4c(N)ncnc43)c2)cc1N] | 524.0 | ¹H NMR (400 MHz, DMSO-d6) δ 9.24-9.15 (br, 1H), 9.12 (s, J = 2.4 Hz, 1H), 8.65 (d, J = 1.7 Hz, 1H), 8.47 (t, J = 2.1 Hz, 1H), 8.26 (s, 1H), 8.16 (s, 1H), 7.79 (d, J = 2.2 Hz, 1H), 7.46-7.38 (br, 1H), 6.96 (d, J = 8.4 Hz, 1H), 6.78 (d, J = 2.3 Hz, 1H), 6.40 (d, J = 2.4 Hz, 1H), 6.29 (dd, J = 8.5, 2.4 Hz, 1H), 5.25 (s, 2H), 5.19 (s, 2H), 3.92 (s, 3H), 3.22-3.14 (m, 2H), 2.97 (s, 3H), 1.01-0.91 (br, 1H), 0.47-0.42 (m, 2H), 0.16-0.09 (m, 2H). | B |
| 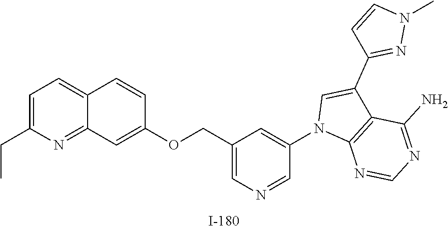 [SMILES: CCc1ccc2ccc(OCc3cncc(-n4cc(-c5ccn(C)n5)c5c(N)ncnc54)c3)cc2n1] | 477.1 | ¹H NMR (400 MHz, DMSO-d6) δ ppm 9.19 (br, 1H ), 9.13 (d, J = 2.8 Hz, 1H), 8.72 (d, J = 1.6 Hz, 1H), 8.54-8.53 (m, 1H), 8.26 (s, 1H), 8.18 (d, J = 8.4 Hz, 1H), 8.14 (s, 1H), 7.86 (d, J = 8.8 Hz, 1H), 7.78 (d, J = 2.4 Hz, 1H), 7.51 (d, J = 2.4 Hz, 1H), 7.41 (br, 1H), 7.31-7.29 (m, 2H), 6.78 (d, J = 2 Hz, 1H), 5.45 (s, 2H), 3.91 (s, 3H), 2.91-2.89 (m, 2H), 1.30 (t, J = 7.6 Hz, 3H). | B |
| 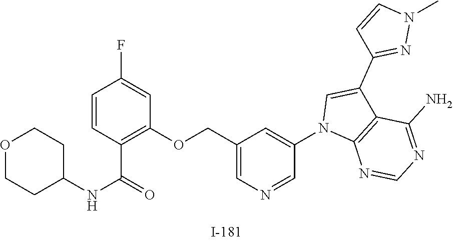 [SMILES: Cn1ccc(-c2cn(-c3cncc(COc4cc(F)ccc4C(=O)NC4CCOCC4)c3)c3ncnc(N)c23)n1] | 543.1 | ¹H NMR (400 MHz, DMSO-d6) δ ppm 9.15 (s, 1H), 9.14 (d, J = 1.6 Hz, 1H), 8.53 (t, J = 2.0 Hz, 1H), 8.22 (s, 1H), 8.14 (s, 1H), 7.99 (d, J = 7.6 Hz, 1H), 7.79 (d, J = 2.4 Hz, 1H), 7.68-7.67 (m, 1H), 7.46 (s, 1H), 7.27 (dd, J = 11.2, 2.4 Hz, 1H), 6.93-6.92 (m, 1H), 6.75 (d, J = 2.0 Hz, 1H), 5.36 (s, 2H), 3.91 (s, 3H), 3.51-3.50 (m, 1H), 3.16-3.10 (m, 1H), 3.49-3.48 (m, 2H), 3.19-3.18 (m, 2H), 1.55 (d, J = 9.6 Hz, 2H), 1.21-1.20 (m, 2H). | B |
| 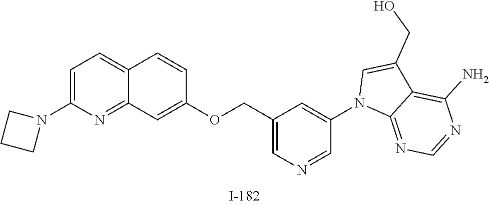 [SMILES: Nc1ncnc2c1c(CO)cn2-c1cncc(COc2ccc3ccc(N4CCC4)nc3c2)c1] | 454.3 | ¹H NMR (400 MHz, DMSO-d6) δ ppm 9.04 (d, J = 2.4 Hz, 1H), 8.65 (d, J = 2.0 Hz, 1H), 8.42 (t, J = 2.0 Hz, 1H), 8.17 (s, 1H), 7.93 (d, J = 8.8 Hz, 1H), 7.67 (s, 1H), 7.61 (d, J = 8.8 Hz, 1H), 7.14 (s, 3H), 6.95 (dd, J = 2.4 Hz 8.8 Hz, 1H), 6.52 (d, J = 8.8 Hz, 1H), 5.91 (t, J = 6.0 Hz, 1H), 5.36 (s, 2H), 4.67 (d, J = 2.4 Hz, 2H), 4.06 (t, J = 7.2 Hz, 4H), 2.38-2.31 (m, 2H). | B |
| 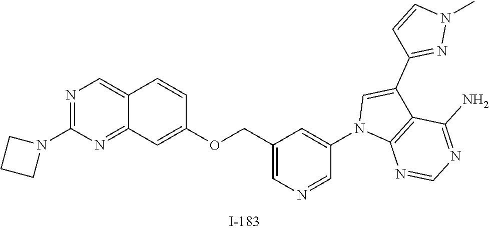 [SMILES: Cn1ccc(-c2cn(-c3cncc(COc4ccc5cnc(N6CCC6)nc5c4)c3)c3ncnc(N)c23)n1] | 505.0 | ¹H NMR (400 MHz, DMSO-d6) δ ppm 9.16 (s, 1H), 9.01 (d, J = 2.4 Hz, 1H), 8.59 (d, J = 1.6 Hz, 1H), 8.45 (t, J = 1.9 Hz, 1H), 8.19 (s, 1H), 8.13 (s, 1H), 8.11 (s, 1H), 7.78 (d, J = 2.2 Hz, 1H), 7.39 (s, 1H), 7.25 (d, J = 9.4 Hz, 1H), 6.75 (d, J = 2.3 Hz, 1H), 6.33 (d, J = 10.2 Hz, 1H), 5.83 (s, 1H), 4.97 (s, 2H), 4.14-4.05 (m, 2H), 3.91 (s, 3H), 3.57 (t, J = 5.7 Hz, 2H), 2.20-2.11 (m, 2H). | B |
| 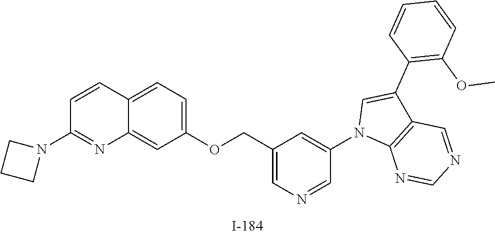 [SMILES: COc1ccccc1-c1cn(-c2cncc(COc3ccc4ccc(N5CCC5)nc4c3)c2)c2ncncc12] | 515.0 | ¹H NMR (400 MHz, DMSO-d6) δ 9.19 (d, J = 2.4 Hz, 1H), 9.16 (s, 1H), 8.94 (s, 1H), 8.76 (d, J = 1.6 Hz, 1H), 8.56 (s, 1H), 8.34 (s, 1H), 7.94 (d, J = 8.8 Hz, 1H), 7.71-7.59 (m, 2H), 7.45-7.37 (m, 1H), 7.22-7.17 (m, 2H), 7.10 (t, J = 7.5 Hz, 1H), 6.98 (dd, J = 8.8, 2.4 Hz, 1H), 6.54 (d, J = 8.8 Hz, 1H), 5.42 (s, 2H), 4.07 (t, J = 7.4 Hz, 4H), 3.87 (s, 3H), 2.39-2.33 (m, 2H). | A |
| 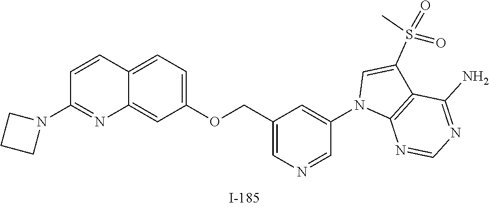 [SMILES: CS(=O)(=O)c1cn(-c2cncc(COc3ccc4ccc(N5CCC5)nc4c3)c2)c2ncnc(N)c12] | 502.0 | ¹H NMR (400 MHz, DMSO-d6) δ ppm 9.03 (d, J = 2.4 Hz, 1H), 8.79 (d, J = 1.6 Hz, 1H), 8.50 (s, 1H), 8.41 (t, J = 2.0 Hz, 1H), 8.30 (s, 1H), 7.93 (d, J = 8.4 Hz, 1H), 7.62 (d, J = 8.8 Hz, 1H), 7.15 (d, J = 2.4 Hz, 1H), 6.95 (dd, J = 8.8, 2.4 Hz, 1H), 6.53 (d, J = 8.8 Hz, 1H), 5.39 (s, 2H), 4.07 (t, J = 7.2 Hz, 4H), 3.38 (s, 3H), 2.39-2.32 (m, 2H). | B |
| 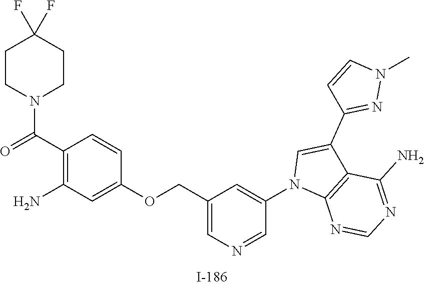 [SMILES: Cn1ccc(-c2cn(-c3cncc(COc4ccc(C(=O)N5CCC(F)(F)CC5)c(N)c4)c3)c3ncnc(N)c23)n1] | 561.2 | ¹H NMR (400 MHz, DMSO-d6) δ 9.20 (s, 1H), 9.12 (d, J = 2.5 Hz, 1H), 8.65 (d, J = 1.7 Hz, 1H), 8.46 (t, J = 2.1 Hz, 1H), 8.26 (s, 1H), 8.16 (s, 1H), 7.79 (d, J = 2.2 Hz, 1H), 7.43 (s, 1H), 7.01 (d, J = 8.4 Hz, 1H), 6.78 (d, J = 2.3 Hz, 1H), 6.39 (d, J = 2.4 Hz, 1H), 6.30 (dd, J = 8.5, 2.4 Hz, 1H), 5.39 (s, 2H), 5.20 (s, 2H), 3.92 (s, 3H), 3.53 (s, 4H), 2.02-1.98 (m, 4H). | B |
| 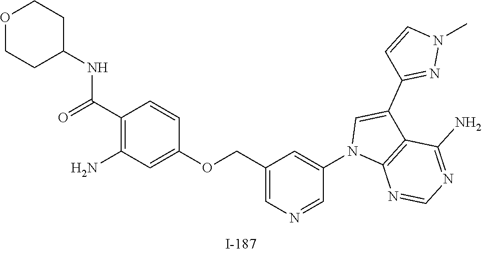 [SMILES: Cn1ccc(-c2cn(-c3cncc(COc4ccc(C(=O)NC5CCOCC5)c(N)c4)c3)c3ncnc(N)c23)n1] | 540.0 | ¹H NMR (400 MHz, DMSO-d6) δ ppm 9.23 (s, 1H), 9.11 (d, J = 2.4 Hz, 1H), 8.64 (d, J = 2.0 Hz, 1H), 8.25 (s, 1H), 8.15 (s, 1H), 7.89 (d, J = 4.0 Hz, 1H), 7.79 (d, J = 2.4 Hz, 1H), 7.50 (d, J = 8.8 Hz, 1H), 7.48 (s, 1H), 6.77 (d, J = 2.0 Hz, 1H), 6.58 (s, 2H), 6.35 (d, J = 2.8 Hz, 1H), 6.28 (d, J = 2.0 Hz, 1H), 5.21 (s, 2H), 3.91 (s, 4H), 3.89 (d, J = 4.0 Hz, 2H), 3.37 (s, 1H), 3.31 (s, 1H), 1.38 (d, J = 2.0 Hz, 2H), 1.29-1.28 (m, 2H). | B |
| 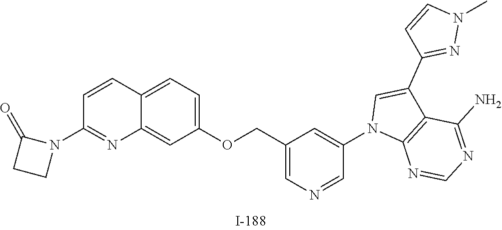 [SMILES: Cn1ccc(-c2cn(-c3cncc(COc4ccc5ccc(N6CCC6=O)nc5c4)c3)c3ncnc(N)c23)n1] | 518.1 | ¹H NMR (400 MHz, DMSO-d6) δ ppm 9.19 (s, 1H), 9.13 (d, J = 2.4 Hz, 1H), 8.71 (d, J = 1.6 Hz, 1H), 8.53 (t, J = 2 Hz, 1H), 8.29 (d, J = 8.8 Hz, 1H), 8.27 (s, 1H), 8.15 (s, 1H), 7.85 (d, J = 8.8 Hz, 1H), 7.79 (d, J = 2 Hz, 1H), 7.70 (d, J = 8.8 Hz, 1H), 7.42 (s, 1H), 7.41 (d, J = 2.4 Hz, 1H), 7.25 (dd, J = 9 Hz, 2.4 Hz, 1H), 6.78 (d, J = 6 Hz, 1H), 5.45 (s, 2H), 3.92 (s, 3H), 3.81 (t, J = 4.6 Hz, 2H), 3.15 (t, J = 4.6 Hz, 2H). | B |
| 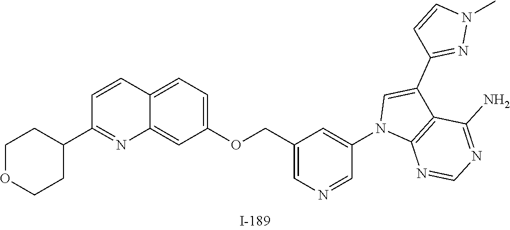 [SMILES: Cn1ccc(-c2cn(-c3cncc(COc4ccc5ccc(C6CCOCC6)nc5c4)c3)c3ncnc(N)c23)n1] | 533.2 | ¹H NMR (400 MHz, DMSO-d6) δ ppm 9.20 (s, 1H), 9.14 (d, J = 2.4 Hz, 1H), 8.73 (d, J = 1.6 Hz, 1H), 8.54 (t, J = 2 Hz, 1H), 8.28 (s, 1H), 8.22 (d, J = 8.4 Hz, 1H), 8.15 (s, 1H), 7.76 (d, J = 8.8 Hz, 1H), 7.79 (d, J = 2.4 Hz, 1H), 7.51 (d, J = 2.4 Hz, 1H), 7.45 (s, 1H), 7.36 (d, J = 15.6 Hz, 1H), 7.32 (dd, J = 8.8, 2.4 Hz, 2H), 6.68 (d, J = 5.6 Hz, 1H), 5.47 (s, 2H), 4.00-3.97 (m, 2H), 3.91 (s, 3H), 3.52-3.41 (m, 2H), 3.12-3.07 (m, 1H), 1.90-1.84 (m, 4H). | B |
| 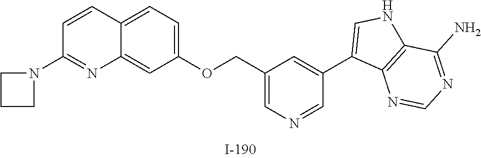 [SMILES: Nc1ncnc2c(-c3cncc(COc4ccc5ccc(N6CCC6)nc5c4)c3)c[nH]c12] | 424.0 | ¹H NMR (400 MHz, DMSO-d6) δ ppm 11.36 (d, J = 2.3 Hz, 1H), 9.31 (d, J = 2.0 Hz, 1H), 8.67 (t, J = 2.0 Hz, 1H), 8.50 (d, J = 1.9 Hz, 1H), 8.24 (s, 1H), 8.20 (d, J = 3.0 Hz, 1H), 7.93 (d, J = 8.8 Hz, 1H), 7.62 (d, J = 8.8 Hz, 1H), 7.14 (d, J = 2.3 Hz, 1H), 6.95 (dd, J = 8.7, 2.4 Hz, 1H), 6.85 (s, 2H), 6.53 (d, J = 8.8 Hz, 1H), 5.30 (s, 2H), 4.07 (t, J = 7.4 Hz, 4H), 2.37-2.32 (m, 2H). | D |
| 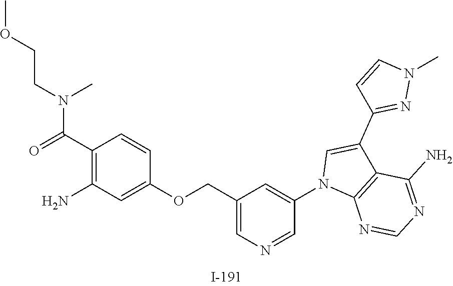 [SMILES: COCCN(C)C(=O)c1ccc(OCc2cncc(-n3cc(-c4ccn(C)n4)c4c(N)ncnc43)c2)cc1N] | 528.1 | ¹H NMR (400 MHz, DMSO-d6) δ 9.23-9.16 (br, 1H), 9.12 (d, J = 2.4 Hz, 1H), 8.64 (d, J = 1.5 Hz, 1H), 8.47-8.46 (m, 1H), 8.26 (s, 1H), 8.16 (s, 1H), 7.79 (d, J = 2.2 Hz, 1H), 7.46-7.39 (m, 1H), 6.97 (d, J = 8.4 Hz, 1H), 6.78 (d, J = 2.2 Hz, 1H), 6.39 (d, J = 2.3 Hz, 1H), 6.29 (dd, J = 8.4, 2.3 Hz, 1H), 5.25 (s, 2H), 5.19 (s, 2H), 3.92 (s, 3H), 3.53-3.45 (m, 4H), 3.22 (s, 3H), 2.92 (s, 3H). | B |
| 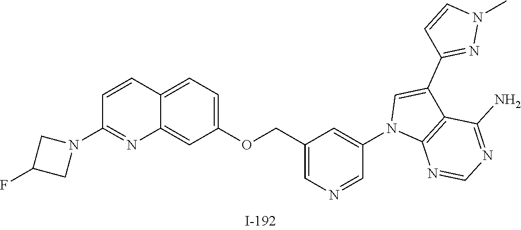 [SMILES: Cn1ccc(-c2cn(-c3cncc(COc4ccc5ccc(N6CC(F)C6)nc5c4)c3)c3ncnc(N)c23)n1] | 522.3 | ¹H NMR (400 MHz, DMSO-d6) δ ppm 9.18 (s, 1H), 9.12 (d, J = 2.4 Hz, 1H), 8.69 (d, J = 2 Hz, 1H), 8.50 (t, J = 2.2 Hz, 1H), 8.26 (s, 1H), 8.15 (s, 1H), 7.99 (d, J = 8.8 Hz, 1H), 7.79 (d, J = 2.4 Hz, 1H), 7.66 (d, J = 9.2 Hz, 1H), 7.42 (s, 1H), 7.20 (d, J = 2.4 Hz, 1H), 7.01 (dd, J = 8.8 Hz, 2.8 Hz, 1H), 6.78 (d, J = 2.4 Hz, 1H), 6.62 (d, J = 8.8 Hz, 1H), 5.60-5.39 (m, 1H), 5.39 (s, 2H), 4.46-4.36 (m, 2H), 4.17-4.08 (m, 2H), 3.90 (s, 3H) | B |
| 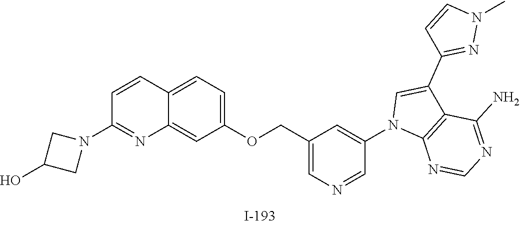 [SMILES: Cn1ccc(-c2cn(-c3cncc(COc4ccc5ccc(N6CC(O)C6)nc5c4)c3)c3ncnc(N)c23)n1] | 520.0 | ¹H NMR (400 MHz, DMSO-d6) δ 9.18 (s, 1H), 9.12 (d, J = 2.4 Hz, 1H), 8.69 (d, J = 1.6 Hz, 1H), 8.50 (d, J = 2.2 Hz, 1H), 8.26 (s, 1H), 8.15 (s, 1H), 7.93 (d, J = 9.2 Hz, 1H), 7.79 (d, J = 2.4 Hz, 1H), 7.63 (d, J = 9.2 Hz, 1H), 7.42 (s, 1H), 7.17 (d, J = 2.4 Hz, 1H), 6.97 (dd, J = 8.8, 2.4 Hz, 1H), 6.78 (d, J = 2.0 Hz, 1H), 6.56 (d, J = 8.8 Hz, 1H), 5.71 (d, J = 6.4 Hz, 1H), 5.34 (s, 2H), 4.62-4.58 (m, 1H), 4.28 (t, J = 8.4 Hz, 2H), 3.91 (s, 3H), 3.82-3.78 (m, 2H). | B |
| 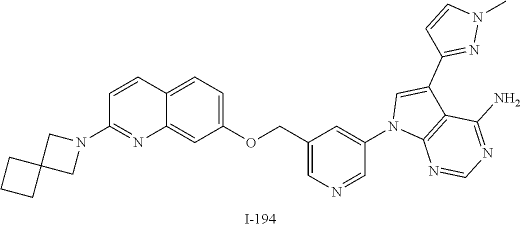 [SMILES: Cn1ccc(-c2cn(-c3cncc(COc4ccc5ccc(N6CC7(CCC7)C6)nc5c4)c3)c3ncnc(N)c23)n1] | 544.0 | ¹H NMR (400 MHz, DMSO-d6) δ ppm 9.20 (brs, 1H), 9.15 (d, J = 2.4 Hz 1H), 8.69 (d, J = 2.0 Hz, 1H), 8.49 (t, J = 2.0 Hz, 1H), 8.26 (s, 1H), 8.15 (s, 1H), 7.92 (d, J = 2.4 Hz, 1H), 7.79 (d, J = 8.8 Hz, 1H), 7.62 (d, J = 8.8 Hz, 1H), 7.41 (brs, 1H), 7.14 (d, J = 2.4 Hz, 1H), 6.97-6.94 (m, 1H), 6.77 (d, J = 2.0 Hz 1H), 6.52 (d, J = 8.8 Hz 1H), 5.38 (s, 2H), 4.00 (s, 4H), 3.91 (s, 3H), 2.19 (t, J = 7.2 Hz, 4H), 1.83 (p, J = 7.6, 15.2 Hz, 2H). | B |
| 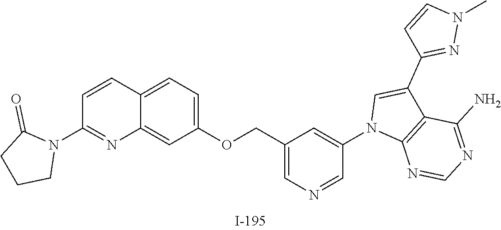 [SMILES: Cn1ccc(-c2cn(-c3cncc(COc4ccc5ccc(N6CCCC6=O)nc5c4)c3)c3ncnc(N)c23)n1] | 532.3 | ¹H NMR (400 MHz, DMSO-d6) δ ppm 9.19 (s, 1H), 9.14 (d, J = 2.4 Hz, 1H), 8.72 (d, J = 2.4 Hz, 1H), 8.53 (t, J = 2.2 Hz, 1H), 8.39 (s, J = 9.2 Hz, 1H), 8.27 (d, J = 8.8 Hz, 1H), 8.26 (s, 1H), 8.15 (s, 1H), 7.86 (m, J = 8.8 Hz, 1H), 7.79 (d, J = 2.4 Hz, 1H), 7.43 (s, 1H), 7.27 (dd, J = 7.6, 2.4 Hz, 1H), 6.78 (m, J = 2.4, 2H), 5.45 (s, 2H), 4.14 (t, J = 7 Hz, 2H), 3.91 (s, 3H), 2.64 (t, J = 8 Hz, 2H), 2.12-2.05 (m, 2H). | B |
| 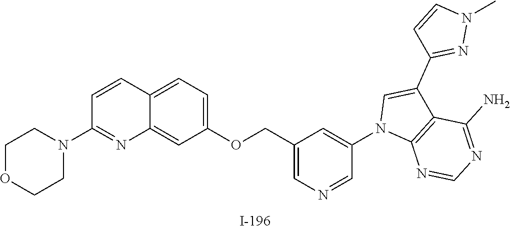 [SMILES: Cn1ccc(-c2cn(-c3cncc(COc4ccc5ccc(N6CCOCC6)nc5c4)c3)c3ncnc(N)c23)n1] | 534.1 | ¹H NMR (400 MHz, DMSO-d6) δ ppm 9.22 (s, 1H), 9.13 (s, 1H), 8.70 (s, 1H), 8.51 (s, 1H), 8.27 (s, 1H), 8.16 (s, 1H), 8.00 (d, J = 8.4 Hz, 1H), 7.79 (s, 1H), 7.65 (d, J = 8.4 Hz, 1H), 7.46 (s, 1H), 7.17 (s, 1H), 7.07 (d, J = 8.4 Hz, 1H), 7.01 (d, J = 8.0 Hz, 1H), 6.78 (s, 1H), 5.39 (s, 2H), 3.92 (s, 3H), 3.72 (s, 4H), 3.64 (s, 4H). | B |
| 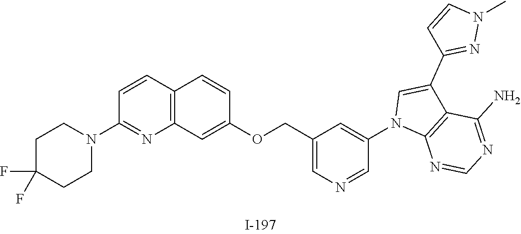 [SMILES: Cn1ccc(-c2cn(-c3cncc(COc4ccc5ccc(N6CCC(F)(F)CC6)nc5c4)c3)c3ncnc(N)c23)n1] | 568.0 | ¹H NMR (400 MHz, DMSO-d6) δ ppm 9.25 (br, 1H), 9.11 (d, J = 2.4 Hz, 1H), 8.70 (s, 1H), 8.51-8.49 (m, 1H), 8.27 (s, 1H), 8.16 (s, 1H), 8.01 (d, J = 9.2 Hz, 1H), 7.79 (d, J = 2.4 Hz, 1H), 7.66 (d, J = 8.4 Hz, 1H), 7.48 (br, 1H), 7.19-7.16 (m, 1H), 7.02 (dd, J = 8.8, 2.4 Hz, 1H), 6.78 (d, J = 2.4 Hz, 1H), 5.39 (s, 2H), 3.96 (s, 3H), 3.87-3.84 (m, 4H), 2.06-1.98 (m, 4H) | B |
| 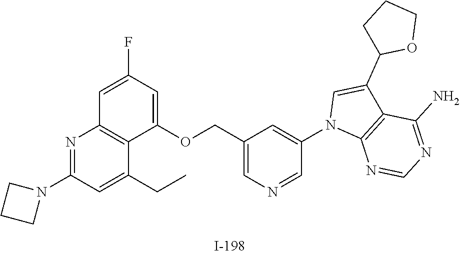 [SMILES: CCc1cc(N2CCC2)nc2cc(F)cc(OCc3cncc(-n4cc(C5CCCO5)c5c(N)ncnc54)c3)c12] | 541.2 | ¹H NMR (400 MHz, DMSO-d6) δ ppm 9.11 (d, J = 2.4 Hz, 1H), 8.69 (s, 1H), 8.58 (t, J = 2.0 Hz, 1H), 8.15 (s, 1H), 7.70 (s, 1H), 7.10 (d, J = 2.4 Hz, 2H), 7.05 (s, 2H), 6.18 (s, 1H), 5.39 (s, 2H), 5.06 (t, J = 2.0 Hz, 1H), 4.00-3.98 (m, 4H), 3.96 (t, J = 18.8 Hz, 1H), 2.90 (d, J = 7.6 Hz, 2H), 2.32 (s, 1H), 2.04-2.03 (m, 5H), 1.16 (t, J = 7.6 Hz, 1H), 1.02 (t, J = 8.0 Hz, 3H). | B |
| 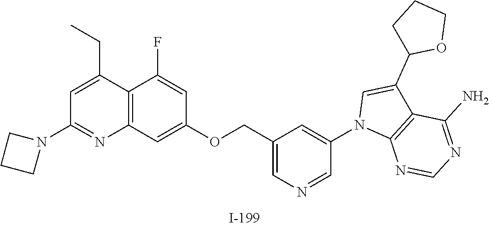 [SMILES: CCc1cc(N2CCC2)nc2cc(OCc3cncc(-n4cc(C5CCCO5)c5c(N)ncnc54)c3)cc(F)c12] | 541.2 | ¹H NMR (400 MHz, DMSO-d6) δ ppm 9.09 (d, J = 2.4 Hz, 1H), 8.68 (d, J = 1.6 Hz, 1H), 8.46 (s, 1H), 8.18 (s, 1H), 7.71 (s, 1H), 7.07 (s, 2H), 6.84 (d, J = 15.2 Hz, 2H), 6.15 (s, 1H), 5.38 (s, 2H), 5.06 (t, J = 2.0 Hz, 1H), 4.00-3.98 (m, 1H), 3.93-3.92 (m, 1H), 3.90-3.83 (m, 2H), 3.83-3.76 (m, 1H), 2.72-2.71 (m, 2H), 2.51-2.28 (m, 1H), 2.06-2.05 (m, 3H), 2.04-2.03 (m, 2H), 1.17 (s, 1H), 1.15 (t, J = 7.2 Hz, 3H). | B |
| 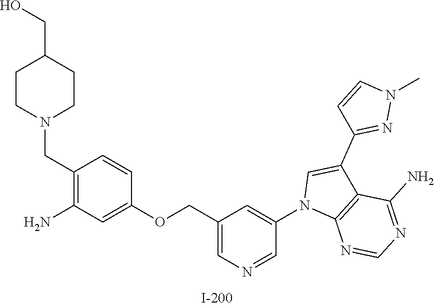 [SMILES: Cn1ccc(-c2cn(-c3cncc(COc4ccc(CN5CCC(CO)CC5)c(N)c4)c3)c3ncnc(N)c23)n1] | 540.3 | ¹H NMR (400 MHz, DMSO-d6) δ ppm 9.18 (br, 1H), 9.10 (s, 1H), 8.63 (s, 1H), 8.44 (s, 1H), 8.25 (s, 1H), 8.15 (s, 1H), 7.90 (s, 1H), 7.42 (br, 1H), 6.83 (br, 1H), 6.78 (s, 1H), 6.33 (s, 1H), 6.21 (d, J = 10.4 Hz, 1H), 5.36 (br, 2H), 5.15 (s, 2H), 4.43 (s, 1H), 3.92 (s, 3H), 3.34-3.32 (m, 2H), 3.23 (t, J = 6.7 Hz, 2H), 2.82-2.73 (m, 2H), 1.93-1.74 (m, 2H), 1.63 (d, J = 12 Hz, 2H), 1.41-1.31 (m, 1H), 1.12-1.06 (m, 2H). | B |
| 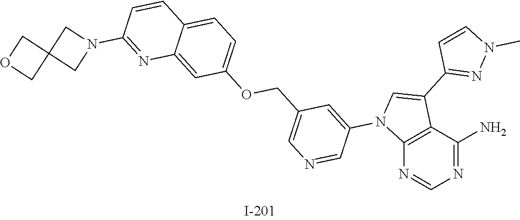 [SMILES: Cn1ccc(-c2cn(-c3cncc(COc4ccc5ccc(N6CC7(COC7)C6)nc5c4)c3)c3ncnc(N)c23)n1] | 546.2 | ¹H NMR (400 MHz, DMSO-d₆) δ ppm 9.19 (s, 1H), 9.12 (d, J = 2.8 Hz, 1H), 8.69 (d, J = 1.6 Hz, 1H), 8.50 (t, J = 2.0 Hz, 1H), 8.25 (s, 1H), 8.14 (s, 1H), 7.95 (m, 1H), 7.79 (d, J = 2.0 Hz, 1H), 7.64 (d, J = 9.2 Hz, 1H), 7.48 (s, 1H), 7.17 (d, J = 2.4 Hz, 1H), 6.98 (dd, J = 8.8 Hz, J = 2.4 Hz, 1H), 6.77 (d, J = 2.0 Hz, 1H), 6.56 (d, J = 8.8 Hz, 1H), 5.38 (s, 2H), 4.74 (s, 4H), 4.23 (s, 4H), 3.91 (s, 3H). | B |
| 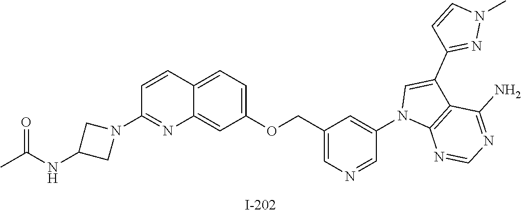 [SMILES: CC(=O)NC1CN(c2ccc3ccc(OCc4cncc(-n5cc(-c6ccn(C)n6)c6c(N)ncnc65)c4)cc3n2)C1] | 561.0 | ¹H NMR (400 MHz, DMSO-d₆) δ 9.19 (s, 1H), 9.12 (d, J = 2.4 Hz, 1H), 8.69 (d, J = 1.7 Hz, 1H), 8.57 (d, J = 7.2 Hz, 1H), 8.50 (s, 1H), 8.26 (s, 1H), 8.14 (d, J = 3.4 Hz, 2H), 7.96 (d, J = 8.8 Hz, 1H), 7.79 (d, J = 2.3 Hz, 1H), 7.64 (d, J = 8.9 Hz, 1H), 7.43 (s, 1H), 7.18 (d, J = 2.5 Hz, 1H), 6.98 (dd, J = 8.7, 2.5 Hz, 1H), 6.78 (d, J = 2.2 Hz, 1H), 6.58 (d, J = 8.8 Hz, 1H), 5.39 (s, 1H), 4.62 (d, J = 7.6 Hz, 1H), 4.32 (t, J = 8.1 Hz, 2H), 3.92 (s, 3H), 3.88-3.84 (m, 2H), 1.84 (s, 3H). | B |
| 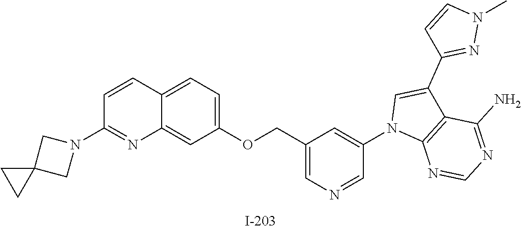 [SMILES: Cn1ccc(-c2cn(-c3cncc(COc4ccc5ccc(N6CC7(CC7)C6)nc5c4)c3)c3ncnc(N)c23)n1] | 530.0 | ¹H NMR (400 MHz, DMSO-d₆) δ 9.19 (s, 1H), 9.13 (d, J = 2.4 Hz, 1H), 8.70 (d, J = 1.7 Hz, 1H), 8.51 (t, J = 2.1 Hz, 1H), 8.26 (s, 1H), 8.16 (s, 1H), 7.95 (d, J = 8.8 Hz, 1H), 7.79 (d, J = 2.2 Hz, 1H), 7.64 (d, J = 8.8 Hz, 1H), 7.43 (s, 1H), 7.17 (d, J = 2.4 Hz, 1H), 6.97 (dd, J = 8.8, 2.5 Hz, 1H), 6.78 (d, J = 2.3 Hz, 1H), 6.56 (d, J = 8.8 Hz, 1H), 5.39 (s, 2H), 4.14 (s, 4H), 3.92 (s, 3H), 0.69 (s, 4H). | B |
| 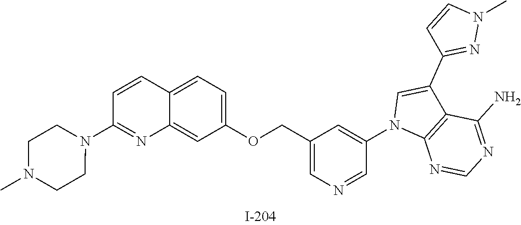 | 547.3 | ¹H NMR (400 MHz, DMSO-d₆) δ ppm 9.19 (br, 1H), 9.12 (s, 1H), 8.70 (s, 1H), 8.51 (s, 1H), 8.26 (s, 1H), 8.15 (s, 1H), 7.96 (d, J = 9.2 Hz, 1H), 7.79 (s, 1H), 7.63 (d, J = 8.8 Hz, 1H), 7.41 (br, 1H), 7.15 (s, 1H), 7.08 (d, J = 9.2 Hz, 1H), 6.99 (d, J = 11.2 Hz, 1H), 6.78 (s, 1H), 5.39 (s, 2H), 3.92 (s, 3H), 3.69 (br, 4H), 2.52-2.50 (m, 4H). 2.21 (s, 3H). | B |
| 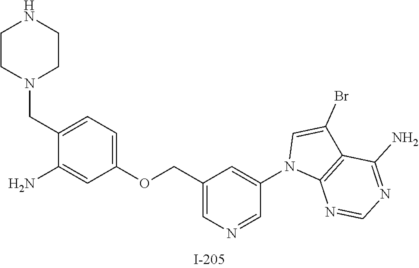 [SMILES: Nc1cc(OCc2cncc(-n3cc(Br)c4c(N)ncnc43)c2)ccc1CN1CCNCC1] | 524.1 | ¹H NMR (400 MHz, DMSO-d₆) δ 9.01 (d, J = 2.3 Hz, 1H), 8.82 (s, 2H), 8.66 (s, 1H), 8.42-8.34 (m, 1H), 8.20 (s, 1H), 8.02 (s, 1H), 7.03 (d, J = 8.5 Hz, 2H), 6.38 (d, J = 2.3 Hz, 1H), 6.30 (dd, J = 8.5, 2.4 Hz, 1H), 5.80-5.35 (m, 1H), 5.19 (s, 2H), 3.70-3.58 (s, 4H), 3.15 (s, 4H). | B |
| 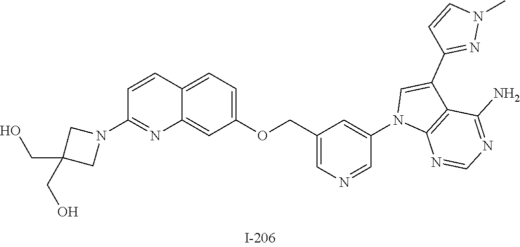 [SMILES: Cn1ccc(-c2cn(-c3cncc(COc4ccc5ccc(N6CC(CO)(CO)C6)nc5c4)c3)c3ncnc(N)c23)n1] | 582.2 | ¹H NMR (400 MHz, DMSO-d₆) δ ppm 9.19 (s, 1H), 9.12 (d, J = 2.8 Hz, 1H), 8.70 (d, J = 1.6 Hz, 1H), 8.50 (t, J = 2.0 Hz, 1H), 8.26 (s, 1H), 8.15 (d, , J = 6.8 Hz, 1H), 7.95 (m, 1H), 7.79 (d, J = 2.4 Hz, 1H), 7.69 (d, J = 8.4 Hz, 1H), 7.48 (s, 1H), 7.19 (s, 1H), 6.79 (d, J = 4.0 Hz, 1H), 6.78 (d, J = 2.4 Hz, 1H), 6.56 (d, J = 8.8 Hz, 1H), 5.39 (s, 2H), 5.16 (s, 1H), 3.96 (m, 9H), 3.66 (d, J = 4.8 Hz, 2H). | B |
| 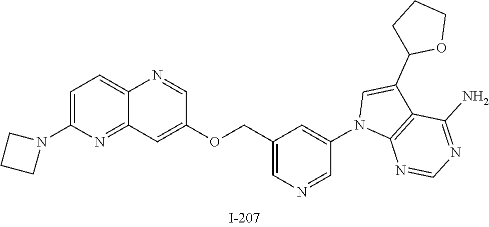 [SMILES: Nc1ncnc2c1c(C1CCCO1)cn2-c1cncc(COc2cnc3ccc(N4CCC4)nc3c2)c1] | 495.1 | ¹H NMR (400 MHz, DMSO-d6) δ ppm 9.09 (d, J = 2.4 Hz, 1H), 8.67 (d, J = 1.5 Hz, 1H), 8.52-8.41 (m, 1H), 8.16 (s, 1H), 8.07 (d, J = 2.2 Hz, 1H), 7.71 (s, 1H), 7.23 (d, J = 1.9 Hz, 1H), 7.14 (d, J = 9.9 Hz, 1H), 7.05 (s, 2H), 6.40 (d, J = 9.9 Hz, 1H), 5.43 (s, 2H), 5.12-4.98 (m, 1H), 4.05-3.95 (m, 1H), 3.94-3.85 (m, 1H), 3.80 (t, J = 8.0 Hz, 2H), 3.42-3.36 (m, 2H), 2.35-2.24 (m, 1H), 2.11-1.96 (m, 3H), 1.94-1.79 (m, 2H). | B |
| 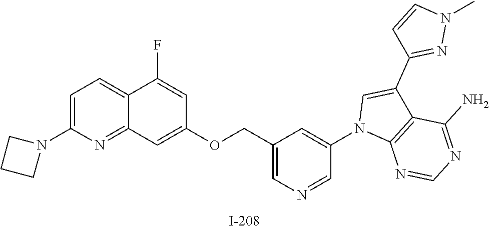 [SMILES: Cn1ccc(-c2cn(-c3cncc(COc4cc(F)c5ccc(N6CCC6)nc5c4)c3)c3ncnc(N)c23)n1] | 523.1 | ¹H NMR (400 MHz, DMSO-d6) δ ppm 9.20-9.19 (m, 1H), 9.12 (d, J = 2.4 Hz, 1H), 8.68 (d, J = 2.4 Hz, 1H), 8.50-8.49 (m, 1H), 8.26 (s, 1H), 8.15 (s, 1H), 8.01 (d, J = 2.4 Hz, 1H), 7.79 (d, J = 2.4 Hz, 1H), 7.45-7.43 (m, 1H), 7.01 (s, 1H), 6.83 (d, J = 11.6 Hz, 1H), 6.77 (d, J = 2.4 Hz, 1H), 6.59 (d, J = 11.6 Hz, 1H), 5.40 (s, 2H), 4.12-4.07 (m, 3H), 3.92 (s, 4H), 2.36-2.32 (m, 2H). | B |
| 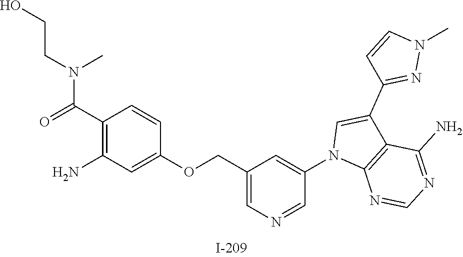 [SMILES: CN(CCO)C(=O)c1ccc(OCc2cncc(-n3cc(-c4ccn(C)n4)c4c(N)ncnc43)c2)cc1N] | 514.3 | ¹H NMR (400 MHz, DMSO-d6) δ ppm 9.19 (s, 1H), 9.12 (d, J = 2.1 Hz, 1H), 8.65 (s, 1H), 8.54-8.36 (m, 4H), 8.25 (s, 1H), 8.16 (s, 1H), 7.79 (d, J = 1.5 Hz, 1H), 7.40 (s, 1H), 7.00 (d, J = 8.6 Hz, 1H), 6.78 (d, J = 1.9 Hz, 1H), 6.39 (d, J = 1.9 Hz, 1H), 6.33-6.21 (m, 1H), 5.26 (s, 2H), 5.19 (s, 2H), 3.92 (s, 3H), 3.60-3.50 (m, 2H), 2.94 (s, 3H). | B |
| 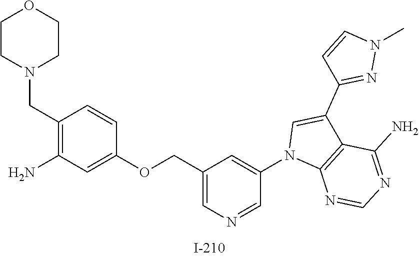 [SMILES: Cn1ccc(-c2cn(-c3cncc(COc4ccc(CN5CCOCC5)c(N)c4)c3)c3ncnc(N)c23)n1] | 512.2 | ¹H NMR (400 MHz, DMSO-d6) δ ppm 9.19 (s, 1H), 9.10 (s, 1H), 8.62 (s, 1H), 8.44 (s, 1H), 8.25 (s, 1H), 8.16 (s, 1H), 7.79 (s, 1H), 7.41 (s, 1H), 6.86 (d, J 7.2 Hz, 1H), 6.78 (s, 1H), 6.35 (s, 1H), 6.22 (d, J = 9.6 Hz, 1H), 5.30 (brs, 2H), 5.16 (s, 2H), 3.92 (s, 3H), 3.55 (s, 4H), 3.31-3.28 (m, 2H), 2.38-2.33 (m, 4H). | B |
| 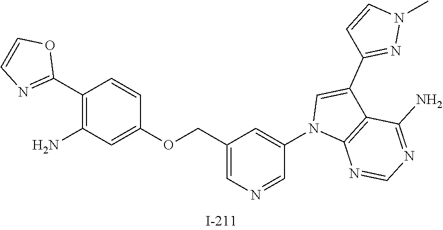 [SMILES: Cn1ccc(-c2cn(-c3cncc(COc4ccc(-c5ncco5)c(N)c4)c3)c3ncnc(N)c23)n1] | 480.1 | ¹H NMR (400 MHz, DMSO-d6) δ ppm 9.18 (s, 1H), 9.13 (d, J = 2.4 Hz, 1H), 8.66 (d, J = 1.6 Hz, 1H), 8.48-8.47 (m, 1H), 8.25 (s, 1H), 8.16 (s, 1H), 8.07 (d, J = 0.7 Hz, 1H), 7.79 (d, J = 2.2 Hz, 1H), 7.67 (d, J = 8.8 Hz, 1H), 7.41 (s, 1H), 7.33 (d, J = 0.7 Hz, 1H), 6.87 (s, 2H), 6.78 (d, J = 2.3 Hz, 1H), 6.51 (d, J = 2.4 Hz, 1H), 6.39 (dd, J = 8.8, 2.5 Hz, 1H), 5.25 (s, 2H), 3.92 (s, 3H). | B |
| 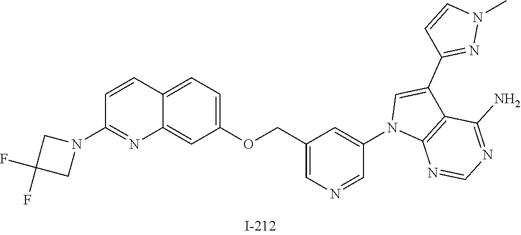 [SMILES: Cn1ccc(-c2cn(-c3cncc(COc4ccc5ccc(N6CC(F)(F)C6)nc5c4)c3)c3ncnc(N)c23)n1] | 540.0 | ¹H NMR (400 MHz, DMSO-d6) δ ppm 9.18 (s, 1H), 9.13 (d, J = 2.4 Hz, 1H), 8.70 (d, J = 1.7 Hz, 1H), 8.51 (t, J = 2.1 Hz, 1H), 8.26 (s, 1H), 8.15 (s, 1H), 8.07 (d, J = 8.8 Hz, 1H), 7.79 (d, J = 2.2 Hz, 1H), 7.72 (d, J = 8.9 Hz, 1H), 7.41 (s, 1H), 7.24 (d, J = 2.4 Hz, 1H), 7.07 (dd, J = 8.8, 2.5 Hz, 1H), 6.78 (d, J = 2.3 Hz, 1H), 6.72 (d, J = 8.8 Hz, 1H), 5.41 (s, 2H), 4.51 (t, J = 12.5 Hz, 4H), 3.92 (s, 3H). | B |
| 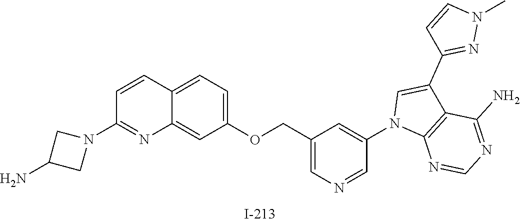 [SMILES: Cn1ccc(-c2cn(-c3cncc(COc4ccc5ccc(N6CC(N)C6)nc5c4)c3)c3ncnc(N)c23)n1] | 519.2 | ¹H NMR (400 MHz, DMSO-d6) δ ppm 9.18 (br, 1H), 9.12 (d, J = 2.4 Hz, 1H), 8.69 (d, J = 1.6 Hz, 1H), 8.50-8.49 (m, 1H), 8.26 (d, J = 2.8 Hz, 1H), 8.15 (s, 1H), 7.91 (d, J = 8.8 Hz, 1H), 7.79 (d, J = 2.2 Hz, 1H), 7.61 (d, J = 8.8 Hz, 1H), 7.42 (br, 1H), 7.16 (d, J = 2.4 Hz, 1H), 6.95 (dd, J = 8.8, 2.5 Hz, 1H), 6.78 (d, J = 2.3 Hz, 1H), 6.54 (d, J = 8.8 Hz, 1H), 5.38 (s, 2H), 4.23 (t, J = 7.8 Hz, 2H), 3.92 (s, 3H), 3.88-3.80 (m, 1H), 3.68-3.65 (m, 2H), 3.34-3.31 (m, 2H). | B |
| 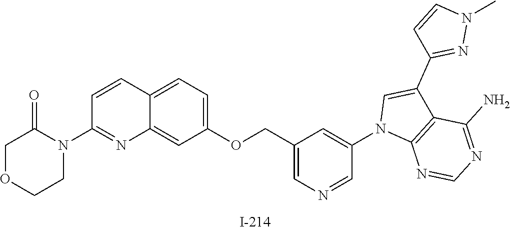 [SMILES: Cn1ccc(-c2cn(-c3cncc(COc4ccc5ccc(N6CCOCC6=O)nc5c4)c3)c3ncnc(N)c23)n1] | 548.0 | ¹H NMR (400 MHz, DMSO-d6) δ ppm 9.19 (brs, 1H), 9.14 (d, J = 2.4 Hz, 1H), 8.73 (d, J = 1.6 Hz, 1H), 8.54 (t, J = 2.0 Hz, 1H), 8.29 (d, J = 8.8 Hz, 1H), 8.27 (s, 1H), 8.15 (s, 1H), 8.01 (d, J = 9.2 Hz, 1H), 7.91 (d, J = 9.2 Hz, 1H), 7.79 (d, J = 2.4 Hz, 1H), 7.47 (d, J = 2.4 Hz, 1H), 7.42 (brs, 1H), 7.34 (dd, J = 8.8, 2.4 Hz, 1H), 6.78 (d, J = 2.4 Hz, 1H), 5.46 (s, 2H), 4.31 (s, 2H), 4.13-4.10 (m, 2H), 4.05-4.03 (m, 2H), 3.92 (s, 3H). | B |
| 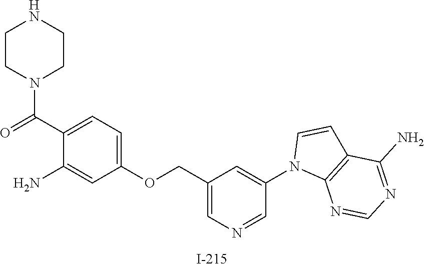 [SMILES: Nc1cc(OCc2cncc(-n3ccc4c(N)ncnc43)c2)ccc1C(=O)N1CCNCC1] | 445.1 | ¹H NMR (400 MHz, DMSO-d6) δ ppm 9.06 (d, J = 2.4 Hz, 1H), 8.62 (d, J = 1.3 Hz, 1H), 8.41 (s, 1H), 8.16 (s, 1H), 7.70 (d, J = 3.7 Hz, 1H), 7.26 (s, 2H), 6.94 (d, J = 8.4 Hz, 1H), 6.84 (d, J = 3.7 Hz, 1H), 6.38 (d, J = 2.3 Hz, 1H), 6.28 (dd, J = 8.4, 2.2 Hz, 1H), 5.32 (s, 2H), 5.18 (s, 2H), 3.43-3.37 (m, 4H), 2.71-2.60 (m, 4H). | B |
| 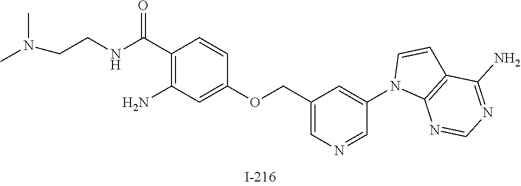 [SMILES: CN(C)CCNC(=O)c1ccc(OCc2cncc(-n3ccc4c(N)ncnc43)c2)cc1N] | 447.3 | ¹H NMR (400 MHz, DMSO-d6) δ ppm 9.06 (d, J = 2.4 Hz, 1H), 8.61 (d, J = 1.6 Hz, 1H), 8.41-8.40 (m, 1H), 8.16 (d, J = 4.2 Hz, 2H), 8.02-7.99 (m, 1H), 7.69 (d, J = 3.6 Hz, 1H), 7.45 (d, J = 8.8 Hz, 1H), 7.24 (s, 2H), 6.85 (d, J = 3.6 Hz, 1H), 6.61-6.57 (m, 1H), 6.34 (d, J = 2.4 Hz, 1H), 6.24 (dd, J = 6.4 Hz 8.8 Hz, 1H), 5.21 (s, 2H), 3.34-3.30 (m, 2H), 2.55-2.53 (m, 2H), 2.30 (s, 6H). | B |
| 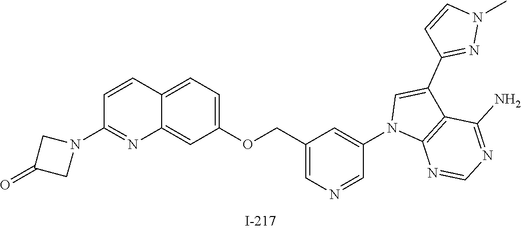 [SMILES: Cn1ccc(-c2cn(-c3cncc(COc4ccc5ccc(N6CC(=O)C6)nc5c4)c3)c3ncnc(N)c23)n1] | 518.1 | ¹H NMR (400 MHz, DMSO-d6) δ ppm 9.18 (s, 1H), 9.12 (d, J = 2.4 Hz, 1H), 8.70 (d, J = 2.4 Hz, 1H), 8.51 (s, 1H), 8.26 (s, 1H), 8.14 (s, 1H), 8.02 (s, J = 9.6 Hz, 1H), 7.79 (d, J = 2.4 Hz 1H), 7.72 (d, J = 9.2 Hz, 1H), 7.39 (s, 1H), 7.25 (d, J = 3.2 Hz, 1H), 7.06 (m, 1H), 6.80 (s, 1H), 6.78 (s, 1H), 6.79 (d, J = 11.2 Hz, 1H), 5.42 (s, 1H), 4.93 (s, 4H), 3.91 (s, 3H). | B |
| 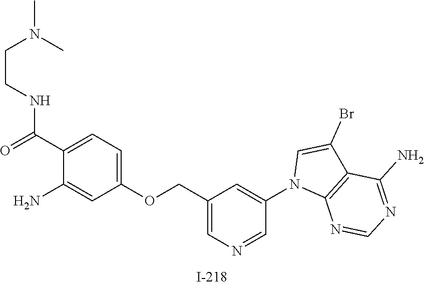 [SMILES: CN(C)CCNC(=O)c1ccc(OCc2cncc(-n3cc(Br)c4c(N)ncnc43)c2)cc1N] | 525.1 | ¹H NMR (400 MHz, MeOD-d4) δ ppm 8.86 (d, J = 2.4 Hz, 1H), 8.56 (s, 1H), 8.24 (s, 2H), 8.09 (s, 1H), 7.64 (s, 1H), 7.39 (d, J = 8.8 Hz, 1H), 7.39 (d, J = 8.8 Hz, 1H), 6.34 (d, J = 2.4 Hz, 1H), 6.27 (d, J = 8.8 Hz, 1H), 5.18 (s, 2H), 3.60-3.57 (m, 1H), 2.87 (s, 6H). | B |
| 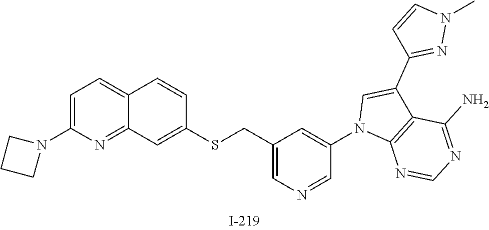 [SMILES: Cn1ccc(-c2cn(-c3cncc(CSc4ccc5ccc(N6CCC6)nc5c4)c3)c3ncnc(N)c23)n1] | 520.6 | ¹H NMR (400 MHz, DMSO-d6) δ ppm 9.24 (br, 1H), 8.98 (d, J = 2.4 Hz, 1H), 8.56 (s, 1H), 8.39 (s, 1H), 8.16 (s, 1H), 8.10 (s, 1H), 8.04-8.01 (m, 1H), 7.79 (d, J = 2 Hz, 1H), 7.69-7.68 (m, 1H), 7.55 (s, 1H), 7.46 (br, 1H), 7.32-7.28 (s, 1H), 6.74 (d, J = 2 Hz, 1H), 6.69-6.68 (m, 1H), 4.51 (s, 2H), 4.15 (br, 4H), 3.91 (s, 3H), 2.39-2.33 (m, 2H) | G |
| 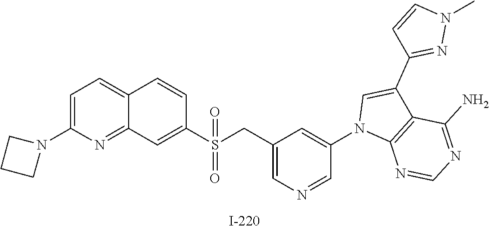 [SMILES: Cn1ccc(-c2cn(-c3cncc(CS(=O)(=O)c4ccc5ccc(N6CCC6)nc5c4)c3)c3ncnc(N)c23)n1] | 551.4 | ¹H NMR (400 MHz, DMSO-d6) δ ppm 9.14 (br, 1H), 9.05 (d, J = 2.4 Hz, 1H), 8.33 (d, J = 2 Hz, 1H), 8.12-8.11 (m, 1H), 8.06 (d, J = 8 Hz, 1H), 8.00 (s, 1H), 7.97 (s, 1H), 7.91 (d, J = 8.4 Hz, 1H), 7.85 (s, 1H), 7.79 (d, J = 2.4 Hz, 1H), 7.46 (dd, J = 8.0 Hz 1.6 Hz, 1H), 7.38 (br, 1H), 6.81 (d, J = 9.2 Hz, 1H), 6.69 (d, J = 2.4 Hz, 1H), 4.94 (s, 2H), 4.07 (t, J = 7.6 Hz, 4H), 3.92 (s, 3H), 2.34-2.30 (m, 2H) | G |
| 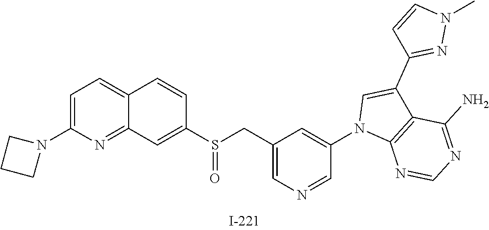 [SMILES: Cn1ccc(-c2cn(-c3cncc(CS(=O)c4ccc5ccc(N6CCC6)nc5c4)c3)c3ncnc(N)c23)n1] | 535.8 | ¹H NMR (400 MHz, DMSO-d6) δ ppm 9.05-8.99 (m, 1H), 8.19-8.16 (m, 4H), 8.20-8.02 (m, 2H), 7.88-7.85 (m, 3H), 7.68-7.67 (m, 1H), 7.56-7.52 (m, 1H), 6.81-6.80 (m, 1H), 6.75-6.74 (m, 1H), 4.93 (s, 2H), 4.25-4.15 (m, 2H), 3.96 (s, 2H), 3.95 (s, 3H), 2.34-2.32 (m, 2H) | G |
| 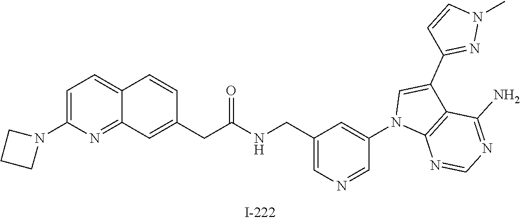 [SMILES: Cn1ccc(-c2cn(-c3cncc(CNC(=O)Cc4ccc5ccc(N6CCC6)nc5c4)c3)c3ncnc(N)c23)n1] | 495.0 | ¹H NMR (400 MHz, DMSO-d6) δ ppm 9.16 (br, 1H), 8.95 (s, 1H), 8.69-8.65 (m, 1H), 8.50 (s, 1H), 8.18 (t, J = 2.0 Hz, 1H), 8.14 (s, 1H), 8.12 (s, 1H), 7.78 (d, J = 2.4 Hz, 1H), 7.39-7.35 (m, 2H), 6.75 (d, J = 2.4 Hz, 1H), 6.55 (d, J = 6.8 Hz, 1H), 6.12 (d, J = 8.0 Hz, 1H), 4.43 (d, J = 6.0 Hz, 2H), 3.92 (s, 3H), 3.79-3.75 (m, 4H), 3.49 (s, 2H), 2.17-2.13 (m, 2H) | F |
| 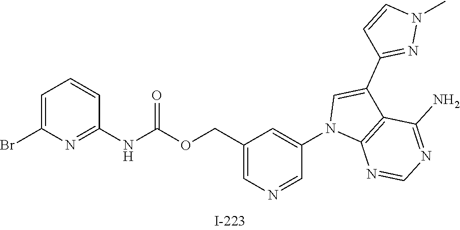 [SMILES: Cn1ccc(-c2cn(-c3cncc(COC(=O)Nc4cccc(Br)n4)c3)c3ncnc(N)c23)n1] | 520.1 | ¹H NMR (400 MHz, DMSO-d6) δ ppm 10.73 (s, 1H), 9.19-9.10 (m, 2H), 8.63 (s, 1H), 8.43 (s, 1H), 8.21 (s, 1H), 8.13 (s, 1H), 7.89-7.85 (m, 1H), 7.78 (s, 1H), 7.74-7.70 (m, 1H), 7.39 (brs, 1H), 7.29 (d, J = 8 Hz, 1H), 6.76 (s, 1H), 5.34 (s, 2H), 3.91 (s, 3H). | F |
| 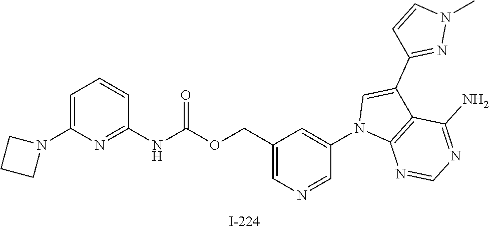 [SMILES: Cn1ccc(-c2cn(-c3cncc(COC(=O)Nc4cccc(N5CCC5)n4)c3)c3ncnc(N)c23)n1] | 497.2 | ¹H NMR (400 MHz, DMSO-d6) δ ppm 9.86 (s, 1H), 9.17 (s, 1H), 9.09 (s, 1H), 8.64 (s, 1H), 8.42 (s, 1H), 8.21 (s, 1H), 8.14 (s, 1H), 7.79 (s, 1H), 7.49-7.39 (m, 2H), 7.08 (d, J = 7.6 Hz, 1H), 6.77 (s, 1H), 6.01 (d, J = 8 Hz 1H), 5.30 (s, 2H), 3.91 (s, 3H), 3.89-3.87 (m, 4H), 2.34-2.24 (m, 2H). | F |
| 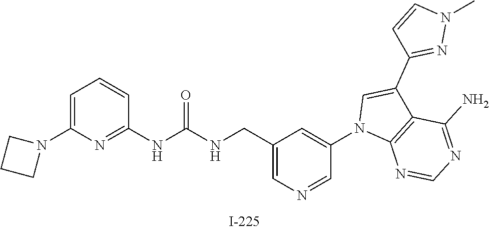 [SMILES: Cn1ccc(-c2cn(-c3cncc(CNC(=O)Nc4cccc(N5CCC5)n4)c3)c3ncnc(N)c23)n1] | 496.2 | ¹H NMR (400 MHz, DMSO-d6) δ ppm 9.19 (s, 3H), 9.01 (s, 1H), 8.54 (s, 1H), 8.34 (s, 1H), 8.12 (s, 1H), 8.10 (s, 1H), 7.78 (s, 1H), 7.39-7.34 (m, 2H), 6.76 (s, 1H), 6.39 (d, J = 8 Hz, 1H), 5.83 (d, J = 8 Hz, 1H), 4.53 (s, 2H), 3.91 (s, 3H), 3.78-3.74 (m, 4H), 2.17-2.11 (m, 2H). | F |
| 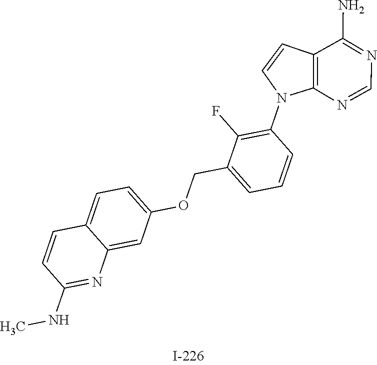 [SMILES: CNc1ccc2ccc(OCc3cccc(-n4ccc5c(N)ncnc54)c3F)cc2n1] | 415.2 | ¹H NMR (400 MHz, DMSO-d6) δ ppm 2.94 (d, J = 4.4 Hz, 3H), 5.34 (s, 2H), 6.68 (d, J = 8.8 Hz, 1H), 6.81 (d, J = 3.4 Hz, 1H), 6.96 (d, J = 8.1 Hz, 1H), 7.17-7.28 (m, 3H), 7.36-7.44 (m, 2H), 7.54-7.74 (m, 4H), 7.87 (d, J = 8.3 Hz, 1H), 8.07 (s, 1H). | B |
| 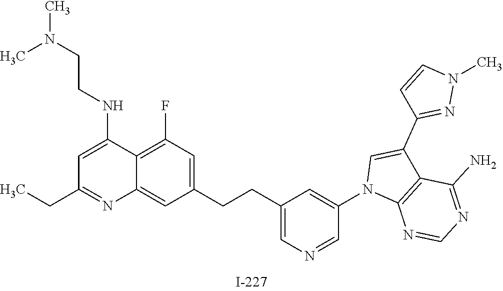 | 579.3 | ¹H NMR (400 MHz, CDCl₃) δ ppm 1.34 (t, J = 7.6 Hz, 3H), 2.30 (s, 6H), 2.64 (t, J = 6.0 Hz, 2H), 2.80 (q, J = 7.6 Hz, 2H), 3.06-3.18 (m, 4H), 3.28 (q, J = 5.4 Hz, 2H), 3.96 (s, 3H), 6.21 (s, 1H), 6.48 (d, J = 2.4 Hz, 1H), 6.64-6.74 (m, 1H), 6.80 (dd, J = 14.8, 1.1 Hz, 1H), 7.38 (d, J = 2.2 Hz, 1H), 7.43 (s, 1H), 7.53 (s, 1H), 8.00 (t, J = 2.0 Hz, 1H), 8.28 (s, 1H), 8.40 (d, J = 1.7 Hz, 1H), 8.77 (d, J = 2.4 Hz, 1H). | C |
| 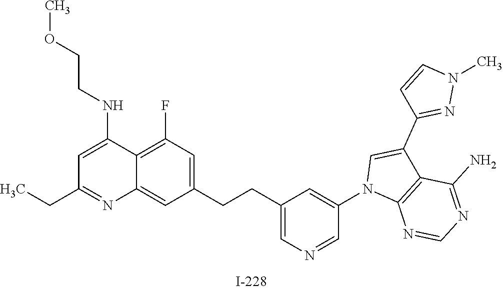 [SMILES: CCc1cc(NCCOC)c2c(F)cc(CCc3cncc(-n4cc(-c5ccn(C)n5)c5c(N)ncnc54)c3)cc2n1] | 566.3 | ¹H NMR (400 MHz, CDCl₃) δ ppm (t, J = 7.7 Hz, 3H), 2.80 (q, J = 7.6 Hz, 2H), 3.07-3.17 (m, 4H), 3.44 (s, 5H), 3.70 (t, J = 5.3 Hz, 2H), 3.96 (s, 3H), 6.24 (s, 1H), 6.38 (dt, J = 17.7, 4.6 Hz, 1H), 6.49 (d, J = 2.2 Hz, 1H), 6.82 (dd, J = 14.8, 1.1 Hz, 1H), 7.38 (d, J = 2.2 Hz, 1H), 7.44 (s, 1H), 7.54 (s, 1H), 8.01 (t, J = 2.2 Hz, 1H), 8.28 (s, 1H), 8.40 (d, J = 1.5 Hz, 1H), 8.77 (d, J = 2.2 Hz, 1H). | C |
| 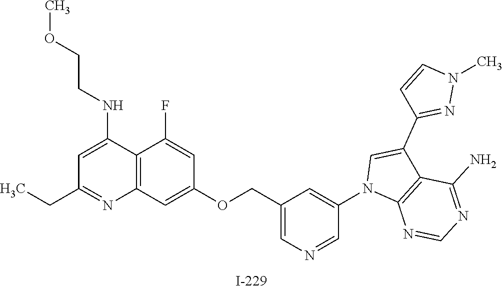 [SMILES: CCc1cc(NCCOC)c2c(F)cc(OCc3cncc(-n4cc(-c5ccn(C)n5)c5c(N)ncnc54)c3)cc2n1] | 568.3 | ¹H NMR (400 MHz, CDCl₃) δ ppm 1.35 (t, J = 7.6 Hz, 3H), 2.81 (q, J = 7.6 Hz, 2H), 3.40-3.49 (m, 5H), 3.67-3.75 (m, 2H), 3.98 (s, 3H), 5.29 (s, 2H), 6.22 (s, 1H), 6.36 (br d, J = 16.6 Hz, 1H), 6.54 (d, J = 2.2 Hz, 1H), 6.74 (dd, J = 15.4, 2.2 Hz, 1H), 7.22 (br s, 1H), 7.40 (d, J = 2.0 Hz, 1H), 7.54 (s, 1H), 8.32 (s, 2H), 8.69 (s, 1H), 8.96 (d, J = 2.0 Hz, 1H). | B |
| 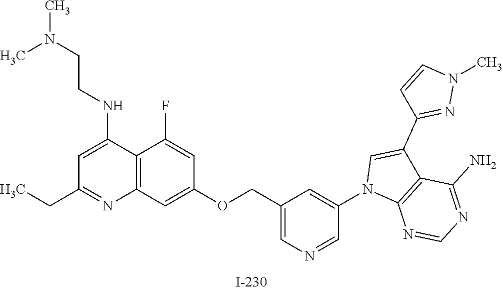 | 581.3 | ¹H NMR (400 MHz, CDCl₃) δ ppm 1.36 (t, J = 7.6 Hz, 3H), 2.31 (s, 6H), 2.65 (br t, J = 5.9 Hz, 2H), 2.80 (q, J = 7.6 Hz, 2H), 3.24-3.36 (m, 2H), 3.98 (s, 3H), 5.29 (s, 2H), 6.18 (s, 1H), 6.54 (d, J = 1.7 Hz, 1H), 6.63-6.80 (m, 2H), 7.19 (br s, 1H), 7.40 (d, J = 1.7 Hz, 1H), 7.53 (s, 1H), 8.33 (s, 2H), 8.69 (s, 1H), 8.96 (d, J = 1.7 Hz, 1H). | B |
| 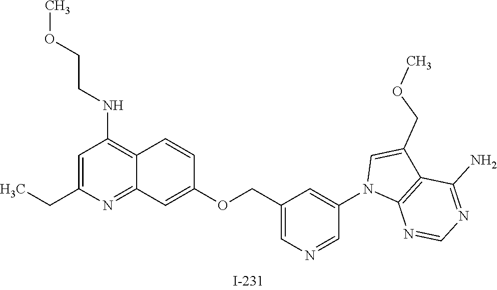 [SMILES: CCc1cc(NCCOC)c2ccc(OCc3cncc(-n4cc(COC)c5c(N)ncnc54)c3)cc2n1] | 514.3 | ¹H NMR (400 MHz, DMSO-d6) δ ppm 1.25 (t, J = 7.6 Hz, 3H), 2.69 (q, J = 7.6 Hz, 2H), 3.29 (s, 3H), 3.35 (s, 3H), 3.44 (q, J = 5.6 Hz, 2H), 3.58 (t, J = 6.1 Hz, 2H), 4.64 (s, 2H), 5.38 (s, 2H), 6.30 (s, 1H), 6.87 (br s, 2H), 6.97 (m, 1H), 7.09 (dd, J = 9.2, 2.6 Hz, 1H), 7.27 (d, J = 2.7 Hz, 1H), 7.76 (s, 1H), 8.09 (d, J = 9.3 Hz, 1H), 8.18 (s, 1H), 8.44 (t, J = 2.2 Hz, 1H), 8.66-8.71 (m, 1H), 9.06 (d, J = 2.4 Hz, 1H). | B |
| 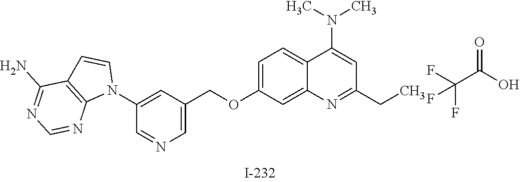 | 440.3 | 1H NMR (400 MHz, DMSO-d6) δ 13.46 (s, 1H), 9.07 (d, J = 2.5 Hz, 1H), 8.78 (d, J = 1.8 Hz, 1H), 8.45 (t, J = 2.3 Hz, 1H), 8.34-8.27 (m, 2H), 8.25-8.14 (m, 1H), 7.83 (d, J = 3.7 Hz, 1H), 7.42 (d, J = 2.7 Hz, 1H), 7.31 (dd, J = 9.5, 2.6 Hz, 1H), 7.01 (d, J = 3.7 Hz, 1H), 6.78 (s, 1H), 5.48 (s, 2H), 3.42 (s, 6H), 2.88 (q, J = 7.6 Hz, 2H), 1.34 (t, J = 7.6 Hz, 3H). | B |
| 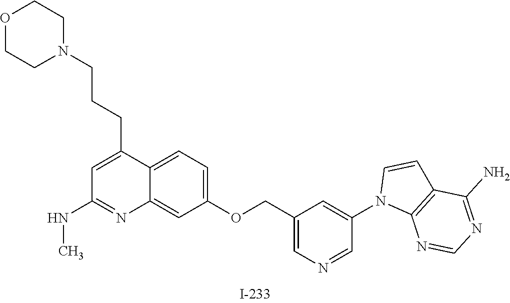 [SMILES: CNc1cc(CCCN2CCOCC2)c2ccc(OCc3cncc(-n4ccc5c(N)ncnc54)c3)cc2n1] | 525.3 | ¹H NMR (400 MHz, DMSO-d6) δ 13.46 (s, 1H), 9.07 (d, J = 2.5 Hz, 1H), 8.78 (d, J = 1.8 Hz, 1H), 8.45 (t, J = 2.3 Hz, 1H), 8.34-8.27 (m, 2H), 8.25-8.14 (m, 1H), 7.83 (d, J = 3.7 Hz, 1H), 7.42 (d, J = 2.7 Hz, 1H), 7.31 (dd, J = 9.5, 2.6 Hz, 1H), 7.01 (d, J = 3.7 Hz, 1H), 6.78 (s, 1H), 5.48 (s, 2H), 3.42 (s, 6H), 2.88 (q, J = 7.6 Hz, 2H), 1.34 (t, J = 7.6 Hz, 3H). | B |
| 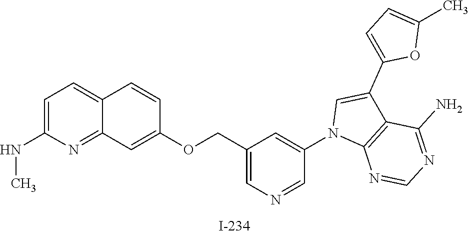 [SMILES: CNc1ccc2ccc(OCc3cncc(-n4cc(-c5ccc(C)o5)c5c(N)ncnc54)c3)cc2n1] | 478.3 | ¹H NMR (400 MHz, DMSO-d6) δ 9.10 (d, J = 2.4 Hz, 1H), 8.71 (s, 1H), 8.48 (s, 1H), 8.21 (s, 1H), 8.12 (s, 1H), 7.88 (br, 1H), 7.63 (d, J = 7.2 Hz, 1H), 7.24 (br, 1H), 7.01 (m, 3H), 6.68 (d, J = 3.2 Hz, 2H), 6.26 (d, J = 2.5 Hz, 1H), 5.39 (s, 2H), 2.94 (d, J = 3.7 Hz, 3H), 2.39 (s, 3H). | B |
| 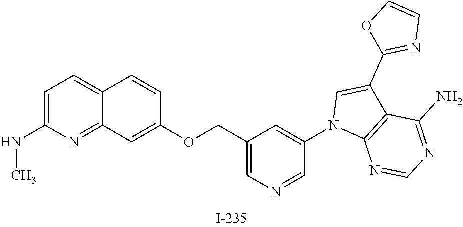 [SMILES: CNc1ccc2ccc(OCc3cncc(-n4cc(-c5ncco5)c5c(N)ncnc54)c3)cc2n1] | 465.3 | ¹H NMR (400 MHz, DMSO-d6) δ 12.52 (br, 1H), 9.61 (br, 2H), 9.11 (d, J = 2.4 Hz, 1H), 8.81 (d, J = 1.7 Hz, 1H), 8.54 (s, 1H), 8.49 (s, 1H), 8.30 (s, H), 8.28 (s, H), 8.24-8.02 (m, 2H), 7.88 (d, J = 8.8 Hz, 1H), 7.58 (br, 1H), 7.48 (s, 1H), 7.26 (dd, J = 8.8, 2.4 Hz, 1H), 6.91 (d, J = 9.1 Hz, 1H), 5.44 (s, 2H), 3.11 (d, J = 4.7 Hz, 3H). | B |
| 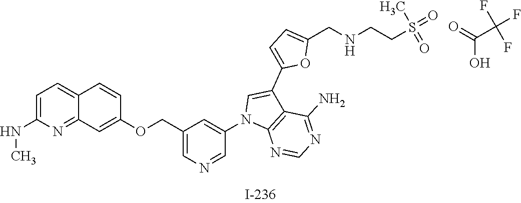 | 599.3 | ¹H NMR (400 MHz, DMSO-d6) δ 12.60 (s, 1H), 9.63 (br, 1H), 9.35 (br, 2H), 9.10 (d, J = 2.4 Hz, 1H), 8.78 (s, 1H), 8.50 (s, 1H), 8.31 (d, J = 1.9 Hz, 2H), 8.20 (d, J = 7.4 Hz, 1H), 7.87 (d, J = 8.8 Hz, 1H), 7.61 (s, 1H), 7.25 (dd, J = 8.8, 2.1 Hz, 1H), 6.99-6.85 (m, 2H), 6.83 (d, J = 3.4 Hz, 1H), 5.43 (s, 2H), 4.45 (s, 2H), 3.56 (m, 2H), 3.44 (m, 2H), 3.14 (s, 3H). 3.11 (d, J = 4.4 Hz, 3H), | B |
| 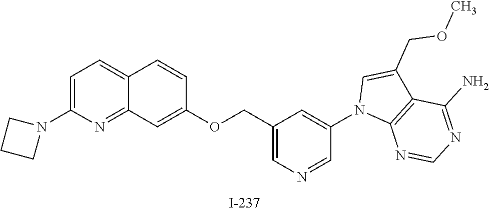 [SMILES: COCc1cn(-c2cncc(COc3ccc4ccc(N5CCC5)nc4c3)c2)c2ncnc(N)c12] | 468.3 | ¹H NMR (400 MHz, DMSO-d6) δ 9.06 (d, J = 2.4 Hz, 1H), 8.69-8.65 (m, 1H), 8.43 (t, J = 2.2 Hz, 1H), 8.18 (s, 1H), 7.93 (d, J = 8.8 Hz, 1H), 7.75 (s, 1H), 7.62 (d, J = 8.8 Hz, 1H), 7.15 (d, J = 2.5 Hz, 1H), 6.96 (dd, J = 8.7, 2.5 Hz, 1H), 6.86 (br, 2H), 6.53 (d, J = 8.8 Hz, 1H), 5.37 (s, 2H), 4.64 (s, 2H), 4.08 (t, J = 7.4 Hz, 4H), 3.35 (s, 3H), 2.38-2.33 (m, 2H). | B |
| 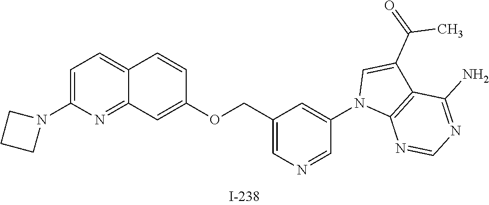 [SMILES: CC(=O)c1cn(-c2cncc(COc3ccc4ccc(N5CCC5)nc4c3)c2)c2ncnc(N)c12] | 466.3 | ¹H NMR (400 MHz, DMSO-d6) δ 9.07 (d, J = 2.4 Hz, 1H), 8.81 (s, H), 8.78 (s, H), 8.45 (t, J = 2.1 Hz, 1H), 8.34 (s, 1H), 8.20 (s, 1H), 7.92 (d, J = 8.9 Hz, 1H), 7.61 (m, 2H), 7.15 (d, J = 2.5 Hz, 1H), 6.95 (dd, J = 8.8, 2.5 Hz, 1H), 6.53 (d, J = 8.8 Hz, 1H), 5.39 (s, 2H), 4.07 (t, J = 7.4 Hz, 4H), 2.59 (s, 3H), 2.39-2.31 (m, 2H). | B |
| 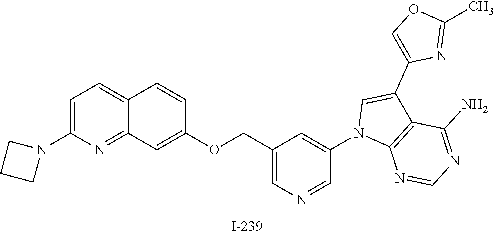 [SMILES: Cc1nc(-c2cn(-c3cncc(COc4ccc5ccc(N6CCC6)nc5c4)c3)c3ncnc(N)c23)co1] | 505.3 | ¹H NMR (400 MHz, DMSO-d6) δ 9.06 (d, J = 2.1 Hz, 1H), 8.81 (s, 1H), 8.50 (s, 1H), 8.43 (m, 2H), 8.39 (s, 1H), 8.30 (d, J = 9.4 Hz, 1H), 7.88 (d, J = 8.9 Hz, 1H), 7.48 (d, J = 2.3 Hz, 1H), 7.23 (dd, J = 8.9, 2.4 Hz, 1H), 6.80 (d, J = 9.3 Hz, 1H), 5.43 (s, 2H), 4.42 (t, J = 7.7 Hz, 4H), 2.58 (s, 3H), 2.46 (d, J = 7.4 Hz, 2H). | B |
| 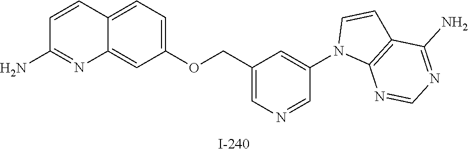 [SMILES: Nc1ccc2ccc(OCc3cncc(-n4ccc5c(N)ncnc54)c3)cc2n1] | 384.1 | ¹H NMR (400 MHz, DMSO-d₆) δ ppm 9.07 (d, J = 2.5 Hz, 1H), 8.67 (d, J = 1.8 Hz, 1H), 8.46-8.45 (m, 1H), 8.15 (s, 1H), 7.79 (d, J = 8.8 Hz, 1H), 7.69 (d, J = 3.7 Hz, 1H), 7.54 (d, J = 8.8 Hz, 1H), 7.23 (s, 2H), 7.00 (d, J = 2.5 Hz, 1H), 6.90 (dd, J = 8.7, 2.5 Hz, 1H), 6.84 (d, J = 3.7 Hz, 1H), 6.58 (d, J = 8.8 Hz, 1H), 6.34 (s, 2H), 5.34 (s, 2H). | B |

### Biological Assays

METTL3-14 Standard Enzyme Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, Mass.). For compound screening METTL3/METTL14 (final concentration, f.c.=2.5 nM) was added using a Multidrop Combi (ThermoFisher Scientific, Waltham, Mass.) and preincubated for 5 min. Reactions were started by adding 3′ biotinylated RNA (UCUGGACUAAA-biotin) (f.c.=100 nM) and 3H-SAM (f.c.=100 nM) substrates. Reactions proceeded for 30 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

METTL1 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 250 μM MgCl2, 1 mM DTT, 0.01% Tween, 0.01% BSG, 0.004 U/μl RNAseOUT (cat. No. 10777019, ThermoFisher Scientific, Waltham, Mass.). For compound screening METTL1/WDR4 (final concentration, f.c.=6.25 nM) was added using a Multidrop Combi (ThermoFisher Scientific, Waltham, Mass.) and preincubated for 5 min. Reactions were started by adding 3′ biotinylated RNA (GCCGAGAUAGCUCAGUUGGGAGAGCGUUAGACUGAAGAUCUAAAGGUCCCUG GUUCAAUCCCGGGUUUCGGCA-biotin) (f.c.=25 nM) and 3H-SAM (f.c.=60 nM) substrates. Reactions proceeded for 20 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

METTL16 Assay

Assays were performed in a 25 μl-volume in 384-well V-bottom polypropylene microplates (Greiner Bio-One, cat. No. 781280) at ambient temperature. Optimized 1× assay buffer was 20 mM HEPES pH 7.5, 50 mM KCl, 1 mM DTT, 0.01% Tween, 0.01% BSG, 0.004 U/μl RNAseOUT (cat. No. 10777019, ThermoFisher Scientific, Waltham, Mass.). For compound screening METTL16 (final concentration, f.c.=100 nM) was added using a Multidrop Combi (ThermoFisher Scientific, Waltham, Mass.) and preincubated for 5 min. Reactions were started by adding 3′ biotinylated RNA (CGAUACAGAGAAGAUUAGCAUACGCAAAUUCGUGAAGCG-biotin) (f.c.=50 nM), 3H-SAM (f.c.=200 nM) and non-radiolabeled SAM (f.c.=800 nM) substrates. Reactions proceeded for 20 minutes and were quenched with excess non-radioactive SAM (f.c.=100 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

PRMT5 Assay

Assays were performed in a 25 μl-volume in 384-well V-bottom polypropylene microplates (Greiner Bio-One, cat. No. 781280) at ambient temperature. Optimized 1× assay buffer was 20 mM Tris-HCl pH 8.0, 1 mM DTT, 0.01% Tween, 0.01%. For compound screening PRMT5-MEP50 (final concentration, f.c.=2.5 nM) was added using a Multidrop Combi (ThermoFisher Scientific, Waltham, Mass.) and preincubated for 5 min. Reactions were started by adding 3′ biotinylated histone H4 peptide acetylated on serine 1 (Ac-SGRGKGGKGLGKGGAKRHRKVGGK-Biotin) (f.c.=100 nM) and 3H-SAM (f.c.=250 nM) substrates. Reactions proceeded for 60 minutes and were quenched with excess non-radioactive SAM (f.c.=15 μM). The reactions were then transferred to streptavidin-coated flashplates and incubated for 2 hours at 25° C. Following two washing cycles with 0.1% Tween-20, the plates were sealed and read on a TopCount (PerkinElmer, Waltham, Mass.) plate-based scintillation counter. For determination of kinetic parameters, reaction times were optimized so that measurements were taken during the initial velocity phase of the reaction.

m⁶ A-mRNA LC-MS/MS Assay

5×10⁶ MOLM-13 (DSMZ) cells were seeded into 10 cm dishes in RPMI 1640 media containing 10% fetal bovine serum and placed in a humidified tissue culture incubator at 37° C. overnight. Compounds were resuspended in 100% DMSO and dosed into each dish at a fixed concentration to comprise an 8-point dose response with a 4-fold serial dilution ranging from 25 μM to 1.5 nM in 0.25% DMSO final and allowed to incubate for 24 hours in a humidified tissue culture incubator at 37° C. Cells were harvested by centrifugation followed by mRNA extraction using DTRECT Dynabeads mRNA DTRECT kit (Life Technologies). mRNA was quantified on NanoDrop spectrophotometer (Thermo Fisher Scientific) and digested into single nucleosides using Nucleoside Digestion Mix (New England Biolabs). Nucleosides are quantified with retention time on a BEH Cis column (Waters) and the nucleoside-to-base ion mass transition of 282.1-150.1 (m⁶ A) and 268-136 (A) on an API 6500+ triple quadrupole mass spectrometer. Quantification is performed in comparison with the standard curve, obtained from pure nucleoside standards (Selleck Chemicals) running with the same batch of samples. Percentage m⁶ A in cellular mRNA is calculated as 100*(m⁶ A/A).

MOLM-13 48 Hour Cell Proliferation Assay

MOLM-13 (DSMZ) cells were seeded at 1000 cells per well in a volume of 44 μL in a Falcon 384-well tissue culture treated clear bottom microplate in RPMI 1640 media containing 10% fetal bovine serum using a Multidrop Combi (ThermoFisher Scientific). Cells were incubated overnight at 37° C. in a humidified tissue culture incubator. The Mosquito® HTS Liquid Handler was used to make a compound/media intermediate plate by aliquoting 1 L of compound from the initial compound dilution plate (concentrations ranging from 10.0 mM to 38.0 nM in 100% DMSO) into a V bottom 384-well screen matrix plate containing 49 L of media containing the appropriate serum (50-fold dilution, 2% DMSO). The Apricot liquid handling system was used to transfer 6.2 μL compounds from the intermediate plate into the Falcon 384-well tissue culture plate containing 44 μL cells (10-point, 4-fold dilution spanning concentrations 25.0 μM to 95.1 μM, 0.25% DMSO final), and placed in a humidified tissue culture incubator at 37° C. After 48 hours, 25 μL of Cell Titer-Glo reagent (Promega) was added to each well using a Multidrop Combi. The plate was protected from light and placed on an IKA plate shaker for at 300 rpm for 10 minutes at room temperature. The plate was read on an EnVision plate reader (Perkin Elmer) using the Ultra Sensitive Luminescence protocol. Data analysis was performed by normalizing the raw luminescence units to an average of the positive control values for staurosporine (100% cell death) and the negative control values for DMSO (0% cell death). An IC₅₀ was calculated using a 4-parameter logistic nonlinear regression model in GraphPad Prism.

MOLM-13 96 Hour Cell Proliferation Assay

MOLM-13 (DSMZ) cells were seeded at 600 cells per well in a volume of 44 μL in a Greiner Bio-One CELLSTAR™ 384 Well Polystyrene Cell Culture clear bottom microplate in RPMI 1640 media containing 10% fetal bovine serum using a Multidrop Combi (ThermoFisher Scientific). Cells were incubated overnight at 37° C. in a humidified tissue culture incubator. To prevent evaporation or to reduce edge effect, add 50 μL or more H2O to an empty plate, cover, and place on top of the cell plates. The Mosquito® HTS Liquid Handler was used to make a compound/media intermediate plate by aliquoting 1 μL of compound from the initial compound dilution plate (concentrations ranging from 10.0 mM to 38.0 nM in 100% DMSO) into Bio-One 384-well polypropylene conical bottom microplate containing 49 μL of media containing the appropriate serum (50-fold dilution, 2% DMSO, compound concentrations ranging from 200.0 μM and 760.8 μM). The Apricot liquid handling system was used to transfer 6.2 μL compounds from the intermediate plate into the seeding plate containing 44 μL cells (8-fold dilution spanning concentrations 25.0 μM to 95.1 μM, 0.25% DMSO final), and placed in a humidified tissue culture incubator at 37° C. After 96 hours, 25 μL of Cell Titer-Glo reagent (Promega) was added to each well using an Integra liquid handling system. The plate was protected from light using TopSeal-A (Black) film over the entire plate, added White Bottom seal film on the bottom of the entire plate for a better reading on the Envision, and placed on an IKA plate shaker for at 300 rpm for 10 minutes at room temperature. The plate was read on an EnVision plate reader (Perkin Elmer) using the Ultra Sensitive Luminescence protocol. Data analysis was performed by normalizing the raw luminescence units to an average of the positive control values for staurosporine (100% cell death) and the negative control values for DMSO (0% cell death). An IC₅₀ was calculated using a 4-parameter logistic nonlinear regression model in GraphPad Prism.

Table 2 shows IC₅₀ values for selected compounds of this invention measured in the METTL3 biochemical assay, PRMT5 biochemical assay, METTL1 biochemical assay, METTL16 biochemical assay, m⁶ A cellular assay and MOLM-13 cell proliferation assay, wherein each compound number corresponds to the compound numbering set forth in Examples 1-240 of Table 1 disclosed above. For METTL3, PRMT5, METTL1 and METTL16 biochemical assays, “A” represents an IC₅₀ of less than 10 nM (i.e., IC₅₀<10 nM); “B” represents an IC₅₀ of equal to or greater than 10 nM and lesser than 100 nM (i.e., 10 nM≤IC₅₀<100 nM); “C” represents an IC₅₀ of equal to or greater than 100 nM and less than 1000 nM (i.e., 100 nM: IC₅₀≤1000 nM); and “D” represents an IC₅₀ of equal to or greater than 1000 nM (i.e., IC₅₀≥1000 nM). For m⁶ A cellular assay and MOLM-13 48 Hour and 96 Hour cell proliferation assay, “*” represents an IC₅ of 4 equal to or greater than 10 μM (i.e., IC₅₀≥10 μM); “**” represents an IC₅₀ value of equal to or greater than 1 μM and less than 10 μM (i.e., 1 μM≤IC₅₀<10 μM); and “***” represents an IC₅₀ of less than 1 μM (i.e., IC₅₀<1 μM).

TABLE 2

| Com- | | | | | | MOLM-13 | MOLM-13 |
|---|---|---|---|---|---|---|---|
| pound | | | | | | 48 Hour cell | 96 Hour cell |
| No. | METTL3 | PRMT5 | METTL1 | METTL16 | m⁶A | proliferation | proliferation |
| I-1 | A | D | D | | *** | *** | |
| I-2 | | D | D | | | | |
| I-3 | B | D | D | | | | |
| I-4 | | D | D | | | | |
| I-5 | | D | D | | | | |
| I-6 | A | D | D | | | * | ** |
| I-7 | A | D | D | | | * | |
| I-8 | A | D | D | | | | |
| I-9 | B | D | D | | ** | ** | |
| I-10 | A | D | D | | * | * | |
| I-11 | A | D | D | | ** | *** | |
| I-12 | A | D | D | | | ** | ** |
| I-13 | B | D | D | | | | |
| I-14 | A | D | D | | ** | ** | |
| I-15 | A | D | D | | | ** | |
| I-16 | A | D | D | | ** | ** | |
| I-17 | A | D | D | | * | *** | |
| I-18 | A | D | D | | ** | ** | |
| I-19 | B | D | D | | | | |
| I-20 | A | D | D | | ** | | |
| I-21 | C | D | D | | | | |
| I-22 | B | D | D | | | | |
| I-23 | D | D | D | | | | |
| I-24 | D | D | D | | | | |
| I-25 | A | D | | D | * | * | |
| I-26 | B | D | | D | | | |
| I-27 | B | D | | D | | | |
| I-28 | B | D | D | D | | | |
| I-29 | A | D | D | D | ** | *** | |
| I-30 | B | D | D | D | ** | ** | |
| I-31 | D | D | | D | | | |
| I-32 | C | D | D | D | | | |
| I-33 | C | D | D | D | | | |
| I-34 | A | D | D | D | * | *** | |
| I-35 | A | D | D | D | * | *** | |
| I-36 | B | D | D | D | ** | ** | |
| I-37 | B | D | D | | | | |
| I-38 | D | D | D | | | | |
| I-39 | B | D | | D | | | |
| I-40 | B | D | | D | | | |
| I-41 | D | D | | D | | | |
| I-42 | B | D | | D | | *** | |
| I-43 | B | D | | D | | | |
| I-44 | B | D | | D | | | |
| I-45 | A | D | | D | * | * | |
| I-46 | A | D | | D | * | ** | |
| I-47 | A | D | | D | * | * | |
| I-48 | C | D | | D | | | * |
| I-49 | D | D | | D | | | |
| I-50 | D | D | | D | | | |
| I-51 | B | D | | D | | | |
| I-52 | A | D | | D | * | | |
| I-53 | B | D | | D | | | |
| I-54 | C | D | | | | | |
| I-55 | C | D | | D | | | ** |
| I-56 | C | D | | D | | | |
| I-57 | C | D | | D | | | |
| I-58 | A | D | | D | ** | *** | *** |
| I-59 | A | D | | D | ** | ** | |
| I-60 | A | D | | D | ** | *** | |
| I-61 | B | D | D | D | | | ** |
| I-62 | D | B | D | D | | | |
| I-63 | D | | | | | | |
| I-64 | D | D | D | D | | | |
| I-65 | B | D | D | D | | | |
| I-66 | C | D | D | D | | | |
| I-67 | C | D | D | D | | | |
| I-68 | B | D | D | D | | | |
| I-69 | D | D | D | D | | | |
| I-70 | C | D | D | D | * | ** | ** |
| I-71 | B | D | D | D | | | |
| I-72 | C | D | D | D | | | |
| I-73 | B | D | D | D | | | |
| I-74 | A | D | D | D | ** | ** | ** |
| I-75 | C | D | D | D | | | ** |
| I-76 | B | D | D | D | | | |
| I-77 | D | D | D | D | | | |
| I-78 | B | D | D | D | | | |
| I-79 | B | D | D | D | | | |
| I-80 | B | D | D | D | | | |
| I-81 | B | D | D | D | | | |
| I-82 | C | D | | D | | | |
| I-83 | D | D | | D | | | |
| I-84 | B | D | D | D | | | |
| I-85 | C | D | D | D | | | |
| I-86 | B | D | D | D | | | |
| I-87 | B | D | D | D | | ** | ** |
| I-88 | D | D | D | | | | |
| I-89 | A | D | D | | | | |
| I-90 | A | D | D | | ** | | |
| I-91 | B | D | D | | | | |
| I-92 | A | D | D | | ** | | *** |
| I-93 | A | D | D | | | | |
| I-94 | A | D | D | | | | |
| I-95 | A | D | D | | ** | | *** |
| I-96 | B | D | D | | | | |
| I-97 | A | D | D | | | | *** |
| I-98 | B | D | D | | | | |
| I-99 | A | D | D | | | | *** |
| I-100 | D | D | D | | | | |
| I-101 | A | D | | | | | |
| I-102 | B | D | | | | | |
| I-103 | B | D | | | | | |
| I-104 | A | D | | | *** | | *** |
| I-105 | C | D | | | | | |
| I-106 | A | D | | | *** | | *** |
| I-107 | A | D | | | | | *** |
| I-108 | A | D | | | ** | | ** |
| I-109 | A | D | | | | | *** |
| I-110 | A | D | D | D | ** | | |
| I-111 | D | D | D | D | | | * |
| I-112 | A | D | D | D | ** | | *** |
| I-113 | A | D | D | D | *** | | ** |
| I-114 | A | D | D | D | | | ** |
| I-115 | A | D | D | D | *** | | *** |
| I-116 | A | D | D | D | | | ** |
| I-117 | A | D | D | D | *** | | *** |
| I-118 | A | D | D | D | | | ** |
| I-119 | C | D | D | D | | | |
| I-120 | A | D | D | D | | | ** |
| I-121 | A | D | D | D | ** | | *** |
| I-122 | A | D | D | D | | | *** |
| I-123 | D | D | D | D | | | ** |
| I-124 | B | D | D | D | | | ** |
| I-125 | B | D | D | D | | | |
| I-126 | A | D | D | D | ** | | ** |
| I-127 | A | D | D | D | | | * |
| I-128 | A | D | D | D | ** | | ** |
| I-129 | B | | | | | | |
| I-130 | B | | | | | | |
| I-131 | A | | | | | | *** |
| I-132 | A | | | | | | *** |
| I-133 | B | | | | | | |
| I-134 | A | | | | | | *** |
| I-135 | A | | | | ** | | *** |
| I-136 | A | | | | ** | | *** |
| I-137 | A | | | | ** | | *** |
| I-138 | A | | | | ** | | *** |
| I-139 | B | | | | | | |
| I-140 | A | | | | | | |
| I-141 | A | | | | | | *** |
| I-142 | B | | | | | | *** |
| I-143 | A | | | | | | *** |
| I-144 | A | | | | | | *** |
| I-145 | A | | | | *** | | *** |
| I-146 | A | | | | | | *** |
| I-147 | A | | | | * | | *** |
| I-148 | B | | | | | | |
| I-149 | A | | | | | | ** |
| I-150 | A | | | | | | *** |
| I-151 | B | | | | | | |
| I-152 | A | | | | | | *** |
| I-153 | A | | | | *** | | *** |
| I-154 | A | | | | | | *** |
| I-155 | A | | | | ** | | *** |
| I-156 | A | | | | | | ** |
| I-157 | B | | | | | | |
| I-158 | A | | | | | | ** |
| I-159 | B | | | | | | |
| I-160 | B | | | | | | |
| I-161 | A | | | | | | |
| I-162 | A | | | | | | ** |
| I-163 | B | | | | | | |
| I-164 | B | | | | | | |
| I-165 | B | | | | | | |
| I-166 | C | | | | | | |
| I-167 | A | | | | | | *** |
| I-168 | B | | | | | | |
| I-169 | A | D | | | | | *** |
| I-170 | A | D | | | | | *** |
| I-171 | D | D | | | | | |
| I-172 | D | D | | | | | |
| I-173 | C | D | | | | | |
| I-174 | B | D | | | | | |
| I-175 | C | D | | | | | |
| I-176 | C | D | | | | | |
| I-177 | D | D | | | | | |
| I-178 | D | D | | | | | |
| I-179 | D | D | | | | | |
| I-180 | B | D | | | | | |
| I-181 | D | D | | | | | |
| I-182 | A | | | | | | |
| I-183 | D | D | | | | | |
| I-184 | D | D | | | | | |
| I-185 | B | D | | | | | ** |
| I-186 | D | D | | | | | |
| I-187 | C | D | | | | | |
| I-188 | D | D | | | | | |
| I-189 | C | D | | | | | |
| I-190 | A | D | | | | | |
| I-191 | D | D | | | | | |
| I-192 | A | D | | | | | *** |
| I-193 | A | D | | | | | ** |
| I-194 | C | D | | | | | |
| I-195 | D | D | | | | | |
| I-196 | B | C | | | | | |
| I-197 | C | D | | | | | |
| I-198 | D | D | | | | | |
| I-199 | D | D | | | | | |
| I-200 | C | D | | | | | |
| I-201 | C | D | | | | | |
| I-202 | B | D | | | | | |
| I-203 | B | D | | | | | |
| I-204 | C | D | | | | | |
| I-205 | D | D | | | | | |
| I-206 | C | D | | | | | |
| I-207 | D | D | | | | | |
| I-208 | B | D | | | | | |
| I-209 | D | D | | | | | |
| I-210 | C | D | | | | | |
| I-211 | D | D | | | | | |
| I-212 | A | D | | | | | ** |
| I-213 | A | D | | | | | *** |
| I-214 | D | D | | | | | |
| I-215 | D | D | | | | | |
| I-216 | D | D | | | | | |
| I-217 | A | D | | | | | ** |
| I-218 | D | D | | | | | |
| I-219 | A | D | | | | | |
| I-220 | D | D | | | | | |
| I-221 | D | C | | | | | |
| I-222 | D | D | | | | | |
| I-223 | D | D | | | | | |
| I-224 | D | D | | | | | |
| I-225 | D | D | | | | | |
| I-226 | B | D | D | D | | | |
| I-227 | A | | | | ** | | *** |
| I-228 | A | | | | | | *** |
| I-229 | A | | | | | | *** |
| I-230 | A | | | | * | | ** |
| I-231 | A | D | | | | | *** |
| I-232 | B | D | | | | | *** |
| I-233 | A | D | | | * | | ** |
| I-234 | A | D | D | D | ** | | ** |
| I-235 | A | D | D | D | | | * |
| I-236 | A | | | | ** | | ** |
| I-237 | A | | | | | | ** |
| I-238 | A | D | | | | | * |
| I-239 | A | D | | | | | ** |
| I-240 | B | D | D | | | | |

In Vivo Studies

The following are various models of AML that will be used to assess the PK/PD relationship and efficacy of compounds in vivo.

### A. Subcutaneous Xenograft* Model:

Several human-derived AML cell lines will be tested in immunocompromised mice to elucidate the PK/PD relationship as well as the efficacy of compounds to inhibit tumor growth. Compounds will be administered to mice using an appropriate route of administration and dosing regimen at various concentrations and samples taken at various timepoints after dosing to evaluate plasma and tumoral exposure (pharmacokinetic measurements) as well as the effect on the m⁶ A-mRNA pharmacodynamic biomarker extracted from tumors at varying timepoints. Body weight will be measured daily to assess tolerability.

### B. Disseminated Xenograft* Model:

Studies analogous to the ones described above will be conducted but rather as a disseminated model of disease achieved by tail-vein injection of various human AML cell lines. Cell lines may be luciferized for whole body imaging to assess disease burden at various doses and timepoints following drug administration. A Kaplan-Meier estimate will be used to assess survival over time. Other measurements of disease burden will be taken such as effects on composition of the bone marrow and spleen size.

*note: xenograft includes both cell-line derived (CDX) and patient-derived (PDX) models

Various genetically engineered mouse models (GEMMs) of AML in immunocompotent mice will also be used for the in vivo PK/PD/efficacy studies described above.

1. A compound of formula (I′): 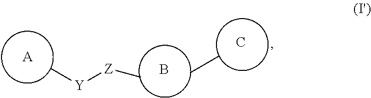
or a pharmaceutically acceptable salt thereof, wherein:
Y is selected from O, S, C(R^{1a})₂; NR^{1b}, S(═O), S(═O)₂, NR^{1b}C(═O)NR^{1b}, NR^{1b}C(═O)O with O linked to Z, and C(R^{1b})₂C(═O)NR^{1b} with NR^{1b} linked to Z;
R^{1a}, for each occurrence, is independently selected from H, C₁₋₆alkyl and halo;
Z is selected from O, S, NR^{1b}, C₁₋₆alkyl, C₂₋₆alkenyl and C₂₋₆ alkynyl, each of which is optionally substituted with 1 to 3 halo, provided when Z is O, S or NR^{1b}, then Y is C(R^{1a})₂;
R^{1b}, for each occurrence, is independently H or C₁₋₆alkyl;
Ring A is selected from benzene, naphthalene, benzene fused with 5 to 6-membered heterocycloalkyl, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
Ring C is selected from 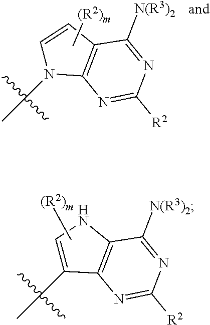
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —S(═O)R^{2a}, —S(═O)₂R^{2a}, —C(═O)R^{2a}1, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl optionally substituted with —OR^{2a} or —N(R^{2a})₂, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, —N(R^{2a})₂;
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b}, —N(R^{2b})₂, and —S(═O)₂R^{2b};
R^{2b}, for each occurrence, is independently selected from H and C₁₋₆alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, oxo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₁₋₆hydroxyalkyl, C₃₋₈cycloalkyl, phenyl, 5- to 6-membered heteroaryl, oxo, halo, —CN, —OR^{5a}, —N(R^{5a})₂, —C(O)N(R^{5a})₂, —N(R^{5a})C(═O)R^{5a}, —C(O)R^{5a}, and —C(O)OR^{5a};
R^{5a}, for each occurrence, is independently selected from H, C₁₋₆alkyl, C₃₋₈cycloalkyl, 4 to 7-membered heterocycloalkyl, phenyl and 5 to 6-membered heteroaryl, wherein the C₁₋₆alkyl, C₃₋₈cycloalkyl, 4 to 7-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, —S(═O)₂R^{5b}, C₁₋₆haloalkyl, C₃₋₆cycloalkyl, phenyl, and 4 to 7-membered heterocycloalkyl;
or two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₄hydroxyalkyl, —OR^{5b}, —N(R^{5b})₂, and —NR^{5b}C(═O)R^{5b};
R^{5b}, for each occurrence, is independently H or C₁₋₆alkyl optionally substituted with phenyl; and
m is 1 or 2.

2. The compound of claim 1, or a pharmaceutically acceptable salt thereof, wherein:
Y is selected from O, S, C(R^{1a})₂; and NR^{1b};
R^{1a}, for each occurrence, is independently selected from H, C₁₋₆alkyl and halo;
Z is selected from O, S, NR^{1b}, C₁₋₆alkyl, C₂₋₆alkenyl and C₂₋₆ alkynyl, each of which is optionally substituted with 1 to 3 halo, provided when Z is O, S or NR^{1b}, then Y is C(R^{1a})₂;
R^{1b} is H or C₁₋₆alkyl;
Ring A is selected from benzene, naphthalene, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
Ring C is selected from 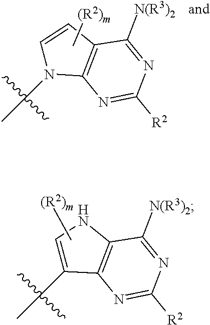
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂,
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b} and —N(R^{2b})₂;
R^{2b}, for each occurrence, is independently selected from H and C₁₋₃alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{5a}, —N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, C₁₋₆haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl;
R^{5b}, for each occurrence, is H or C₁₋₆alkyl; and
m is 1 or 2.

3. The compound of claim 1 or 2, wherein the compound is represented by formula (I): 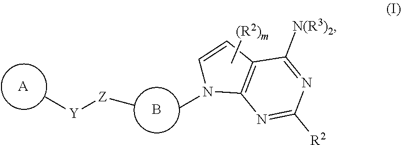
or a pharmaceutically acceptable salt thereof, wherein:
Y is selected from O, S, C(R^{1a})₂ and NR^{1b};
R^{1a}, for each occurrence, is independently selected from H, C₁₋₆alkyl and halo;
R^{1b} is H or C₁₋₆alkyl;
Z is selected from C₁₋₆alkyl, C₂₋₆alkenyl and C₂₋₆ alkynyl, each of which is optionally substituted with 1 to 3 halo;
Ring A is selected from benzene, naphthalene, 5 to 6-membered monocyclic heteroaromatic ring and 8- to 10-membered bicyclic heteroaromatic ring, each of which is optionally substituted with 1 to 4 independently selected R⁵;
Ring B is benzene, naphthalene, 5 to 6-membered heteroaromatic ring, each of which is optionally substituted with 1 to 4 independently selected R⁴;
R², for each occurrence, is independently selected from H, C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl, 5 to 6-membered heteroaryl, halo, —CN, —OR^{2a}, —N(R^{2a})₂, —C(═O)N(R^{2a})₂; and —N(R^{2a})—C(═O)—(R^{2a}), wherein the C₁₋₆alkyl, C₂₋₆alkenyl, C₂₋₆alkynyl, C₃₋₈cycloalkyl, C₅₋₈cycloalkenyl, 4 to 7-membered heterocycloalkyl, 4 to 7-membered heterocycloalkenyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{2a}, —C(═O)N(R^{2a})₂, and —N(R^{2a})₂;
R^{2a}, for each occurrence, is independently selected from H, C₁₋₆alkyl, C₂₋₆alkenyl, C₃₋₈cycloalkyl, and 4 to 6-membered heterocycloalkyl, wherein the C₁₋₆alkyl, C₂₋₆alkenyl, C₃₋₈cycloalkyl, and 4 to 6-membered heterocycloalkyl are each optionally substituted with one to three substituents independently selected from halo, C₁₋₃alkyl, —C(═O)OR^{2b}, —OR^{2b} and —N(R^{2b})₂;
R^{2b}, for each occurrence, is independently selected from H and C₁₋₃alkyl;
R³, for each occurrence, is H or C₁₋₆alkyl optionally substituted with 1 to 3 substituents independently selected from C₃₋₆cycloalkyl, phenyl and halo;
R⁴, for each occurrence, is independently selected from H, C₁₋₆alkyl, C₂₋₆alkenyl, C₂₋₆alkynyl, halo, —CN, —OR^{4a}, —N(R^{4a})₂, and —C(═O)N(R^{4a})₂, wherein the C₁₋₆alkyl, C₂₋₆alkenyl and C₂₋₆alkynyl are each optionally substituted with 1 to 3 substituents independently selected from halo, —OR^{4a}, —N(R^{4a})₂, and —C(═O)N(R^{4a})₂;
R^{4a}, for each occurrence, is independently selected from H and C₁₋₆alkyl;
R⁵, for each occurrence, is independently selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{5a}, —N(R^{5a})₂; —NR^{5a}C(═O)R^{5a}, —NR^{5a}C(═O)N(R^{5a})₂, —C(═O)N(R^{5a})₂, —C(═O)R^{5a}, and —C(═O)OR^{5a}, wherein the C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{5a}, —N(R^{5a})₂, —C(O)N(R^{5a})₂, —C(O)R^{5a}, and —C(O)OR^{5a};
R^{5a}, for each occurrence, is independently selected from H, C₁₋₆alkyl, C₃₋₈cycloalkyl, 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OR^{5b}, —N(R^{5b})₂, —CN, C₁₋₆alkyl, C₁₋₆haloalkyl, C₃₋₆cycloalkyl, phenyl, and 4 to 7-membered heterocycloalkyl; or two R^{5a} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl;
R^{5b}, for each occurrence, is H or C₁₋₆alkyl; and
m is 1 or 2.

4. The compound of claim 1, or a pharmaceutically acceptable salt thereof, wherein Y is S, S(═O), S(═O)₂, NHC(═O)NH, NHC(═O)O with O linked to Z, or CH₂C(═O)NH with NH linked to Z.

5. The compound of any one of claims 1-3, or a pharmaceutically acceptable salt thereof, wherein Y is O or C(R^{1a})₂, and R^{1a}, for each occurrence, is independently H or halo.

6. The compound of any one of claims 1-3, or a pharmaceutically acceptable salt thereof, wherein Y is O or CH₂.

7. The compound of any one of the claims 1-6, or a pharmaceutically acceptable salt thereof, wherein Z is selected from C₁₋₄alkyl and C₂₋₄ alkenyl, each of which is optionally substituted with 1 to 3 halo.

8. The compound of any one of claims 1-3 and 5, or a pharmaceutically acceptable salt thereof, wherein Z is selected from O and NR^{1b}; and Y is C(R^{1a})₂, wherein R^{1a}, for each occurrence, is independently H or halo and R^{1b} is H or C₁₋₆alkyl.

9. The compound of any one of claims 1-3 and 5-7, or a pharmaceutically acceptable salt thereof, wherein Z is CH₂; and Y is O or CH₂.

10. The compound of any one of the claims 1-9, or a pharmaceutically acceptable salt thereof, wherein ring C is

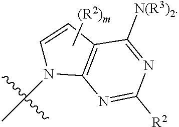

11. The compound of any one of the claims 1-9, or a pharmaceutically acceptable salt thereof, wherein ring C is

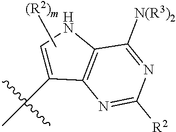

12. The compound of any one of claims 1-3, wherein the compound is represented by the following formula: 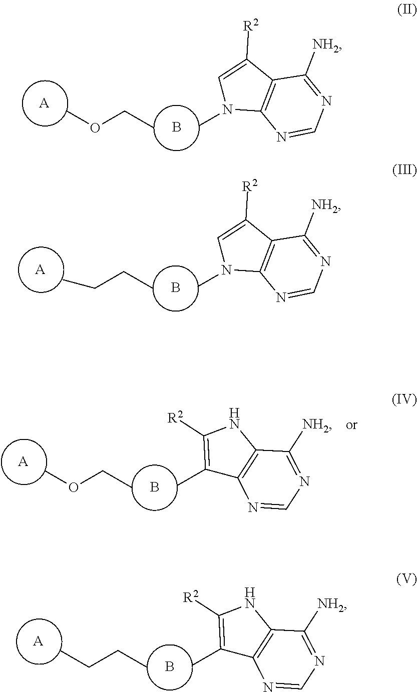
or a pharmaceutically acceptable salt thereof.

13. The compound of any one of claims 1-3, wherein the compound is represented by the following formula: 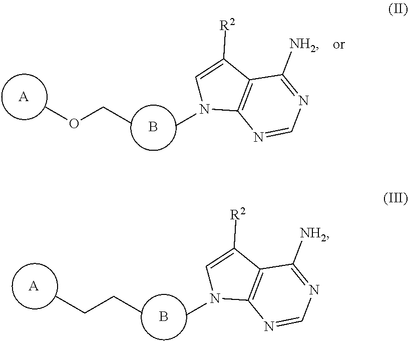
or a pharmaceutically acceptable salt thereof.

14. The compound of any one of claims 1-13, or a pharmaceutically acceptable salt thereof, wherein ring B is selected from benzene, pyridine, pyridazine, pyrimidine, pyrazine, triazine, pyrazole, imidazole, thiazole, oxazole, isoxazole, triazole and tetrazole, each of which is optionally substituted with 1 to 2 independently selected R⁴.

15. The compound of claim 14, or a pharmaceutically acceptable salt thereof, wherein ring B is selected from benzene, pyridine, pyrazine, pyrimidine, pyrazole, thiazole and thiadiazole, each of which is optionally substituted with 1 to 2 independently selected R⁴.

16. The compound of claim 14, or a pharmaceutically acceptable salt thereof, wherein ring B is selected from: 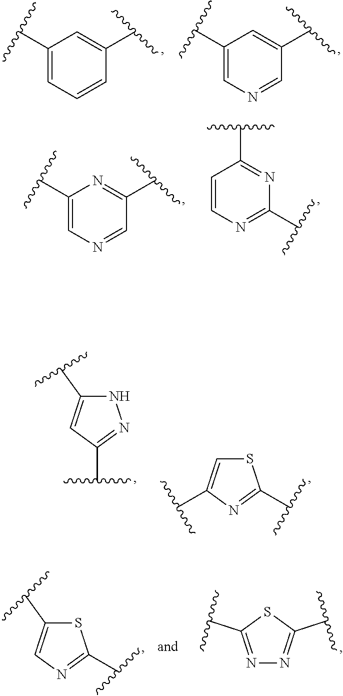
each of which is optionally substituted with 1 to 2 independently selected R⁴.

17. The compound of claim 16, or a pharmaceutically acceptable salt thereof, wherein ring B is 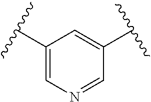 [SMILES: *c1cncc(*)c1]
optionally substituted with 1 to 2 independently selected R⁴.

18. The compound of any one of claims 1-17, or a pharmaceutically acceptable salt thereof, wherein R⁴ is H, C₁₋₆alkyl, halo, —N(R^{4a})₂; or —C(═O)N(R^{4a})₂, wherein the C₁₋₆alkyl is optionally substituted with 1 to 3 substituents independently selected from halo and —R^{4a}; and R^{4a}, for each occurrence, is independently selected from H and C₁₋₃alkyl.

19. The compound of claim 18, or a pharmaceutically acceptable salt thereof, wherein R⁴ is H.

20. The compound of any one of claims 1-17, or a pharmaceutically acceptable salt thereof, wherein R⁴ is —CH₂OH, —NHCH₃, —C(═O)NH₂, —NH₂, —Br, —F, —OH, and —OCH₃.

21. The compound of any one of claims 1-20, or a pharmaceutically acceptable salt thereof, wherein ring A is a 9- to 10-membered bicyclic heteroaromatic ring optionally substituted with 1 to 4 independently selected R⁵ groups.

22. The compound of claim 21, or a pharmaceutically acceptable salt thereof, wherein ring A is selected from quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵.

23. The compound of any one of claims 1-20, or a pharmaceutically acceptable salt thereof, wherein ring A is selected from benzene, naphthalene, pyridine, 3,4-dihydro-2H-benzo[b][1,4]oxazine, quinoline, quinazoline, phthalazine, quinoxaline, cinnoline, naphthyridine, pyridoprimidine, pyridopyrazine, pteridine, indole, isoindole, indolizine, indazole, benzoimidazole, benzotriazole, benzooxazole, benzoisoxazole, benzothiazole, benzofuran, isobenzofuran, benzothiophene, benzothiadiazole, azaindole, purine, imidazopyridine, pyrrolopyrimidine, imidazopyridazine, imidazopyrazine, pyrazolopyrimidine, pyrazolopyridine, pyrazolotriazine, oxazolopyridine, isoxazolopyridine, thiazolopyridine, and isothiazolopyridine, each of which is optionally substituted with 1 to 3 independently selected R⁵.

24. The compound of any one of claims 1-23, or a pharmaceutically acceptable salt thereof, wherein ring A is selected from quinoline, quinozaline, quinoxaline, benzoimidiazole, benzothiazole, napththyridine, indole, pyrrolopyrimidine and indazole, each of which is optionally substituted with 1 to 3 independently selected R⁵.

25. The compound of any one of claims 1-24, or a pharmaceutically acceptable salt thereof, wherein ring A is quinoline optionally substituted with 1 to 3 independently selected R⁵.

26. The compound of any one of claims 1 and 4-25, or a pharmaceutically acceptable salt thereof, wherein ring A is represented by the following formula: 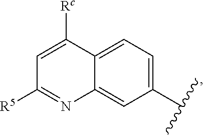
wherein R^{c} is selected from H, halo, C₁₋₆alkyl, —OR^{c1}, —N(R^{c1})₂; —NR^{c1}C(═O)R^{c1}, and 4 to 7-membered heterocycloalkyl, and R^{c1}, for each occurrence, is independently H, C₁₋₆alkyl, or C₃₋₆cycloalkyl, wherein the C₁₋₆alkyl, C₃₋₆cycloalkyl, and 4 to 7-membered heterocycloalkyl are each optionally substituted with 1 to 3 substituents independently selected from halo, C₁₋₄alkyl, C₃₋₆cycloalkyl, 4 to 7-membered heterocycloalkyl, —OR^{c2} and —N(R^{c2})₂; and R^{c2}, for each occurrence, is independently H or C₁₋₆alkyl optionally substituted with phenyl.

27. The compound of any one of claims 1-26, or a pharmaceutically acceptable salt thereof, wherein ring A is represented by the following formula: 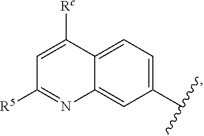
wherein R^{c} is selected from H, halo, C₁₋₄alkyl, —OR^{c1} and —N(R^{c1})₂, and R^{c1}, for each occurrence, is independently H or C₁₋₄alkyl optionally substituted with halo, —OR^{c2} and —N(R^{c2})₂; and R^{c2}, for each occurrence, is independently H or C₁₋₆alkyl.

28. The compound of claim 27, or a pharmaceutically acceptable salt thereof, wherein R^{c} is selected from H, —NHCH₂CH₂N(CH₃)₂, and —N(CH₃)CH₂CH₂OCH₃.

29. The compound of any one of claims 1 and 4-28, or a pharmaceutically acceptable salt thereof, wherein:
R⁵, for each occurrence, is independently selected from H, C₁₋₆alkyl, C₃₋₆cycloalkyl, 4 to 7-membered heterocycloalkyl, 5 to 6-membered heteroaryl, halo, oxo, —OR^{5a}, —N(R^{5a})₂; —NR^{5a}C(═O)R^{5a}, and —NR^{5a}C(═O)N(R^{5a})₂, wherein the C₁₋₆alkyl, C₃₋₆cycloalkyl, 4 to 7-membered heterocycloalkyl, and 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl, —S(═O)₂R^{5b}, and C₃₋₈cycloalkyl, or
two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₃hydroxyalkyl, —OH, —NH₂, and —NHC(═O)CH₃; and
R^{5b} is H or C₁₋₆alkyl optionally substituted with phenyl.

30. The compound of any one of claims 1-29, or a pharmaceutically acceptable salt thereof, wherein:
R⁵, for each occurrence, is independently selected from H, C₁₋₆alkyl, 4 to 6-membered heterocycloalkyl, 5 to 6-membered heteroaryl, halo, —OR^{5a}, —N(R^{5a})₂; —NR^{5a}C(═O)R^{5a}, and —NR^{5a}C(═O)N(R^{5a})₂, wherein the C₁₋₆alkyl, 4 to 6-membered heterocycloalkyl, 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl and C₃₋₈cycloalkyl, or two R^{5a} together with the N atom from which they are attached form a 4 to 6-membered heterocycloalkyl optionally containing an additional heteroatom selected from O, N and S; and
R^{5b} is H or C₁₋₃alkyl.

31. The compound of claim 30, or a pharmaceutically acceptable salt thereof, wherein:
R⁵, for each occurrence, is independently selected from H, C₁₋₆alkyl, —N(R^{5a})₂, and 4 to 6-membered heterocycloalkyl containing 1 or 2 heteroatoms independently selected from O, N and S;
R^{5a}, for each occurrence, is independently H, C₁₋₆alkyl, and C₃₋₆cycloalkyl, wherein the C₁₋₆alkyl and C₃₋₆cycloalkyl are each optionally substituted with one to three substituents independently selected from halo, C₁₋₆alkyl, C₃₋₆cycloalkyl, —OR^{5b} and —N(R^{5b})₂, or two R^{5a} together with the N atom from which they are attached form a 4 to 6-membered heterocycloalkyl optionally containing an additional heteroatom selected from O, N and S; and
R^{5b} is each independently H or C₁₋₃alkyl.

32. The compound of any one of claims 1 and 4-29, or a pharmaceutically acceptable salt thereof, wherein R⁵, for each occurrence, is independently selected from H, —F, —Br, NH₂, —NHCH₃, —N(CH₃)₂, —NHCH₂CH₃, —NHCH(CH₃)₂, —NHCH₂CH₂N(CH₃)₂, —NH(CH₂)₃N(CH₃)₂, —NHCH₂-cyclopropyl, —N(CH₃)CH₂cyclopropyl, —N(CH₃)cyclopropyl, —NHC(═O)CH₃, —N(CH₃)CH₂CH₂SO₂CH₃, —NHCH₂CH₂OCH₃, —N(CH₃)CH₂CH₂OCH₃, —N(CH₃)CH₂CH₂OH, —N(CH₃)CH₂CH₂N(CH₃)₂, —NHCH₂CH₂N(CH₃)(CH₂)₃phenyl, —NHCH₂CH₂N(CH₃)(CH₂)₅phenyl, —CH₃, —CH₂CH₃, —CH₂CH₂CH₃, —CH(CH₃)₂, —C(CH₃)₃, —OCH₃, cyclobutyl, —(CH₂)₃morpholine, azetidine, pyrrolidine, piperazine, piperidine, morpholine, tetrahydro-2H-pyran, 1,3-oxazinane, 5-azaspiro[2.3]hexane, 2-azaspiro[3.3]heptane, and 2-oxa-6-azaspiro[3.3]heptane, wherein the azetidine is optionally substituted with 1 to 3 substituents selected from oxo, —OH, NH₂, —F, —NHC(═O)CH₃, —CH₃, and —CH₂OH; the 1,3-oxazinane and pyrrolidine are each optionally substituted with oxo; the piperazine is optionally substituted with methyl; and the piperidine is optionally substituted with 1 to 3 —F.

33. The compound of claim any one of claims 1-32 or a pharmaceutically acceptable salt thereof, wherein R⁵, for each occurrence, is independently selected from H, —NH₂, —NHCH₃, —NHCH₂CH₃, —NHCH(CH₃)₂, —NHCH₂CH₂N(CH₃)₂, —NHCH₂CH₂OCH₃, —NHCH₂-cyclopropyl, —CH₃, —CH₂CH₃, —CH(CH₃)₂, azetidine, and pyrrolidinyl.

34. The compound of claim 33, or a pharmaceutically acceptable salt thereof, wherein R⁵, for each occurrence, is independently selected from —NHCH₃, —NHCH₂CH₃, cyclobutyl, —CH₂CH₃, and azetidine.

35. The compound of any one of claims 1-34, or a pharmaceutically acceptable salt thereof, wherein:
R² is H, halo, —OR^{2a}, —N(R^{2a})₂, —N(R^{2a})—C(═O)—(R^{2a}), C₁₋₆alkyl, C₃₋₈cycloalkyl, C₅₋₈cycloalkenyl, 4 to 6-membered heterocycloalkyl, 4 to 6-membered heterocycloalkenyl, phenyl, or 5 to 6-membered heteroaryl, wherein the C₁₋₆alkyl, C₃₋₈cycloalkyl, C₅₋₈cycloalkenyl, 4 to 6-membered heterocycloalkyl, 4 to 6-membered heterocycloalkenyl and 5 to 6-membered heteroaryl are each optionally substituted with 1 to 3 substituents independently selected from halo, N(R^{2a})₂, C₁₋₄alkyl, C₁₋₄haloalkyl and C₃₋₆cycloalkyl;
R^{2a}, for each occurrence, is independently selected from H, C₁₋₆alkyl and C₂₋₆alkenyl, wherein the C₁₋₆alkyl and C₂₋₆alkenyl are each optionally substituted with one to three substituents independently selected from halo, —C(═O)OR^{2b}, —OR^{2b} and —N(R^{2b})₂; and
R^{2b} is H or C₁₋₃alkyl.

36. The compound of claim 35, or a pharmaceutically acceptable salt thereof, wherein R² is selected from halo, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl.

37. The compound of claim 35, or a pharmaceutically acceptable salt thereof, wherein:
R² is H, halo, —N(R^{2a})₂, —N(R^{2a})—C(═O)—(R^{2a}), C₁₋₆alkyl, C₃₋₆cycloalkyl, 4 to 6-membered heterocycloalkyl, or 5 to 6-membered heteroaryl, wherein the C₁₋₆alkyl, C₃₋₆cycloalkyl, 4 to 6-membered heterocycloalkyl, and 5 to 6-membered heteroaryl are each optionally substituted with 1 to 3 substituents independently selected from halo, C₁₋₃alkyl, C₃₋₆cycloalkyl and —N(R^{2a})₂,
R^{2a}, for each occurrence, is independently selected from H, C₁₋₆alkyl and C₂₋₆alkenyl, wherein the C₁₋₆alkyl and C₂₋₆alkenyl are each optionally substituted with one to three substituents independently selected from halo and —C(═O)OR^{2b}; and
R^{2b} is H or C₁₋₃alkyl.

38. The compound of any one of claims 1 and 4-34, or a pharmaceutically acceptable salt thereof, wherein R² is selected from H, —F, —Cl, —Br, —CN, —CH₃, —CH₂CH₃, —CH₂OH, —CH₂OCH₃, C(═O)CH₃, SO₂CH₃, —S(═O)CH₃, —N(CH₃)₂, —NHCH₃, —CH₂CH₂N(CH₃)₂, —CH₂-cyclopropyl, cyclopropyl, cyclopentyl, N-difluorocyclohexyl, N-methylpiperidine, —N(C(═O)CH═CH₂)(CH₂CH₂CO2H), 1-methylpyrazole, 1-(2-methoxyethyl)-1H-pyrazole, tetrahydrofuran, o-methoxyphenyl, 1-(2-methoxyethyl)-1H-pyrazole, 2-methylfuran, furan substituted with —CH₂NHCH₂CH₂SO₂CH₃, oxazole, —CH(CH₃)-cyclopropyl, —NHC(═O)CH₂Cl, and C(═O)CH₂CH₂CH₃.

39. The compound of claim 38, or a pharmaceutically acceptable salt thereof, wherein R² is selected from H, 1-methylpyrazole, and tetrahydrofuran.

40. The compound of claim 38, or a pharmaceutically acceptable salt thereof, wherein R² is selected from H, Cl, —Br, —N(CH₃)₂, —NHCH₃, —CH₃, —CH₂CH₃, —CH₂CH₂N(CH₃)₂, —CH₂-cyclopropyl, cyclopropyl, cyclopentyl, N-difluorocyclohexyl, N-methylpiperidine, 1-methylpyrazole and —N(C(═O)CH═CH₂)(CH₂CH₂CO2H).

41. The compound of claim 1, wherein the compound is represented by the following formula: 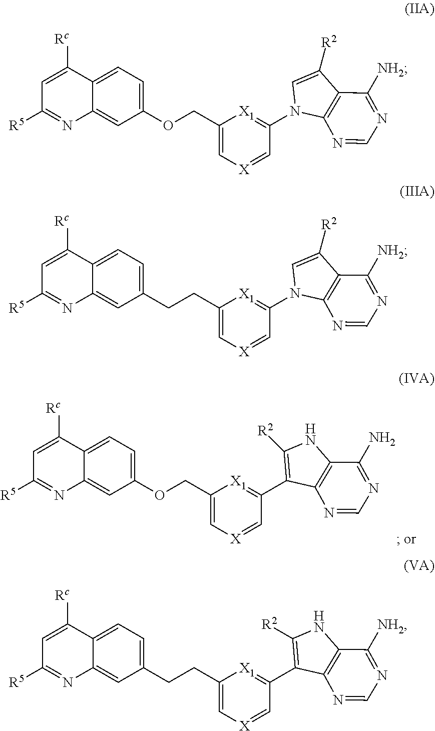
or a pharmaceutically acceptable salt thereof, wherein:
X is N or CH;
X₁ is N or CH;
R⁵, for each occurrence, is independently selected from H, C₁₋₆alkyl, C₃₋₆cycloalkyl, 4 to 7-membered heterocycloalkyl, 5 to 6-membered heteroaryl, halo, —OR^{5a}, —N(R^{5a})₂, —NR^{5a}C(═O)R^{5a}, and —NR^{5a}C(═O)N(R^{5a})₂, wherein the C₁₋₆alkyl, C₃₋₆cycloalkyl, 4 to 7-membered heterocycloalkyl, 5 to 6-membered heteroaryl are each optionally substituted with 1 to 3 substituents independently selected from C₁₋₆alkyl, C₁₋₆haloalkyl, C₃₋₆cycloalkyl, phenyl, halo and —CN; and
R^{5a}, for each occurrence, is independently selected from H, C₁₋₆alkyl, C₃₋₈cycloalkyl, phenyl, and 4 to 6-membered heterocycloalkyl, wherein the C₁₋₆alkyl, C₃₋₈cycloalkyl, phenyl and 4 to 6-membered heterocycloalkyl are each optionally substituted with 1 to 3 substituents independently selected from halo, —OR^{5b}, —N(R^{5b})₂, C₁₋₃alkyl, —SO₂C₁₋₃alkyl, and C₃-8cycloalkyl, or two R^{5a} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, oxo, C₁₋₄alkyl, C₁₋₄haloalkyl, C₁₋₃hydroxyalkyl, —OH, —NH₂, and —NHC(═O)CH₃; and
R^{5b} is H or C₁₋₆alkyl, wherein the C₁₋₆alkyl is optionally substituted with phenyl.
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂; —NR^{c1}C(═O)R^{1c}, —NR^{c1}C(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, 4 to 7-membered heterocycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1};
R^{c1}, for each occurrence, is independently selected from H, C₁₋₆alkyl, C₃₋₈cycloalkyl, and 4 to 7-membered heterocycloalkyl, phenyl and 5 to 6-membered heteroaryl, wherein the C₁₋₆alkyl, C₃₋₈cycloalkyl, 4 to 7-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₃₋₆cycloalkyl, phenyl, 4 to 7-membered heterocycloalkyl, —OR^{c2}, and —N(R^{c2})₂, or two R^{c1} together with the N atom from which they are attached form a 4 to 7-membered heterocycloalkyl optionally containing an additional heteroatom selected from O, N and S, wherein the 4 to 7-membered heterocycloalkyl is optionally substituted with 1 to 3 substituents independently selected from halo, C₁₋₄alkyl and C₁₋₄haloalkyl; and
R^{c2}, for each occurrence, is independently H or C₁₋₆alkyl optionally substituted with phenyl.

42. The compound of claim 1 or 2, wherein the compound is represented by the following formula: 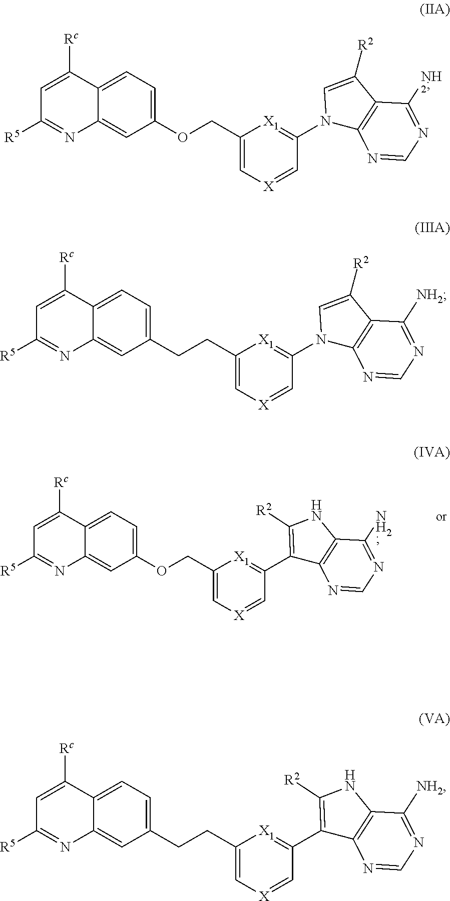
or a pharmaceutically acceptable salt thereof, wherein:
X is N or CH;
X₁ is N or CH;
R² is selected from H, halo, C₁₋₄alkyl, C₃₋₆cycloalkyl and 5 to 6-membered heteroaryl, wherein the C₃₋₆cycloalkyl and 5 to 6-membered heteroaryl are each optionally substituted with halo, C₁₋₄alkyl and C₁₋₄haloalkyl; and
R⁵ is —N(R^{5a})₂;
R^{5a}, for each occurrence, is independently selected from H and C₁₋₆alkyl, or two R^{5a} together with the N atom from which they are attached form a 4 to 6-membered heterocycloalkyl optionally containing an additional heteroatom selected from O and N;
R^{c} is selected from H, C₁₋₆alkyl, C₂₋₆alkenyl, C₂₋₆alkynyl, C₃₋₈cycloalkyl, 4 to 7-membered heterocycloalkyl, phenyl, 5 to 6-membered heteroaryl, halo, —CN, —OR^{c1}, —N(R^{c1})₂; —NR^{c1}(═O)R^{c1}, —NR^{c1}(═O)N(R^{c1})₂, —C(O)N(R^{c1})₂, —C(O)R^{c1}, and —C(O)OR^{c1}, wherein C₁₋₆alkyl, C₂₋₆alkenyl, C₂₋₆alkynyl, C₃₋₈cycloalkyl, 4 to 7-membered heterocycloalkyl, phenyl and 5 to 6-membered heteroaryl are each optionally substituted with 1 to 3 substituents independently selected from C₁₋₆alkyl, C₁₋₆haloalkyl, C₃₋₈cycloalkyl, phenyl, 5- to 6-membered heteroaryl, halo, —CN, —OR^{c1}, —N(R^{c1})₂, —C(O)N(R^{c1})₂, —C(O)R^{c1}, and —C(O)OR^{c1}; and
R^{c1}, for each occurrence, is independently selected from H, C₁₋₆alkyl, C₃₋₈cycloalkyl, and 4 to 6-membered heterocycloalkyl, phenyl and 5 to 6-membered heteroaryl, wherein the C₁₋₆alkyl, C₃₋₈cycloalkyl, 4 to 6-membered heterocycloalkyl, phenyl and 5 to 6-membered heteroaryl are each optionally substituted with 1 to 3 substituents independently selected from halo, —OH, —CN, C₁₋₆alkyl, C₁₋₆haloalkyl, C₃₋₆cycloalkyl, phenyl and 4 to 7-membered heterocycloalkyl, or two R^{c1} together with the N atom from which they are attached form a 4 to 6-membered heterocycloalkyl optionally containing an additional heteroatom selected from O, N and S, wherein the 4 to 6-membered heterocycloalkyl is optionally substituted with 1 to 3 substituents independently selected from halo, C₁₋₄alkyl and C₁₋₄haloalkyl.

43. The compound of claim 41 or 42, or a pharmaceutically acceptable salt thereof, wherein X is CH and X₁ is CH.

44. The compound of claim 41 or 42, or a pharmaceutically acceptable salt thereof, wherein X is N and X₁ is CH.

45. The compound of claim 41 or 42, or a pharmaceutically acceptable salt thereof, wherein X is N and X₁ is N.

46. The compound of any one of claims 41-45, or a pharmaceutically acceptable salt thereof, wherein R^{c} is selected from H, halo, C₁₋₄alkyl, —OR^{c1} and —N(R^{c1})₂, and R^{c1}, for each occurrence, is independently H or C₁₋₄alkyl optionally substituted with halo, —OR^{c2} or —N(R^{c2}); and R^{c2}, for each occurrence, is independently H or C₁₋₄alkyl.

47. The compound of any one of claims 41-46, or a pharmaceutically acceptable salt thereof, wherein R^{c} is H.

48. The compound of claim 1 or 2, wherein the compound is represented by the following formula: 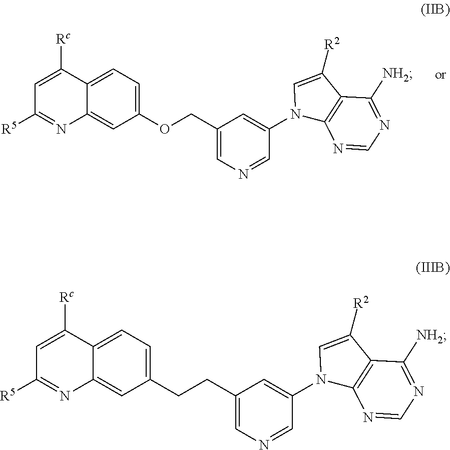
or a pharmaceutically acceptable salt thereof, wherein:
R² is H, 5-membered heteroaryl or 5-membered heterocycloalkyl;
R^{c} is H or —N(R^{c1})₂;
R^{c1} is C₁₋₃alkyl optionally substituted with —OR^{c2} or —N(R^{c2})₂;
R^{c2} is C₁₋₃alkyl;
R⁵ is C₁₋₃alkyl, C₃₋₆cycloalkyl or —N(R^{5a})₂; and
R^{5a}, for each occurrence, is independently H or C₁₋₃alkyl; or two R^{5a} together with the N atom from which they are attached form a 4 to 6-membered heterocycloalkyl.

49. The compound of claim 48, or a pharmaceutically acceptable salt thereof, wherein R² is H, 1-methylpyrazole, or tetrahydrofuran.

50. The compound of claim 48 or 49, or a pharmaceutically acceptable salt thereof, wherein R⁵ is —NHCH₃, —NHCH₂CH₃, cyclobutyl, —CH₂CH₃, or azetidine,

51. The compound of any one of claims 48-50, or a pharmaceutically acceptable salt thereof, wherein R^{c} is H, —NHCH₂CH₂N(CH₃)₂, or —N(CH₃)CH₂CH₂OCH₃.

52. A pharmaceutical composition comprising a compound of any one of claims 1-51, or a pharmaceutically acceptable salt thereof, and a pharmaceutically acceptable excipient.

53. A method of treating a disease or disorder responsive to inhibition of METTL3 activity in a subject comprising administering to the subject an effective amount of the compound according any one of claims 1-51, or a pharmaceutically acceptable salt thereof.

54. The method of claim 53, wherein the disease or disorder is an infection.

55. The method of claim 54, wherein the infection is a viral infection.

56. The method of claim 53, wherein the disease or disorder is cancer.

57. The method of claim 56, wherein the cancer is selected from glioblastoma, leukemia, stomach cancer, prostate cancer, colorectal cancer, endometrial cancer, breast cancer, pancreatic cancer, kidney cancer, lung cancer, bladder cancer, ovarian cancer, liver cancer, bone cancer, acute lymphocytic leukemia, esophageal/upper aerodigestive cancer, non-Hodgkin's lymphoma (NHL), multiple myeloma, mesothelioma and sarcoma.

58. The method of claim 57, wherein the cancer is acute myeloid leukemia.
